(19) United States
(12) Patent Application Publication
(10) Pub. No.: US 2025/0250262 A1
(43) Pub. Date: Aug. 7, 2025

(54) ORGANIC COMPOUND, OPTO-ELECTRONIC DEVICE INCLUDING THE SAME, AND ELECTRONIC DEVICE AND ELECTRONIC APPARATUS INCLUDING THE OPTO-ELECTRONIC DEVICE

(71) Applicant: Samsung Display Co., Ltd., Yongin-si (KR)

(72) Inventors: Hyejin JUNG, Yongin-si (KR); Hwasook RYU, Yongin-si (KR); Seokgyu YOON, Yongin-si (KR)

(73) Assignee: Samsung Display Co., Ltd., Yongin-si (KR)

(21) Appl. No.: 18/966,374

(22) Filed: Dec. 3, 2024

(30) Foreign Application Priority Data
Feb. 1, 2024 (KR) 10-2024-0016198

(51) Int. Cl. C07D 409/14; C07B 59/00; C07D 417/14; C07D 495/04; C07D 495/14; C07F 7/08; G02F 1/1335; H10K 85/40; H10K 85/60

(52) U.S. Cl. CPC C07D 409/14; C07B 59/004; C07D 417/14; C07D 495/04; C07D 495/14; C07F 7/0816; G02F 1/133514; H10K 85/40; H10K 85/623; H10K 85/653; H10K 85/654; H10K 85/6572; H10K 85/6574; C07B 2200/05

(57) ABSTRACT

Embodiments provide an organic compound, and an opto-electronic device including the organic compound. The opto-electronic device includes a first electrode, a second electrode facing the first electrode, and a photoactive layer between the first electrode and the second electrode, and the organic compound. The organic compound is represented by Formula 1, which is explained in the specification:

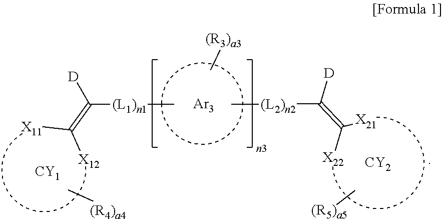

### CROSS-REFERENCE TO RELATED APPLICATION(S)

This application claims priority to and benefits of Korean Patent Application No. 10-2024-0016198 under 35 U.S.C. § 119, filed on Feb. 1, 2024, in the Korean Intellectual Property Office, the entire contents of which are incorporated herein by reference.

### BACKGROUND

### 1. Technical Field

Embodiments relate to an organic compound, an opto-electronic device including the same, and an electronic device and an electronic apparatus including the opto-electronic device.

### 2. Description of the Related Art

Opto-electronic devices are devices that convert optical energy or optical signals into electrical energy or electrical signals. Examples of an opto-electronic device are an optical or solar cell, which converts optical energy into electrical energy, an optical detector or sensor, which detects and converts optical energy into electrical signals, and the like.

An opto-electronic device may include organic compounds that can be excited by light. An opto-electric device may have a structure in which a first electrode is disposed on a substrate, and an interlayer and a second electrode are sequentially formed on the first electrode. The interlayer of the opto-electronic device is called a photoactive layer and may include an electron donating layer containing an electron donor and an electron accepting layer containing an electron acceptor. A p-type semiconductor material (for example, SubPC) may be used as an electron donor, and an n-type semiconductor material (for example, Fullerene) may be used as an electron acceptor.

When light is radiated to the opto-electronic device, due to absorption of light, electrons are excited and holes are generated, and the excited electrons and newly created holes may constitute a pair to form excitons. The excitons move to the interface of the interlayer and are separated into electrons and holes depending on the characteristics of the interface. Separated electrons and holes move to their respective electrodes, thereby generating a current.

Electronic apparatuses including opto-electronic devices and light-emitting devices are in development. Light emitted from a light-emitting device may be reflected from an object (for example, a finger of a user) that contacts an electronic apparatus, and becoming incident light onto an opto-electronic device. As an opto-electronic device detects incident light energy and converts the same into electrical signals, the contact of the object with the electronic apparatus may be recognized. Opto-electronic devices can be used as a fingerprint recognition sensor.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

Embodiments include an organic compound with excellent deposition stability and heat resistance, an opto-electronic device with excellent external quantum efficiency, and a high-quality electronic device and electronic apparatus using the same.

Additional aspects will be set forth in part in the description which follows and, in part, will be apparent from the description, or may be learned by practice of the embodiments of the disclosure.

According to embodiments, an organic compound may be represented by Formula 1:

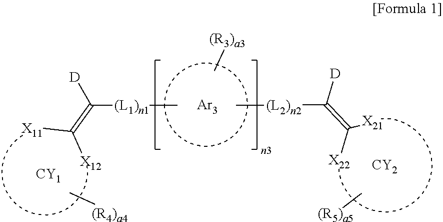

In Formula 1,
Ar₃ may be a C₁-C₆₀ heteroarylene group or a divalent non-aromatic condensed heteropolycyclic group,
n3 may be an integer from 1 to 3,
L₁ may be a C₃-C₆₀ carbocyclic group unsubstituted or substituted with at least one R₁ or a C₁-C₆₀ heterocyclic group unsubstituted or substituted with at least one R₁,
L₂ may be a C₃-C₆₀ carbocyclic group unsubstituted or substituted with at least one R₂ or a C₁-C₆₀ heterocyclic group unsubstituted or substituted with at least one R₂,
n1 and n2 may each independently be an integer from 1 to 3,
CY₁ and CY₂ may each independently be a C₃-C₆₀ carbocyclic group or a C₁-C₆₀ heterocyclic group,
X₁₁ and X₁₂ may each independently be C(R₄₁)(R₄₂), Si(R₄₁)(R₄₂), N(R₄₁), P(R₄₁), O, S, C(R₄₁), Si(R₄₁), N, P, C(═O), C(═S), or C═C(R₄₃)(R₄₄),
at least one of X₁₁ and X₁₂ may each independently be C(═O), C(═S), or C═C(R₄₃)(R₄₄),
X₂₁ and X₂₂ may each independently be C(R₅₁)(R₅₂), Si(R₅₁)(R₅₂), N(R₅₁), P(R₅₁), O, S, C(R₅₁), Si(R₅₁), N, P, C(═O), C(═S), or C═C(R₅₃)(R₅₄),
at least one of X₂₁ and X₂₂ may each independently be C(═O), C(═S), or C═C(R₅₃)(R₅₄),
a3, a4, and a5 may each independently be an integer from 0 to 10,
R₁ to R₅, R₄₁, R₄₂, R₅₁, and R₅₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₄₃, R₄₄, R₅₃, and R₅₄ may each independently be an electron-accepting group,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, the organic compound may be represented by Formula 1-1, which is explained below.

In an embodiment, the organic compound may be represented by Formula 1-2, which is explained below.

In an embodiment, R₄₃, R₄₄, R₅₃, and R₅₄ may each independently be —F, —Cl, —Br, —I, a cyano group, a nitro group, —C(═O)(Q₁), —S(═O)₂(Q₁), or —P(═O)(Q₁)(Q₂).

In an embodiment, in Formula 1, a moiety represented by

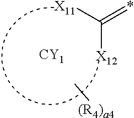

may be a moiety represented by one of Formulae 1A to 1K, which are explained below.

In an embodiment, in Formula 1, a moiety represented by

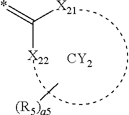

may be a moiety represented by one of Formulae 2A to 2K, which are explained below.

In an embodiment, Y₁ and Y₂ may each independently be N(R₆), P(R₆), O, or S.

In an embodiment, in Formula 1, a moiety represented by

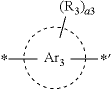

may be a moiety represented by one of Formulae 3A to 3D, which are explained below.

In an embodiment, R₁ to R₅, R₄₁, R₄₂, R₅₁, and R₅₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkenyl group unsubstituted or substituted with at least one R₁₀ₐ, a C₁-C₂₀ alkynyl group unsubstituted or substituted with at least one R₁₀ₐ, a C₃-C₃₀ carbocyclic group unsubstituted or substituted with at least one R₁₀ₐ, a C₁-C₃₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and Q₁ to Q₃ and R₁₀ₐ may each be the same as defined in Formula 1.

In an embodiment, in Formula 1, a moiety represented by

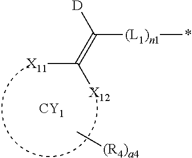

and a moiety represented by

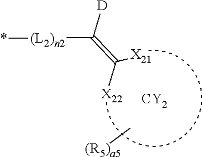

may be identical to each other.

In an embodiment, the organic compound may be one of Compounds P1 to P55, which are explained below.

According to embodiments, an opto-electronic device may include a first electrode, a second electrode facing the first electrode, a photoactive layer between the first electrode and the second electrode, and the organic compound, wherein the organic compound may be a first compound.

In an embodiment, the opto-electronic device may further include a second compound represented by one of Formulae 2-1 to 2-6, which are explained below.

In an embodiment, Z₅₁ and Z₅₂ may each independently be a C₁-C₆₀ alkyl group, a C₃-C₁₀ cycloalkyl group, a C₆-C₆₀ aryl group, or a C₁-C₆₀ heteroaryl group, each unsubstituted or substituted with deuterium, —F, —Cl, —Br, —I, a cyano group, a C₁-C₆₀ alkyl group, a C₁-C₆₀ alkoxy group, or any combination thereof.

In an embodiment, the second compound may be one of Compounds N1 to N43, which are explained below.

In an embodiment, the photoactive layer may include the first compound.

In an embodiment, the photoactive layer may include a first layer adjacent to the first electrode and a second layer adjacent to the second electrode.

In an embodiment, the first layer may include the first compound.

According to embodiments, an electronic device may include: the opto-electronic device; a light-emitting device; and a color filter, a color change layer, a touch screen layer, a polarizing layer, or any combination thereof, wherein the light-emitting device and the opto-electronic device may not overlap each other.

According to embodiments, an electronic apparatus may include the opto-electronic device, wherein the electronic apparatus may be a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall including multiple displays tiled together, a theater screen, a stadium screen, a phototherapy device, a signboard, an automotive sensor, a home sensor, or a solar cell.

It is to be understood that the embodiments above are described in a generic and explanatory sense only and not for the purpose of limitation, and the disclosure is not limited to the embodiments described above.

### BRIEF DESCRIPTION OF THE DRAWINGS

The accompanying drawings are included to provide a further understanding of the embodiments, and are incorporated in and constitute a part of this specification.

The drawings illustrate embodiments of the disclosure and principles thereof. The above and other aspects and features of the disclosure will be more apparent by describing in detail embodiments thereof with reference to the accompanying drawings, in which:

FIG. 1 is a schematic cross-sectional view of an opto-electronic device according to an embodiment;

FIG. 2 is a schematic cross-sectional view of a light-emitting device included in an electronic apparatus according to an embodiment;

FIG. 3 is a schematic cross-sectional view of an opto-electronic device according to another embodiment;

FIG. 4 is a schematic cross-sectional view of an opto-electronic device according to another embodiment;

FIG. 5 is a schematic cross-sectional view of an electronic apparatus according to an embodiment;

FIG. 6 is a schematic cross-sectional view of an electronic device according to another embodiment;

FIG. 7 is a schematic perspective view of an electronic apparatus including an opto-electronic device according to an embodiment;

FIG. 8 is a schematic perspective view of an exterior of a vehicle as an electronic apparatus including an opto-electronic device according to an embodiment; and

FIGS. 9A to 9C are each a schematic diagram of an interior of the vehicle of FIG. 8 according to embodiments.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like reference numbers and/or like reference characters refer to like elements throughout.

In the description, it will be understood that when an element (or region, layer, part, etc.) is referred to as being “on”, “connected to”, or “coupled to” another element, it can be directly on, connected to, or coupled to the other element, or intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element.

Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within +20%, 10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

According to an embodiment, an organic compound may be provided.

The organic compound may be represented by Formula 1:

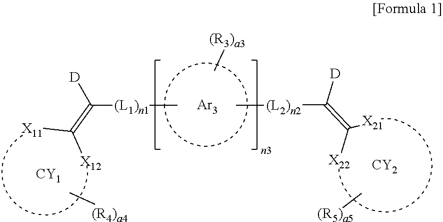

In Formula 1,
Ar₃ may be a C₁-C₆₀ heteroarylene group or a divalent non-aromatic condensed heteropolycyclic group,
n3 may be an integer from 1 to 3,
L₁ may be a C₃-C₆₀ carbocyclic group unsubstituted or substituted with at least one R₁ or a C₁-C₆₀ heterocyclic group unsubstituted or substituted with at least one R₁,
L₂ may be a C₃-C₆₀ carbocyclic group unsubstituted or substituted with at least one R₂ or a C₁-C₆₀ heterocyclic group unsubstituted or substituted with at least one R₂,
n1 and n2 may each independently be an integer from 1 to 3,
CY₁ and CY₂ may each independently be a C₃-C₆₀ carbocyclic group or a C₁-C₆₀ heterocyclic group,
X₁₁ and X₁₂ may each independently be C(R₄₁)(R₄₂), Si(R₄₁)(R₄₂), N(R₄₁), P(R₄₁), O, S, C(R₄₁), Si(R₄₁), N, P, C(═O), C(═S), or C═C(R₄₃)(R₄₄),
at least one of X₁₁ and X₁₂ may each independently be C(═O), C(═S), or C═C(R₄₃)(R₄₄),
X₂₁ and X₂₂ may each independently be C(R₅₁)(R₅₂), Si(R₅₁)(R₅₂), N(R₅₁), P(R₅₁), O, S, C(R₅₁), Si(R₅₁), N, P, C(═O), C(═S), or C═C(R₅₃)(R₅₄),
at least one of X₂₁ and X₂₂ may each independently be C(═O), C(═S), or C═C(R₅₃)(R₅₄),
a3, a4, and a5 may each independently be an integer from 0 to 10,
R₁ to R₅, R₄₁, R₄₂, R₅₁, and R₅₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
R₄₃, R₄₄, R₅₃, and R₅₄ may each independently be an electron-accepting group.

For example, the electron-accepting group may be:
—F, —Cl, —Br, —I, a cyano group, a nitro group, —C(═O)(Q₁), —S(═O)₂(Q₁), —P(═O)(Q₁)(Q₂), or —B(Q₁)(Q₂); or
a first group substituted with at least one of —F, —Cl, —Br, —I, —CF₃, —CF₂H, —CFH₂, a cyano group, a nitro group, —C(═O)(Q₃₁), —S(═O)₂(Q₃₁), —P(═O)(Q₃₁)(Q₃₂), and —B(Q₃₁)(Q₃₂), wherein
the first group may be:
a C₁-C₂₀ alkyl group, a C₂-C₂₀ alkenyl group, a C₂-C₂₀ alkynyl group, or a C₁-C₂₀ alkoxy group;
a C₁-C₂₀ alkyl group, a C₂-C₂₀ alkenyl group, a C₂-C₂₀ alkynyl group, or a C₁-C₂₀ alkoxy group, each substituted with at least one of deuterium, —CD₃, —CD₂H, —CDH₂, a hydroxyl group, an amidino group, a hydrazino group, a hydrazono group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, —Si(Q₃₁)(Q₃₂)(Q₃₃), —N(Q₃₁)(Q₃₂), —C(═O)(Q₃₁), —S(═O)₂(Q₃₁), and —P(═O)(Q₃₁)(Q₃₂);
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; or
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one of deuterium, —CD₃, —CD₂H, —CDH₂, a hydroxyl group, an amidino group, a hydrazino group, a hydrazono group, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₁)(Q₃₂)(Q₃₃), —N(Q₃₁)(Q₃₂), —C(═O)(Q₃₁), —S(═O)₂(Q₃₁), and —P(═O)(Q₃₁)(Q₃₂).

In Formula 1, R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —CI, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In Formula 1, Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to an embodiment, a highest occupied molecular orbital (HOMO) energy level of the organic compound may be in a range of about −5.8 eV to about −5.0 eV, and a lowest unoccupied molecular orbital (LUMO) energy level of the organic compound may be in a range of about −3.9 eV to about −2.7 eV.

According to an embodiment, the organic compound may absorb visible light. For example, the organic compound may absorb green light and/or red light. In an embodiment, a maximum absorption wavelength of the organic compound may be in a range of about 490 nm to about 750 nm. For example, the maximum absorption wavelength of the organic compound may be in a range of about 490 nm to about 570 nm. For example, the maximum absorption wavelength of the organic compound may be in a range of about 500 nm to about 560 nm. For example, the maximum absorption wavelength of the organic compound may be in a range of about 510 nm to about 550 nm. For example, the maximum absorption wavelength of the organic compound may be in a range of about 570 nm to about 750 nm. For example, the maximum absorption wavelength of the organic compound may be in a range of about 570 nm to about 700 nm. For example, the maximum absorption wavelength of the organic compound may be in a range of about 600 nm to about 680 nm.

According to an embodiment, the organic compound may not substantially emit light. For example, the organic compound may substantially be a non-luminescent compound.

According to an embodiment, the organic compound may be represented by Formula 1-1:

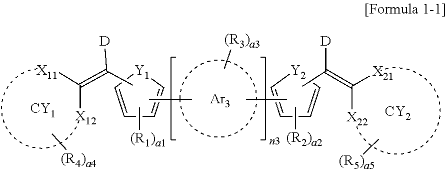

In Formula 1-1,
Y₁ and Y₂ may each independently be C(R₆)(R₇), Si(R₆)(R₇), N(R₆), P(R₆), O, or S,
a1 and a2 may each independently be an integer from 0 to 2, and

R₆ and R₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and

Ar₃, n3, CY₁, CY₂, X₁₁, X₁₂, X₂₁, X₂₂, a3, a4, a5, R₁ to R₅, Q₁ to Q₃, and R₁₀ₐ may each be the same as described herein.

According to an embodiment, the organic compound may be represented by Formula 1-2:

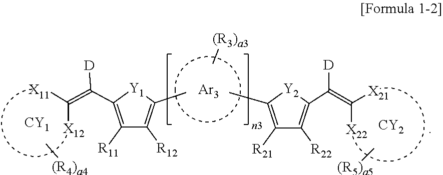

In Formula 1-2,
Y₁ and Y₂ may each independently be C(R₆)(R₇), Si(R₆)(R₇), N(R₆), P(R₆), O, or S,
R₆ and R₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₁ and R₁₂ may each independently be the same as described in connection with R₁,
R₂₁ and R₂₂ may each independently be the same as described in connection with R₂, and
Ar₃, n3, CY₁, CY₂, X₁₁, X₁₂, X₂₁, X₂₂, a3, a4, a5, R₃ to R₅, Q₁ to Q₃, and R₁₀ₐ may each be the same as defined herein.

According to an embodiment, in Formula 1, Formula 1-1, and Formula 1-2,
R₄₃, R₄₄, R₅₃, and R₅₄ may each independently be —F, —Cl, —Br, —I, a cyano group, a nitro group, —C(═O)(Q₁), —S(═O)₂(Q₁), or —P(═O)(Q₁)(Q₂).

According to an embodiment, in Formula 1, Formula 1-1, and Formula 1-2,
a moiety represented by

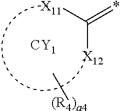

may be a moiety represented by one of Formula 1A to Formula 1K:

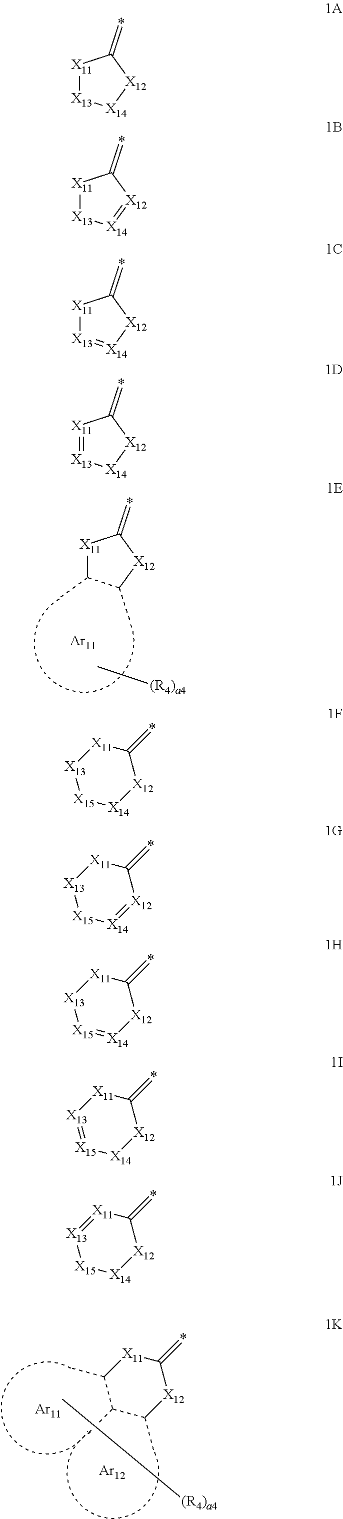

In Formulae 1A to 1K,
Ar₁₁ and Ar₁₂ may each independently be a C₃-C₃₀ carbocyclic group or a C₁-C₃₀ heterocyclic group,
X₁₃, X₁₄, and X₁₅ may each independently be C(R₄₅)(R₄₆), Si(R₄₅)(R₄₆), N(R₄₅), P(R₄₅), O, S, C(R₄₅), Si(R₄₅), N, P, C(═O), C(═S), or C═C(R₄₃)(R₄₄),
X₁₁, X₁₂, R₄, a4, R₄₃, and R₄₄ may each be the same as described herein,
R₄₅ and R₄₆ may each independently be the same as described in connection with R₄, and
* indicates a binding site to a neighboring atom.

According to an embodiment, in Formula 1A to Formula 1K, Ar₁₁ and Ar₁₂ may each independently be a benzene group, a pyridine group, a pyrimidine group, a triazine group, a naphthalene group, an anthracene group, a phenanthrene group, a phenalene group, a thiophene group, a furan group, a benzothiophene group, a benzofuran group, a dibenzothiophene group, or a dibenzofuran group.

According to an embodiment, in Formula 1, Formula 1-1, and Formula 1-2, when a moiety represented by

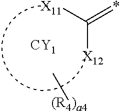

is represented by Formula 1E, the moiety represented by Formula 1E may be a moiety represented by one of Formula 1E-1 to Formula 1E-16:

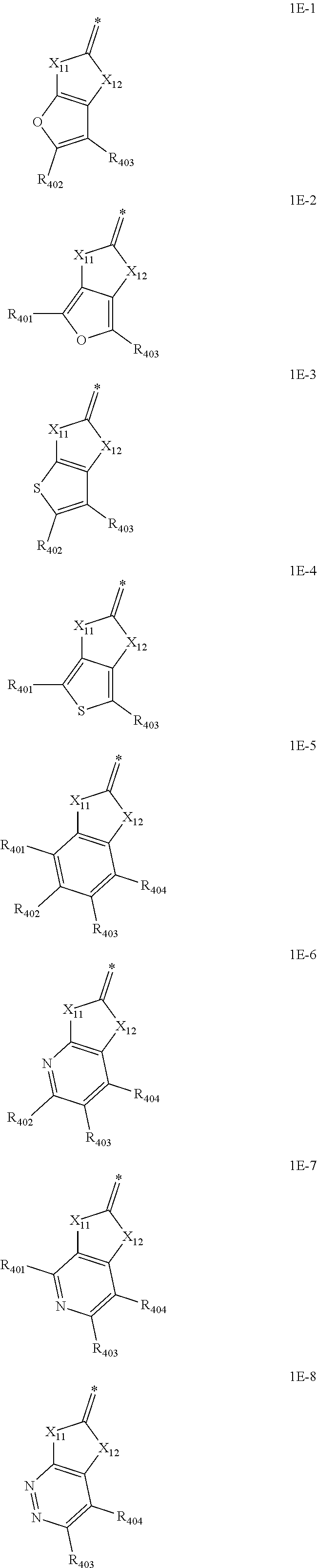

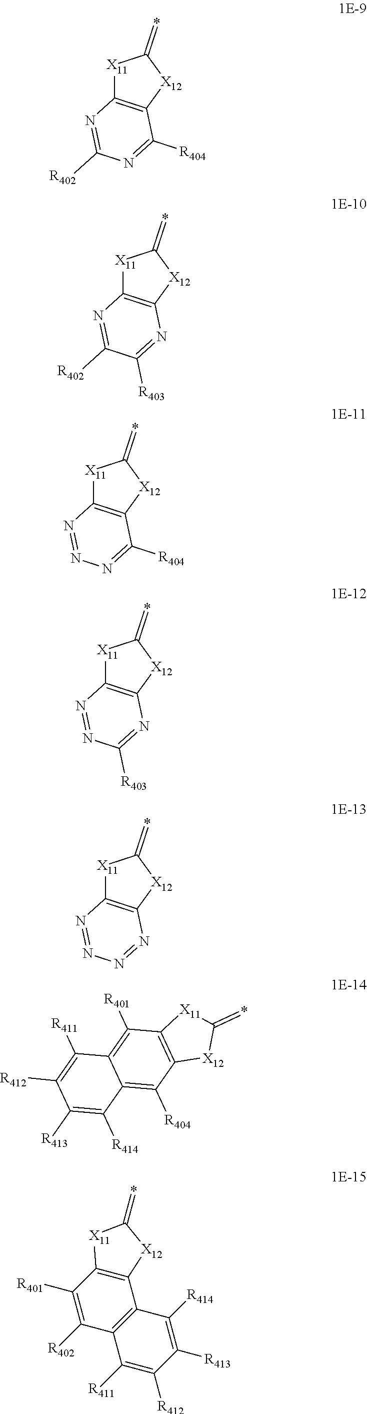

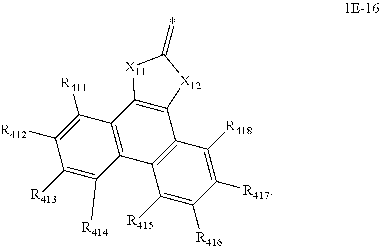

In Formulae 1E-1 to 1E-16,
X₁₁ and X₁₂ may be each be the same as described herein,
R₄₀₁ to R₄₀₄ and R₄₁₁ to R₄₁₈ may each independently be the same as described in connection with R₄, and
* indicates a binding site to a neighboring atom.

According to an embodiment, in Formula 1, Formula 1-1, and Formula 1-2, when a moiety represented by

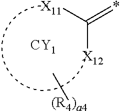

is a moiety represented by one of Formulae 1F to 1J, two or more of X₁₁, X₁₂, and X₁₅ may each independently be C(═O), C(═S), or C═C(R₄₃)(R₄₄).

According to an embodiment, in Formula 1, Formula 1-1, and Formula 1-2 a moiety represented by

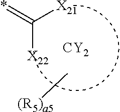

may be a moiety represented by one of Formulae 2A to 2K:

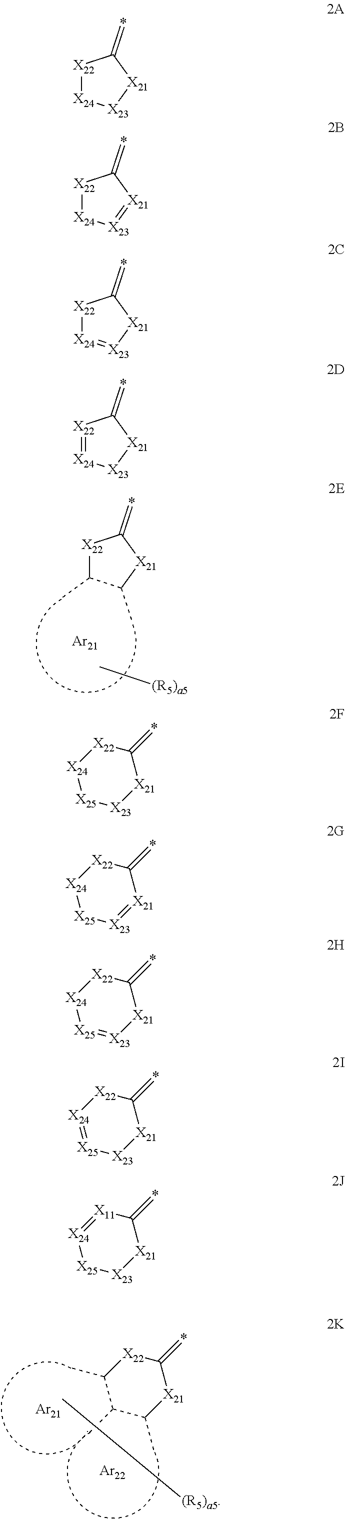

In Formulae 2A to 2K,
Ar₂₁ and Ar₂₂ may each independently be a C₃-C₃₀ carbocyclic group or a C₁-C₃₀ heterocyclic group,
X₂₃, X₂₄, and X₂₅ may each independently be C(R₅₅)(R₅₆), Si(R₅₅)(R₅₆), N(R₅₅), P(R₅₅), O, S, C(R₅₅), Si(R₅₅), N, P, C(═O), C(═S), or C═C(R₅₃)(R₅₄),
X₂₁, X₂₂, R₅, a5, R₅₃, and R₅₄ may each be the same as described herein,
R₅₅ and R₅₆ may each independently be the same as described in connection with R₅, and
* indicates a binding site to a neighboring atom.

According to an embodiment, in Formula 1A to Formula 1K, Ar₂₁ and Ar₂₂ may each independently be a benzene group, a pyridine group, a pyrimidine group, a triazine group, a naphthalene group, an anthracene group, a phenanthrene group, a phenalene group, a thiophene group, a furan group, a benzothiophene group, a benzofuran group, a dibenzothiophene group, or a dibenzofuran group.

According to an embodiment, in Formula 1, Formula 1-1, and Formula 1-2, when a moiety represented by

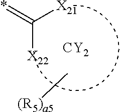

is a moiety represented by Formula 2E, the moiety represented by Formula 2E may be represented by one of Formula 2E-1 to Formula 2E-16:

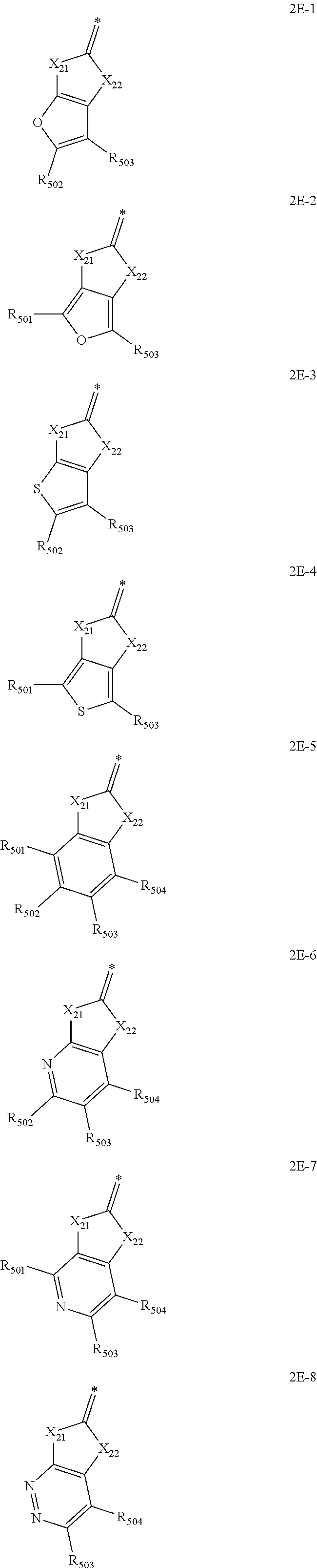

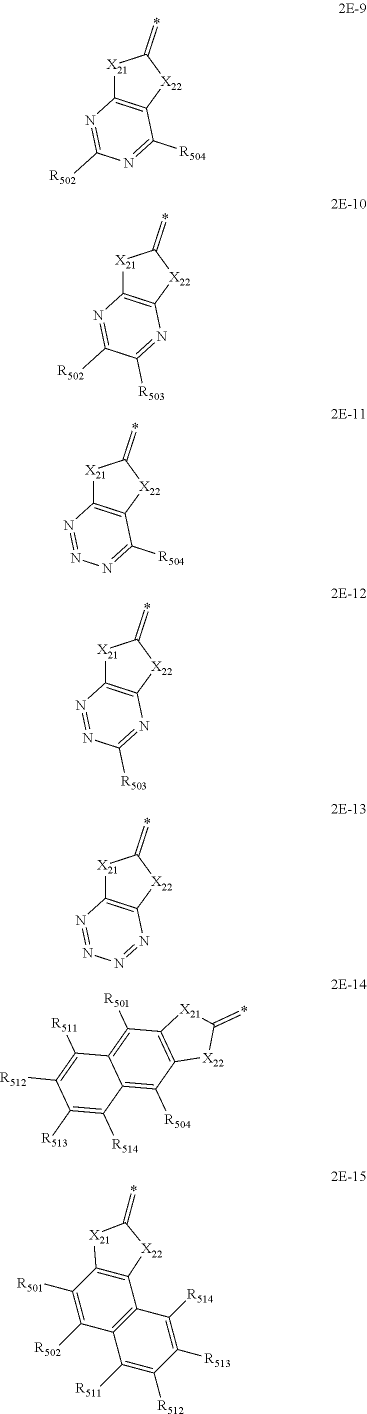

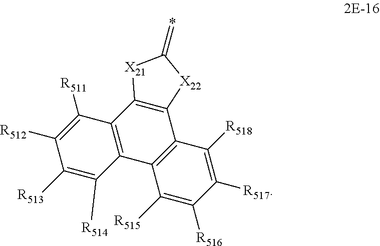

In Formulae 2E-1 to 2E-16,
X₂₁ and X₂₂ may each be the same as described herein,
R₅₀₁ to R₅₀₄, and R₅₁₁ to R₅₁₈ may each independently be the same as described in connection with R₅, and
* indicates a binding site to a neighboring atom.

According to an embodiment, in Formula 1, Formula 1-1, and Formula 1-2, when a moiety represented by

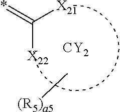

is a moiety represented by one of Formulae 2F to 2J, two or more of X₂₁, X₂₂, and X₂₅ may each independently be C(═O), C(═S), or C═C(R₅₃)(R₅₄).

According to an embodiment, in Formula 1-1 and Formula 1-2, Y₁ and Y₂ may each independently be N(R₆), P(R₆), O, or S.

According to an embodiment, in Formula 1, Formula 1-1, and Formula 1-2, a moiety represented by

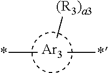

may be a moiety represented by one of Formula 3A to 3D:

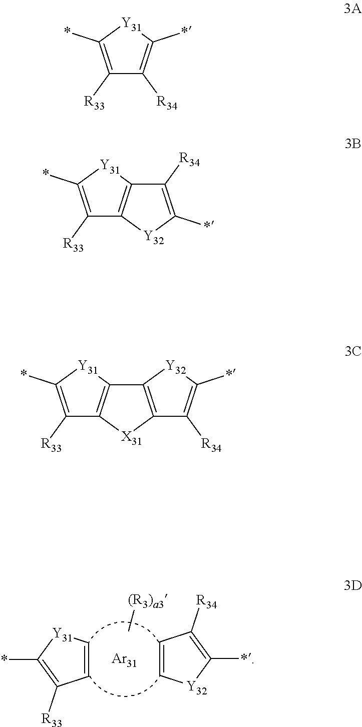

In Formulae 3A to 3D,
Ar₃₁ may be a C₃-C₃₀ carbocyclic group or C₁-C₃₀ heterocyclic group,
Y₃₁ and Y₃₂ may each independently be N(R₃₁), P(R₃₁), O, or S,
X₃₁ may be C(R₃₁)(R₃₂), Si(R₃₁)(R₃₂), N(R₃₁), P(R₃₁), O or S,
a3′ may be an integer from 0 to 8,
R₃ may be the same as described herein,
R₃₁ to R₃₄ may each independently be the same as described in connection with R₃, and
* and *′ are each a binding site to a neighboring atom.

According to an embodiment, in Formula 3A to 3D, Ar₃₁ may be a benzene group, a pyridine group, a pyrimidine group, a triazine group, a naphthalene group, an anthracene group, a phenanthrene group, a phenalene group, a thiophene group, a furan group, a benzothiophene group, a benzofuran group, a dibenzothiophene group, or a dibenzofuran group.

According to an embodiment, a moiety represented by Formula 3D may be represented by Formula 3D-1:

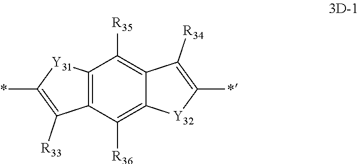

In Formula 3D-1,
Y₃₁, Y₃₂, R₃₃, and R₃₄ may each independently be the same as described herein,
R₃₅ and R₃₆ may each independently be the same as described in connection with R₃, and
* and *′ are each a binding site to a neighboring atom.

According to an embodiment, in Formulae 3A to 3D, Y₃₁ and Y₃₂ may each independently be O or S.

According to an embodiment, in Formula 1, Formula 1-1, and Formula 1-2, n3 may be 1 or 2.

In an embodiment, in Formula 1, R₁ to R₅, R₄₁, R₄₂, R₅₁, and R₅₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkenyl group unsubstituted or substituted with at least one R₁₀ₐ, a C₁-C₂₀ alkynyl group unsubstituted or substituted with at least one R₁₀ₐ, a C₃-C₃₀ carbocyclic group unsubstituted or substituted with at least one R₁₀ₐ, a C₁-C₃₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

According to an embodiment, in Formula 1, a moiety represented by

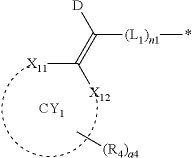

and a moiety represented by

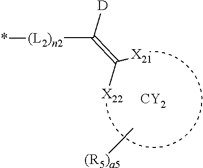

may be identical to each other.

According to an embodiment, the organic compound may be one of Compounds P1 to P55:

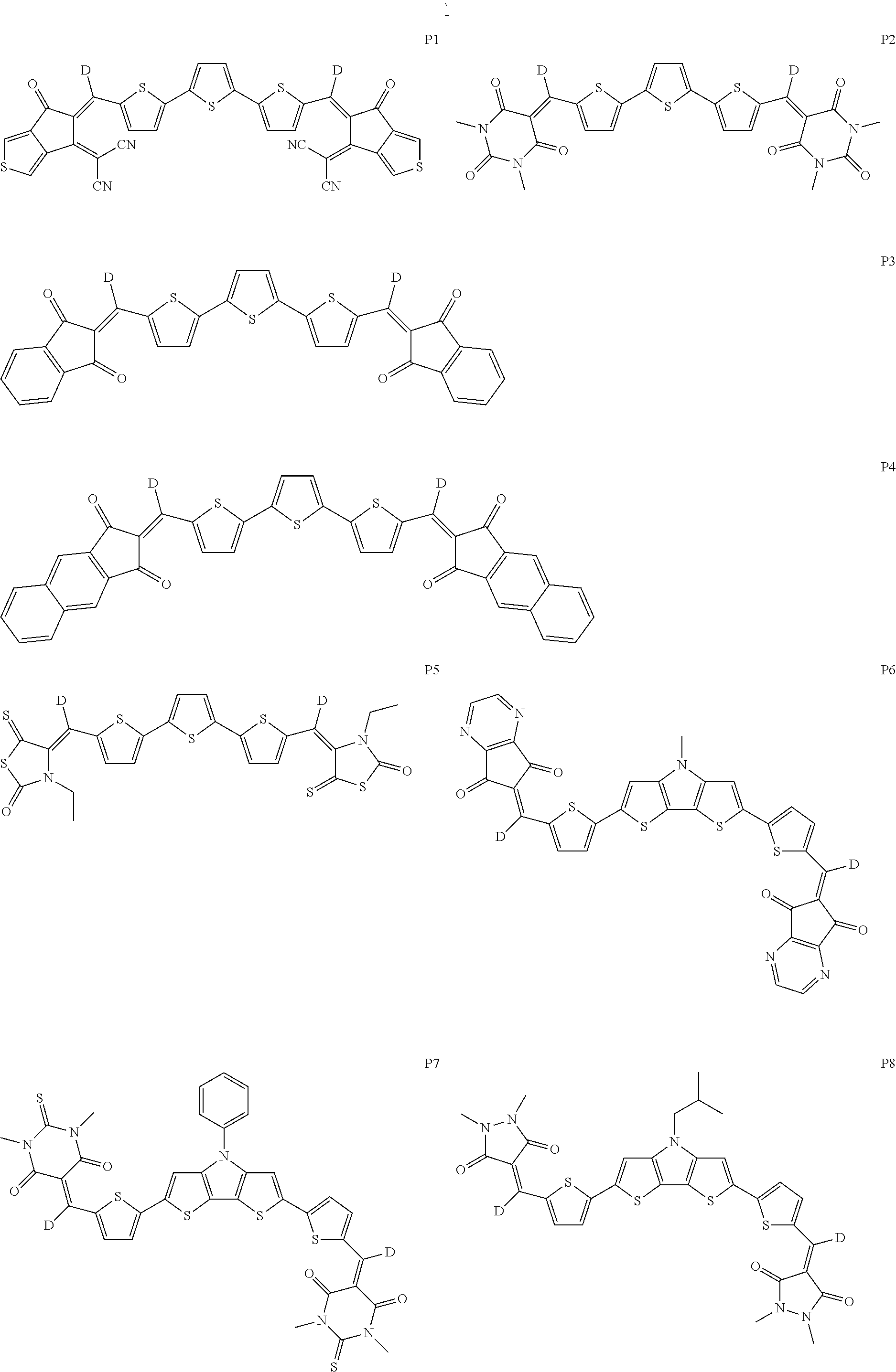

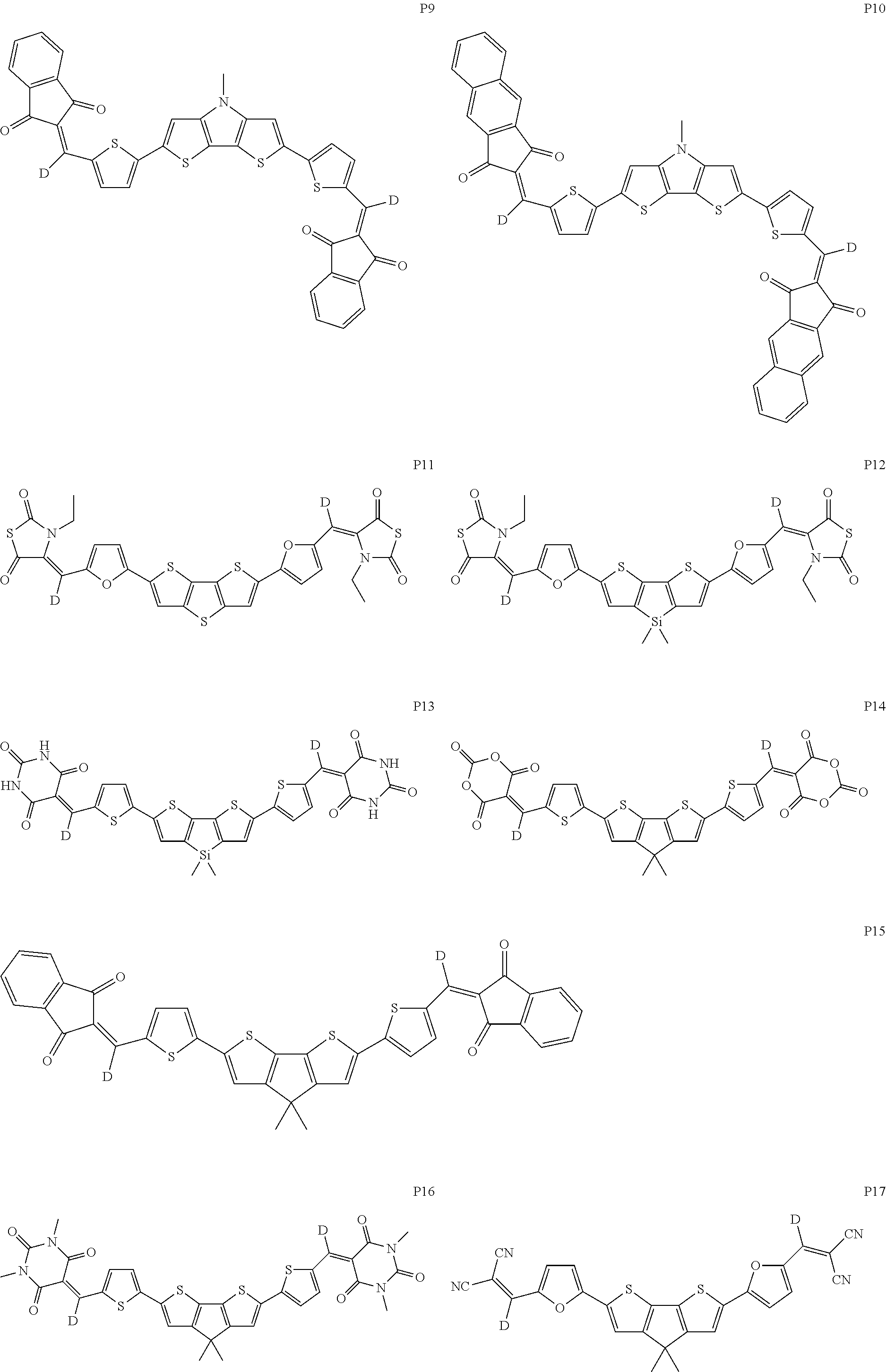

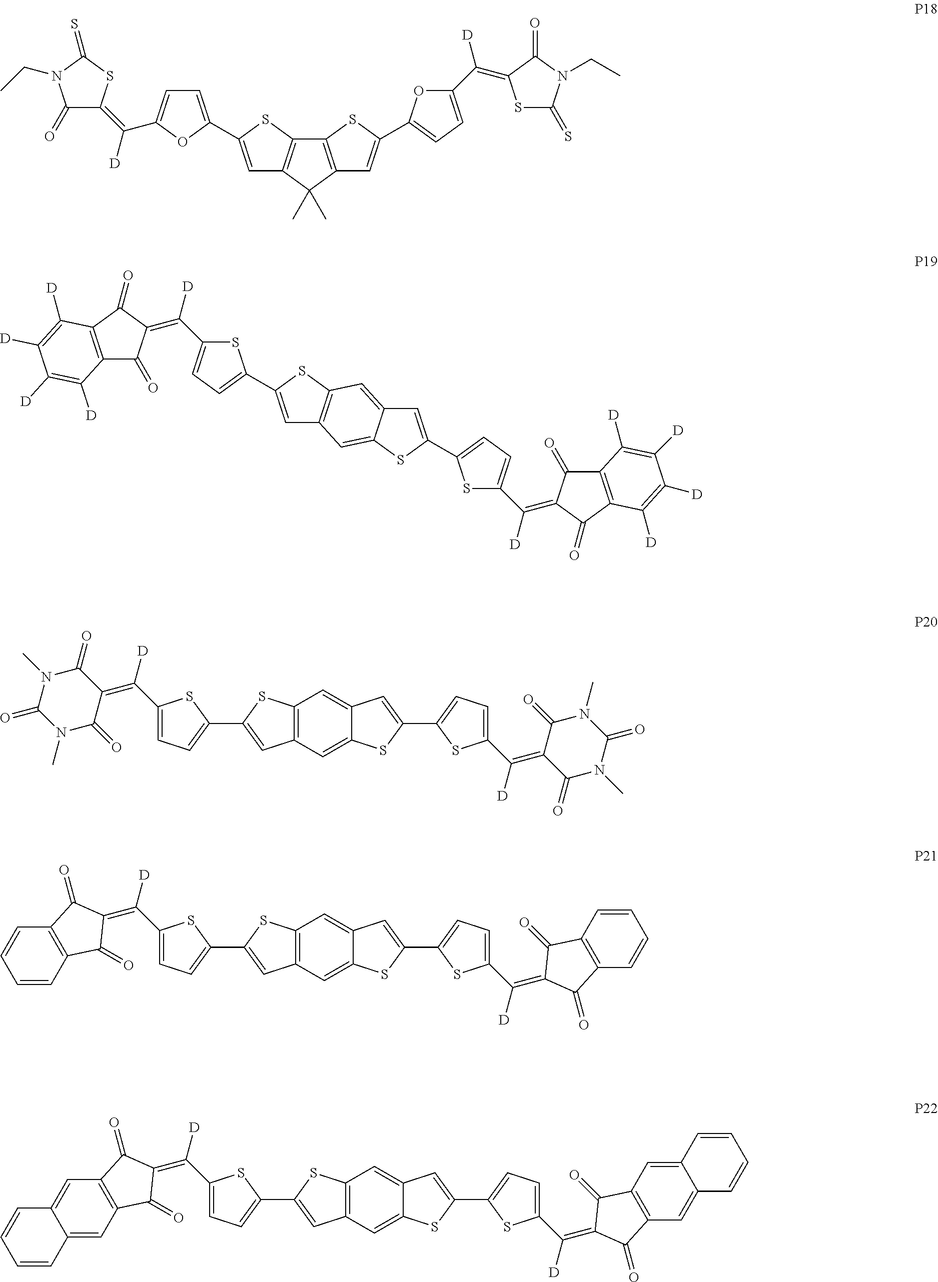

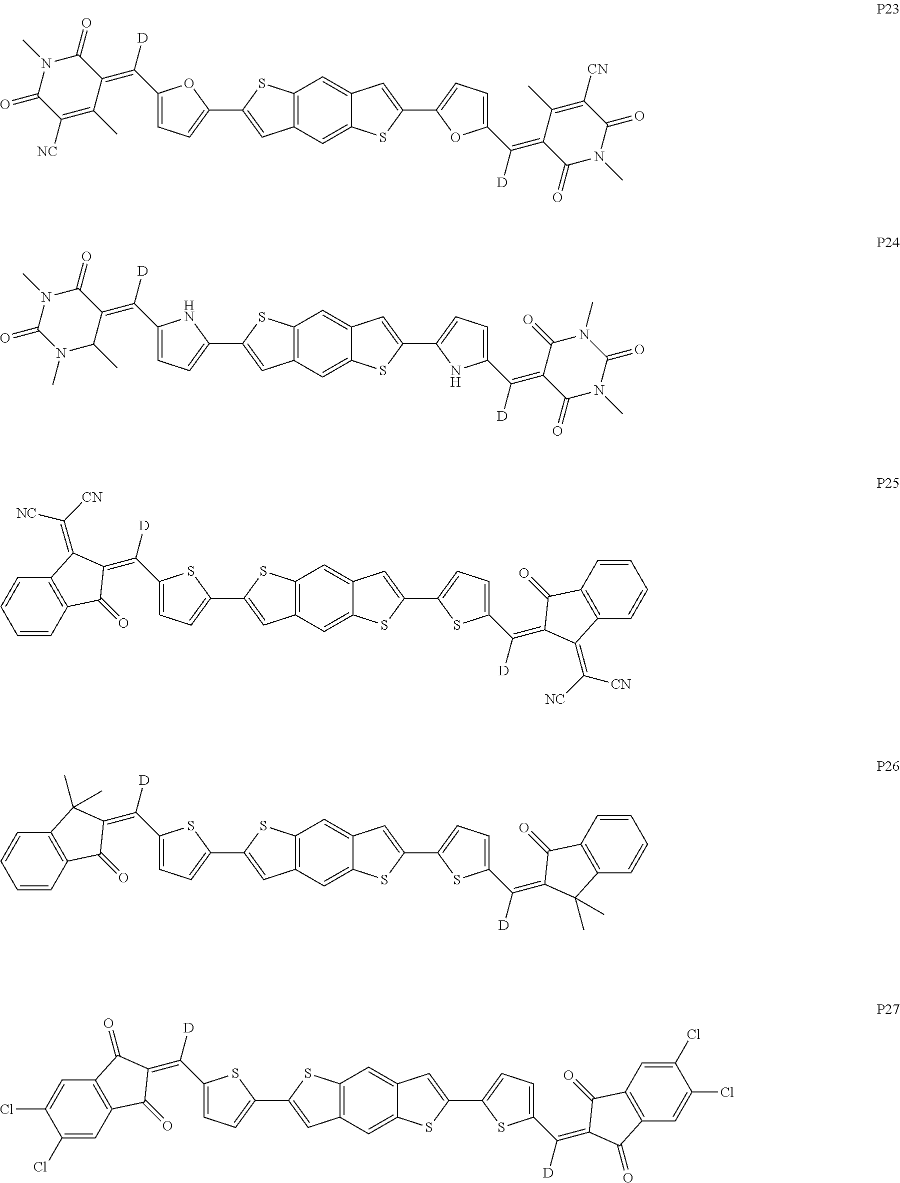

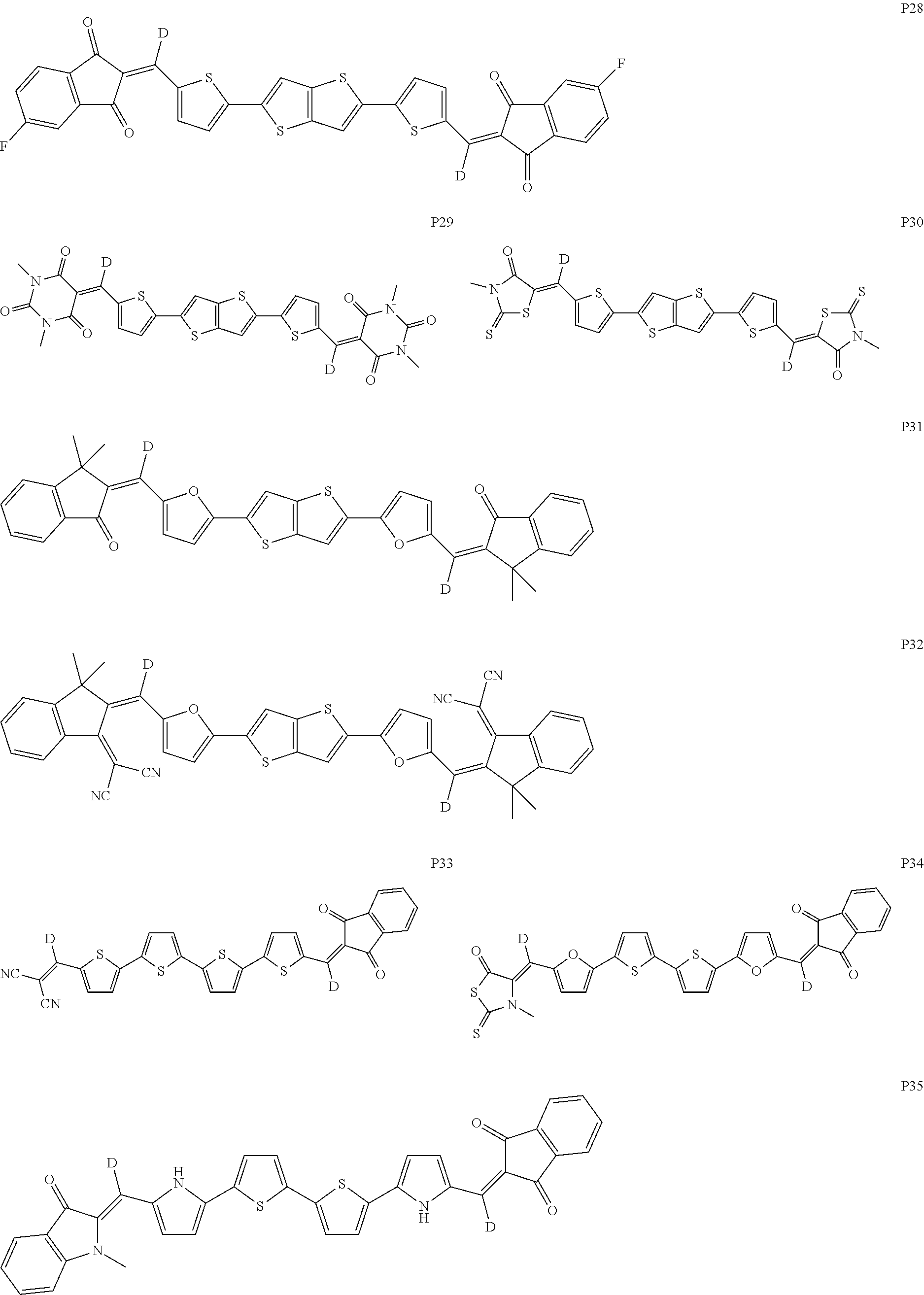

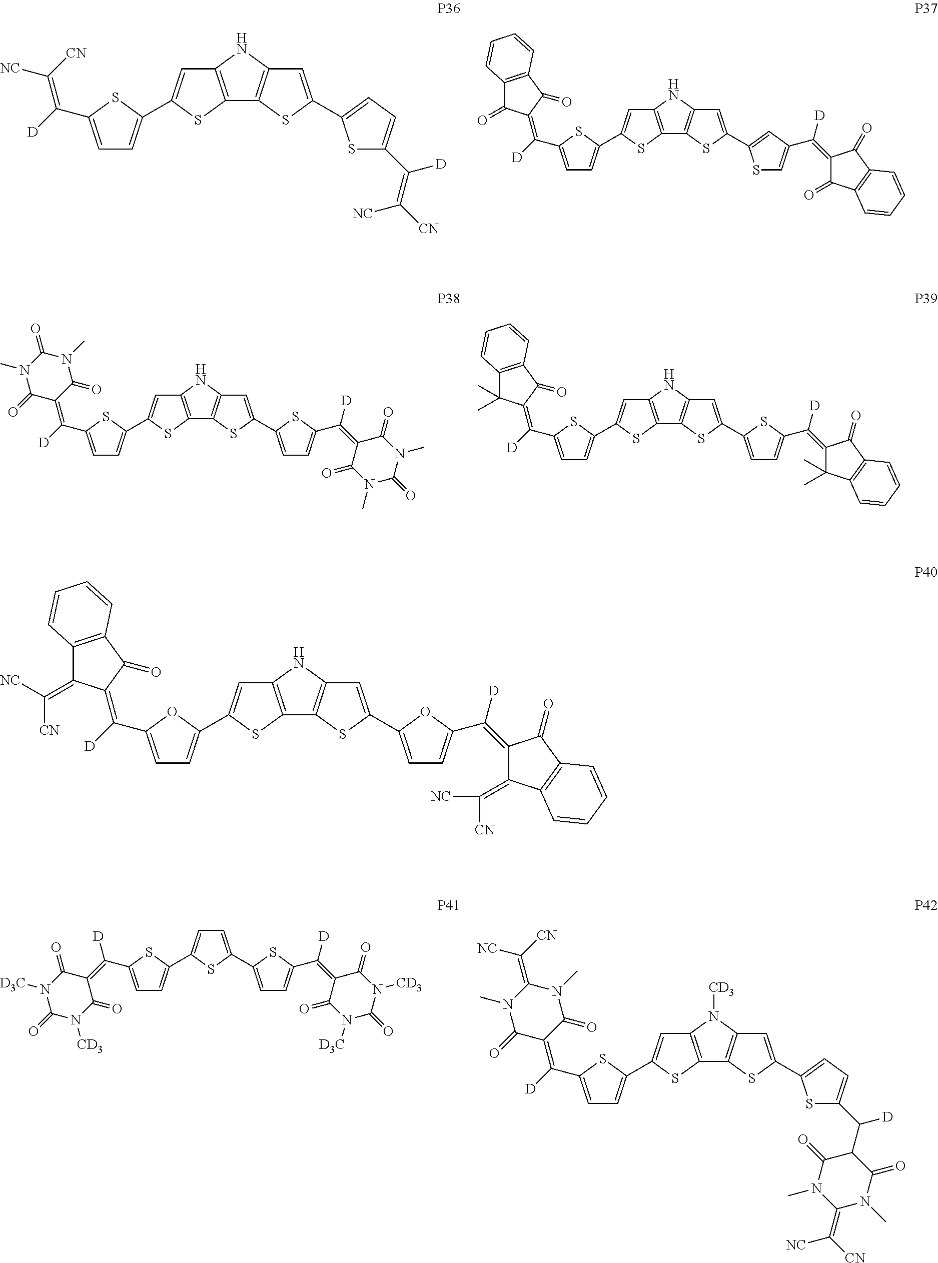

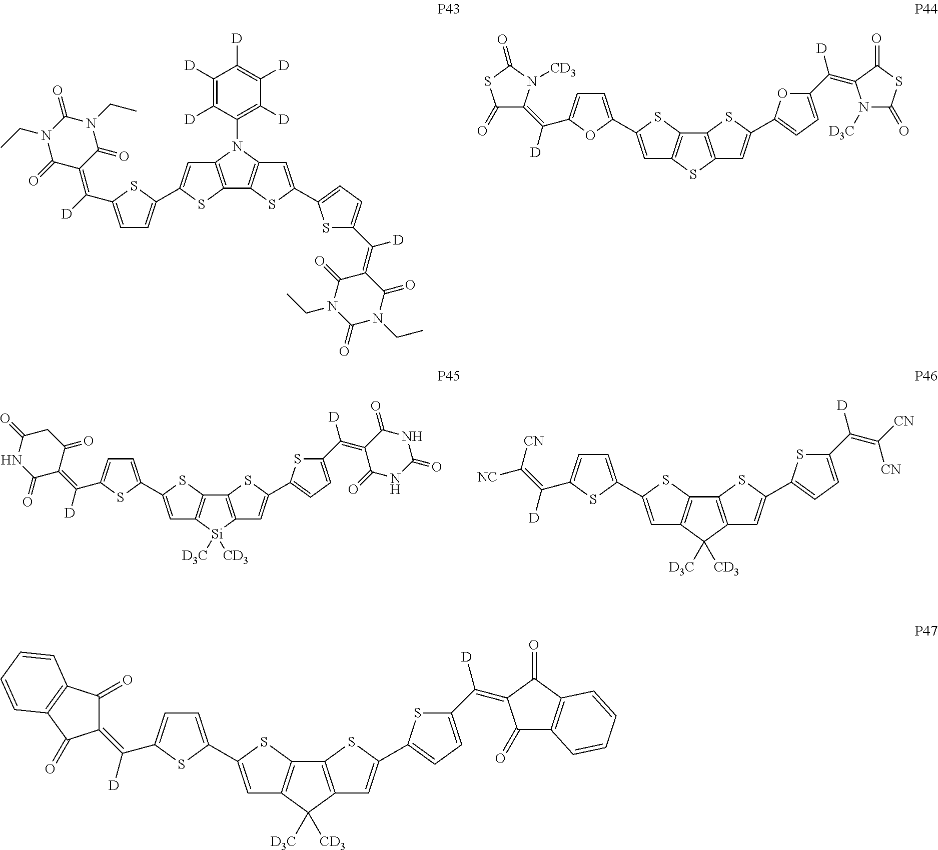

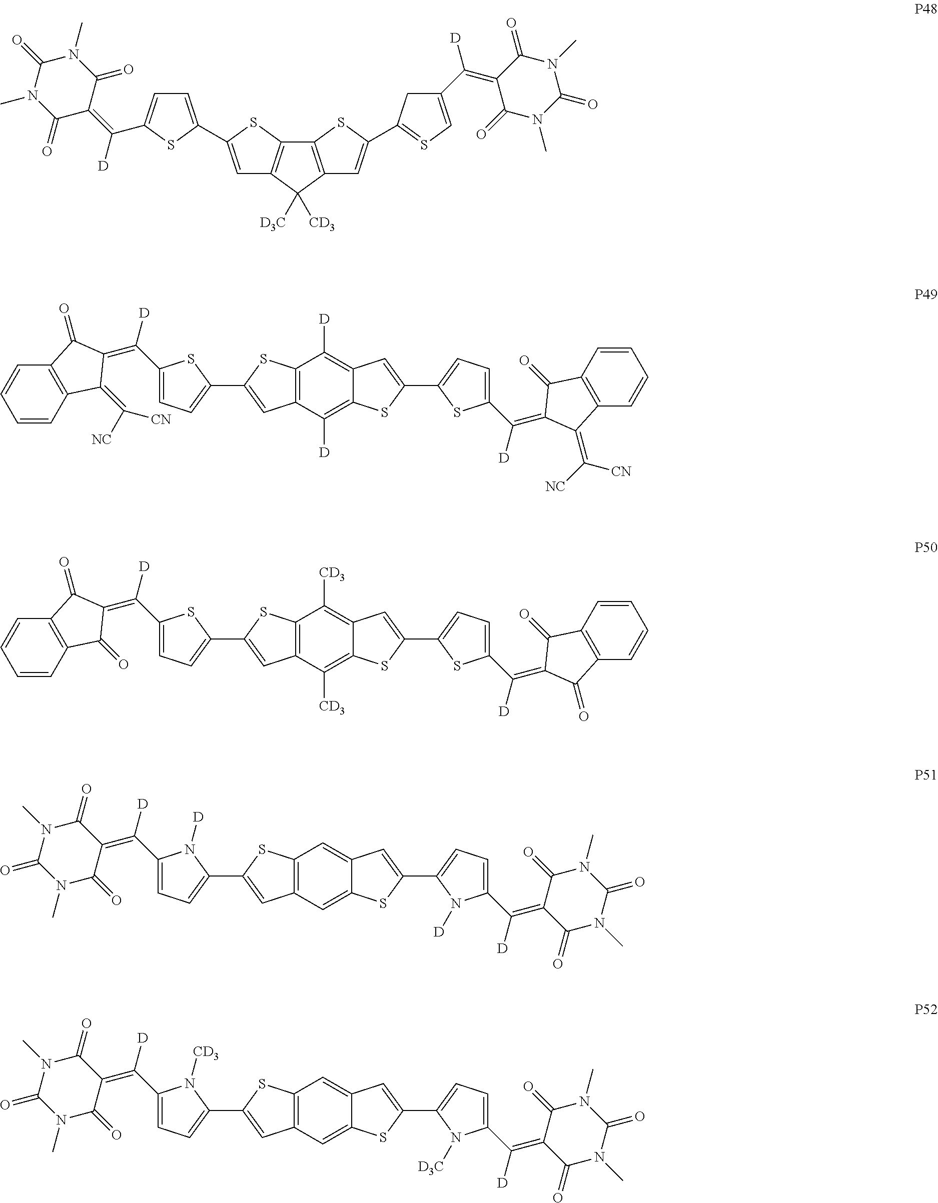

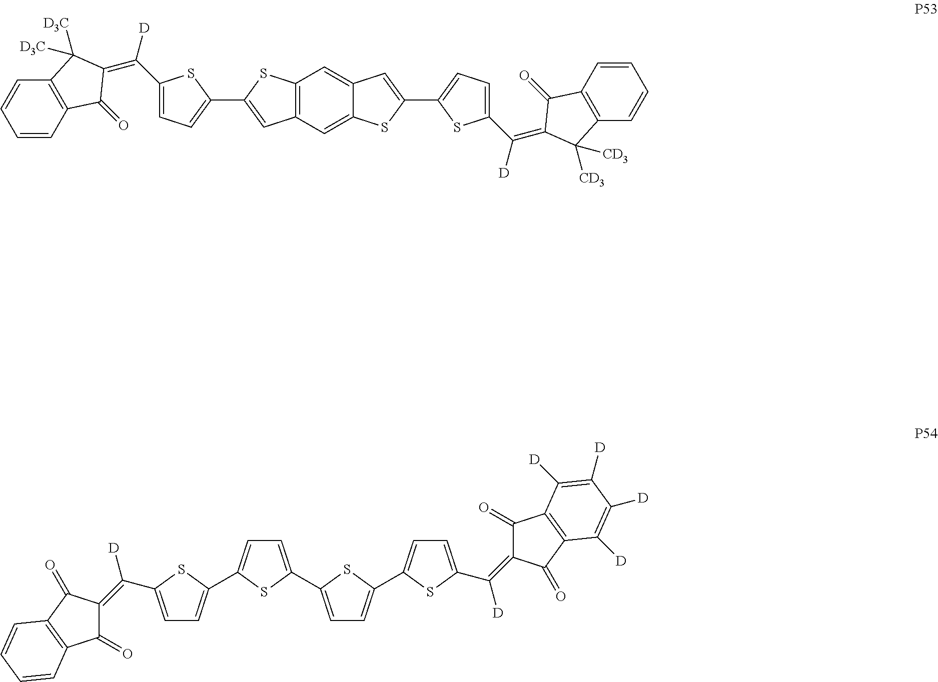 [SMILES: [2H]/C(=C1/C(=O)c2ccccc2C1(C([2H])([2H])[2H])C([2H])([2H])[2H])c1ccc(-c2cc3cc4sc(-c5ccc(/C([2H])=C6\C(=O)c7ccccc7C6(C([2H])([2H])[2H])C([2H])([2H])[2H])s5)cc4cc3s2)s1 ; [2H]C(=C1C(=O)c2ccccc2C1=O)c1ccc(-c2ccc(-c3ccc(-c4ccc(C([2H])=C5C(=O)c6c([2H])c([2H])c([2H])c([2H])c6C5=O)s4)s3)s2)s1]

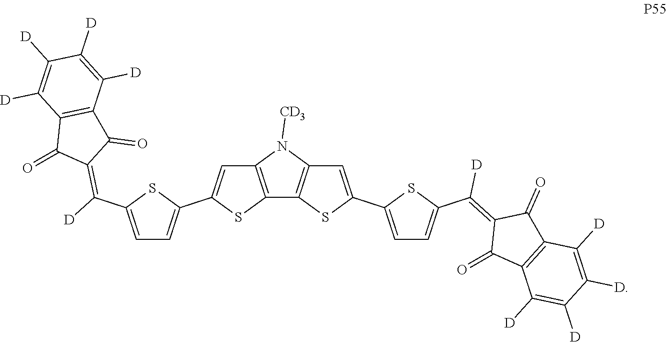

The organic compound represented by Formula 1 includes an electron-donor group represented by

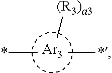

electron-acceptor groups represented by

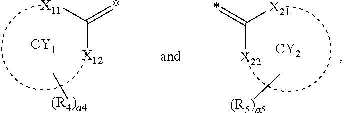

and a conjugation group having an appropriate length between the electron-donor group and the electron-acceptor groups, resulting in having a highest occupied molecular orbital (HOMO) energy level in a range of about −5.0 eV to about −5.8 eV and a lowest unoccupied molecular orbital (LUMO) energy level in a range of about −3.9 eV to about −2.7 eV, and may effectively absorb green light and/or red light. Therefore, the organic compound may be applied as a p-type semiconductor compound for opto-electronic devices.

In an embodiment, the organic compound represented by Formula 1 includes a vinyl deuterium, so that the vibration energy of the organic compound itself may be reduced. Due to the reduction in the vibrational energy of molecules, intermolecular reactions are minimized and excellent deposition stability and heat resistance may be achieved.

A photoactive layer (for example, a first layer) formed by depositing the organic compound has excellent purity. Accordingly, the opto-electronic device including the organic compound may have excellent external quantum efficiency and excellent dark current density. For example, when the organic compound represented by Formula 1 is included as the p-type semiconductor compound in the photoactive layer, an opto-electronic device with high external quantum efficiency and low dark current density may be implemented.

The method of synthesizing the organic compound represented by Formula 1 may be recognized by those skilled in the art by referring to the Examples, which will be described later.

According to an embodiment, an opto-electronic device may include a first electrode, a second electrode facing the first electrode, and a photoactive layer between the first electrode and the second electrode, wherein the opto-electronic device may include one or more types of the organic compounds (hereinafter, also referred to as a first compound). For example, the opto-electronic device may include at least one organic compound, each independently represented by Formula 1.

In an embodiment, the opto-electronic device may further include a hole transport region between the first electrode and the photoactive layer and an electron transport region between the photoactive layer and the second electrode. The hole transport region may include a hole injection layer, a hole transport layer, an emission auxiliary layer, an electron-blocking layer, or any combination thereof. The electron transport region may include a buffer layer, a hole-blocking layer, an electron control layer, an electron transport layer, an electron injection layer, or any combination thereof.

According to an embodiment, the opto-electronic device may further include a second compound represented by one of Formulae 2-1 to 2-6:

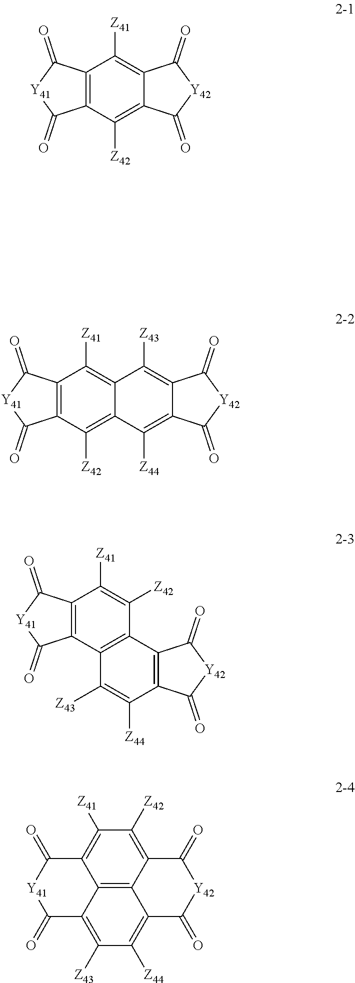

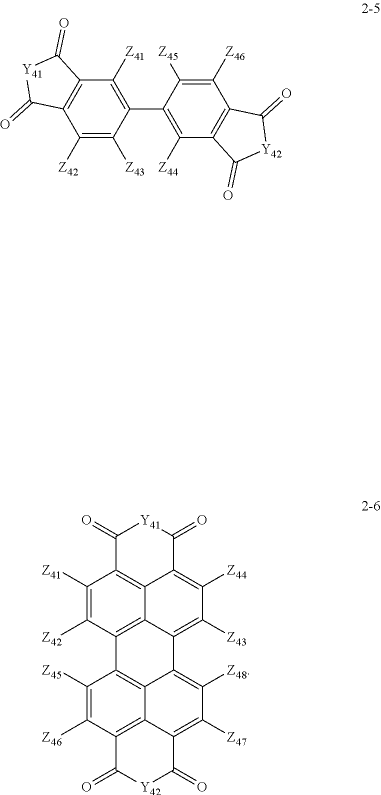

In Formulae 2-1 to 2-6,
Y₄₁ and Y₄₂ may each independently be C(Z₅₁)(Z₅₂), Si(Z₅₁)(Z₅₂), N(Z₅₁), P(Z₅₁), O, S, C(═O), C(═S), or C═C(Z₅₁)(Z₅₂),
Z₄₁ to Z₄₈, Z₅₁, and Z₅₂ may each independently be hydrogen, deuterium, —F, —C₁, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
R₁₀ₐ, Q₁ to Q₃, Q₁₁ to Q₁₃, Q₂₁ to Q₂₃, and Q₃₁ to Q₃₃ may each be the same as described herein.

According to an embodiment, the photoactive layer may not include a fullerene-based compound or a subphthalocyanine-based compound. For example, the photoactive layer may not include fullerene 60, fullerene 70, SubPC, or SubNC:

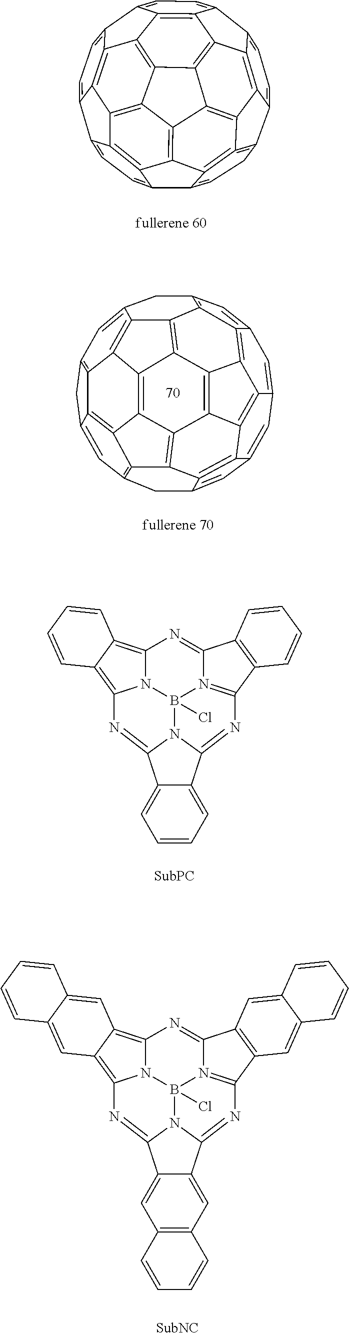

For example, the first compound may not be fullerene 60 or fullerene 70. For example, the second compound may not be SubPC or SubNC.

According to an embodiment, in Formulae 2-1 to 2-6,
Z₅₁ and Z₅₂ may each independently be a C₁-C₆₀ alkyl group, a C₃-C₁₀ cycloalkyl group, a C₆-C₆₀ aryl group, or a C₁-C₆₀ heteroaryl group, each unsubstituted or substituted with deuterium, —F, —Cl, —Br, —I, a cyano group, a C₁-C₆₀ alkyl group, a C₁-C₆₀ alkoxy group, or any combination thereof.

According to an embodiment, the second compound may be one of Compounds N1 to N43:

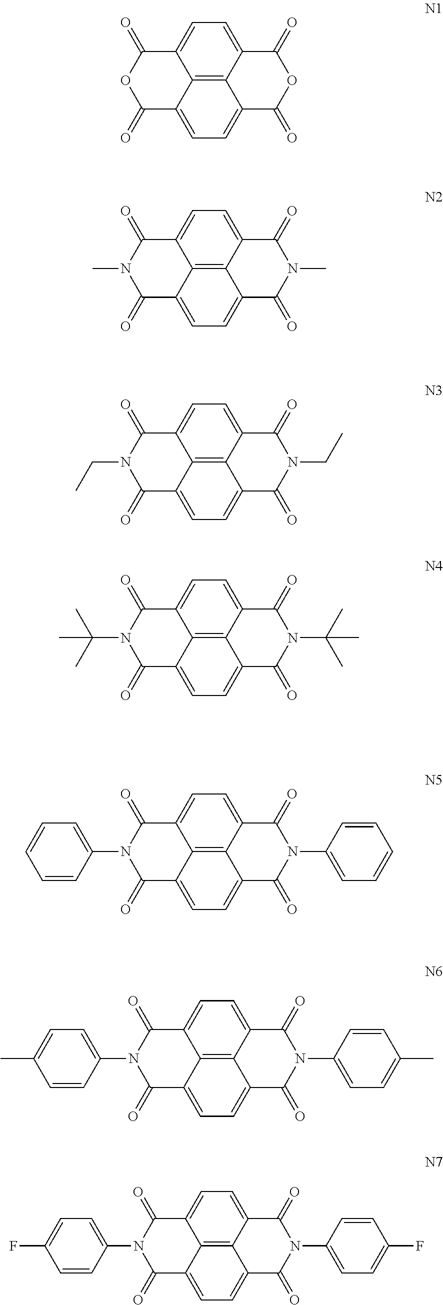

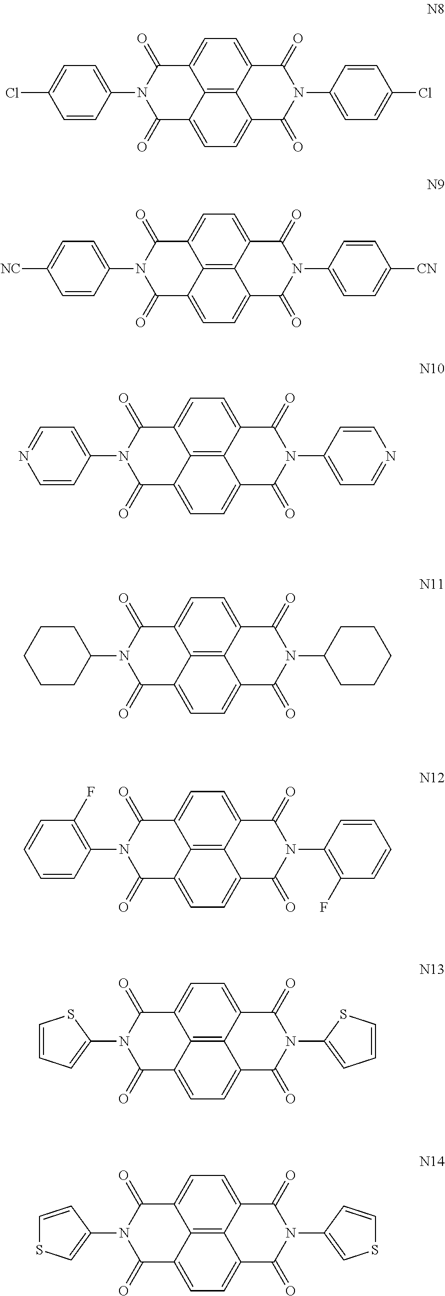

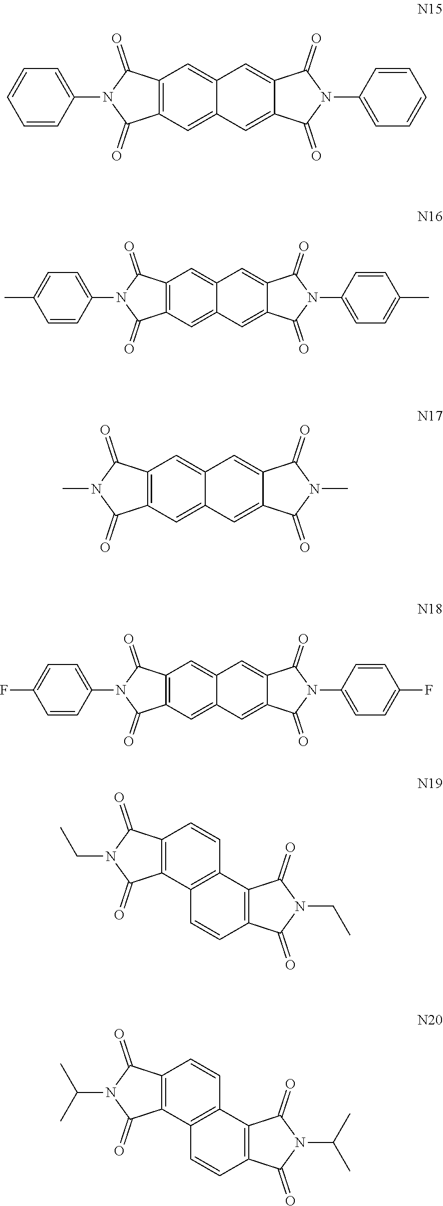

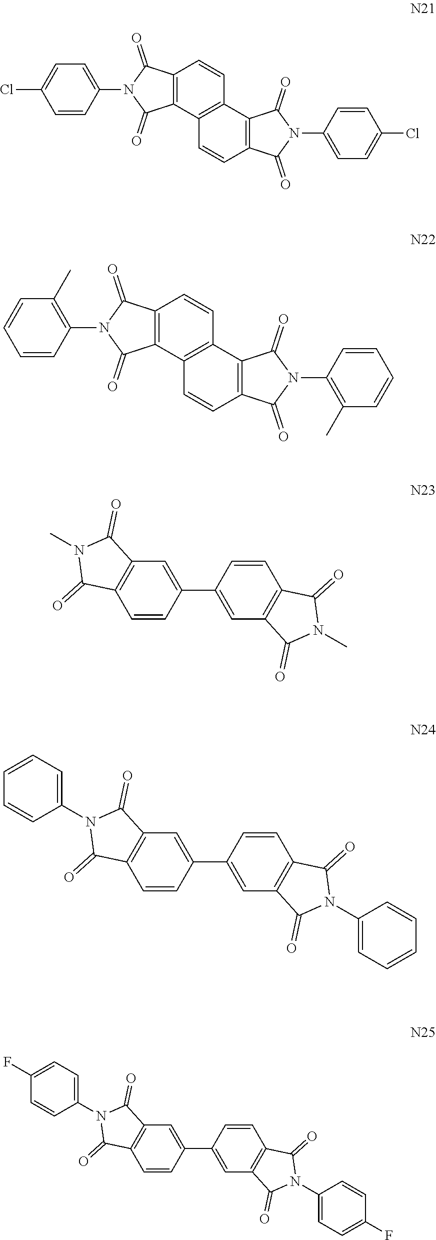

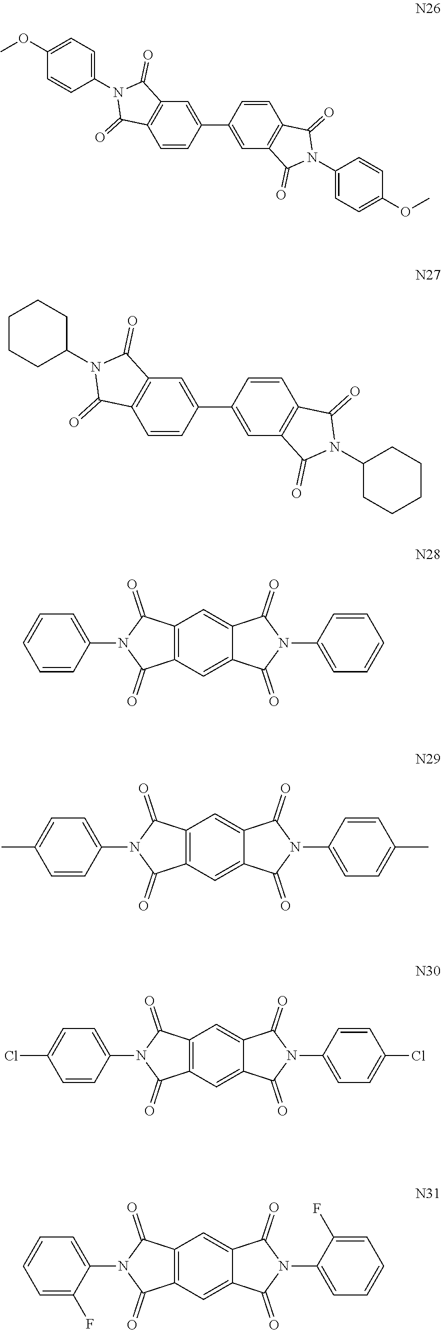

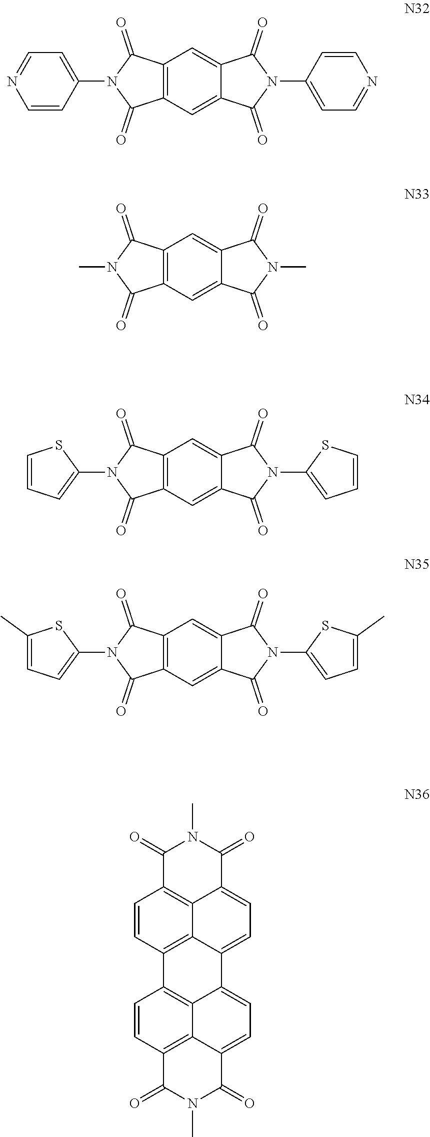

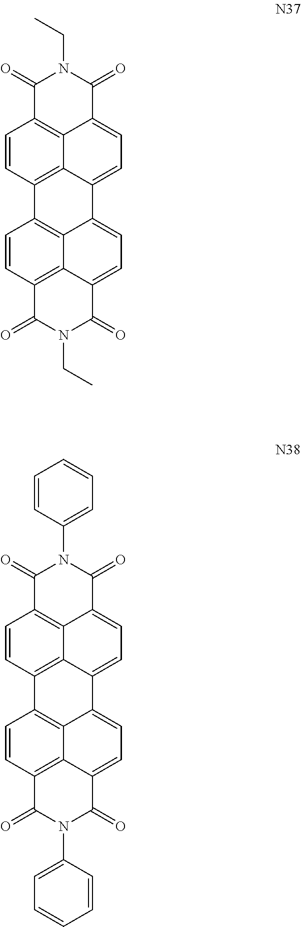 [SMILES: CCN1C(=O)c2ccc3c4ccc5c6c(ccc(c7ccc(c2c37)C1=O)c64)C(=O)N(CC)C5=O ; O=C1c2ccc3c4ccc5c6c(ccc(c7ccc(c2c37)C(=O)N1c1ccccc1)c64)C(=O)N(c1ccccc1)C5=O]

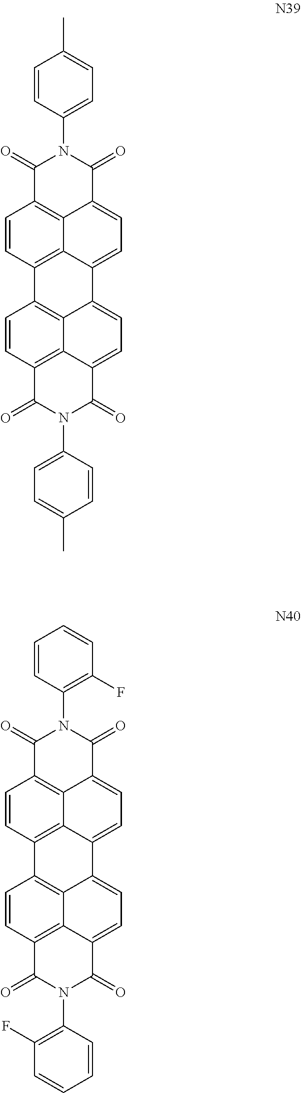 [SMILES: Cc1ccc(N2C(=O)c3ccc4c5ccc6c7c(ccc(c8ccc(c3c48)C2=O)c75)C(=O)N(c2ccc(C)cc2)C6=O)cc1 ; O=C1c2ccc3c4ccc5c6c(ccc(c7ccc(c2c37)C(=O)N1c1ccccc1F)c64)C(=O)N(c1ccccc1F)C5=O]

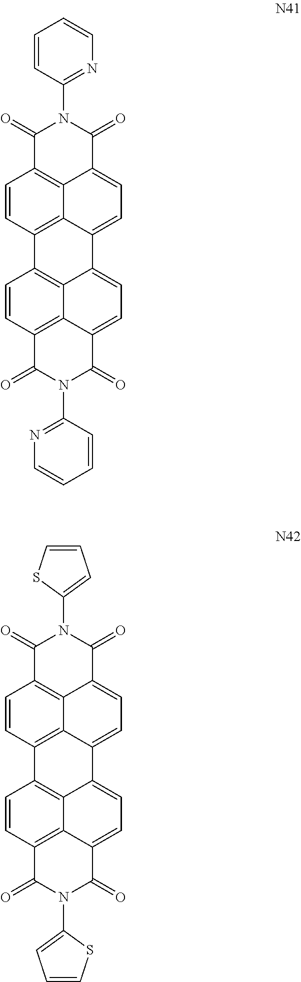 [SMILES: O=C1c2ccc3c4ccc5c6c(ccc(c7ccc(c2c37)C(=O)N1c1ccccn1)c64)C(=O)N(c1ccccn1)C5=O ; O=C1c2ccc3c4ccc5c6c(ccc(c7ccc(c2c37)C(=O)N1c1cccs1)c64)C(=O)N(c1cccs1)C5=O]

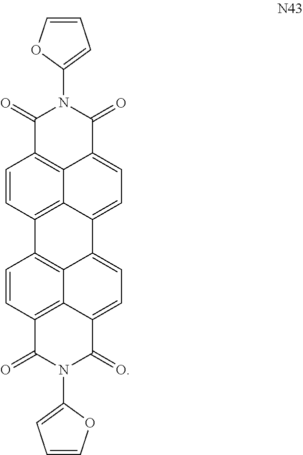 [SMILES: O=C1c2ccc3c4ccc5c6c(ccc(c7ccc(c2c37)C(=O)N1c1ccco1)c64)C(=O)N(c1ccco1)C5=O]

According to an embodiment, the photoactive layer may include the first compound. In an embodiment, the photoactive layer may further include the second compound. For example, the photoactive layer may include the first compound and the second compound. The photoactive layer may include a mixture of the first compound and the second compound.

The photoactive layer may be a single layer. For example, the first compound and the second compound may exist in a mixed state in the photoactive layer.

According to an embodiment, the photoactive layer may include a first layer adjacent to the first electrode, and a second layer adjacent to the second electrode. For example, the first layer may be disposed between the first electrode and the second layer. As another example, the first layer may be disposed between the hole transport region and the second layer. For example, the second layer may be disposed between the first layer and the second electrode. As another example, the second layer may be disposed between the first layer and the electron transport region.

According to an embodiment, the first layer may include the first compound, wherein the first layer may not include the second compound.

According to an embodiment, the second layer may include the second compound, wherein the second layer may not include the first compound.

For example, the photoactive layer may have a two-layer structure including the first layer including the first compound and the second layer including the second compound. For example, the first compound and the second compound may exist in an unmixed state in the photoactive layer.

According to an embodiment, the photoactive layer may further include a third layer between the first layer and the second layer. The third layer may include the first compound and the second compound. The third layer may include a mixture of the first compound and the second compound. For example, the first compound and the second compound may exist in a mixed state in the third layer. For example, the photoactive layer may have a three-layer structure including the first layer including the first compound and not including the second compound, the third layer including the first compound and the second compound, and the second layer including the second compound and not including the first compound.

Embodiments provide an electronic device which may include: the opto-electronic device; a light-emitting device; and a color filter, a color conversion layer, a touch screen layer, a polarizing layer, or any combination thereof, wherein the light-emitting device and the opto-electronic device may not overlap each other. For example, the light-emitting device and the opto-electronic device may not overlap each other in a cross-sectional view. For example, the light-emitting device and the opto-electronic device may not overlap each other in a plan view.

According to an embodiment, the light-emitting device may include an emission layer.

According to an embodiment, an electronic apparatus may include the opto-electronic device, wherein the electronic apparatus may be a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a mobile phone, a tablet computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall including multiple displays tiled together, a theater screen, a stadium screen, a phototherapy device, a signboard, an automotive sensor, a home sensor, or a solar cell.

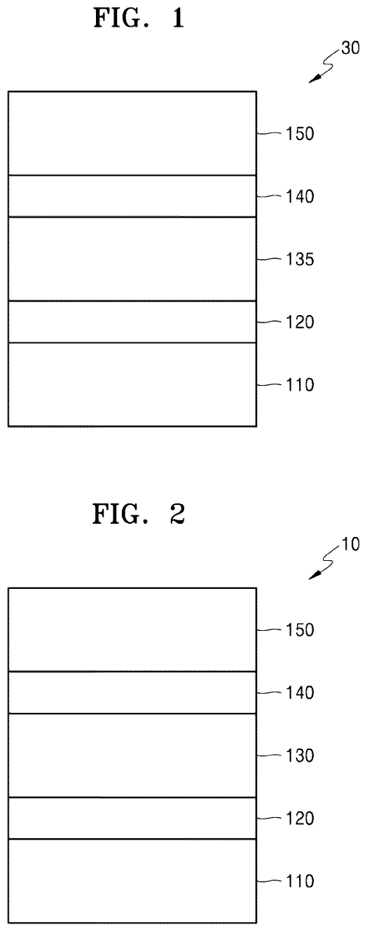

### [Description of FIGS. 1 and 2]

FIG. 1 is a schematic cross-sectional view of an opto-electronic device 30 according to an embodiment. The opto-electronic device 30 may include a first electrode 110, a hole transport region 120, a photoactive layer 135, an electron transport region 140, and a second electrode 150.

FIG. 2 is a schematic cross-sectional view of a light-emitting device 10. The light-emitting device 10 may include a first electrode 110, a hole transport region 120, an emission layer 130, an electron transport region 140, and a second electrode 150.

In an embodiment, the first electrode 110, the hole transport region 120, the electron transport region 140, and the second electrode 150 of the opto-electronic device 30 may be substantially integrated as one body with respect to the first electrode 110, the hole transport region 120, the electron transport region 140, and the second electrode 150 of the light-emitting device 10. In an embodiment, the first electrode 110, the hole transport region 120, the electron transport region 140, and the second electrode 150 of the opto-electronic device 30 may be respectively separated from the first electrode 110, the hole transport region 120, the electron transport region 140, and the second electrode 150 of the light-emitting device 10, but corresponding elements may include substantially a same material, and may be formed at about a same time.

Hereinafter, the structures of and manufacturing methods for the opto-electronic device 30 and the light-emitting device 10 according to embodiments are described with reference to FIGS. 1 and 2.

### [First Electrode 110]

In FIG. 1, a substrate may be further included under the first electrode 110 or on the second electrode 150. In an embodiment, the substrate may be a glass substrate or a plastic substrate. The substrate may be a flexible substrate. For example, the substrate may include plastics with excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a transflective electrode, or a transmissive electrode. When the first electrode 110 is a transmissive electrode, a material for forming the first electrode 110 may include indium tin oxide (ITO), indium zinc oxide (IZO), tin oxide (SnO2), zinc oxide (ZnO), or any combination thereof. In an embodiment, when the first electrode 110 is a transflective electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a structure consisting of a single layer or a structure including multiple layers. In an embodiment, the first electrode 110 may have a three-layer structure of ITO/Ag/ITO.

### [Hole Transport Region 120]

The hole transport region 120 may have a structure consisting of a layer including a single material, a structure consisting of a layer including different materials, or a structure including multiple layers including different materials.

The hole transport region 120 may include a hole injection layer, a hole transport layer, an emission auxiliary layer, an electron blocking layer, or any combination thereof.

In embodiments, the hole transport region 120 may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, wherein the layers in each structure may be stacked from the first electrode 110 in its respective stated order, but the structure of the hole transport region 120 is not limited thereto.

In embodiments, the hole transport region 120 may include a compound represented by Formula 201, a compound represented by Formula 202, or any combination thereof:

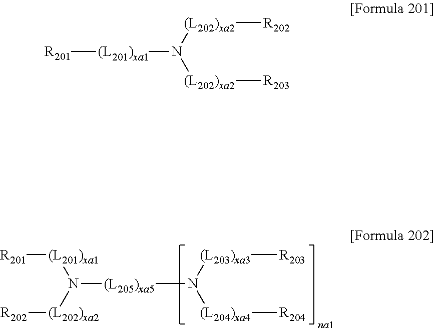

In Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group (for example, a carbazole group) that is unsubstituted or substituted with at least one R₁₀ₐ (for example, Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

In an embodiment, the compound represented by Formula 201 and the compound represented by Formula 202 may each independently include at least one of groups represented by Formulae CY201 to CY217:

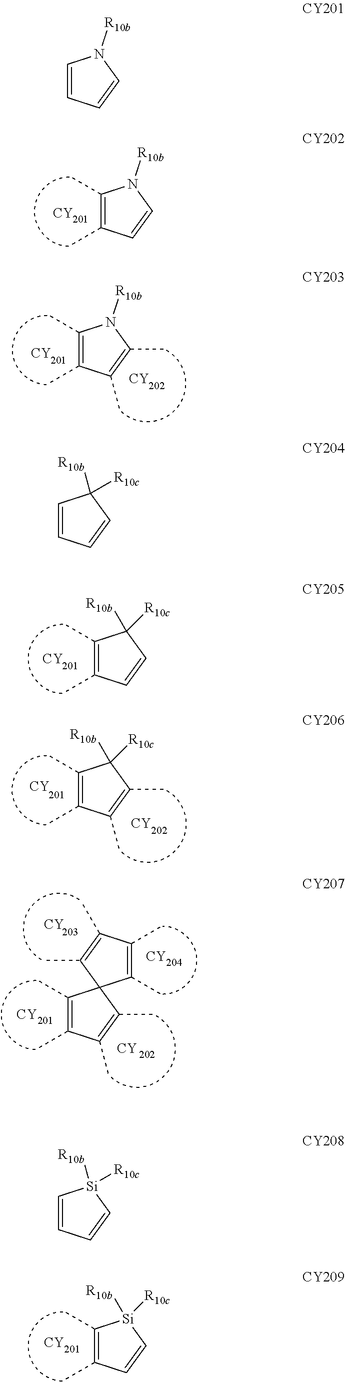

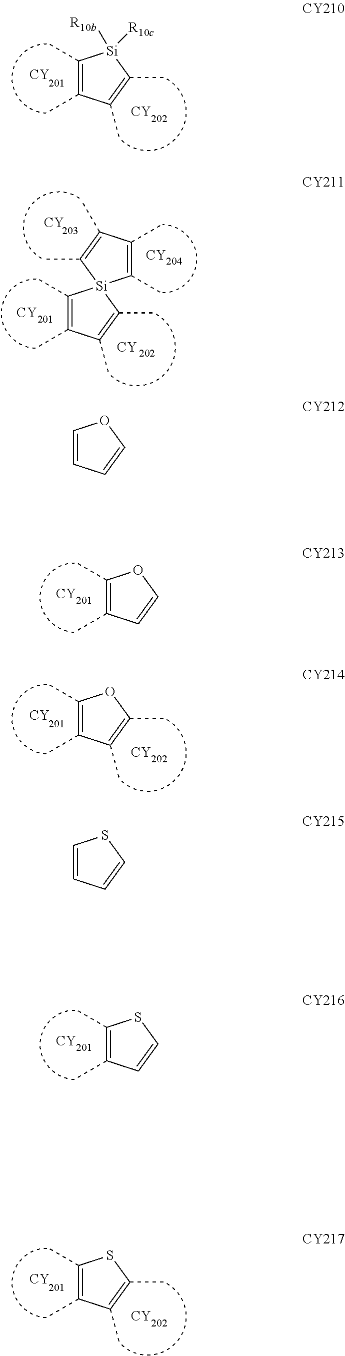

In Formulae CY201 to CY217, R_{10b} and R_{10c} may each independently be the same as described in connection with R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In an embodiment, in Formulae CY201 to CY217, ring CY201 to ring CY204 may each independently be a benzene group, a naphthalene group, a phenanthrene group, or an anthracene group.

In an embodiment, the compound represented by Formula 201 and the compound represented by Formula 202 may include at least one of groups represented by Formulae CY201 to CY203.

In an embodiment, the compound represented by Formula 201 may include at least one of groups represented by Formulae CY201 to CY203 and at least one of groups represented by Formulae CY204 to CY217.

In an embodiment, in Formula 201, xa1 may be 1, R₂₀₁ may be a group represented by one of Formulae CY201 to CY203, xa2 may be 0, and R₂₀₂ may be a group represented by one of Formulae CY204 to CY207.

In an embodiment, the compound represented by Formula 201 and the compound represented by Formula 202 may each not include groups represented by Formulae CY201 to CY203.

In an embodiment, the compound represented by Formula 201 and the compound represented by Formula 202 may each not include groups represented by Formulae CY201 to CY203 and may each independently include at least one of groups represented by Formulae CY204 to CY217.

In an embodiment, the compound represented by Formula 201 and the compound represented by Formula 202 may each not include groups represented by Formulae CY201 to CY217.

In an embodiment, the hole transport region 120 may include one of Compounds HT1 to HT46, m-MTDATA, TDATA, 2-TNATA, NPB (NPD), β-NPB, TPD, spiro-TPD, spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

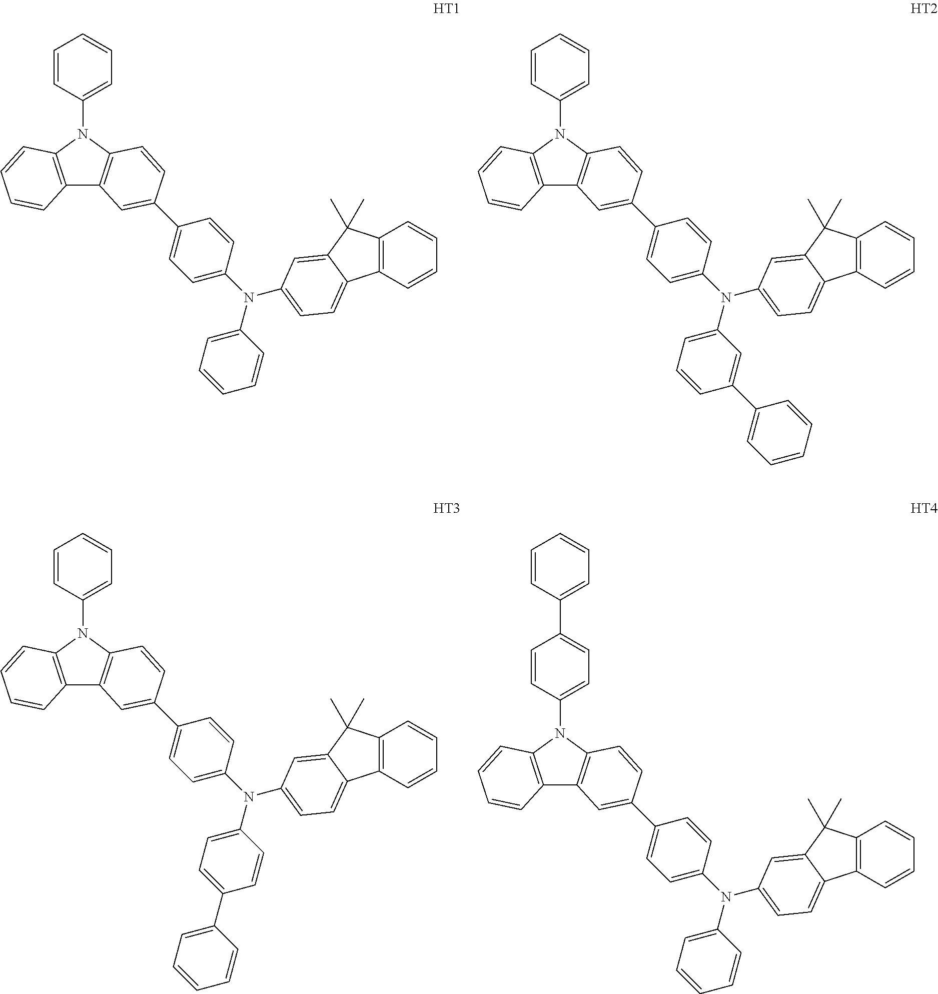

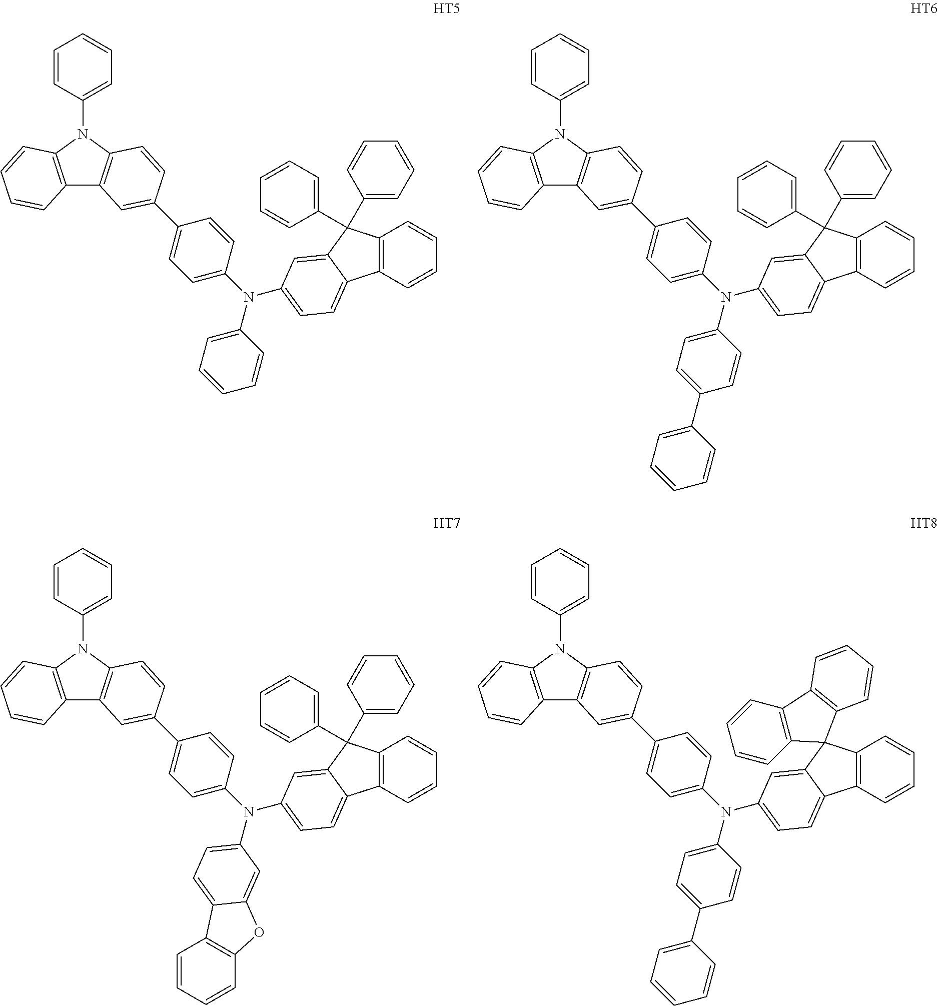 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

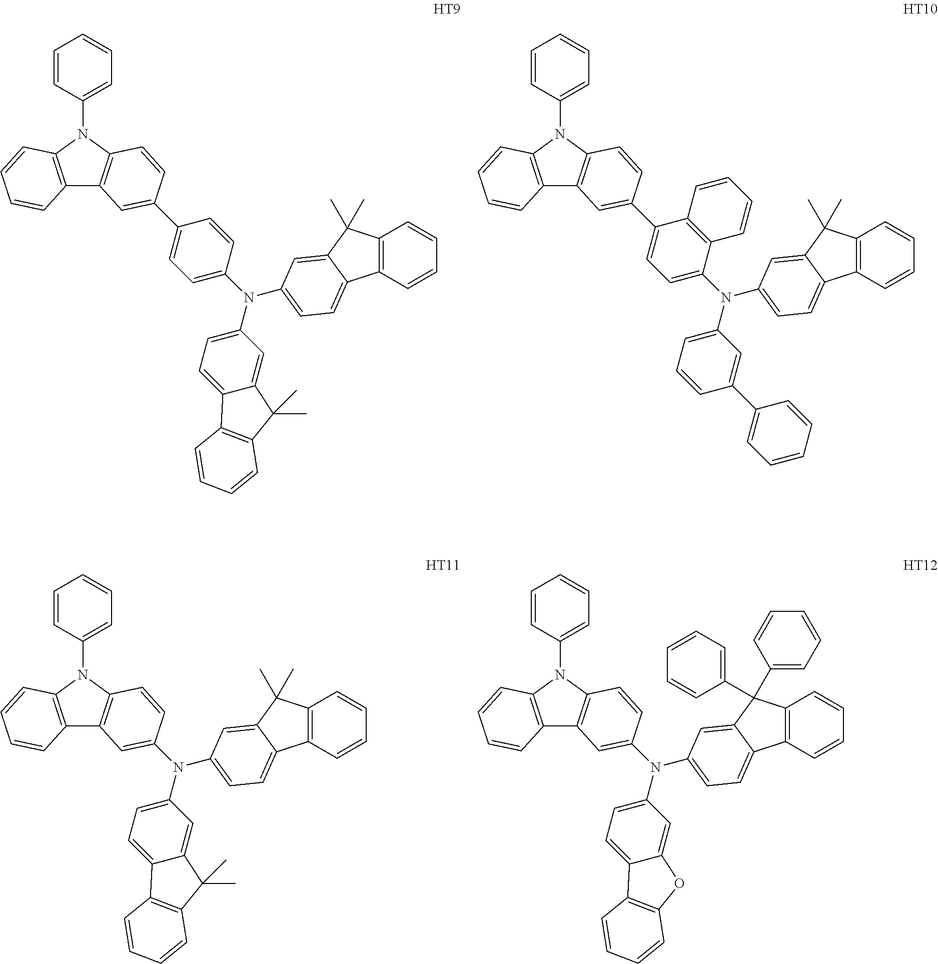 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

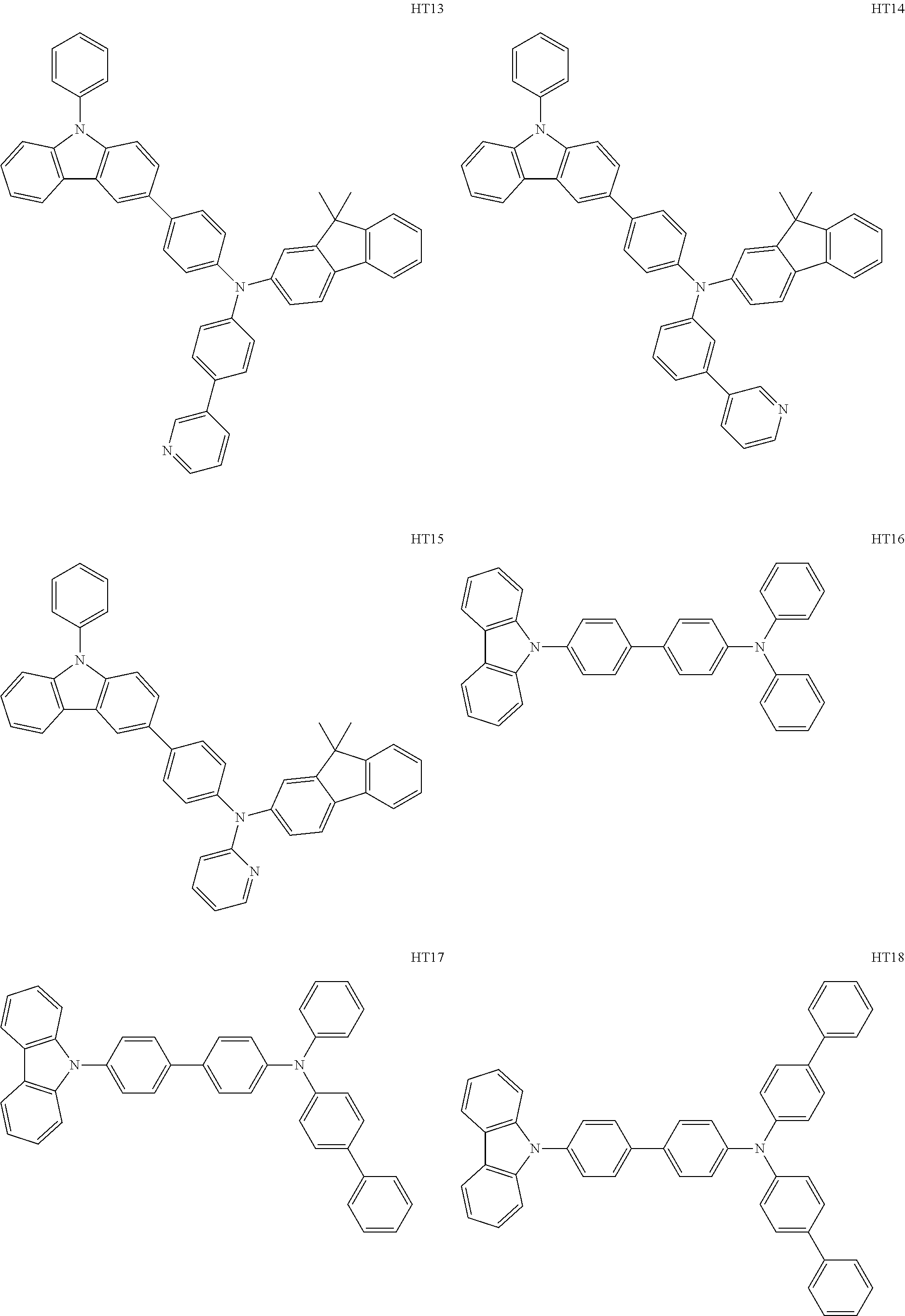 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

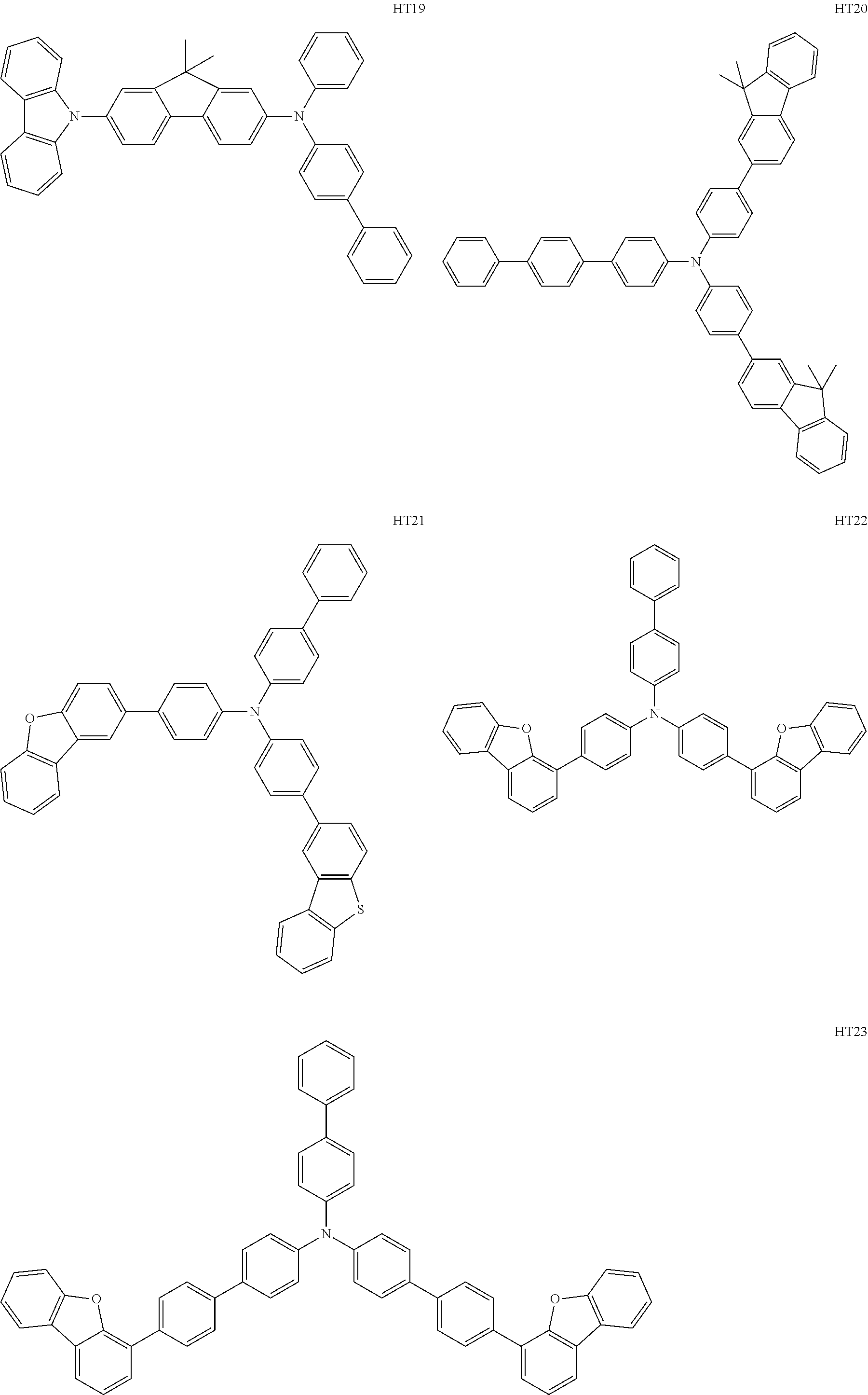

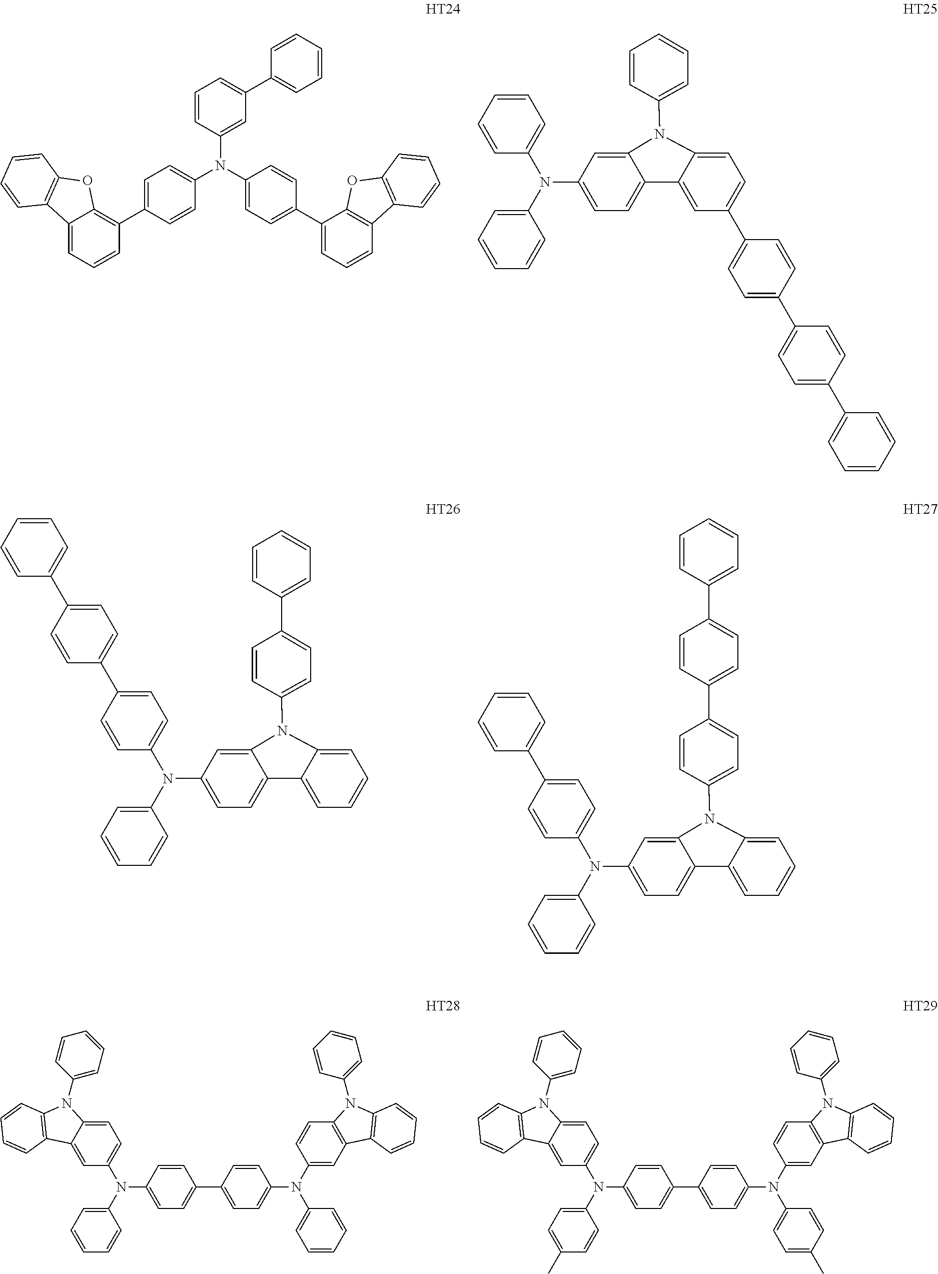

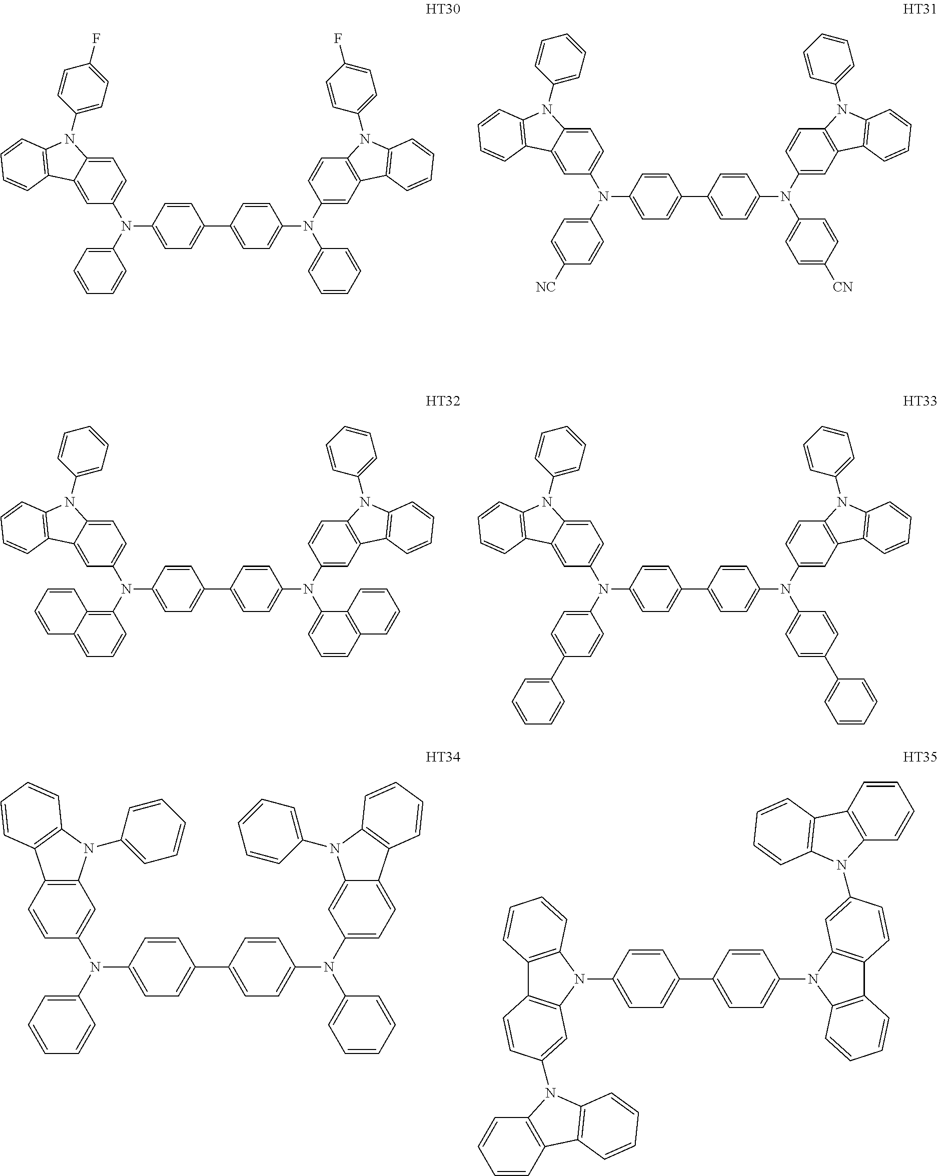

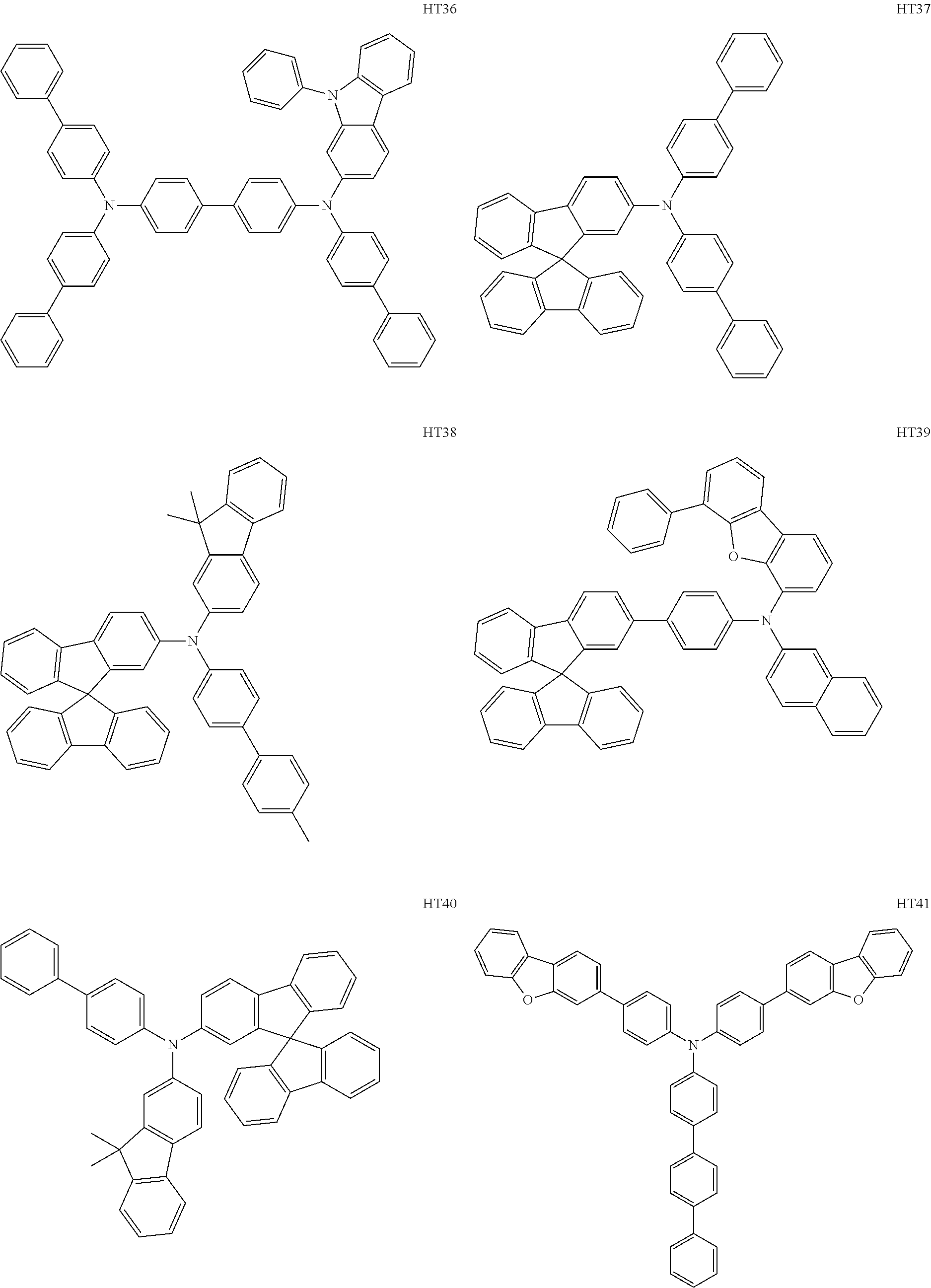

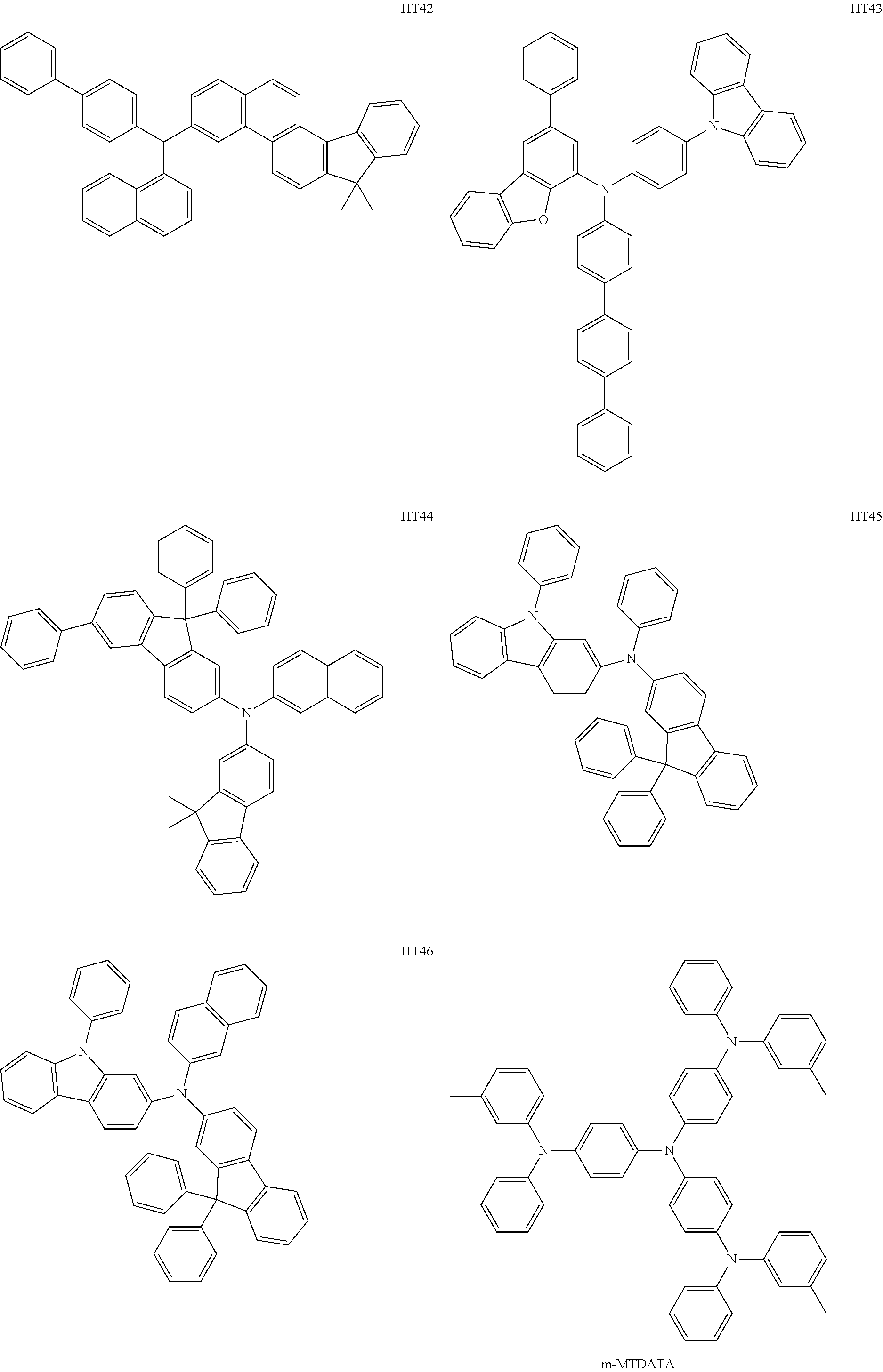

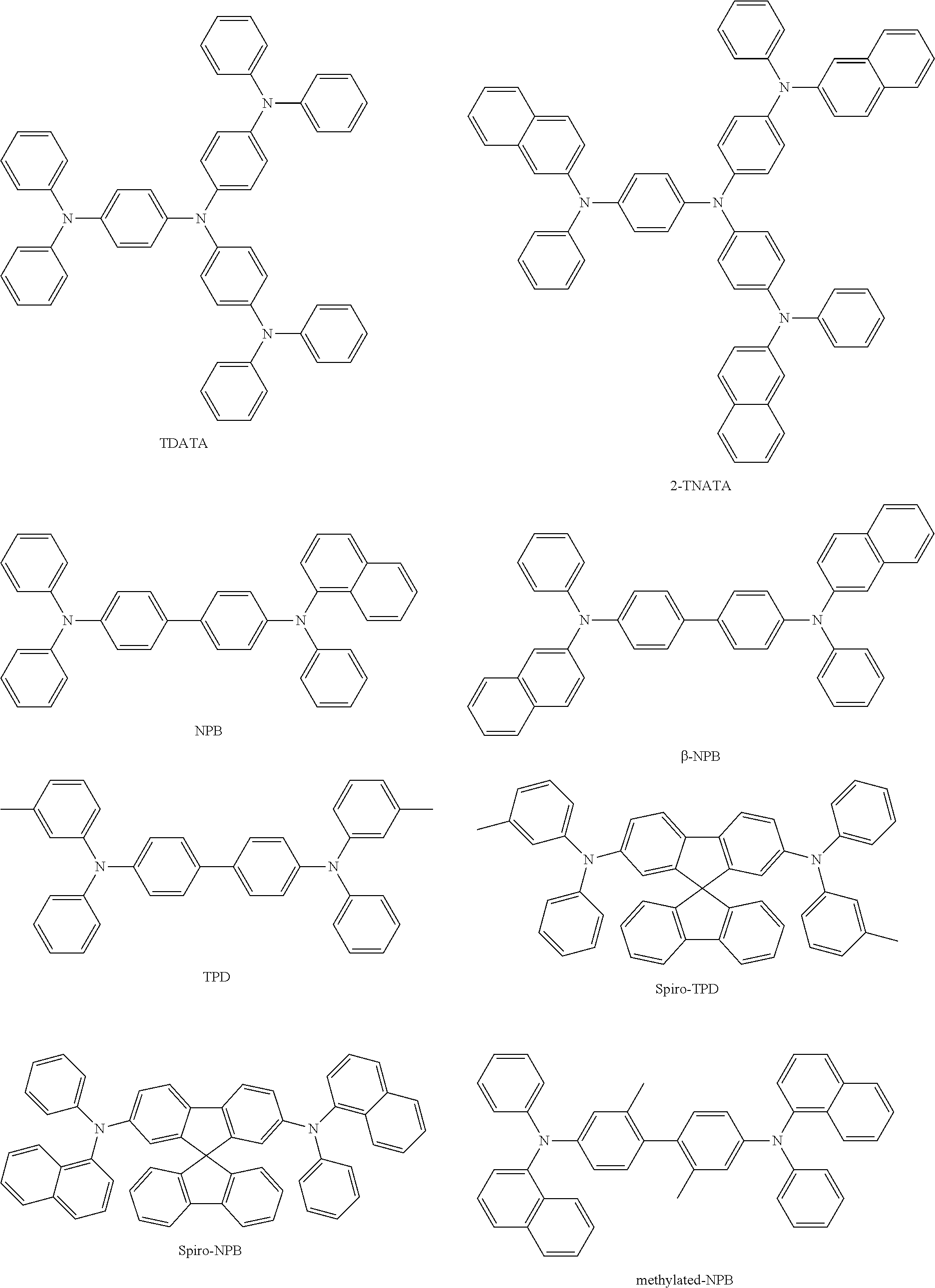

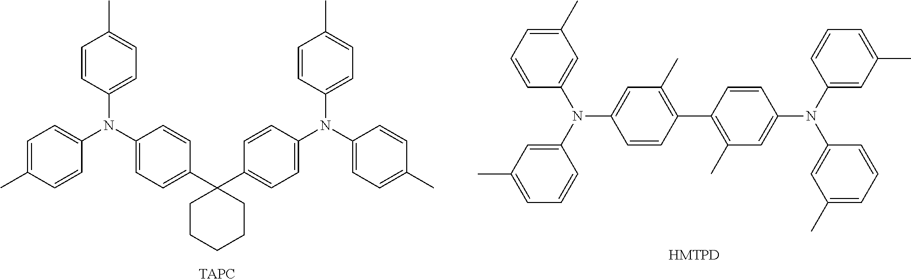

A thickness of the hole transport region 120 may be in a range of about 50 Å to about 10,000 Å. For example, the thickness of the hole transport region 120 may be in a range of about 100 Å to about 4,000 Å. When the hole transport region 120 includes a hole injection layer, a hole transport layer, or any combination thereof, a thickness of the hole injection layer may be about 100 Å to about 9,000 Å, and a thickness of the hole transport layer may be about 50 Å to about 2,000 Å. For example, the thickness of the hole injection layer may be in a range of about 100 Å to about 1,000 Å. For example, the thickness of the hole transport layer may be in a range of about 100 Å to about 1,500 Å. When the thicknesses of the hole transport region 120, the hole injection layer, and the hole transport layer are within the ranges described above, satisfactory hole transporting characteristics may be obtained without a substantial increase in driving voltage.

The emission auxiliary layer may increase light emission efficiency by compensating for an optical resonance distance according to a wavelength of light emitted by the emission layer 130, and the electron blocking layer may block the leakage of electrons from the emission layer 130 to the hole transport region 120. Materials that may be included in the hole transport region 120 may be included in the emission auxiliary layer and the electron blocking layer.

[p-Dopant]

The hole transport region 120 may further include, in addition to these materials, a charge-generation material for the improvement of conductive properties. The charge-generation material may be uniformly or non-uniformly dispersed in the hole transport region 120 (for example, in the form of a single layer consisting of a charge-generation material).

The charge-generation material may be, for example, a p-dopant.

For example, the lowest unoccupied molecular orbital (LUMO) energy of the p-dopant may be less than or equal to about −3.5 eV.

In an embodiment, the p-dopant may include a quinone derivative, a cyano group-containing compound, a compound including an element EL1 and an element EL2, or any combination thereof.

Examples of a quinone derivative may include TCNQ and F4-TCNQ.

Examples of a cyano group-containing compound may include HAT-CN and a compound represented by Formula 221.

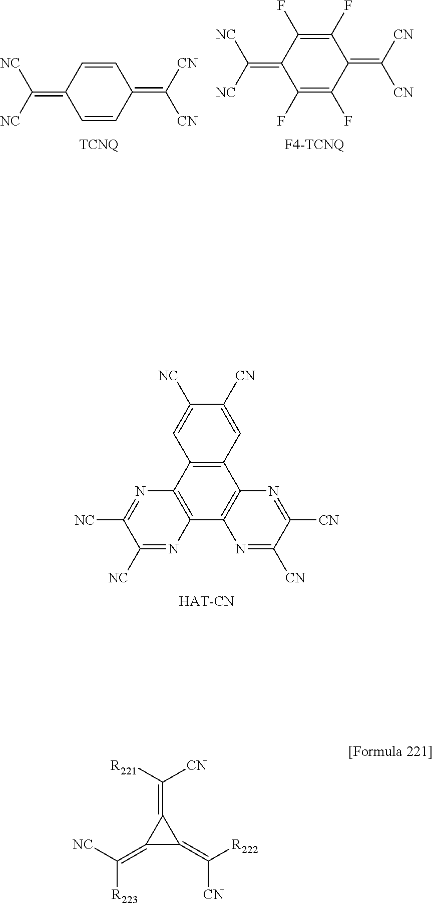

In Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be a metal, a metalloid, or any combination thereof, and element EL2 may be a non-metal, a metalloid, or any combination thereof.

Examples of a metal may include an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of a metalloid may include silicon (Si), antimony (Sb), and tellurium (Te).

Examples of a non-metal may include oxygen (O) and halogen (for example, F, Cl, Br, I, etc.).

Examples of a compound including element EL1 and element EL2 may include a metal oxide, a metal halide (for example, a metal fluoride, a metal chloride, a metal bromide, a metal iodide, etc.), a metalloid halide (for example, a metalloid fluoride, a metalloid chloride, a metalloid bromide, a metalloid iodide, etc.), a metal telluride, or any combination thereof.

Examples of a metal oxide may include a tungsten oxide (for example, WO, W2O3, WO2, WO3, W2O5, etc.), a vanadium oxide (for example, VO, V2O3, VO2, V2O5, etc.), a molybdenum oxide (for example, MoO, Mo2O3, MoO2, MoO3, Mo2O5, etc.), a rhenium oxide (for example, ReO3, etc.), etc.

Examples of a metal halide may include an alkali metal halide, an alkaline earth metal halide, a transition metal halide, a post-transition metal halide, and a lanthanide metal halide.

Examples of an alkali metal halide may include LiF, NaF, KF, RbF, CsF, LiCl, NaCl, KCl, RbCI, CsCl, LiBr, NaBr, KBr, RbBr, CsBr, Lil, NaI, KI, RbI, and CsI.

Examples of an alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and BaI₂.

Examples of a transition metal halide may include a titanium halide (for example, TiF₄, TiCl₄, TiBr₄, TiI₄, etc.), a zirconium halide (for example, ZrF₄, ZrCl₄, ZrBr₄, ZrI₄, etc.), a hafnium halide (for example, HfF₄, HfCl₄, HfBr₄, HfI₄, etc.), a vanadium halide (for example, VF₃, VCl₃, VBr₃, VI₃, etc.), a niobium halide (for example, NbF₃, NbCl₃, NbBr₃, NbI₃, etc.), a tantalum halide (for example, TaF₃, TaCl₃, TaBr₃, TaI₃, etc.), a chromium halide (for example, CrF₃, CrO₃, CrBr₃, CrI₃, etc.), a molybdenum halide (for example, MoF₃, MoCl₃, MoBr₃, MoI₃, etc.), a tungsten halide (for example, WF₃, WCl₃, WBr₃, WI₃, etc.), a manganese halide (for example, MnF₂, MnCl₂, MnBr₂, MnI₂, etc.), a technetium halide (for example, TcF₂, TcCl₂, TcBr₂, TcI₂, etc.), a rhenium halide (for example, ReF₂, ReCl₂, ReBr₂, ReI₂, etc.), an iron halide (for example, FeF₂, FeCl₂, FeBr₂, FeI₂, etc.), a ruthenium halide (for example, RuF₂, RuCl₂, RuBr₂, RuI₂, etc.), an osmium halide (for example, OsF₂, OsCl₂, OsBr₂, OsI₂, etc.), a cobalt halide (for example, CoF₂, COCl₂, CoBr₂, CoI₂, etc.), a rhodium halide (for example, RhF₂, RhCl₂, RhBr₂, RhI₂, etc.), an iridium halide (for example, IrF₂, IrCl₂, IrBr₂, IrI₂, etc.), a nickel halide (for example, NiF₂, NiCl₂, NiBr₂, NiI₂, etc.), a palladium halide (for example, PdF₂, PdCl₂, PdBr₂, PdI₂, etc.), a platinum halide (for example, PtF₂, PtCl₂, PtBr₂, PtI₂, etc.), a copper halide (for example, CuF, CuCl, CuBr, CuI, etc.), a silver halide (for example, AgF, AgCl, AgBr, AgI, etc.), and a gold halide (for example, AuF, AuCl, AuBr, Aul, etc.).

Examples of a post-transition metal halide may include a zinc halide (for example, ZnF₂, ZnCl₂, ZnBr₂, ZnI₂, etc.), an indium halide (for example, InI₃, etc.), a tin halide (for example, SnI₂, etc.), etc.

Examples of a lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, etc.

Examples of a metalloid halide may include an antimony halide (for example, SbCl5, etc.).

Examples of a metal telluride may include an alkali metal telluride (for example, Li₂Te, Na₂Te, K₂Te, Rb₂Te, Cs₂Te, etc.), an alkaline earth metal telluride (for example, BeTe, MgTe, CaTe, SrTe, BaTe, etc.), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (for example, ZnTe, etc.), and a lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### [Emission Layer 130]

The light-emitting device 10 may include an emission layer 130 on the hole transport region 120.

In an embodiment, the emission layer 130 may further include, in addition to various organic materials, a metal-containing compound such as an organometallic compound, an inorganic material such as a quantum dot, and the like.

The emission layer 130 may include two or more emitting units stacked between the first electrode 110 and the second electrode 150, and at least one charge generation layer located between adjacent units among the two or more emitting units. When the emission layer 130 includes the two or more emitting units as described above and the at least one charge generation layer as described above, the light-emitting device 10 may be a tandem light-emitting device.

When the light-emitting device 10 is a full-color light-emitting device, the emission layer 130 may be patterned into a red emission layer, a green emission layer, and/or a blue emission layer, according to a subpixel. In an embodiment, the emission layer may have a stacked structure of two or more layers of a red emission layer, a green emission layer, and a blue emission layer, in which the two or more layers may contact each other or may be separated from each other, to emit white light. In embodiments, the emission layer 130 may include two or more materials of a red light-emitting material, a green light-emitting material, and a blue light-emitting material, in which the two or more materials may be mixed with each other in a single layer, to emit white light.

The emission layer 130 may include a host and a dopant. The dopant may include a phosphorescent dopant, a fluorescent dopant, or any combination thereof.

An amount of the dopant in the emission layer 130 may be in a range of about 0.01 part by weight to about 15 parts by weight based on 100 parts by weight of the host.

In an embodiment, the emission layer 130 may include a quantum dot.

In an embodiment, the emission layer 130 may include a delayed fluorescence material. The delayed fluorescence material may serve as a host or as a dopant in the emission layer 130.

A thickness of the emission layer 130 may be about 100 Å to about 1,000 Å.

For example, the thickness of the emission layer 130 may be in a range of about 200 Å to about 600 Å. When the thickness of the emission layer 130 is within any of the ranges described above, excellent luminescence characteristics may be obtained without a substantial increase in driving voltage.

### [Host]

In an embodiment, the host may include a compound represented by Formula 301:

[Ar₃₀₁]_{xb11}—[(L₃₀₁)_{xb1}-R₃₀₁]_{xb21} [Formula 301]

In Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each independently be the same as described in connection with Q₁.

In an embodiment, in Formula 301, when xb11 is 2 or more, two or more of Ar₃₀₁ may be linked to each other via a single bond.

In an embodiment, the host may include a compound represented by Formula 301-1, a compound represented by Formula 301-2, or any combination thereof:

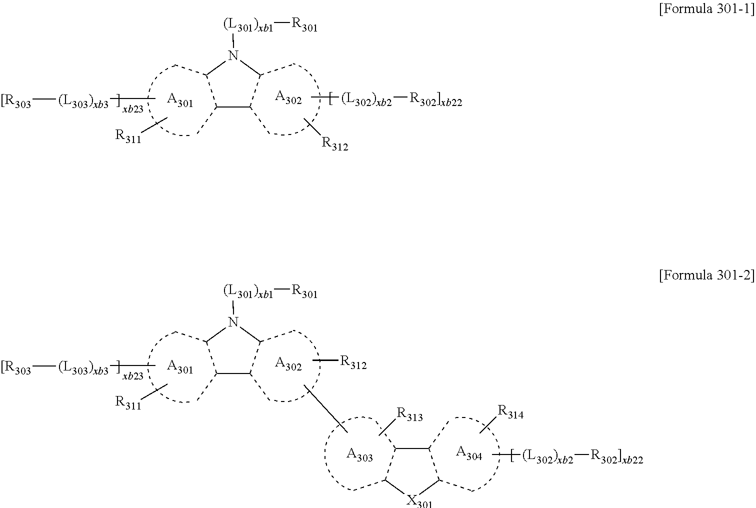

In Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described in the specification,
L₃₀₂ to L₃₀₄ may each independently be the same as described in connection with L₃₀₁,
xb2 to xb4 may each independently be the same as described in connection with xb1, and
R₃₀₂ to R₃₀₅ and R₃₁₁ to R₃₁₄ may each independently be the same as described in connection with R₃₀₁.

In an embodiment, the host may include an alkali earth metal complex, a post-transition metal complex, or any combination thereof. In an embodiment, the host may include a Be complex (for example, Compound H55), an Mg complex, a Zn complex, or any combination thereof.

In an embodiment, the host may include one of Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl)benzene (TCP), or any combination thereof:

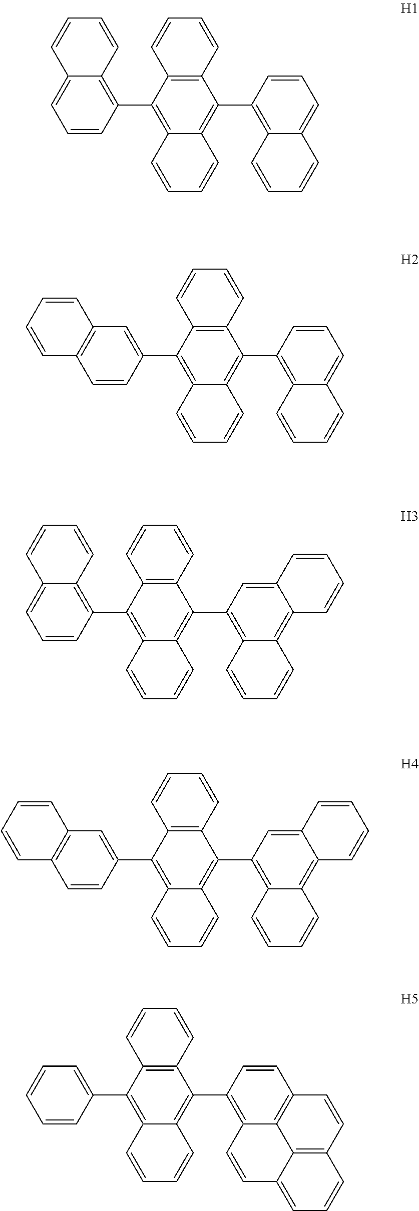

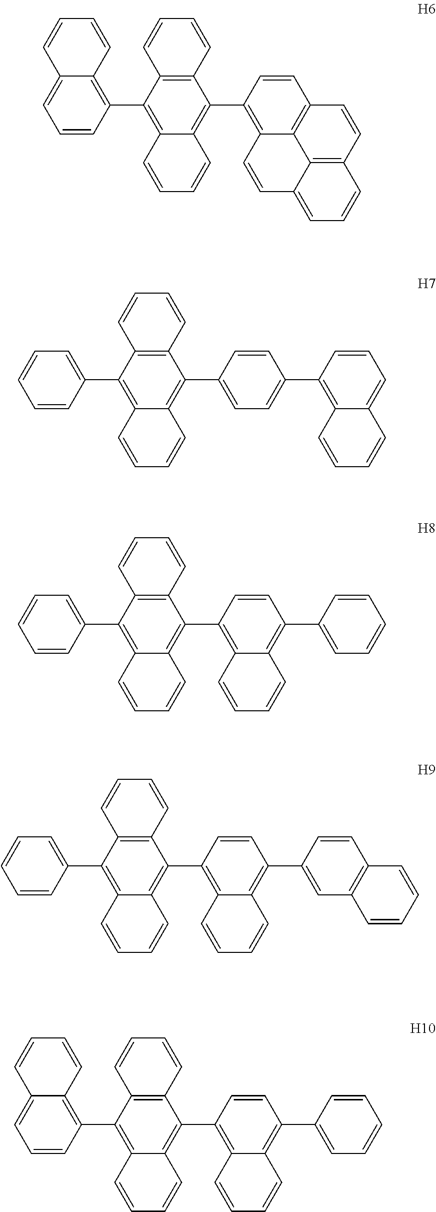

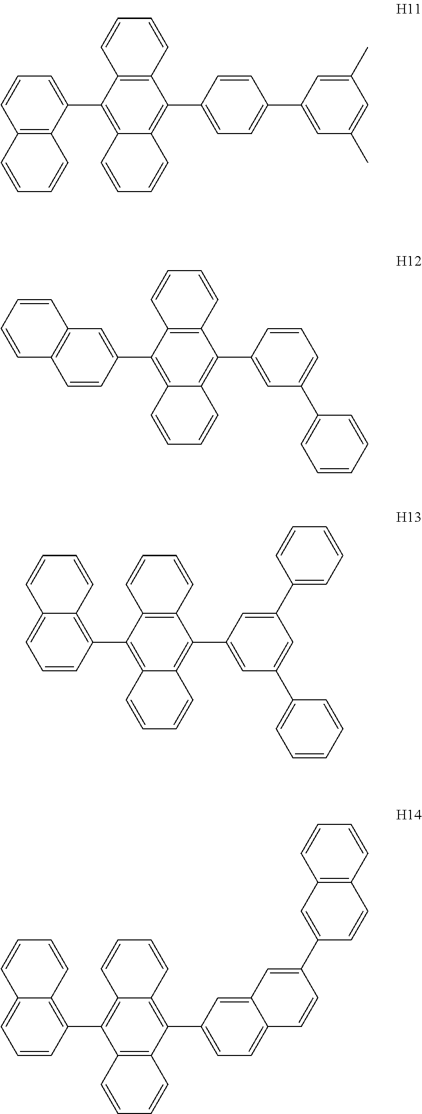

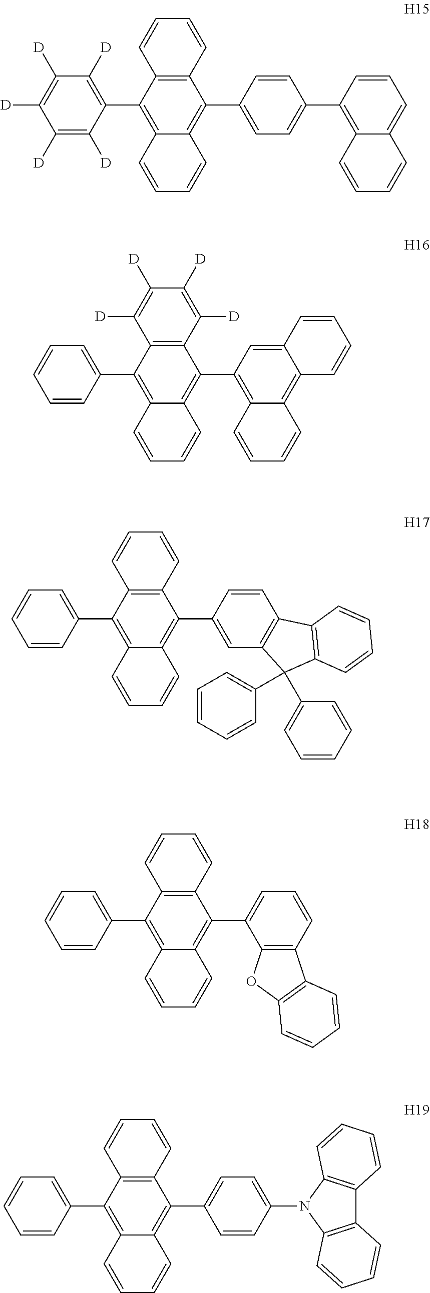

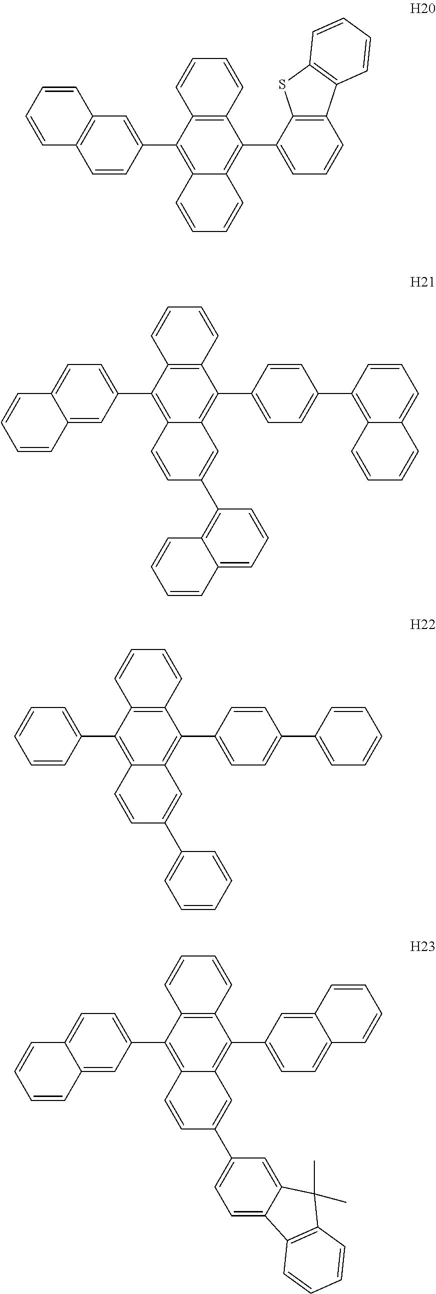

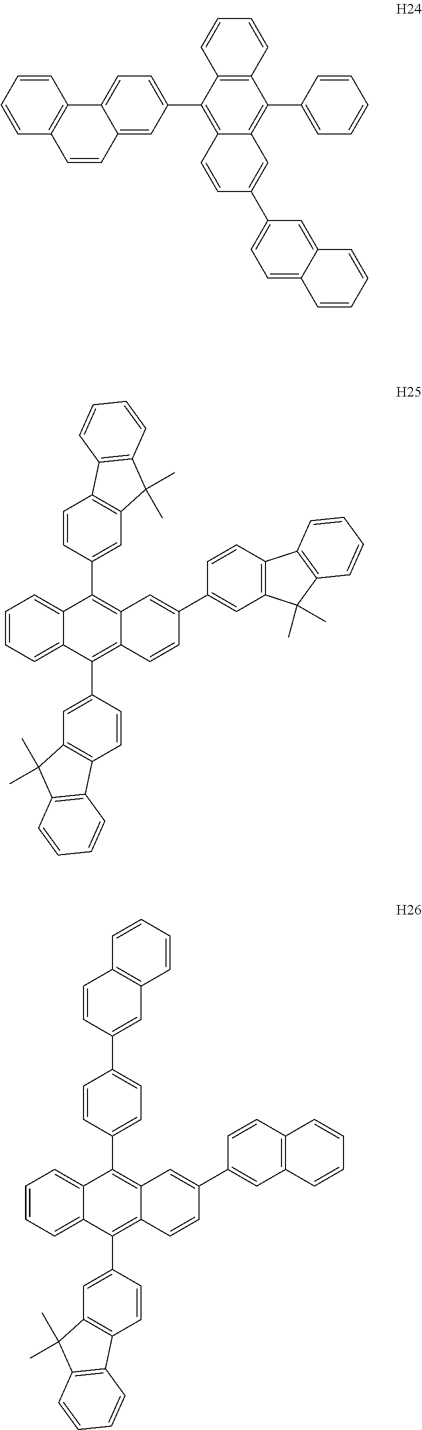

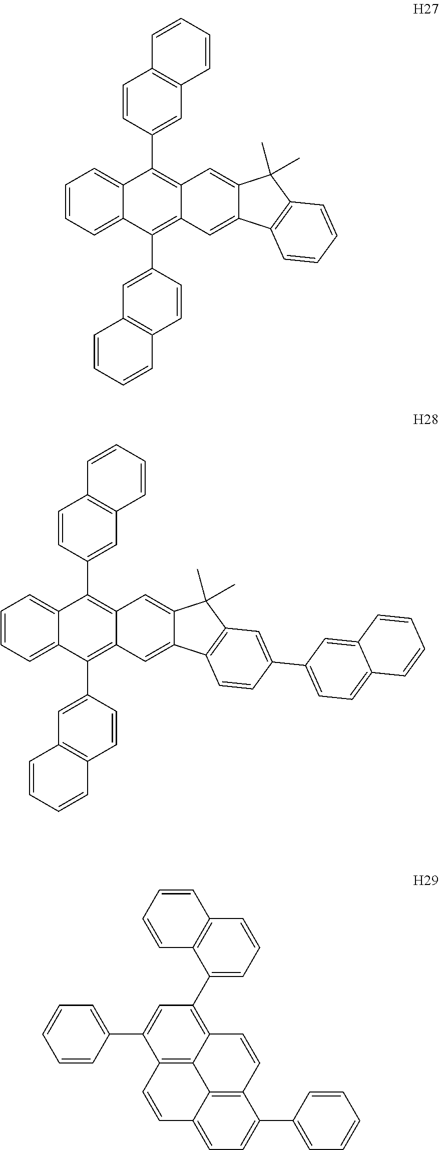

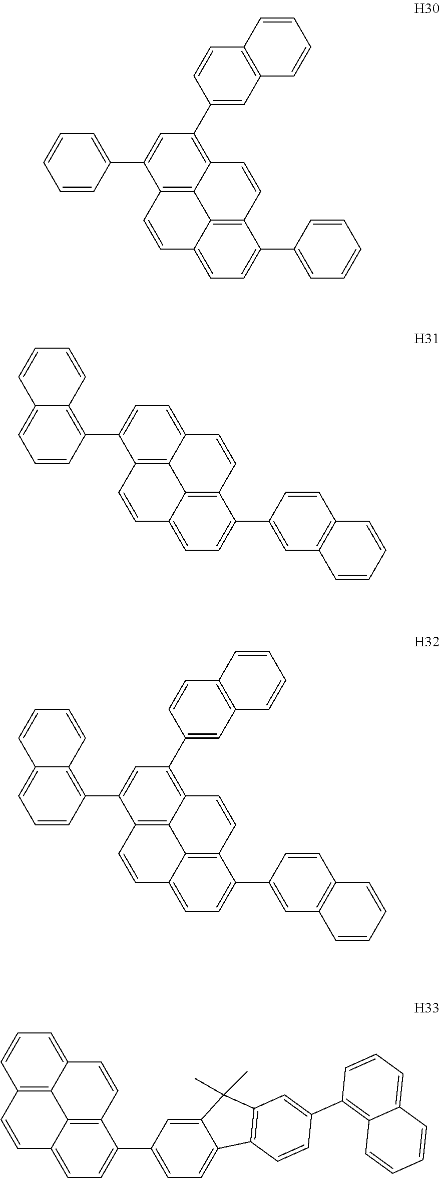

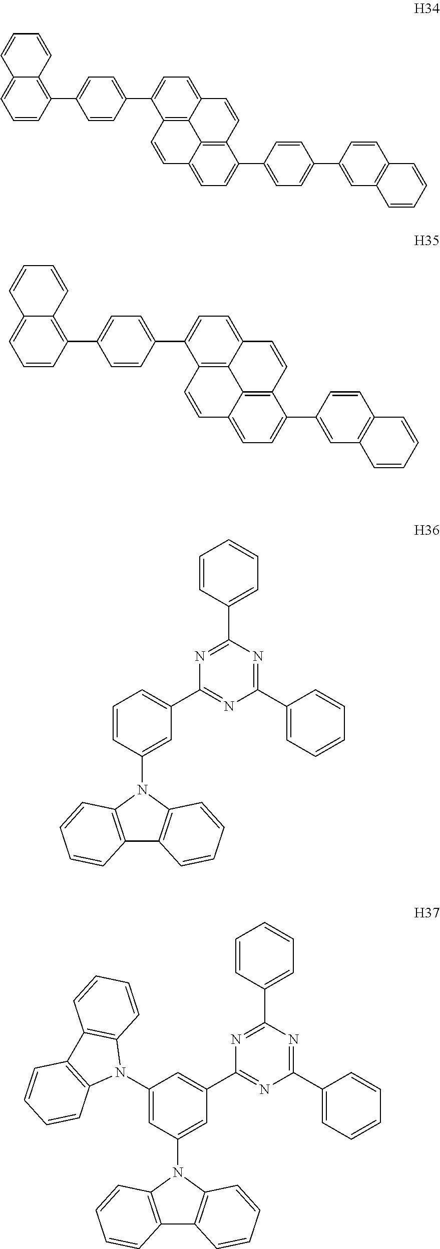

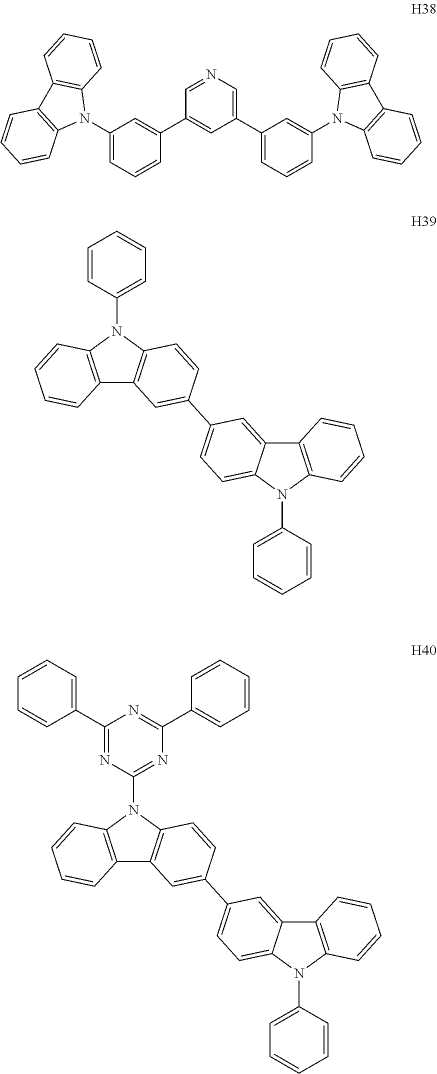 [SMILES: c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

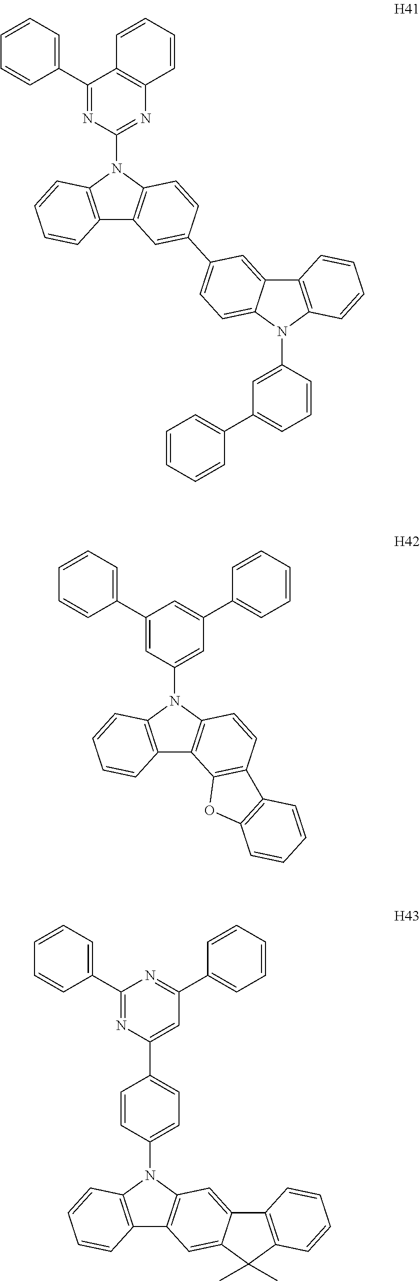 [SMILES: c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1]

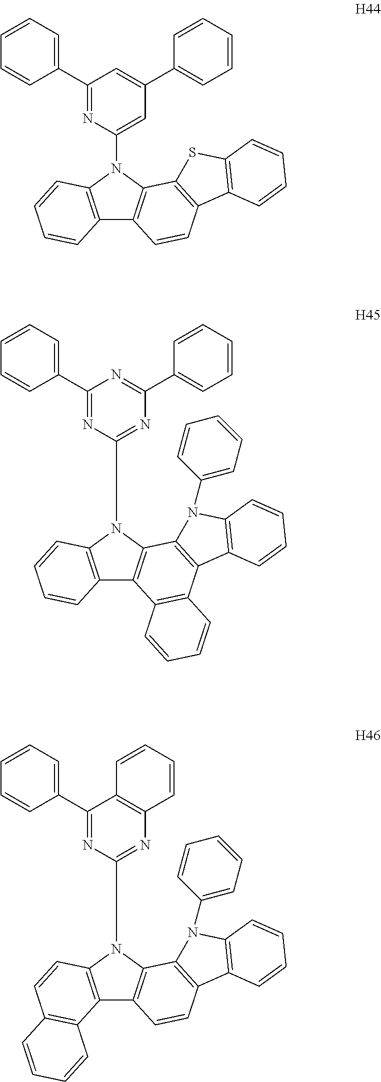 [SMILES: c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

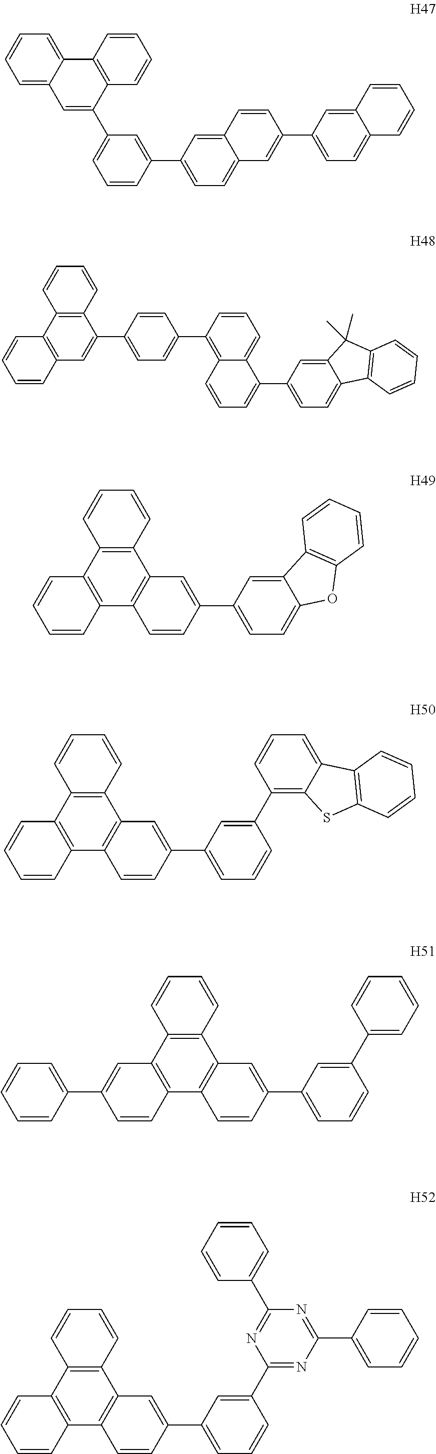

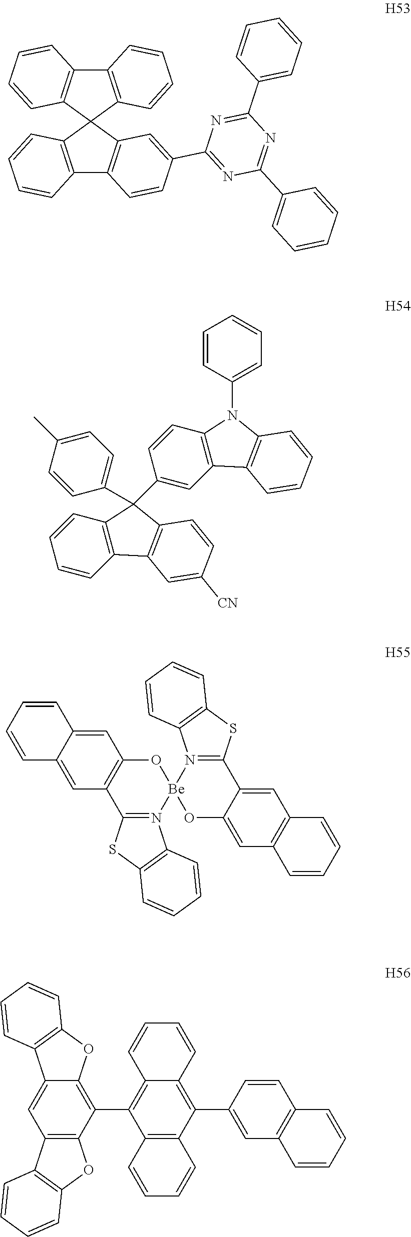

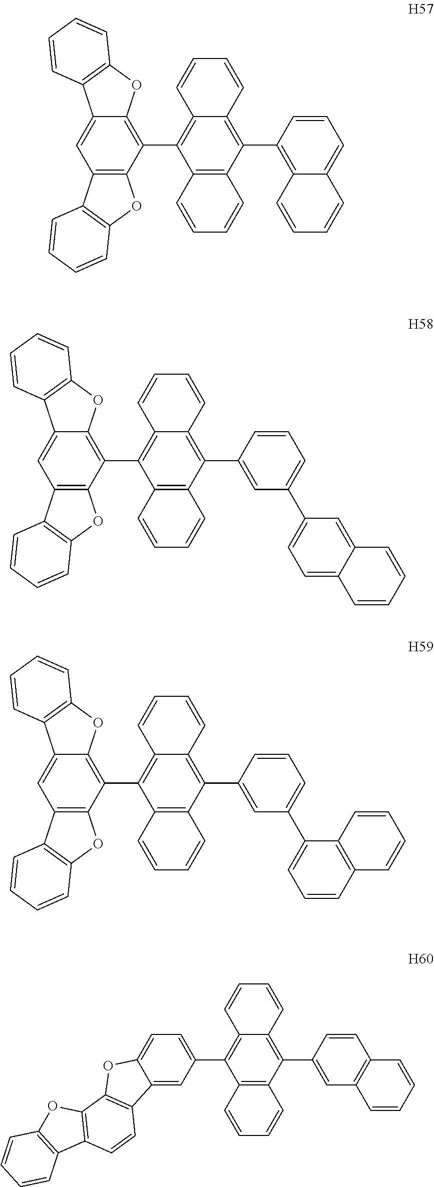 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1]

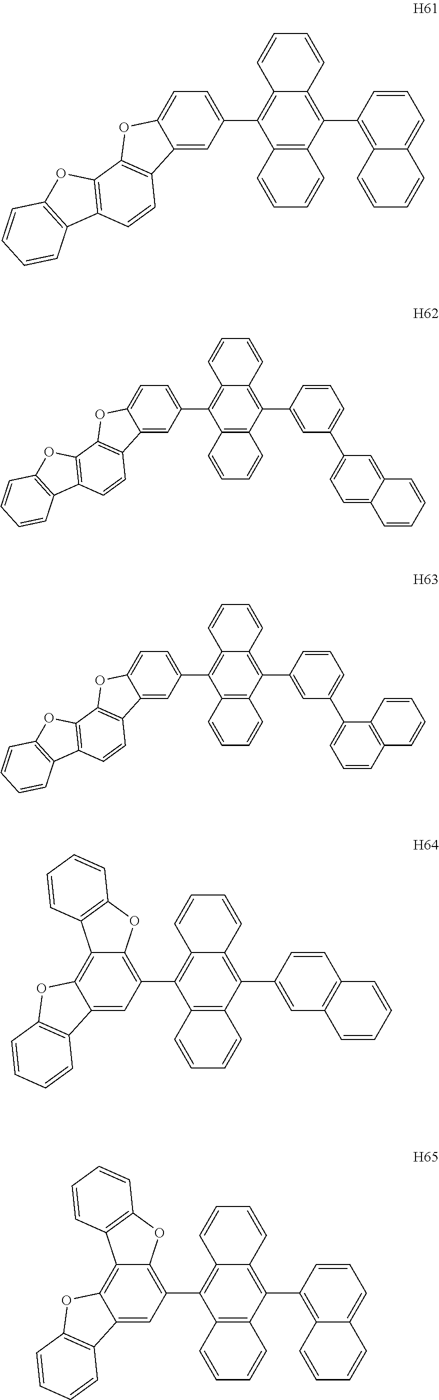 [SMILES: c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1]

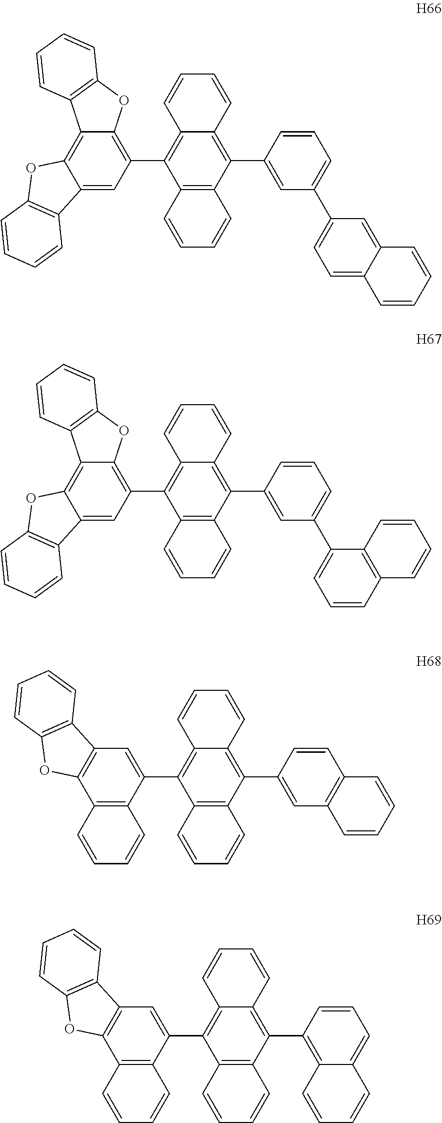 [SMILES: c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1]

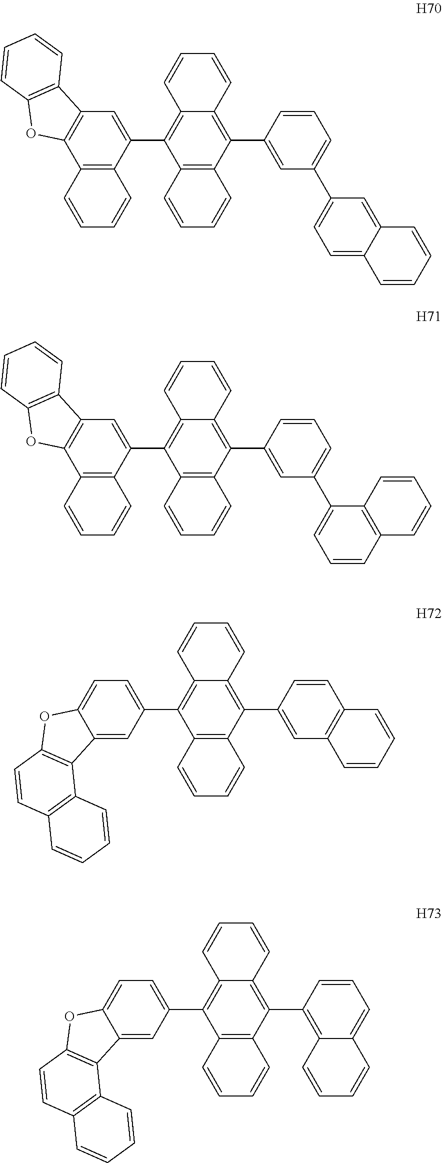 [SMILES: c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1]

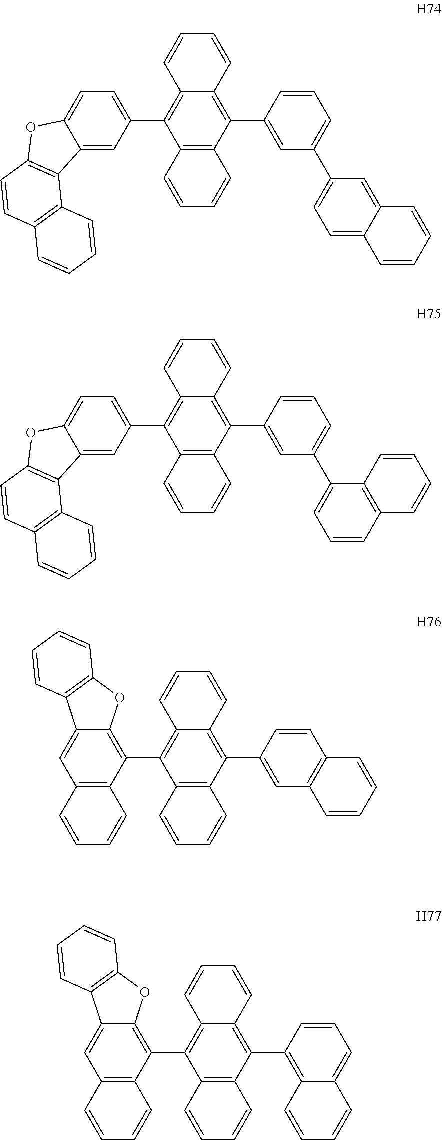 [SMILES: c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1]

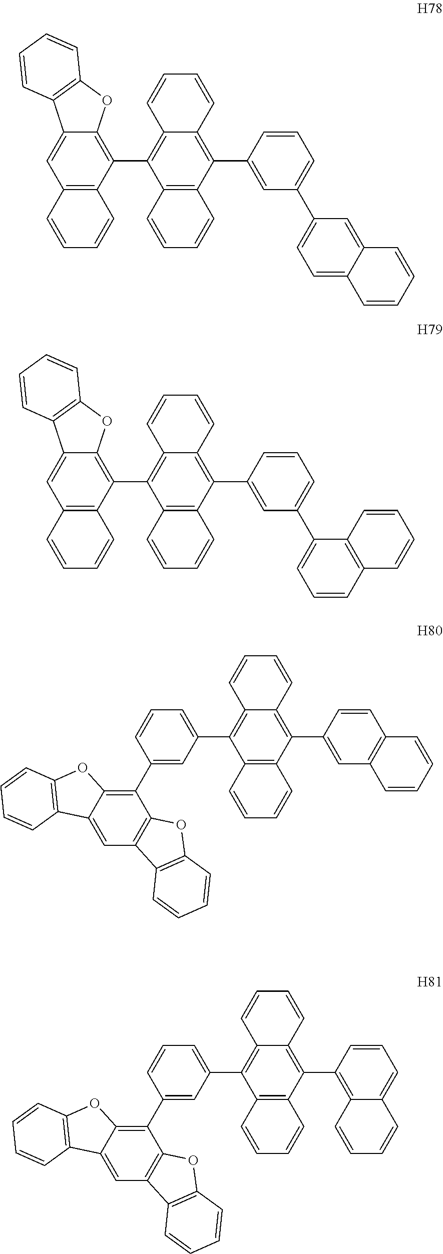 [SMILES: c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

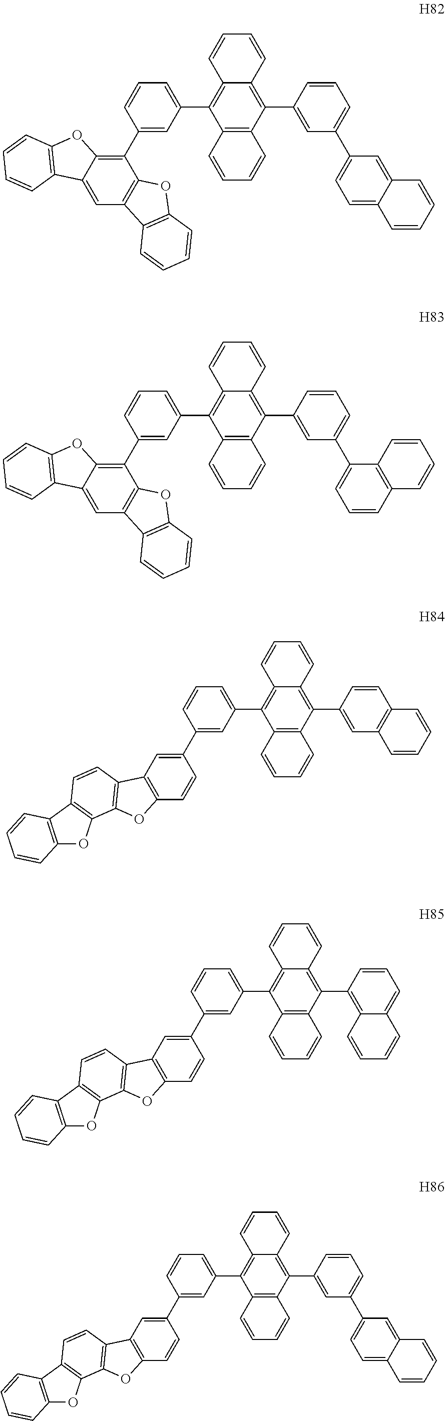 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1]

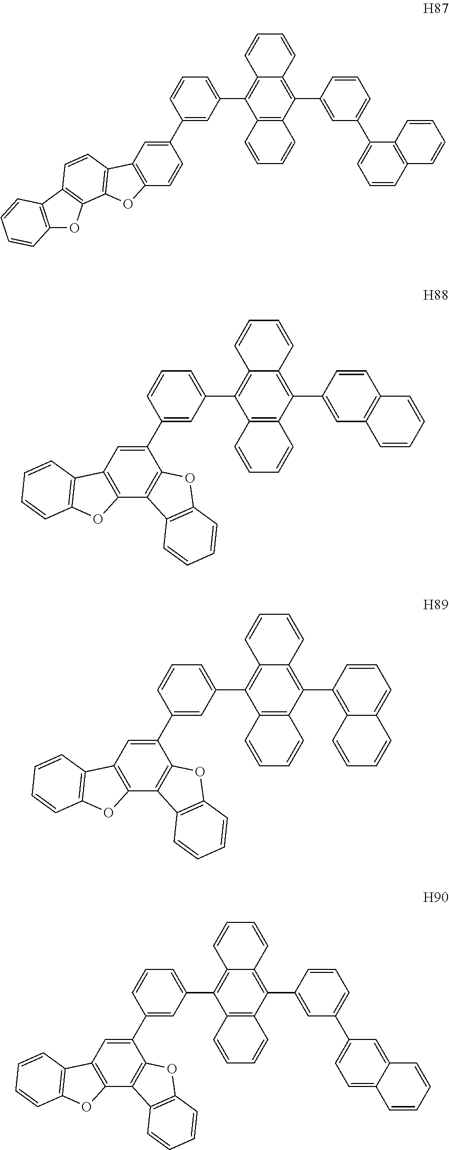 [SMILES: c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1]

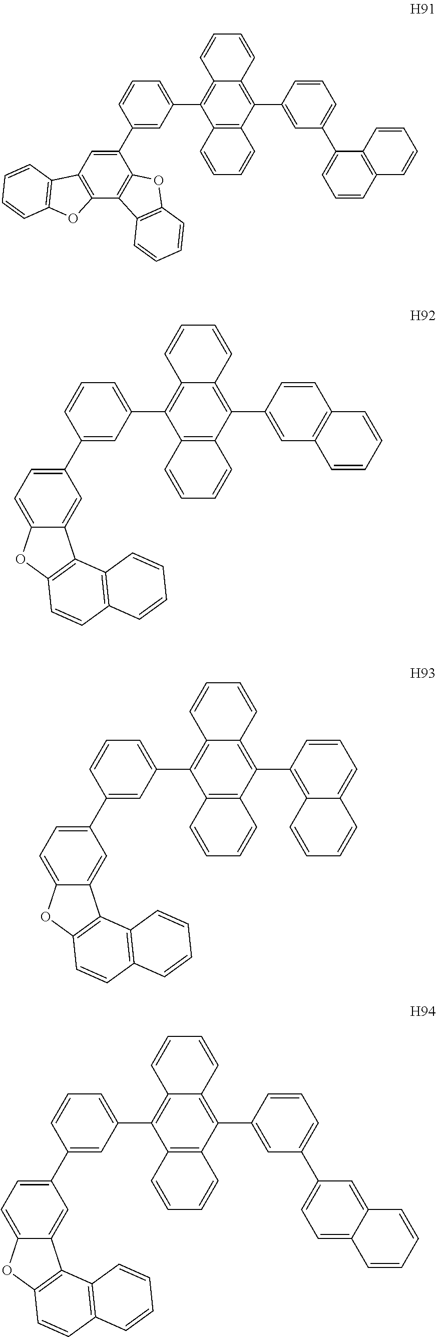 [SMILES: c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1]

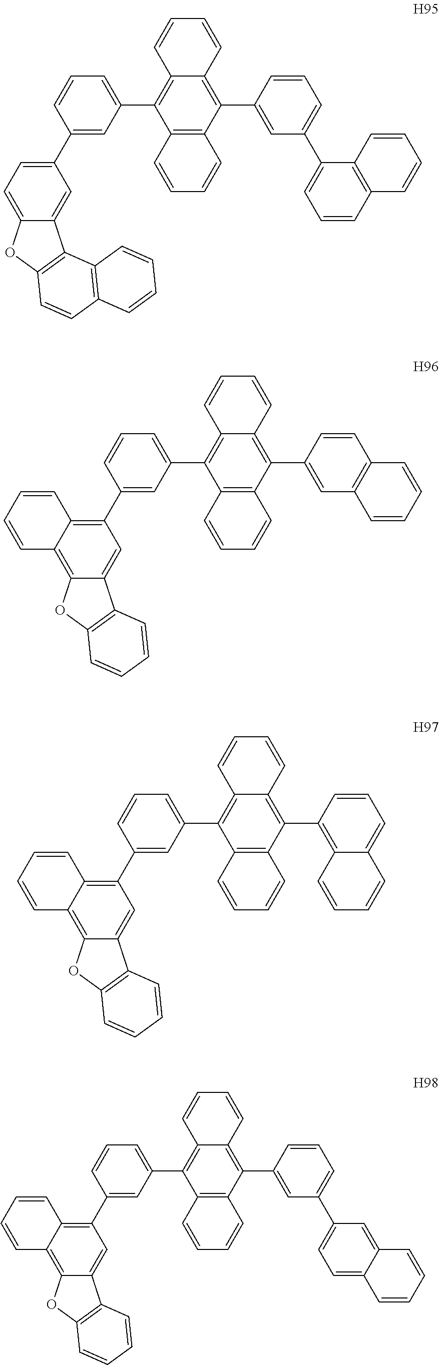 [SMILES: c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1]

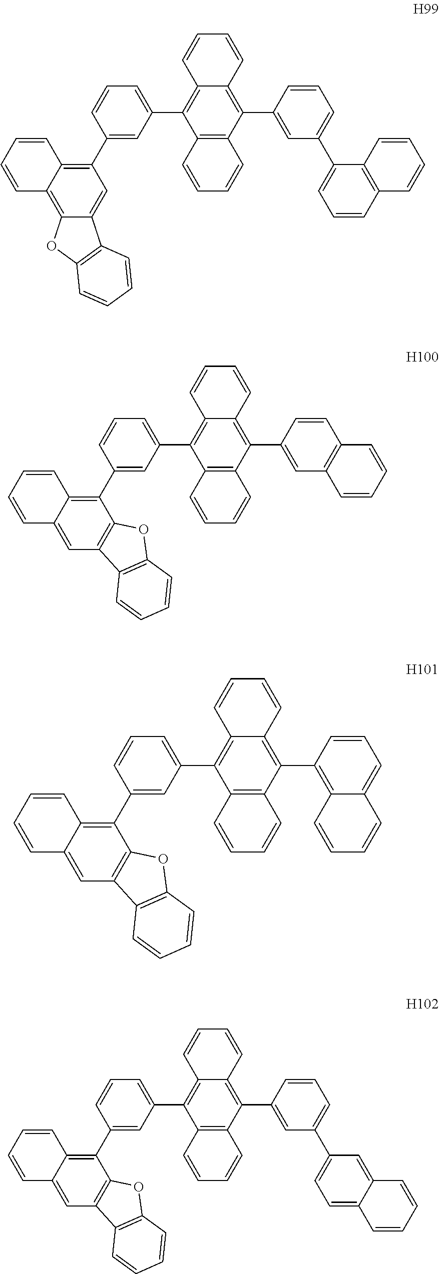 [SMILES: c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1]

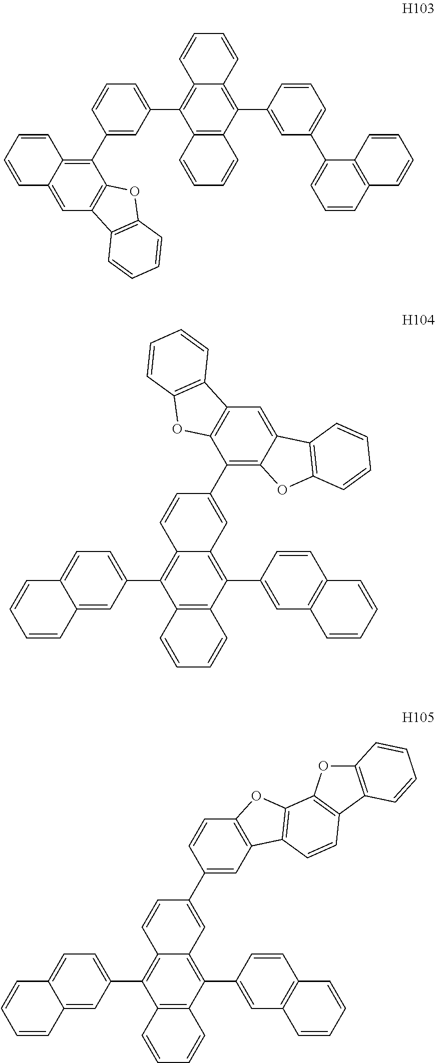 [SMILES: c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1]

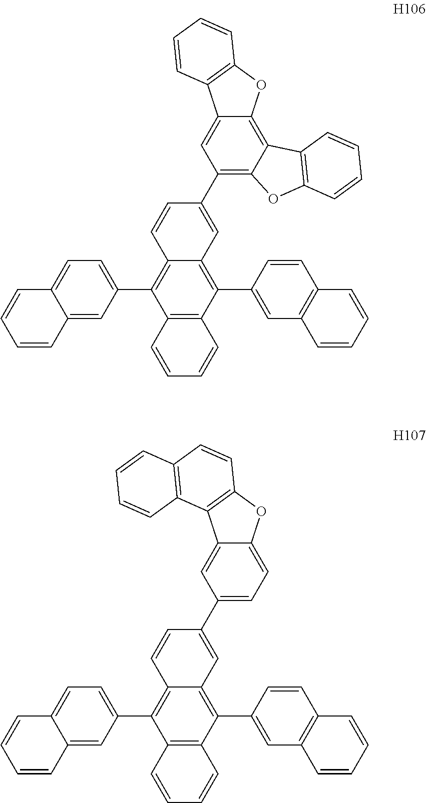 [SMILES: c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

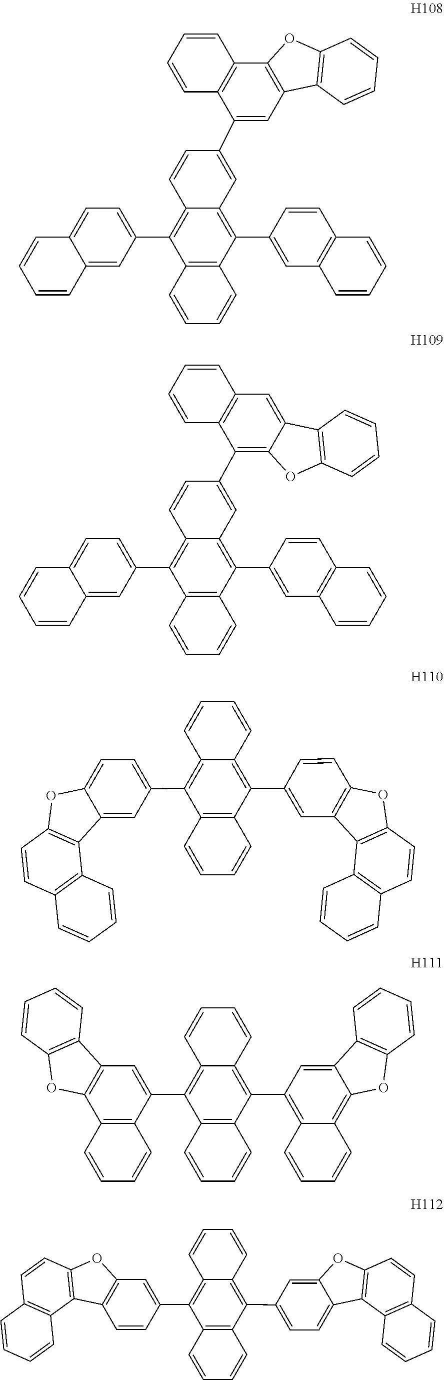

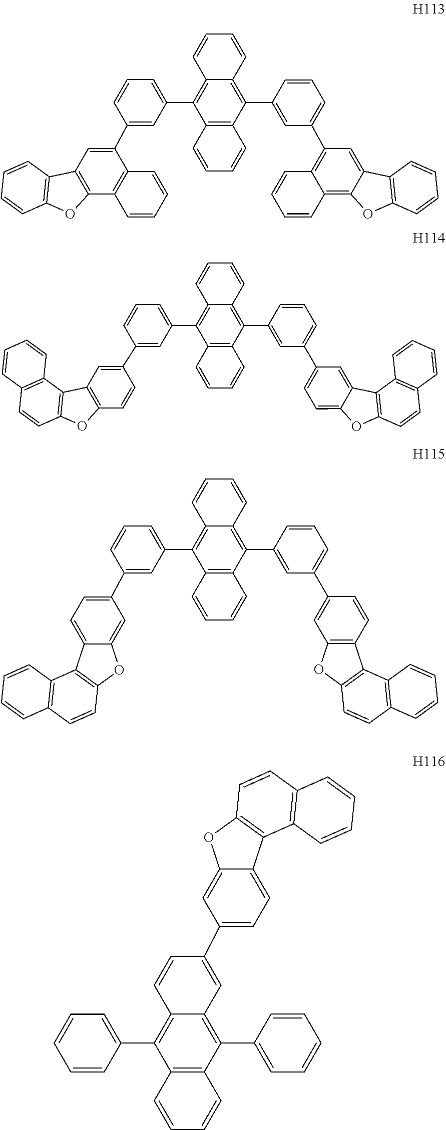 [SMILES: c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1]

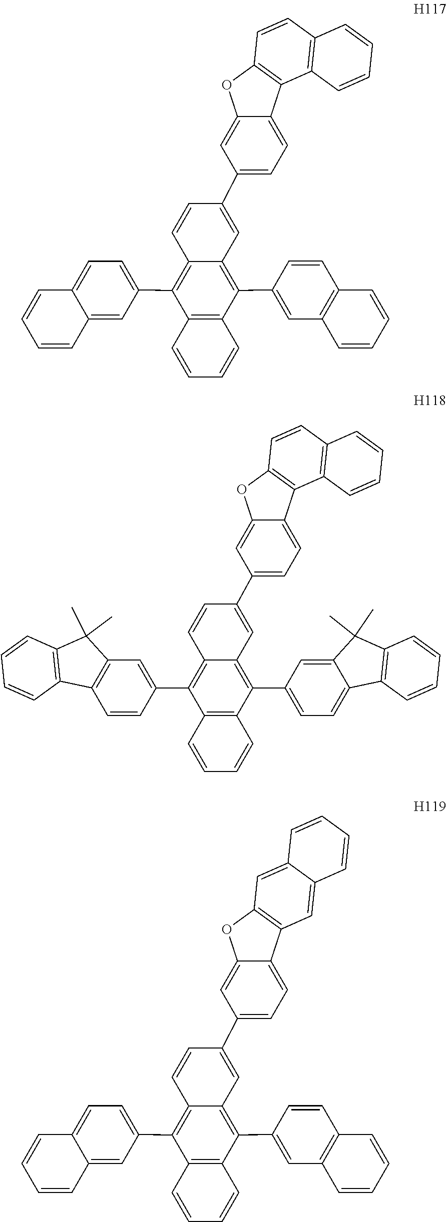 [SMILES: c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1]

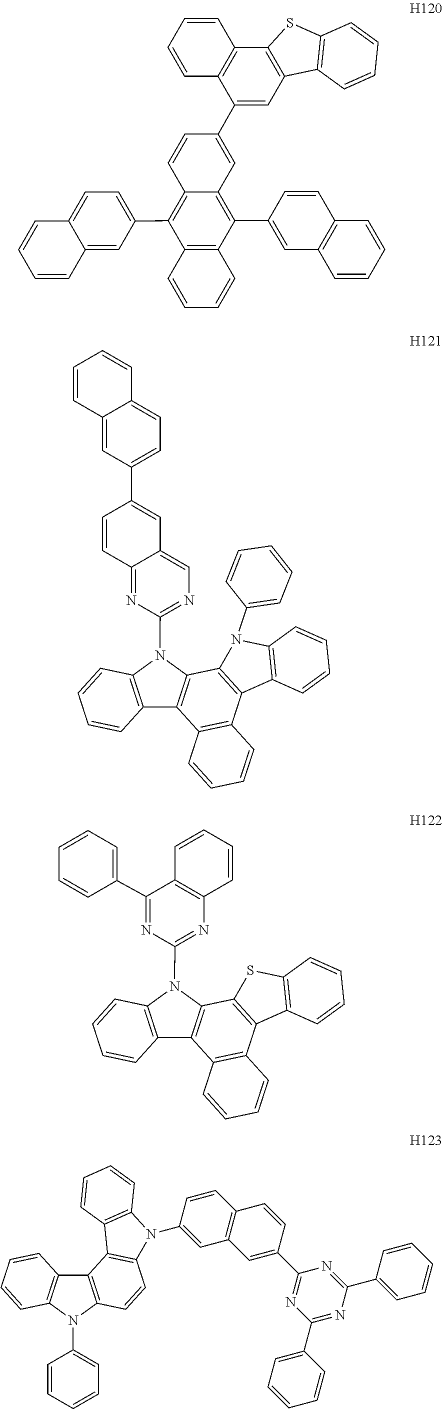

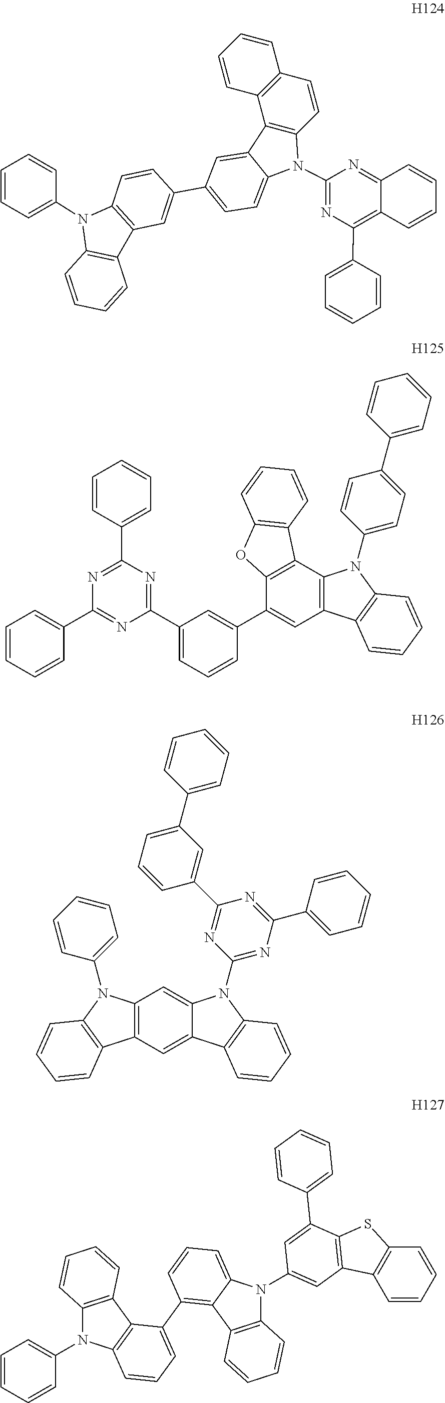 [SMILES: c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1]

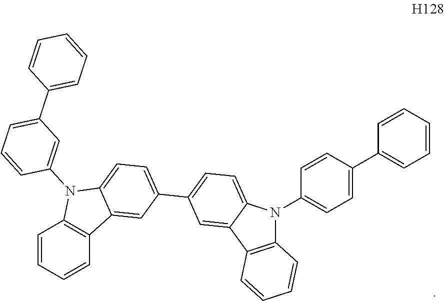 [SMILES: c1ccc(-c2ccc(-n3c4ccccc4c4cc(-c5ccc6c(c5)c5ccccc5n6-c5cccc(-c6ccccc6)c5)ccc43)cc2)cc1]

### [Phosphorescent Dopant]

In an embodiment, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In an embodiment, the phosphorescent dopant may include an organometallic compound represented by Formula 401:

M(L₄₀₁)_{xc1}(L₄₀₂)_{xc2} [Formula 401]

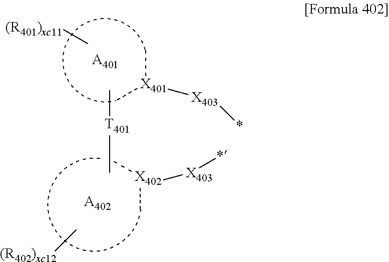

In Formulae 401 and 402,
M may be a transition metal (e.g., Ir, Pt, Pd, Os, Ti, Au, Hf, Eu, Tb, Rh, Re, or Tm),
L₄₀₁ may be a ligand represented by Formula 402, and xc1 may be 1, 2, or 3, wherein when xc1 is 2 or more, two or more of L₄₀₁ may be identical to or different from each other,
L₄₀₂ may be an organic ligand, and xc2 may be 0, 1, 2, 3, or 4, wherein when xc2 is 2 or more, two or more of L₄₀₂ may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C═*′,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each independently be the same as described in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each independently be the same as described in connection with Q₁,
xc11 and xc12 may each independently be an integer from 0 to 10, and
* and *′ in Formula 402 each indicate a binding site to M in Formula 401.

In an embodiment, in Formula 402, X₄₀₁ may be nitrogen, and X₄₀₂ may be carbon, or X₄₀₁ and X₄₀₂ may each be nitrogen.

In an embodiment, in Formula 401, when xc1 is 2 or more, two ring A₄₀₁ among two or more of L₄₀₁ may be optionally linked together via T₄₀₂, which is a linking group, and two ring A₄₀₂ may be optionally linked together via T₄₀₃, which is a linking group (see Compounds PD1 to PD4 and PD7). T₄₀₂ and T₄₀₃ may each independently be the same as described in connection with T₄₀₁.

In Formula 401, L₄₀₂ may be an organic ligand. In an embodiment, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, etc.), or any combination thereof.

In an embodiment, the phosphorescent dopant may include, for example, one of Compounds PD1 to PD39, or any combination thereof:

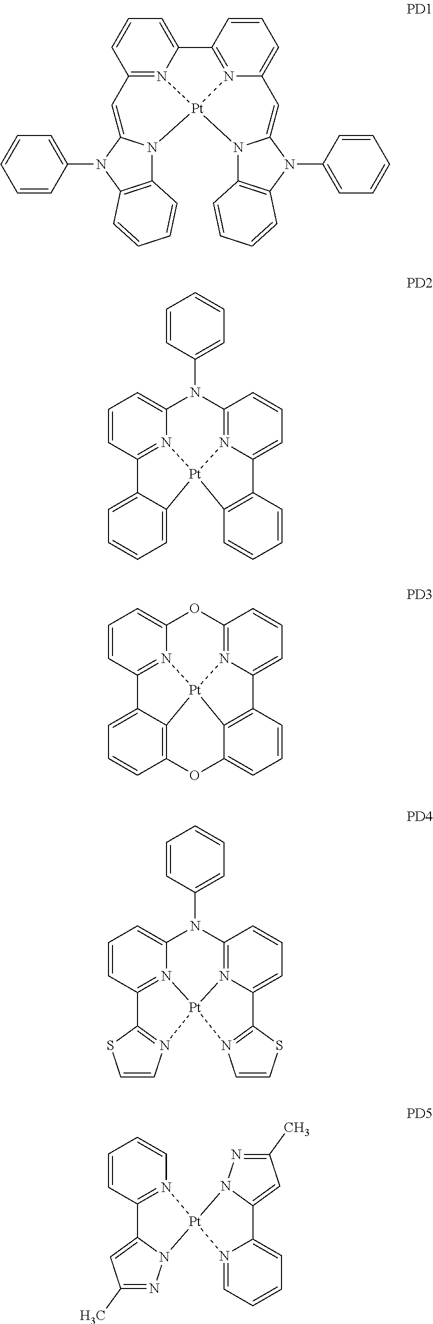

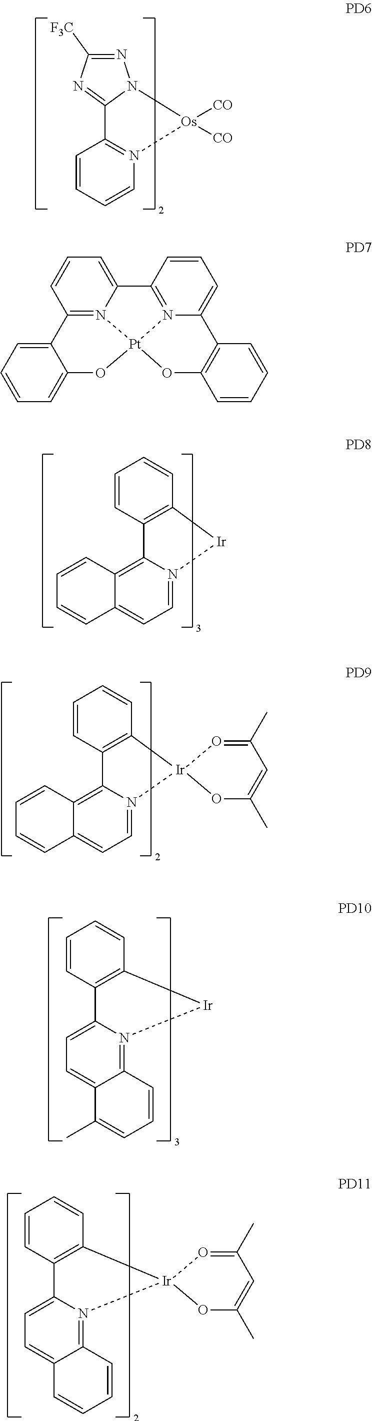

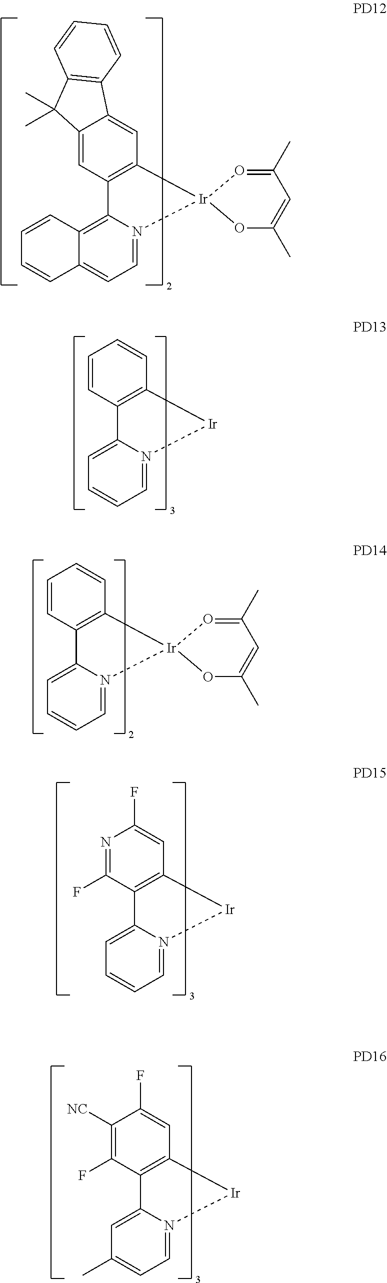

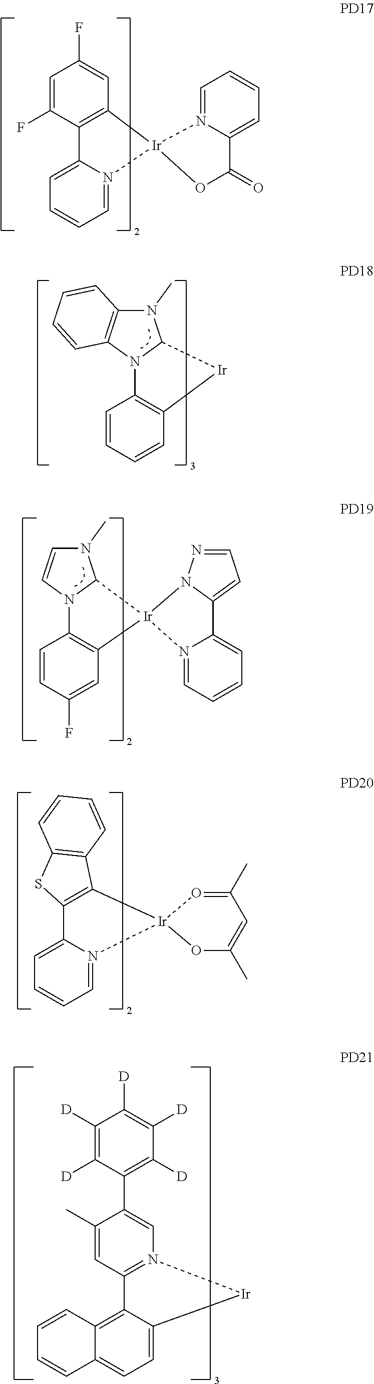

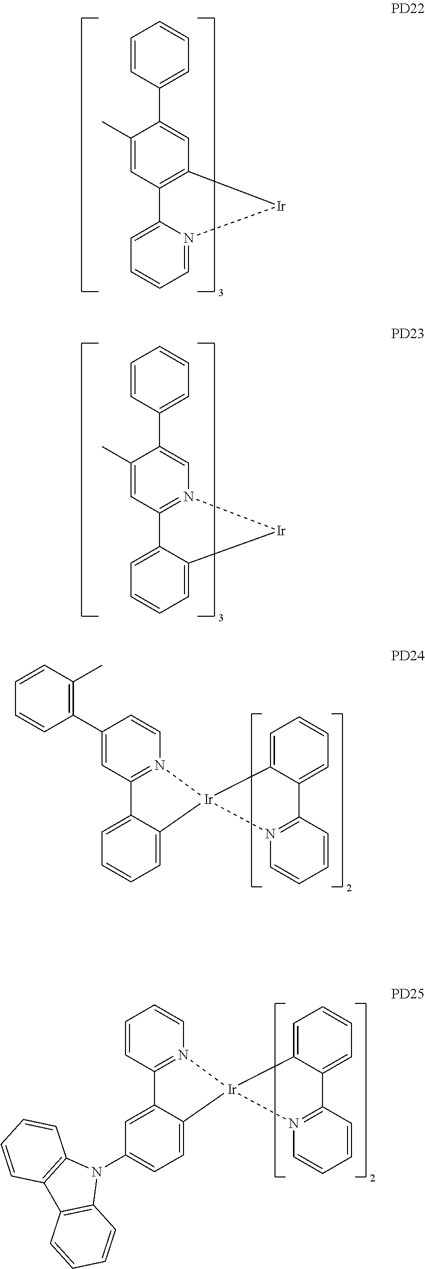

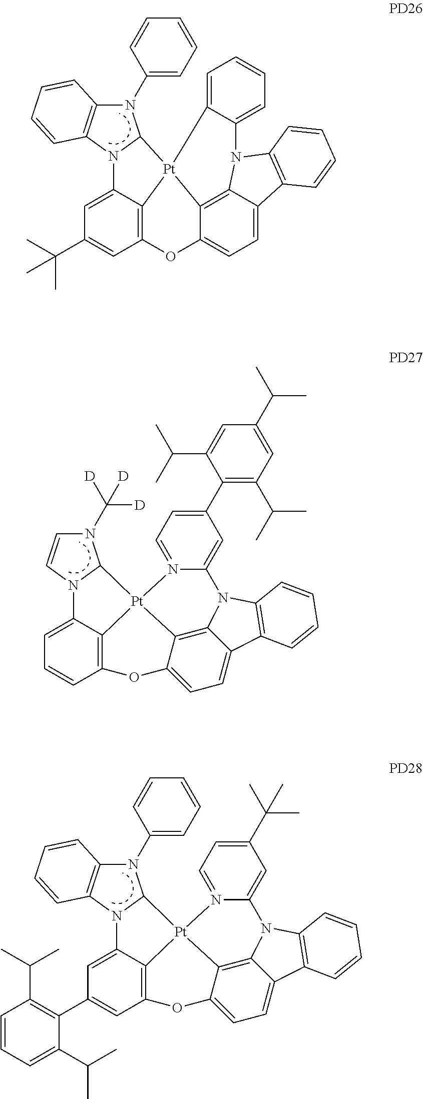

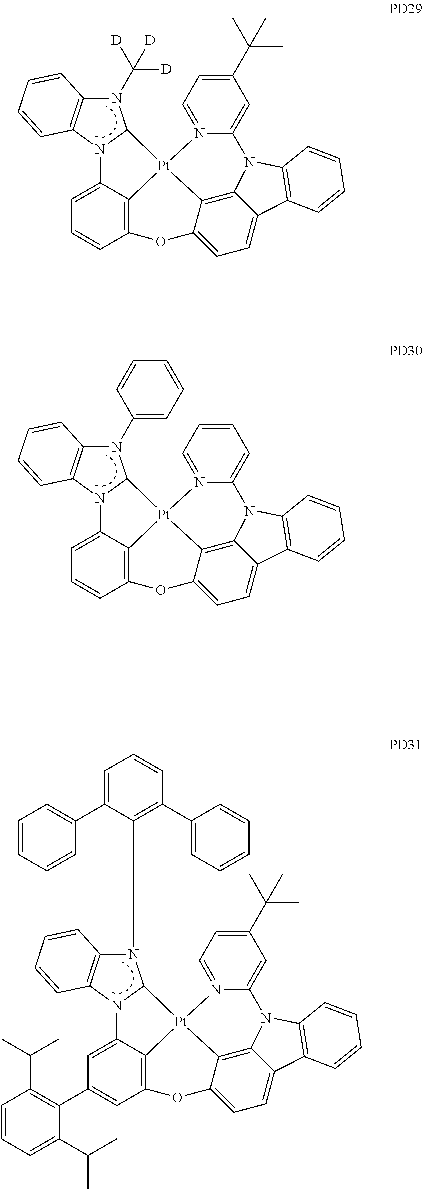

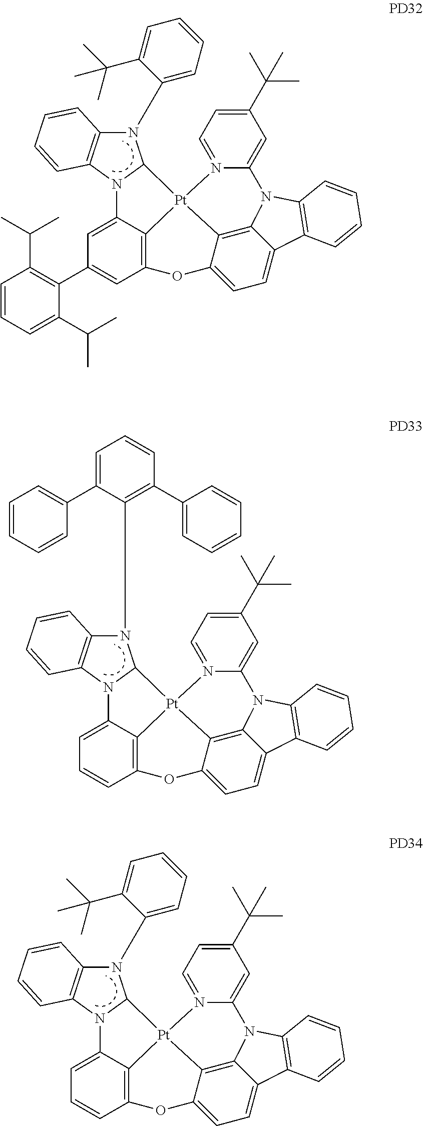

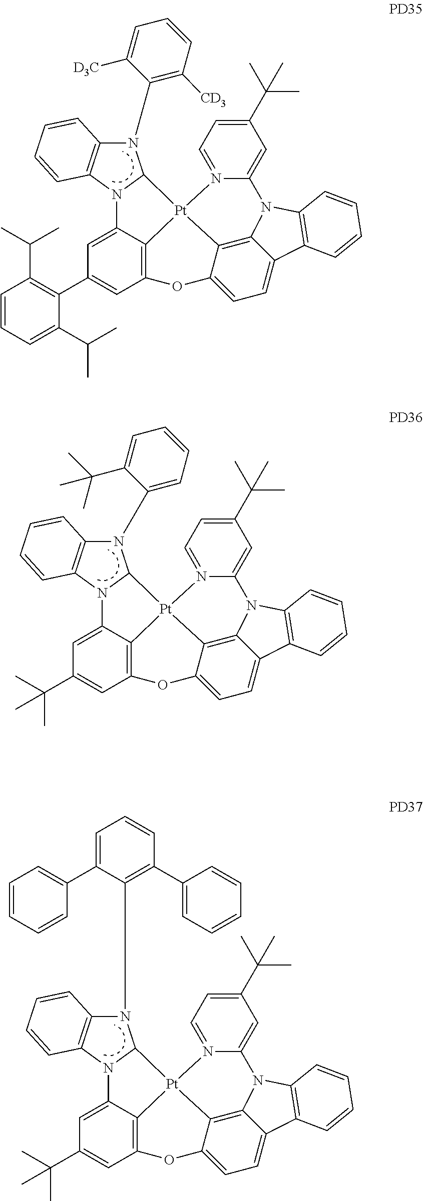

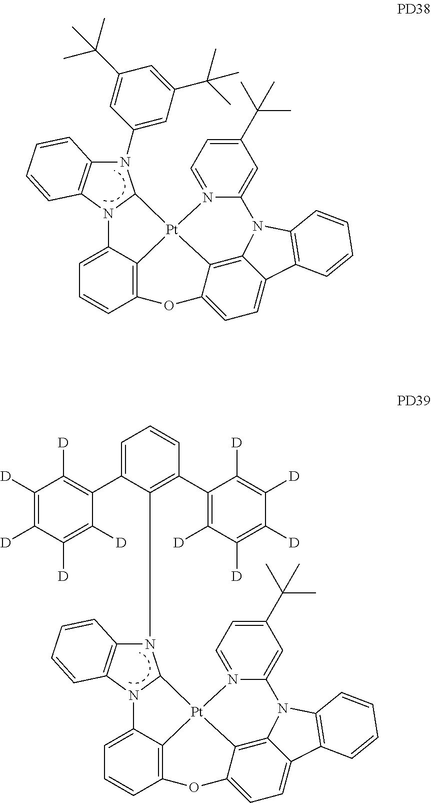

### [Fluorescent Dopant]

The fluorescent dopant may include an amine group-containing compound, a styryl group-containing compound, or any combination thereof.

In an embodiment, the fluorescent dopant may include a compound represented by Formula 501:

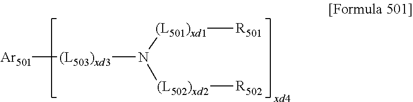

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In an embodiment, in Formula 501, Ar₅₀₁ may be a condensed cyclic group (for example, an anthracene group, a chrysene group, a pyrene group, etc.) in which three or more monocyclic groups are condensed together.

In an embodiment, xd4 in Formula 501 may be 2.

In an embodiment, the fluorescent dopant may include one of Compounds FD1 to FD37, DPVBi, DPAVBi, or any combination thereof:

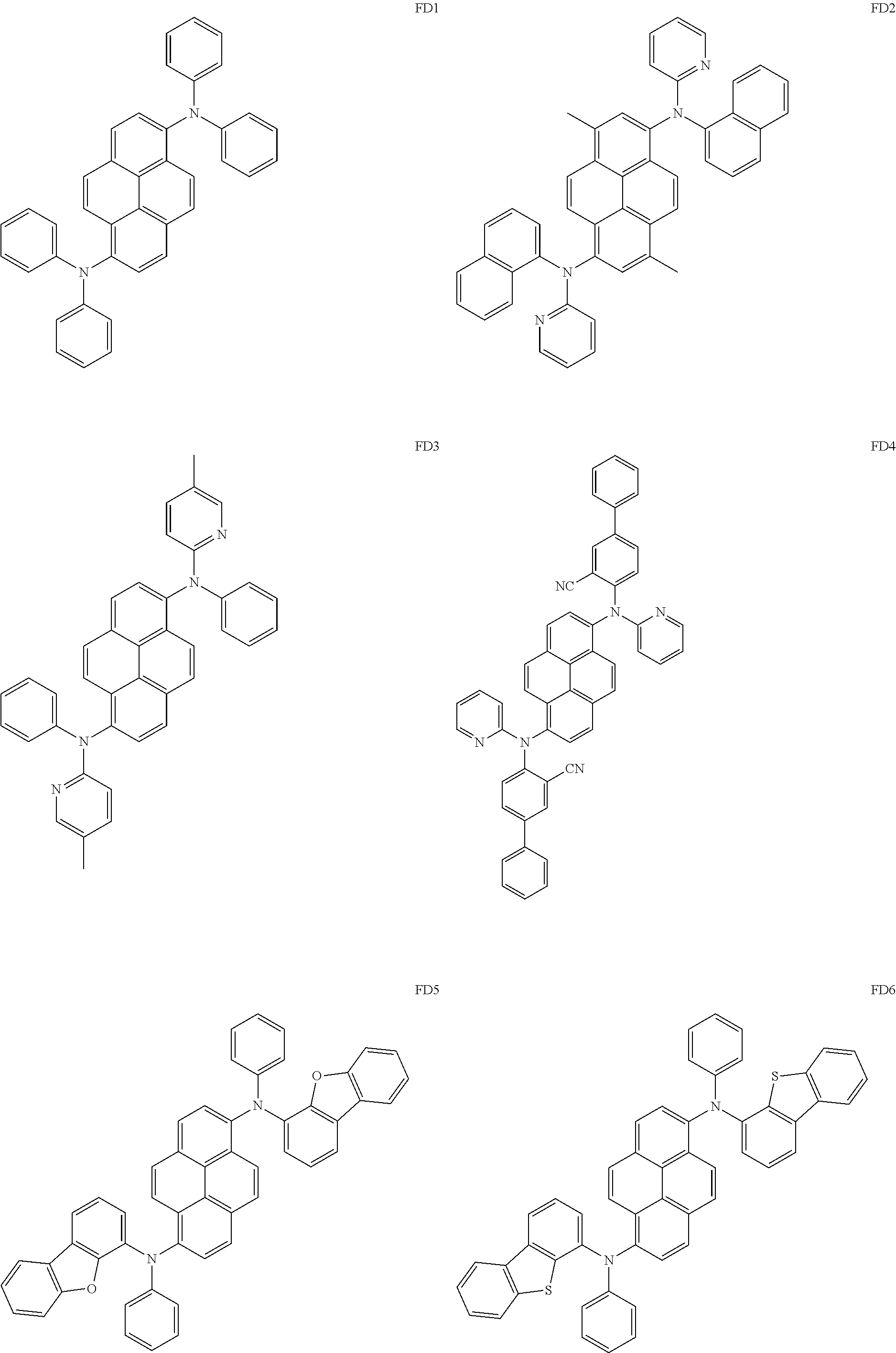

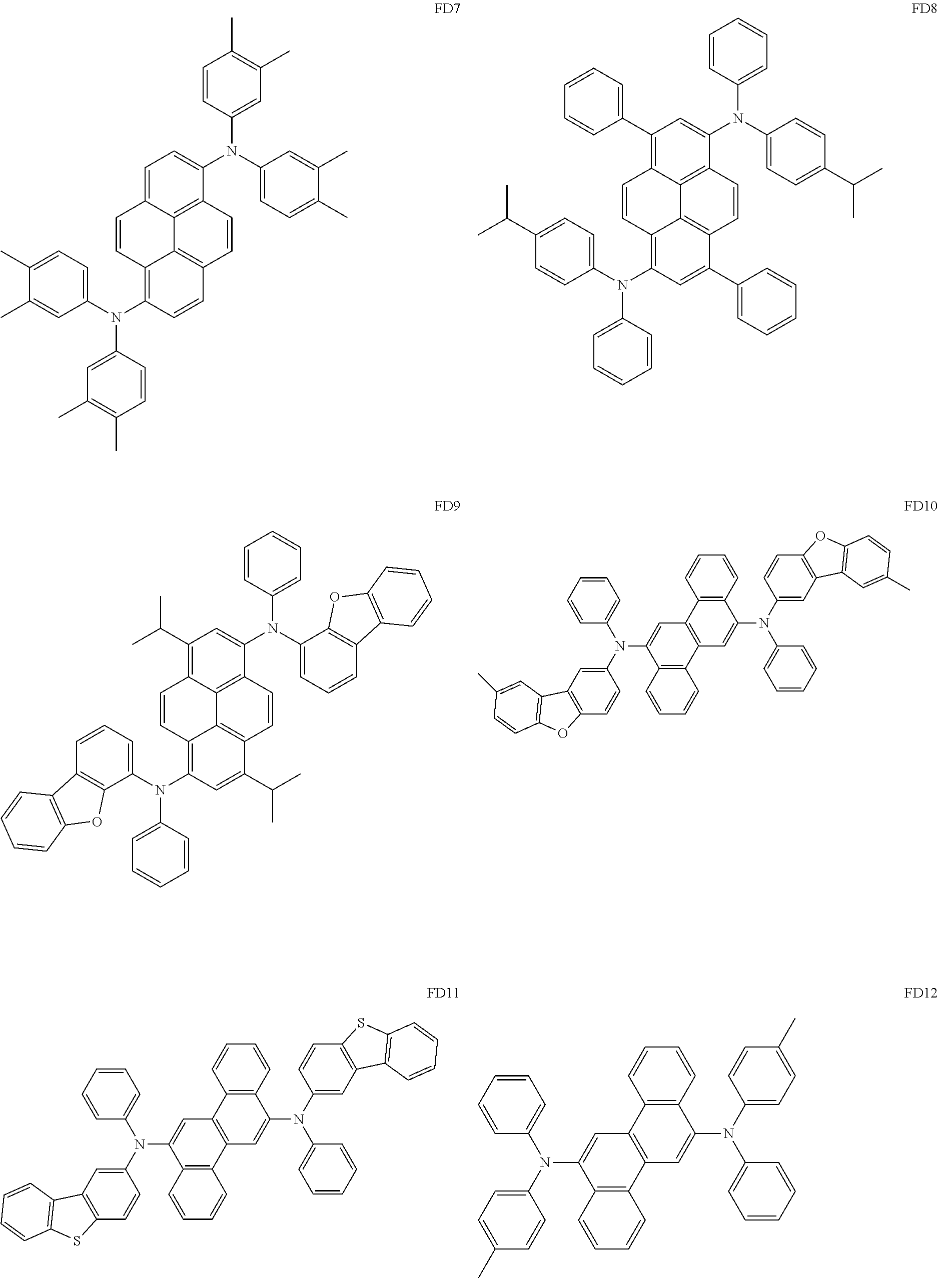

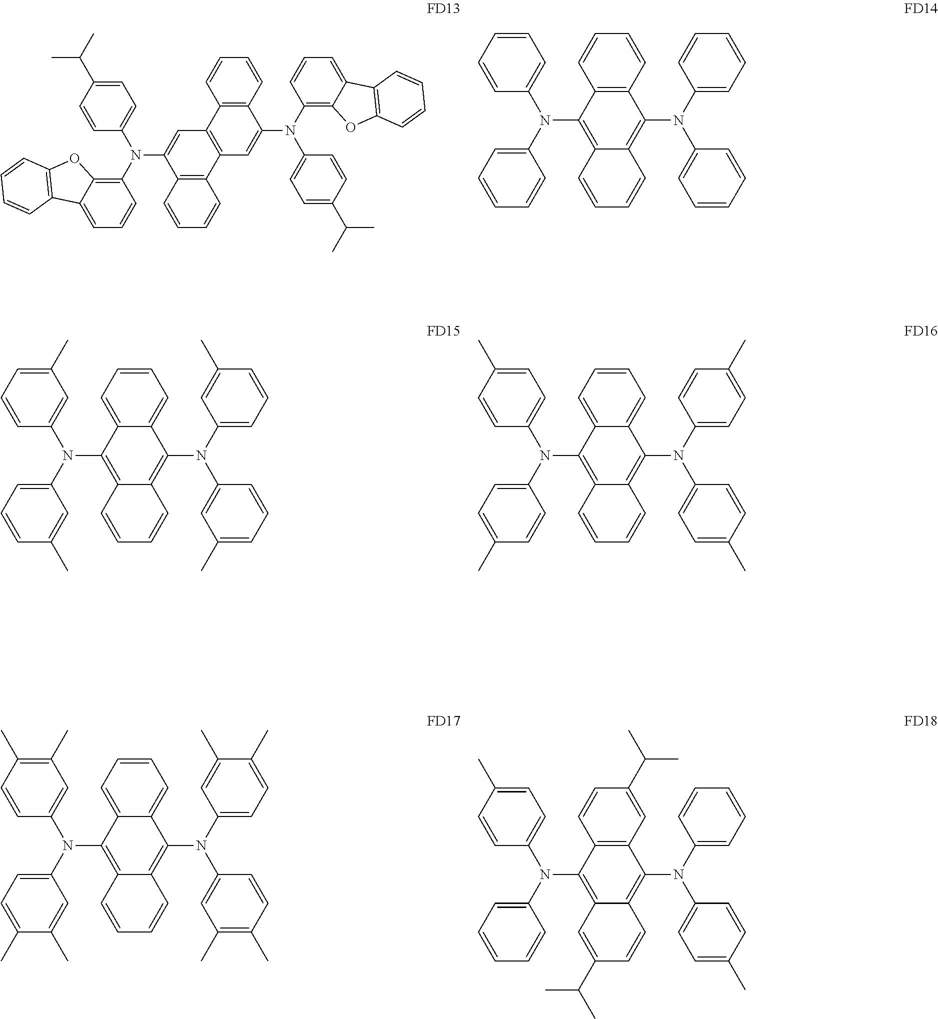 [SMILES: CC(C)c1ccc(N(c2cc3c4ccccc4c(N(c4ccc(C(C)C)cc4)c4cccc5c4oc4ccccc45)cc3c3ccccc23)c2cccc3c2oc2ccccc23)cc1 ; c1ccc(N(c2ccccc2)c2c3ccccc3c(N(c3ccccc3)c3ccccc3)c3ccccc23)cc1 ; Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

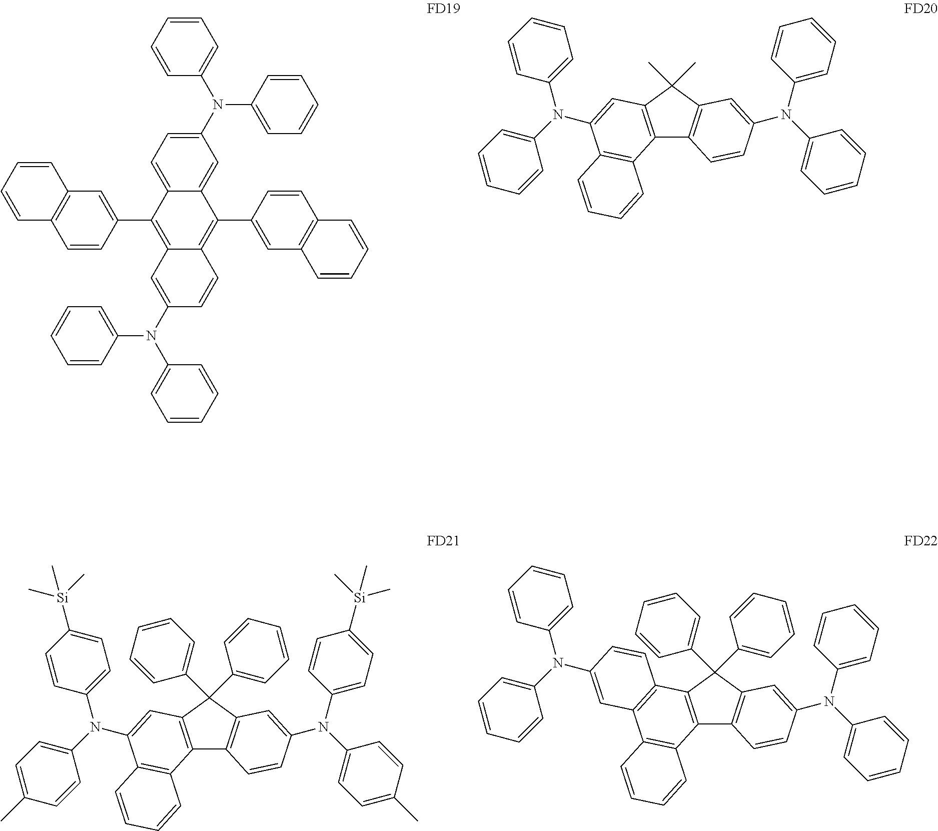

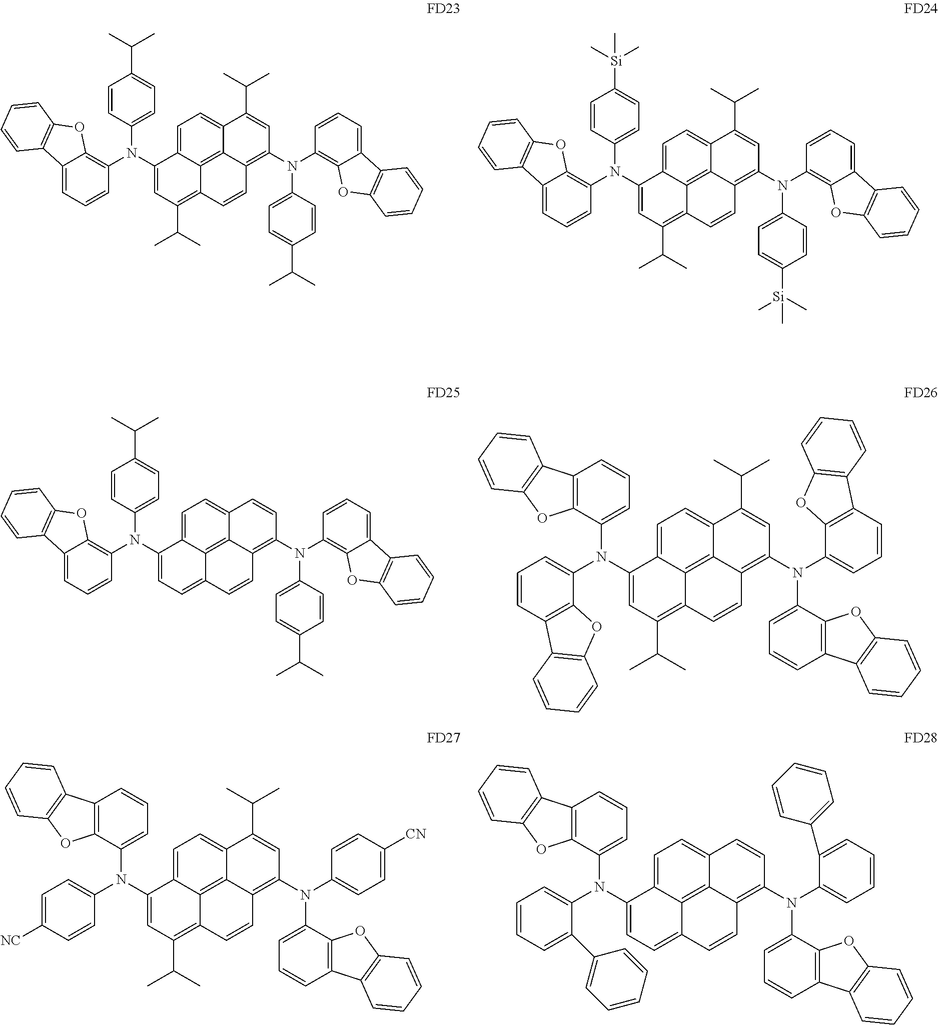

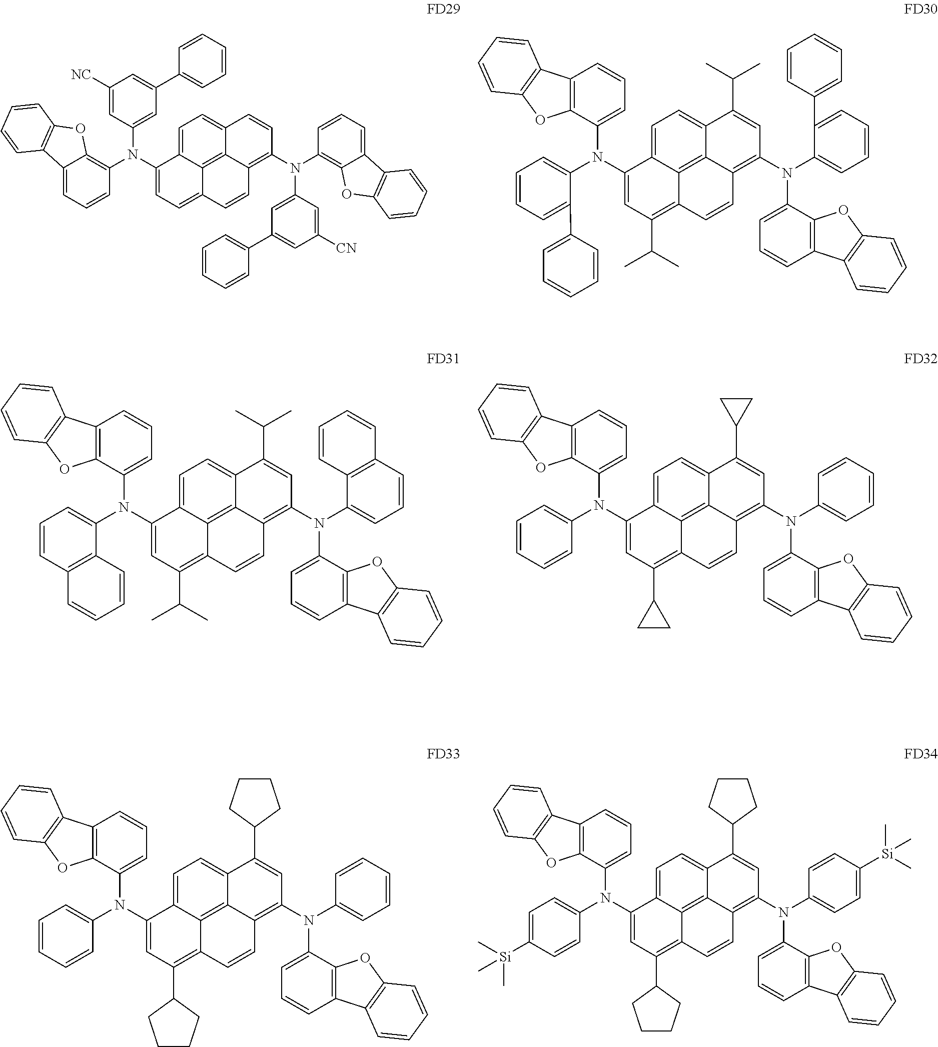

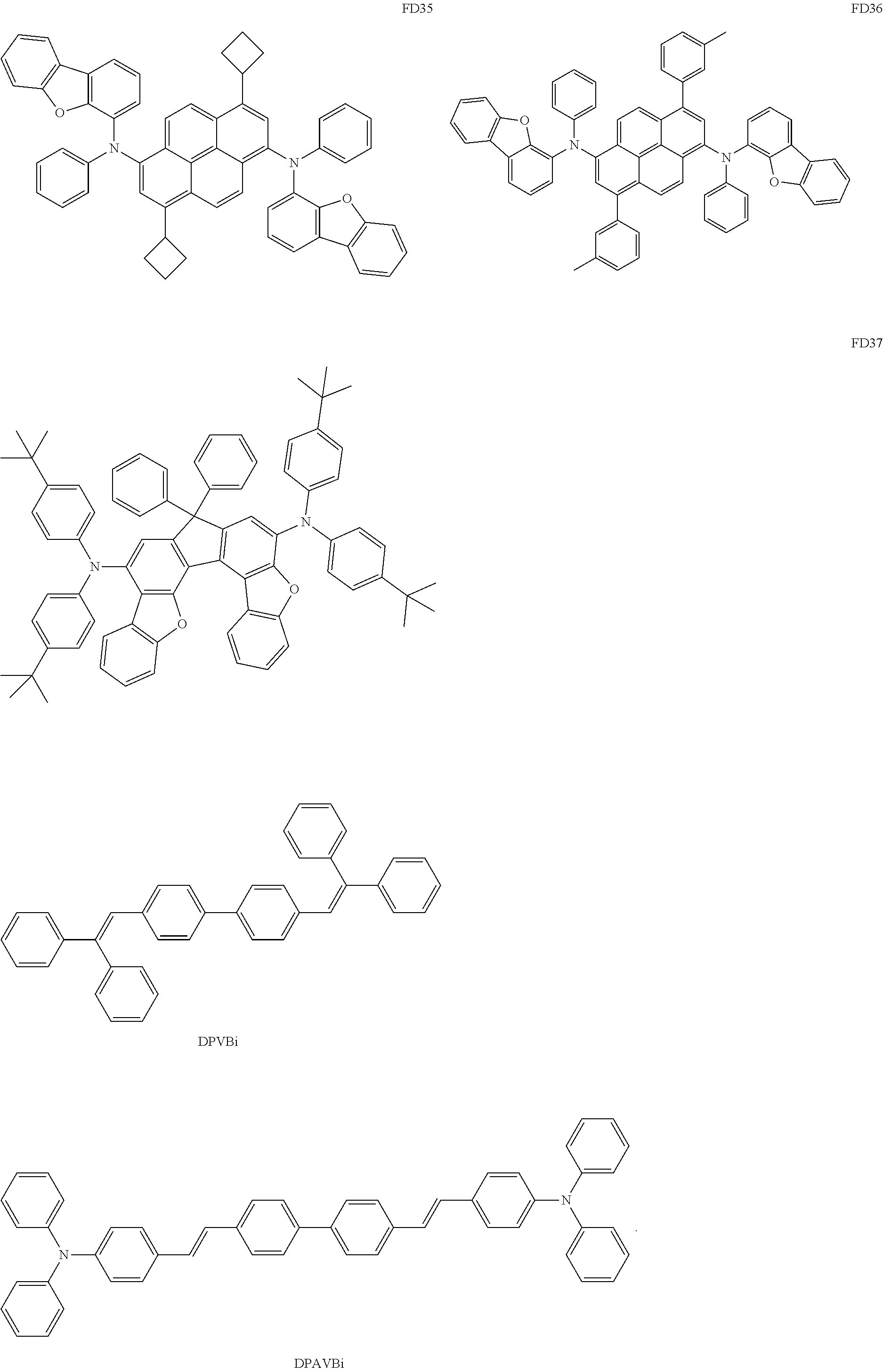

### [Delayed Fluorescence Material]

In an embodiment, the emission layer 130 may include a delayed fluorescence material.

In the specification, a delayed fluorescence material may be selected from compounds capable of emitting delayed fluorescence, based on a delayed fluorescence emission mechanism.

The delayed fluorescence material included in the emission layer 130 may serve as a host or as a dopant depending on the types of other materials included in the emission layer 130.

In an embodiment, a difference between a triplet energy level (eV) of the delayed fluorescence material and the singlet energy level (eV) of the delayed fluorescence material may be in a range of about 0 eV to about 0.5 eV. When the difference between the triplet energy level (eV) of the delayed fluorescence material and the singlet energy level (eV) of the delayed fluorescence material is satisfied within the range above, up-conversion from a triplet state to a singlet state of the delayed fluorescence materials may effectively occur, and thus, the light-emitting device 10 may have improved luminescence efficiency.

In an embodiment, the delayed fluorescence material may include: a material including at least one electron donor (for example, a π electron-rich C₃-C₆₀ cyclic group such as a carbazole group) and at least one electron acceptor (for example, a sulfoxide group, a cyano group, a π electron-deficient nitrogen-containing C₁-C₆₀ cyclic group, and the like); or a material including a C₈-C₆₀ polycyclic group including at least two cyclic groups that are condensed with each other while sharing boron (B).

In an embodiment, the delayed fluorescence material may include, for example, at least one of Compounds DF1 to DF14:

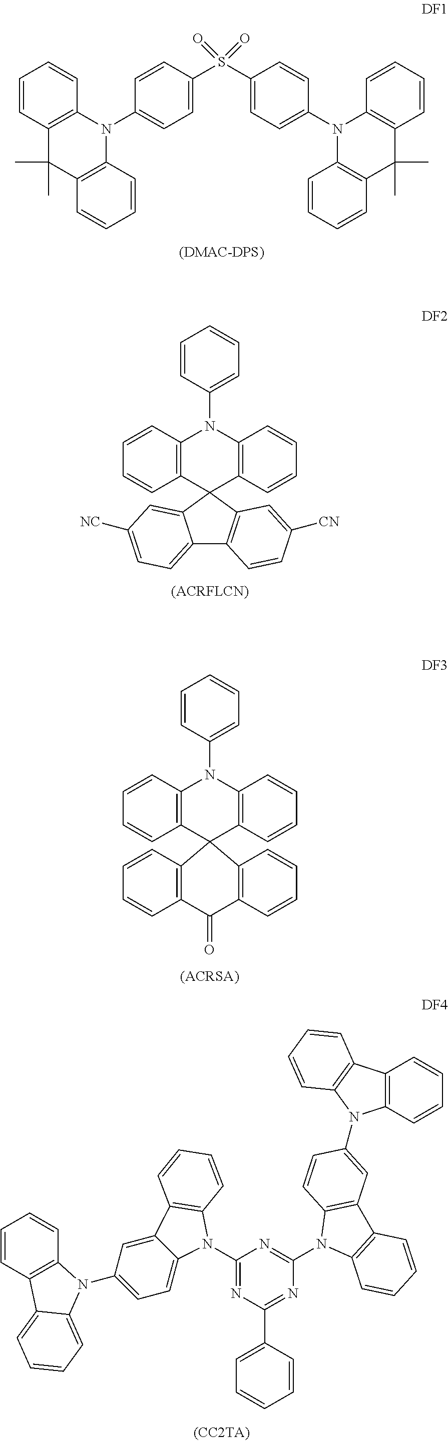

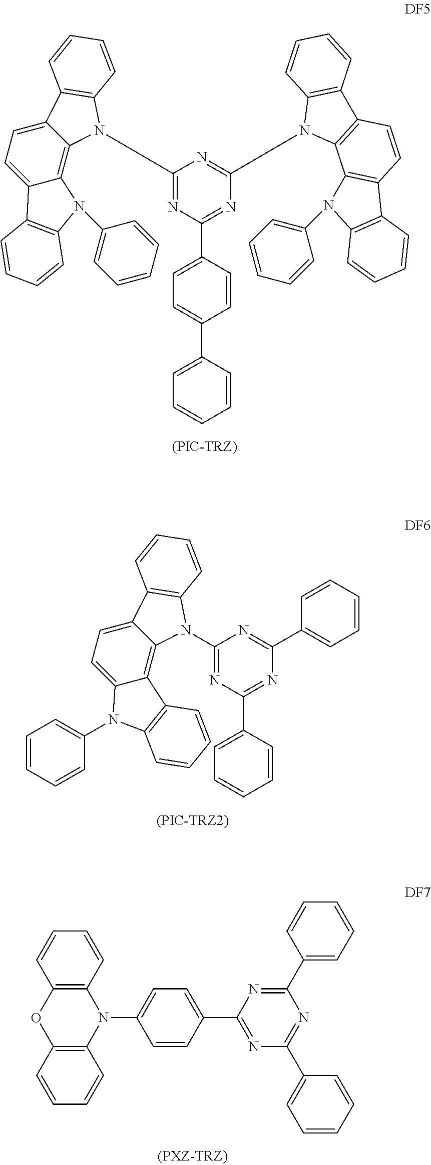 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

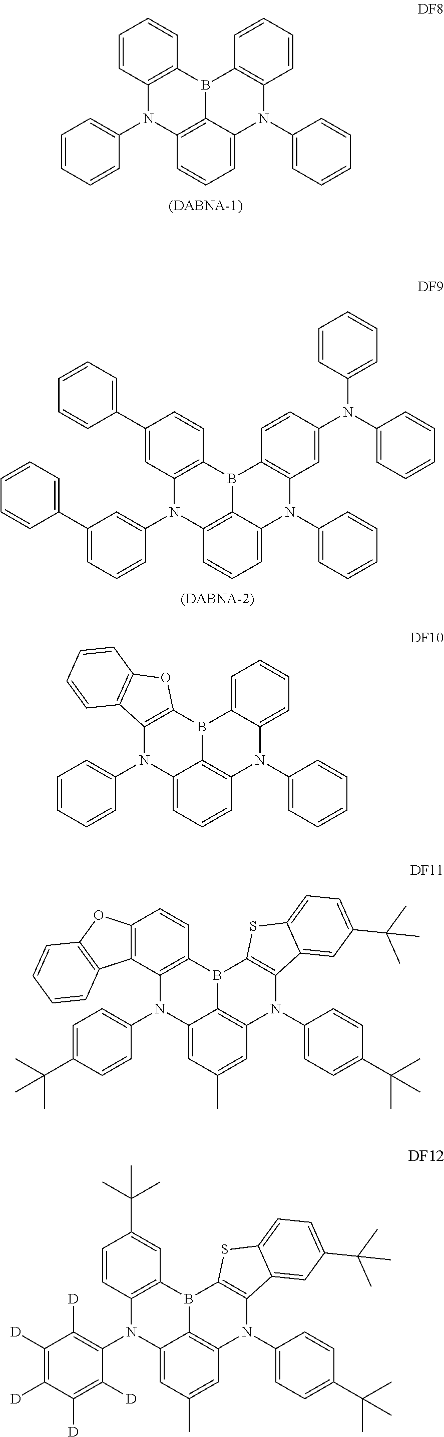

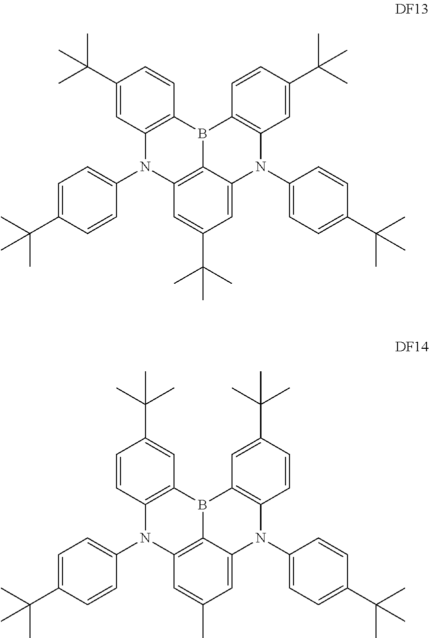

### [Quantum Dot]

In an embodiment, the emission layer 130 may include a quantum dot.

In the specification, a quantum dot may be a crystal of a semiconductor compound, and may include any material capable of emitting light of various emission wavelengths according to a size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method that includes mixing a precursor material with an organic solvent and growing a quantum dot particle crystal. When the crystal grows, the organic solvent naturally serves as a dispersant coordinated on the surface of the quantum dot crystal and controls the growth of the crystal. Accordingly, the growth of quantum dot particles can be controlled through a process which costs less and may be more readily performed than vapor deposition methods, such as metal organic chemical vapor deposition (MOCVD) or molecular beam epitaxy (MBE).

The quantum dot may include a Group II-VI semiconductor compound, a Group III-V semiconductor compound, a Group III-VI semiconductor compound, a Group I-III-VI semiconductor compound, a Group IV-VI semiconductor compound, a Group IV element or compound, or any combination thereof.

Examples of a Group II-VI semiconductor compound may include: a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of a Group III-V semiconductor compound may include: a binary compound, such as GaN, GaP, GaAs, GaSb, AlN, AlP, AlAs, AlSb, InN, InP, InAs, InSb, and the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and the like; or any combination thereof. The Group III-V semiconductor compound may further include a Group II element.

Examples of a Group III-V semiconductor compound further including a Group II element may include InZnP, InGaZnP, InAlZnP, etc.

Examples of a Group III-VI semiconductor compound may include: a binary compound, such as GaS, GaSe, Ga₂Se₃, GaTe, InS, InSe, In₂S₃, In₂Se₃, or InTe; a ternary compound, such as InGaS₃, or InGaSe₃; or any combination thereof.

Examples of a Group I-III-VI semiconductor compound may include: a ternary compound such as AgInS, AgInS₂, CuInS, CuInS₂, CuGaO₂, AgGaO₂, or AgAlO₂; a quaternary compound such as AgInGaS or AgInGaS₂; or any combination thereof.

Examples of a Group IV-VI semiconductor compound may include: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or any combination thereof.

Examples of a Group IV element or compound may include: a single element material, such as Si, Ge, etc.; a binary compound, such as SiC, SiGe, etc.; or any combination thereof.

Each element included in a compound such as a binary compound, a ternary compound, or a quaternary compound may be present in a particle at a uniform concentration or non-uniform concentration.

In an embodiment, the quantum dot may have a single structure in which the concentration of each element in the quantum dot is uniform, or the quantum dot may have a core-shell structure. In an embodiment, in case that the quantum dot has a core-shell structure, a material included in the core and a material included in the shell may be different from each other.

The shell of the quantum dot may serve as a protective layer that prevents chemical degeneration of the core to maintain semiconductor characteristics, and/or may serve as a charging layer that imparts electrophoretic characteristics to the quantum dot. The shell may be a single layer or a multi-layer. An interface between the core and the shell may have a concentration gradient in which the concentration of material that is present in the shell decreases toward the core.

Examples of a shell of a quantum dot may include a metal oxide, a metalloid oxide, or a non-metal oxide, a semiconductor compound, or any combination thereof.

Examples of a metal oxide, a metalloid oxide, or a non-metal oxide may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; or any combination thereof.

Examples of a semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. Examples of a semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be less than or equal to about 45 nm. For example, the FWHM of an emission wavelength spectrum of the quantum dot may be less than or equal to about 40 nm. For example, the FWHM of an emission wavelength spectrum of the quantum dot may be less than or equal to about 30 nm. When the quantum dot has an FWHM of an emission wavelength spectrum within any of these ranges, color purity or color reproducibility may be increased. Light emitted through the quantum dot may be emitted in all directions, so that a wide viewing angle may be improved.

In an embodiment, the quantum dot may be in the form of a spherical particle, a pyramidal particle, a multi-arm particle, a cubic nanoparticle, a nanotube particle, a nanowire particle, a nanofiber particle, or a nanoplate particle.

Since an energy band gap may be adjusted by controlling the size of the quantum dot, light having various wavelength bands may be obtained from the quantum dot emission layer. Accordingly, by using quantum dots of different sizes, a light-emitting device that emits light of various wavelengths may be implemented. In embodiments, the size of the quantum dot may be selected to emit red light, green light, and/or blue light. The size of the quantum dot may be configured to emit white light by combination of light of various colors.

### [Photoactive Layer 135]

The opto-electronic device 30 may include the photoactive layer 135, disposed on the hole transport region 120. The photoactive layer 135 may be between the hole transport region 120 and the electron transport region 140. For example, the photoactive layer 135 may be disposed between a hole transport layer included in the hole transport region 120 and a buffer layer included in the electron transport region 140. In an embodiment, the photoactive layer 135 may be disposed between an emission auxiliary layer included in the hole transport region 120 and a buffer layer included in the electron transport region 140.

In an embodiment, the photoactive layer 135 may include the first compound and the second compound. The first compound may be referred to as an electron-donor compound or a p-type compound, and the second compound may be referred to as an electron-acceptor compound or an n-type compound.

In an embodiment, the first compound and the second compound may be mixed and included in the photoactive layer 135. For example, the photoactive layer 135 may be a single layer including the first compound and the second compound.

The photoactive layer 135 may generate excitons by absorbing incident light. The excitons may generate holes and electrons. The holes generated by the photoactive layer 135 may move to the first electrode 110 through the hole transport region 120. The electrons generated by the photoactive layer 135 may move to the second electrode 150 through the electron transport region 140.

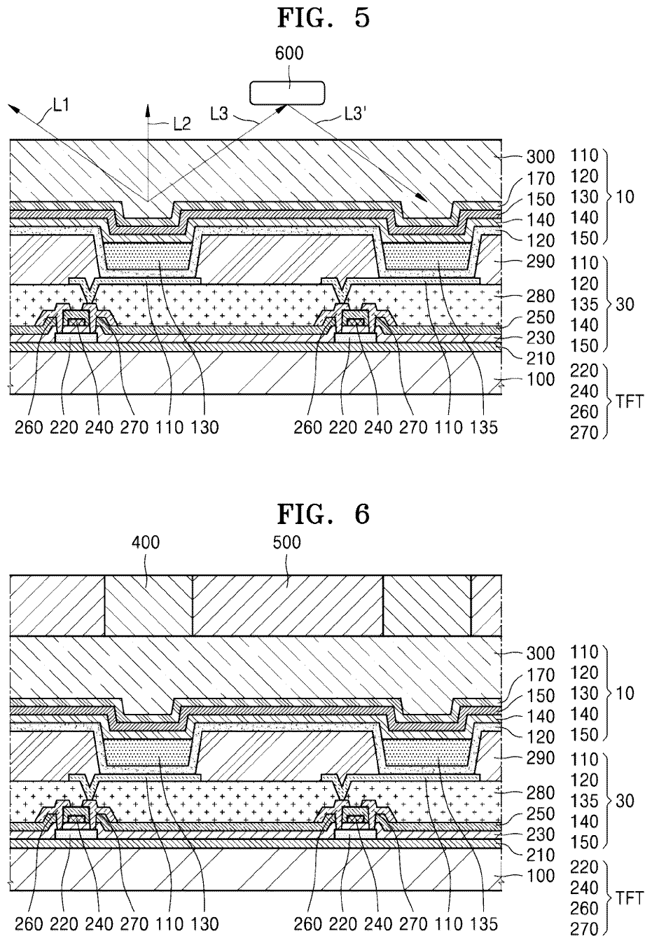

For example, the photoactive layer 135 may absorb light to generate electric signal. The first compound included in the photoactive layer 135 may serve as a donor to supply electrons, and the second compound included in the photoactive layer 135 may serve as an acceptor to receive electrons. Thus, the opto-electronic device 30 including the photoactive layer 135 may serve as an optical sensor. For example, the opto-electronic device 30 may serve as a fingerprint recognition sensor, which will be described later with reference to FIG. 5.

### [Electron Transport Region 140]

The electron transport region 140 may have a structure consisting of a layer consisting of a single material, a structure consisting of a layer including different materials, or a structure including multiple layers including different materials.

The electron transport region 140 may include a buffer layer, a hole-blocking layer, an electron control layer, an electron transport layer, an electron injection layer, or any combination thereof.

In an embodiment, the electron transport region 140 may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, wherein the layers of each structure may be stacked from the emission layer 130 and/or the photoactive layer 135 in its respective stated order, but the structure of the electron transport region 140 is not limited thereto.

The electron transport region 140 (e.g., a buffer layer, a hole-blocking layer, an electron control layer, or an electron transport layer in the electron transport region 140) may include a metal-free compound including at least one π electron-deficient nitrogen-containing C₁-C₆₀ cyclic group.

In an embodiment, the electron transport region 140 may include a compound represented by Formula 601:

[Ar₆₀₁]ₓₑ₁₁-[(L₆₀₁)ₓₑ₁-R₆₀₁]ₓₑ₂₁. [Formula 601]

In Formula 601,
Ar₆₀₁ and L₆₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be the same as described in connection with Q₁,
xe21 may be 1, 2, 3, 4, or 5, and
at least one of Ar₆₀₁, L₆₀₁, and R₆₀₁ may each independently be a π electron-deficient nitrogen-containing C₁-C₆₀ cyclic group unsubstituted or substituted with at least one R₁₀ₐ.

In an embodiment, in Formula 601, when xe11 is 2 or more, two or more of Ar₆₀₁ may be linked together via a single bond.

In an embodiment, in Formula 601, Ar₆₀₁ may be an anthracene group that is unsubstituted or substituted with at least one R₁₀ₐ.

In an embodiment, the electron transport region 140 may include a compound represented by Formula 601-1:

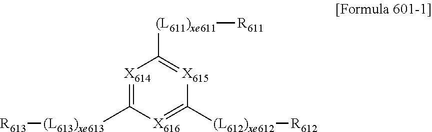

In Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may each be N,
L₆₁₁ to L₆₁₃ may each independently be the same as described in connection with L₆₀₁,
xe611 to xe613 may each independently be the same as described in connection with xe1,
R₆₁₁ to R₆₁₃ may each independently be the same as described in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

In an embodiment, in Formulae 601 and 601-1, xe1 and xe611 to xe613 may each independently be 0, 1, or 2.

The electron transport region 140 may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

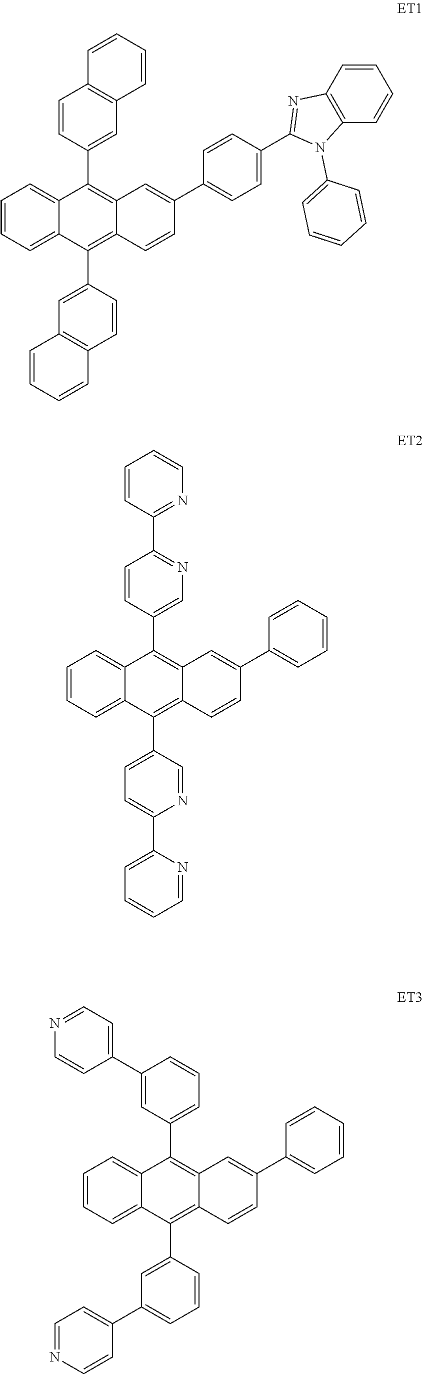 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

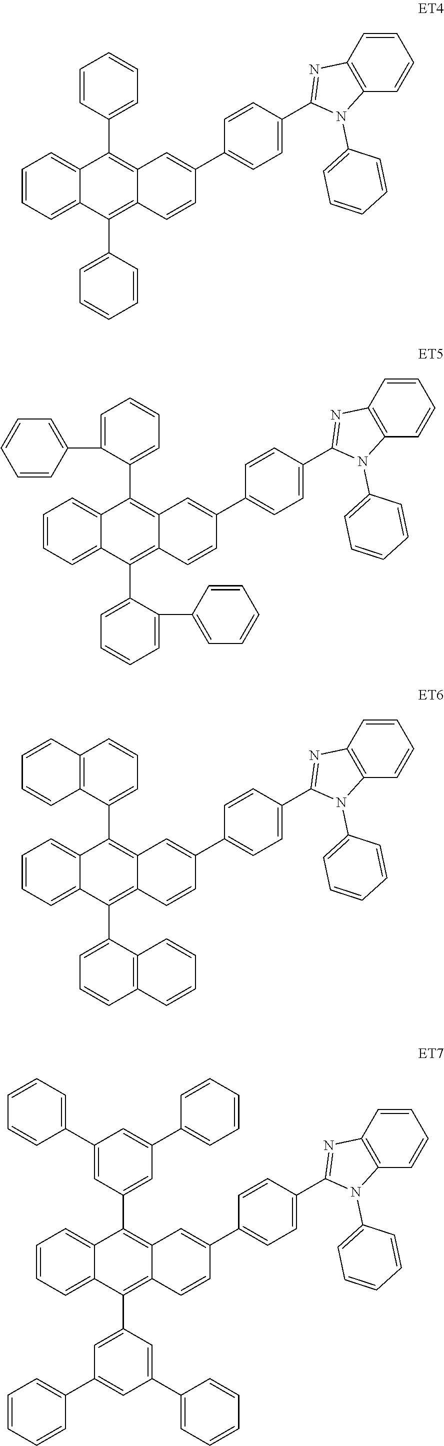 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

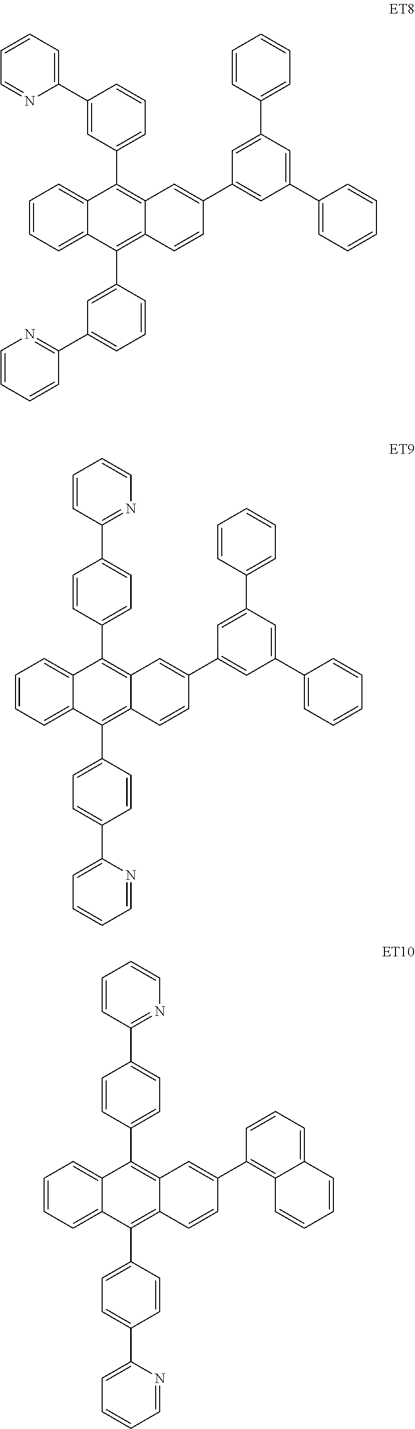

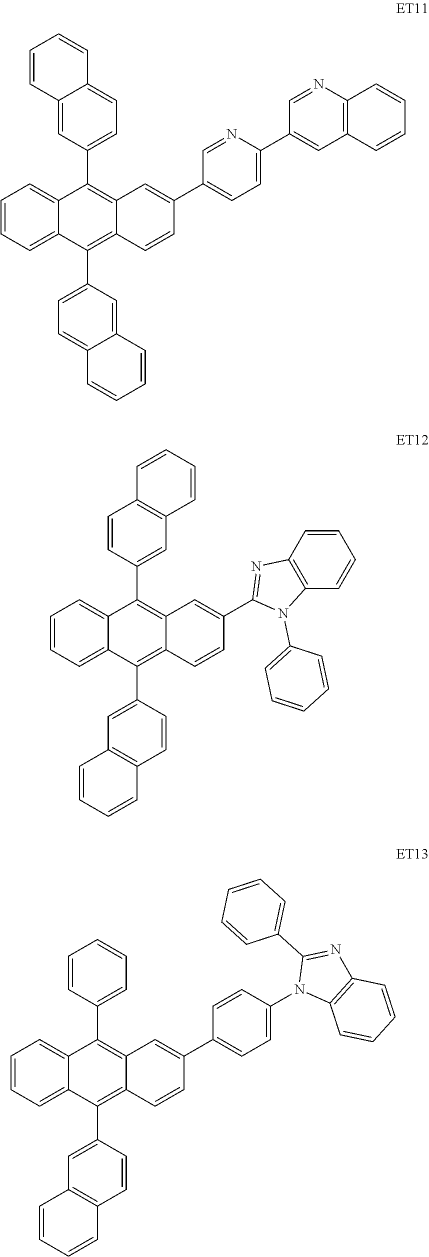 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

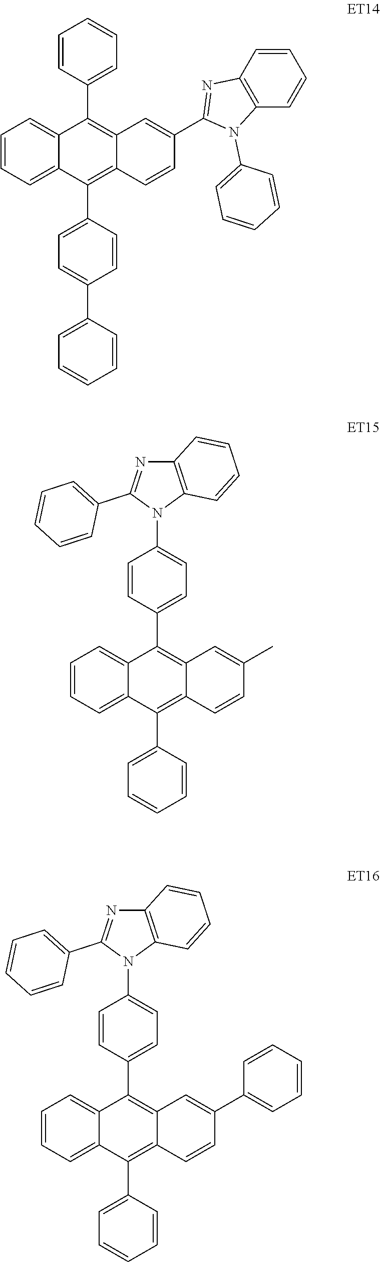 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

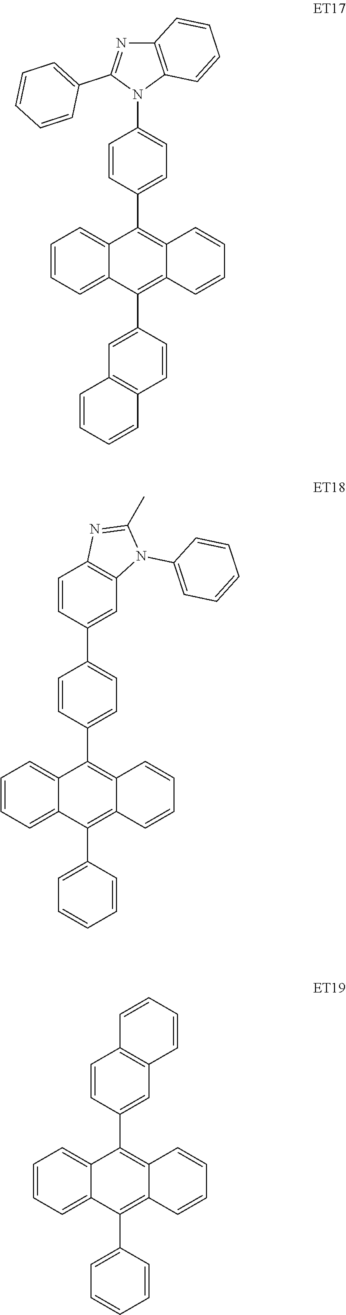

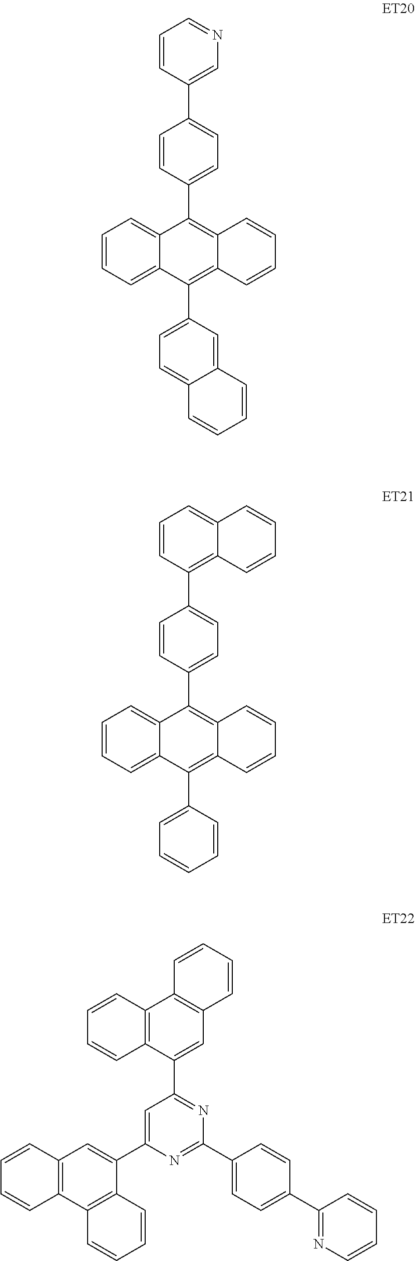

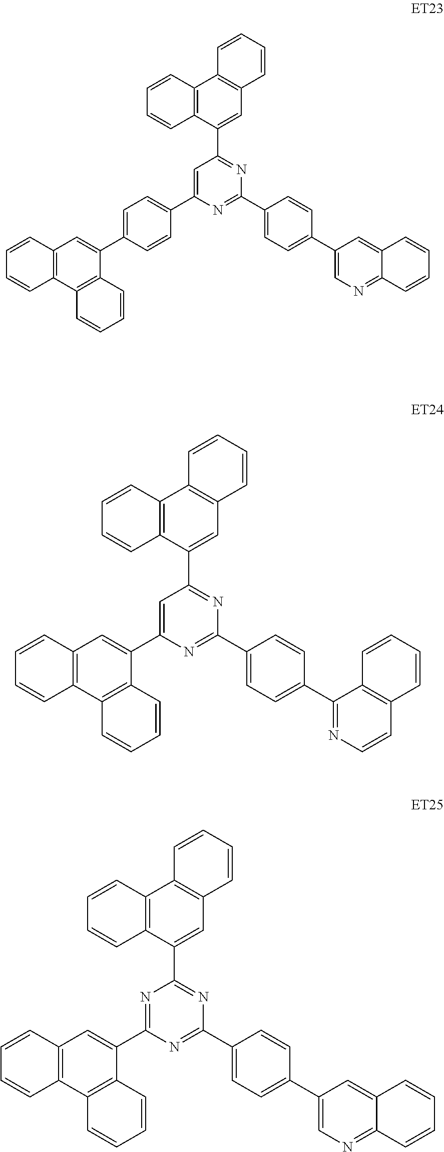 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

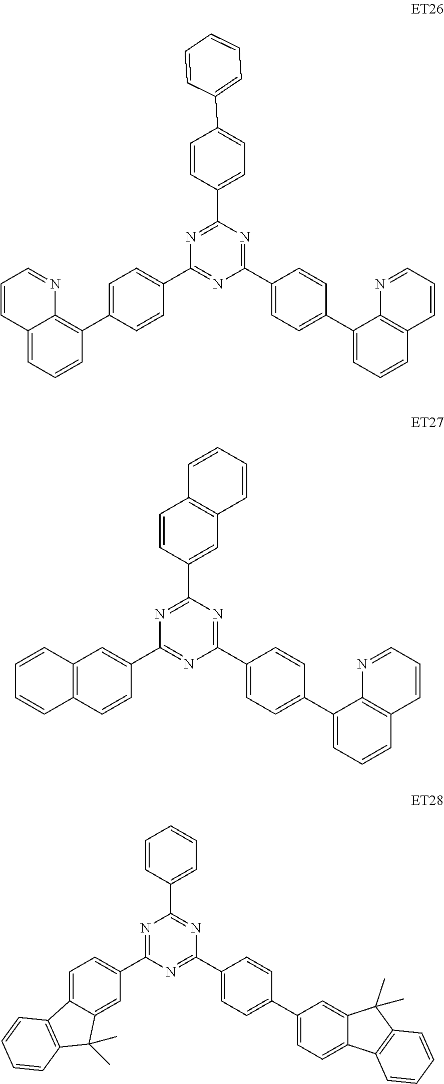 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc6c(c5)C(C)(C)c5ccccc5-6)n4)cc3)cc21]

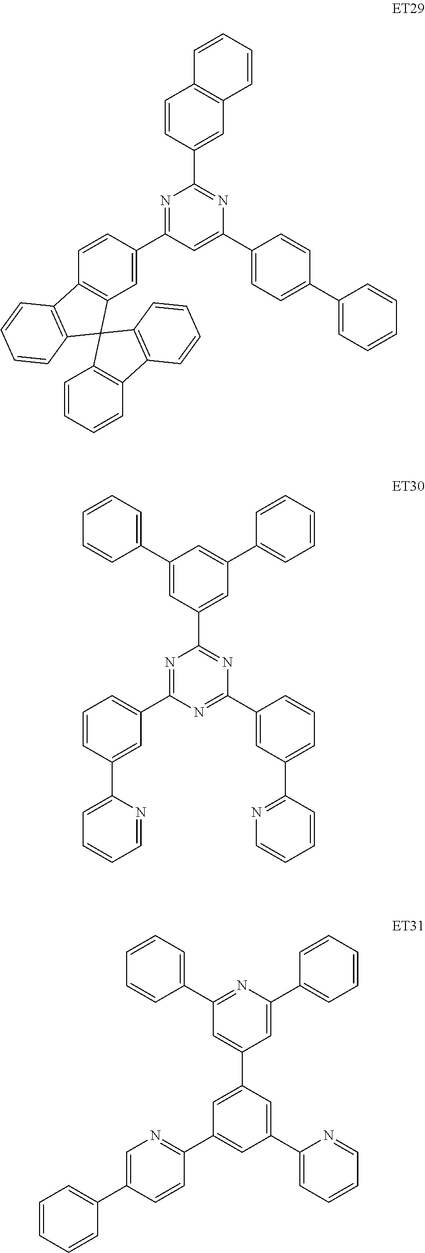 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

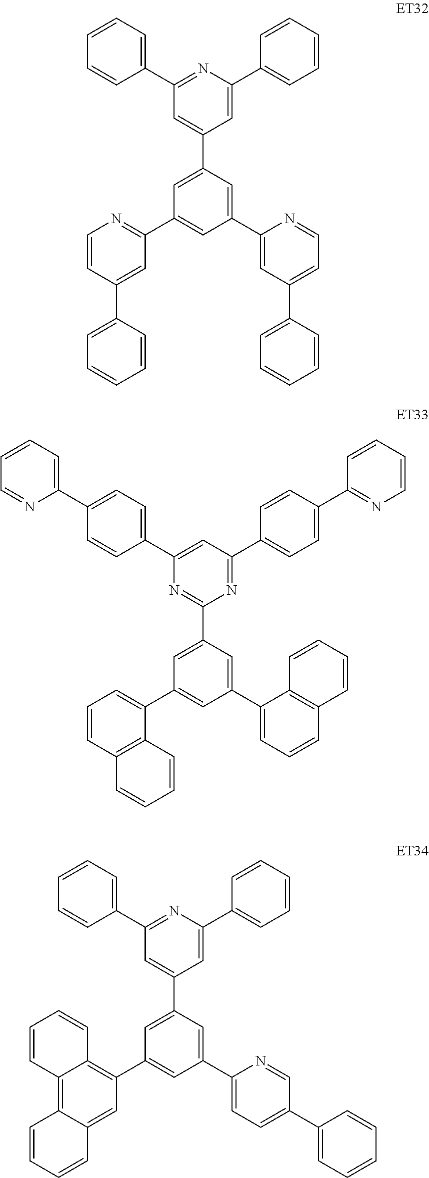 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

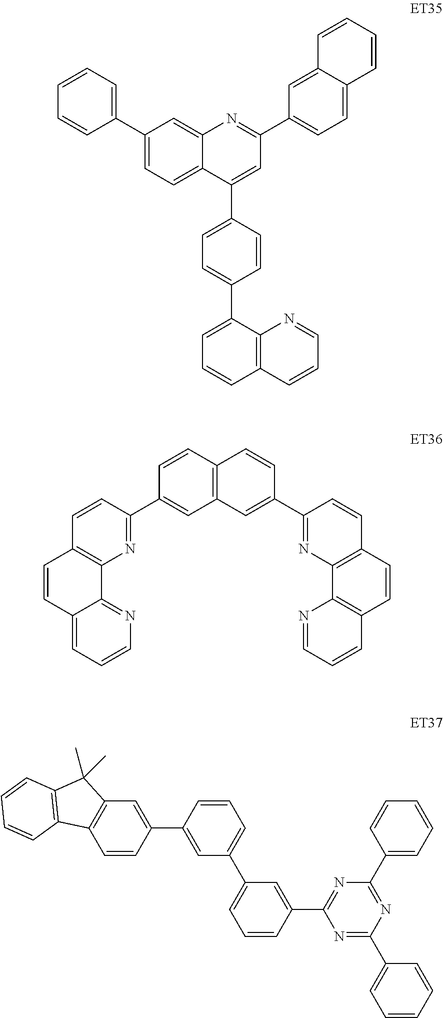 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21]

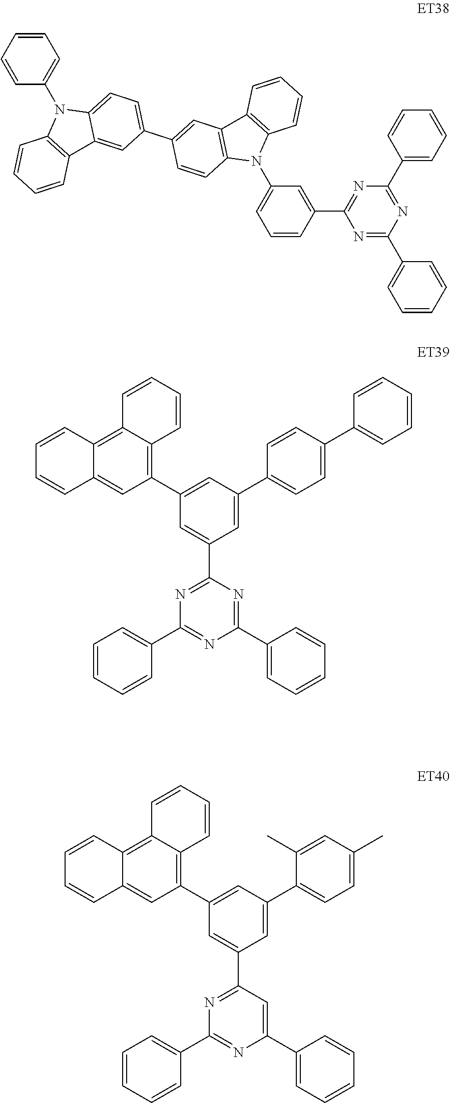 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

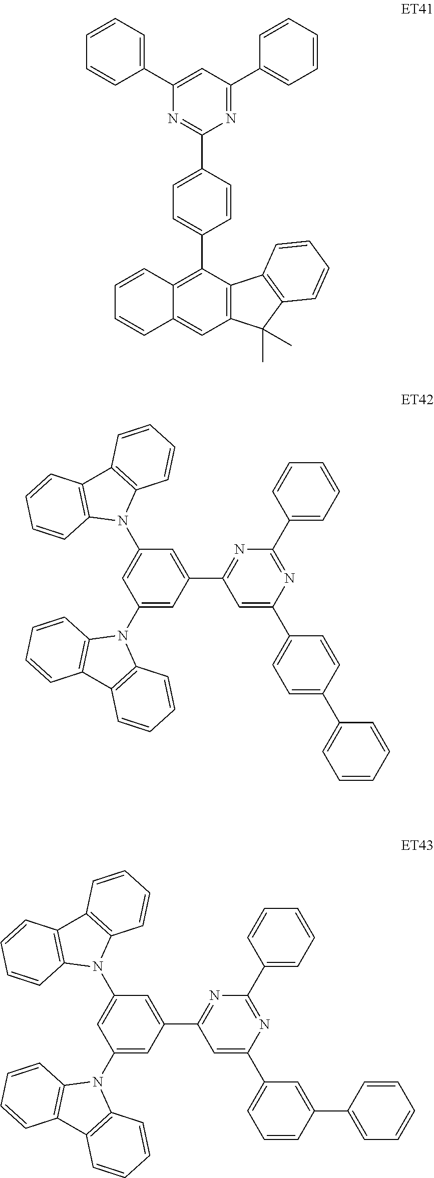 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

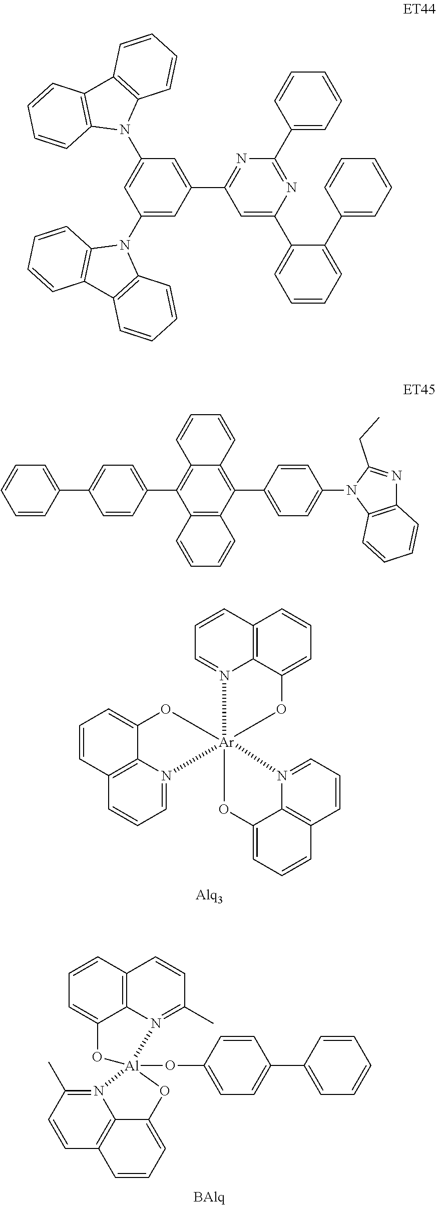

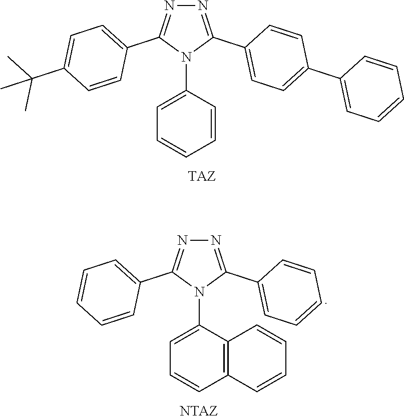 [SMILES: CC(C)(C)c1ccc(-c2nnc(-c3ccc(-c4ccccc4)cc3)n2-c2ccccc2)cc1 ; c1ccc(-c2nnc(-c3ccccc3)n2-c2cccc3ccccc23)cc1]

A thickness of the electron transport region 140 may be in a range of about 100 Å to about 5,000 Å. For example, the thickness of the electron transport region 140 may be in a range of about 160 Å to about 4,000 Å. When the electron transport region includes a buffer layer, a hole-blocking layer, an electron control layer, an electron transport layer, or any combination thereof, a thickness of the buffer layer, the hole-blocking layer, or the electron control layer may each independently be in a range of about 20 Å to about 1,000 Å, and a thickness of the electron transport layer may be in a range of about 100 Å to about 1,000 Å. For example, the thickness of the buffer layer, the hole-blocking layer, or the electron control layer may each independently be in a range of about 30 Å to about 300 Å. For example, the thickness of the electron transport layer may be in a range of about 150 Å to about 500 Å. When the thicknesses of the buffer layer, the hole-blocking layer, the electron control layer, the electron transport layer, and/or the electron transport region 140 are within the ranges described above, satisfactory electron transporting characteristics may be obtained without a substantial increase in driving voltage.

The electron transport region 140 (for example, an electron transport layer in the electron transport region 140) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In an embodiment, the metal-containing material may include a Li complex.

The Li complex may include, for example, Compound ET-D1 (LiQ) or Compound ET-D2:

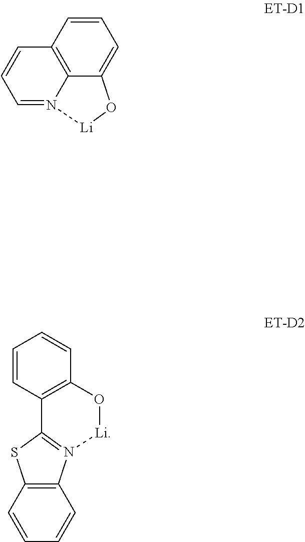

The electron transport region 140 may include an electron injection layer that facilitates the injection of electrons from the second electrode 150. The electron injection layer may contact (e.g., directly contact) with the second electrode 150, but is not limited thereto.

The electron injection layer may have a structure consisting of a layer consisting of a single material, a structure consisting of a layer including different materials, or a structure including multiple layers including different materials.

In an embodiment, the electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, iodides, etc.), or tellurides of the alkali metal, the alkaline earth metal, and the rare earth metal, or any combination thereof.

The alkali metal-containing compound may include: an alkali metal oxides, such as Li₂O, Cs₂O, or K₂O; an alkali metal halides, such as LiF, NaF, CsF, KF, Lil, NaI, CsI, or KI; or any combination thereof. The alkaline earth metal-containing compound may include an alkaline earth metal oxide, such as BaO, SrO, CaO, BaₓSr₁₋ₓO (wherein x is a real number satisfying 0<x<1), or BaₓCa₁₋ₓO (wherein x is a real number satisfying 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In an embodiment, the rare earth metal-containing compound may include a lanthanide metal telluride. Examples of a lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include: an alkali metal ion, an alkaline earth metal ion, or a rare earth metal; and a ligand bonded to the metal ion (for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof.

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In an embodiment, the electron injection layer may further include an organic material (for example, a compound represented by Formula 601).

In an embodiment, the electron injection layer may consist of an alkali metal-containing compound (for example, an alkali metal halide), an alkali metal-containing compound (for example, an alkali metal halide); and an alkali metal, an alkaline earth metal, a rare earth metal, or any combination thereof. In an embodiment, the electron injection layer may be a KI:Yb co-deposited layer, an RbI:Yb co-deposited layer, a LiF:Yb co-deposited layer, or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly or non-uniformly dispersed in a matrix including the organic material.

A thickness of the electron injection layer may be about 1 Å to about 100 Å. For example, the thickness of the electron injection layer may be in a range of about 3 Å to about 90 Å. When the thickness of the electron injection layer is within any of the ranges as described above, satisfactory electron injection characteristics may be obtained without a substantial increase in driving voltage.

### [Second Electrode 150]

The second electrode 150 may be arranged on the electron transport region 140. The second electrode 150 may be a cathode, which is an electron injection electrode. When the second electrode 150 is a cathode, a material for forming the second electrode may include a material having a low-work function, such as a metal, an alloy, an electrically conductive compound, or any combination thereof.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a transflective electrode, or a reflective electrode.

The second electrode 150 may have a single-layer structure or a multilayer structure.

### [Capping Layer]

The light-emitting device 10 may include a first capping layer outside the first electrode 110, and/or a second capping layer outside the second electrode 150. In embodiments, the light-emitting device 10 may have a structure in which the first capping layer, the first electrode 110, the emission layer 130, and the second electrode 150 are stacked in this stated order, a structure in which the first electrode 110, the emission layer 130, the second electrode 150, and the second capping layer are stacked in this stated order, or a structure in which the first capping layer, the first electrode 110, the emission layer 130, the second electrode 150, and the second capping layer are stacked in this stated order.

Light generated in the emission layer 130 of the light-emitting device 10 may pass through the first electrode 110, which may be a transflective electrode or a transmissive electrode, and through the first capping layer to the outside. Light generated in the emission layer 130 of the light-emitting device 10 may pass through the second electrode 150, which may be a transflective electrode or a transmissive electrode, and through the second capping layer to the outside.

The first capping layer and the second capping layer may each increase external emission efficiency according to the principle of constructive interference. Accordingly, the light extraction efficiency of the light-emitting device 10 may be increased, such that the luminescence efficiency of the light-emitting device 10 may be increased.

The first capping layer and the second capping layer may each include a material having a refractive index greater than or equal to about 1.6 (with respect to a wavelength in a range of about 520 nm to about 630 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In an embodiment, at least one of the first capping layer and the second capping layer may each independently include an amine group-containing compound.

In an embodiment, at least one of the first capping layer and the second capping layer may each independently include a compound represented by Formula 201, a compound represented by Formula 202, or any combination thereof.

In an embodiment, at least one of the first capping layer and the second capping layer may each independently include one of Compounds HT28 to HT33, one of Compounds CP1 to CP6, p-NPB, or any combination thereof:

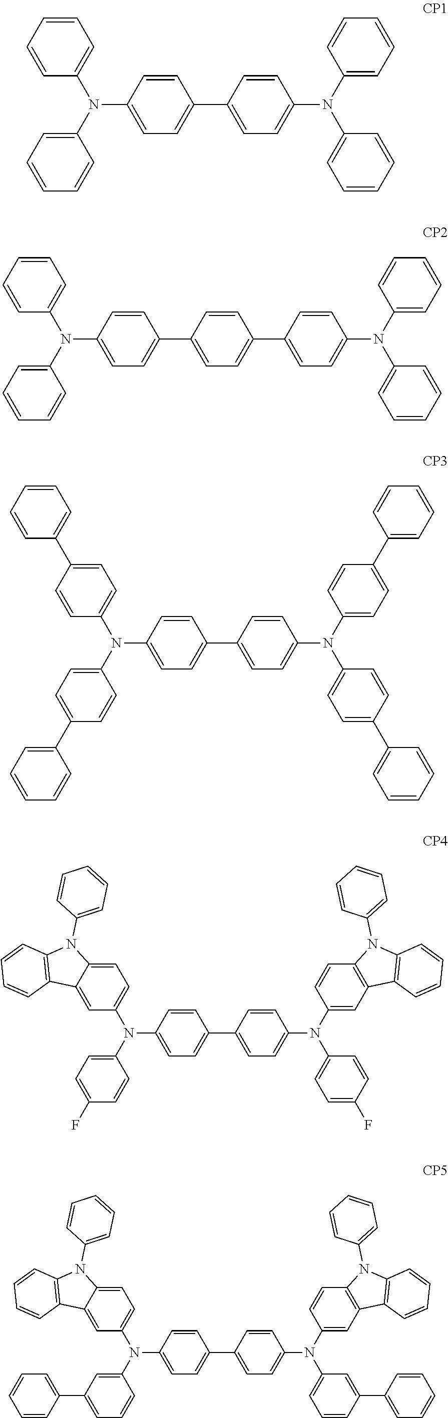 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

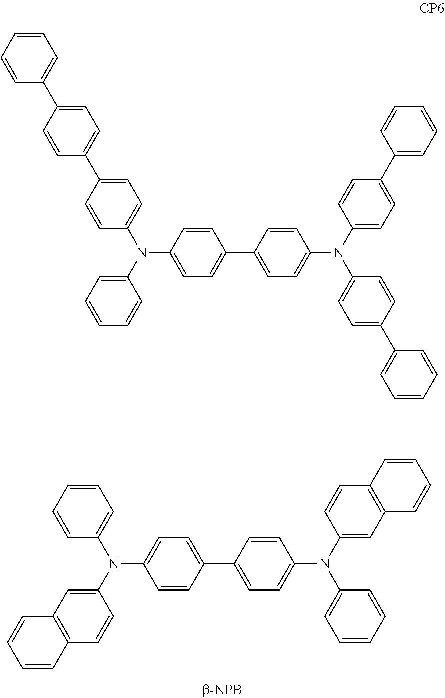 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### [Film]

In an embodiment, the electronic apparatus may include a film. The film may be, for example, an optical member (or a light control means) (e.g., a color filter, a color conversion member, a capping layer, a light extraction efficiency enhancement layer, a selective light absorbing layer, a polarizing layer, a quantum dot-containing layer, etc.), a light blocking member (e.g., a light reflective layer, a light absorbing layer, etc.), a protective member (e.g., an insulating layer, a dielectric layer, etc.), or the like.

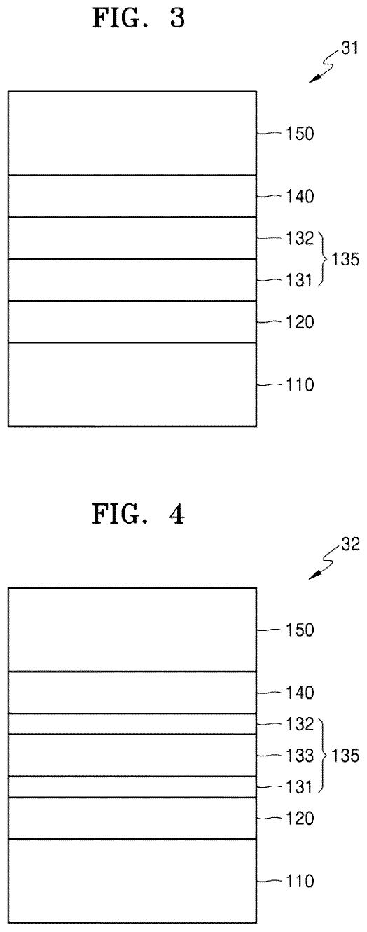

### [Description of FIG. 3]

FIG. 3 is a schematic cross-sectional view of an opto-electronic device 31 according to another embodiment.

The opto-electronic device 31 shown in FIG. 3 may differ from the opto-electronic device 30 shown in FIG. 1 at least with respect to the photoactive layer 135.

The opto-electronic device 31 may include the photoactive layer 135 disposed between the hole transport region 120 and the electron transport region 140. For example, the photoactive layer 135 may be disposed between a hole transport layer included in the hole transport region 120 and a buffer layer included in the electron transport region 140. In an embodiment, the photoactive layer 135 may be disposed between an emission auxiliary layer included in the hole transport region 120 and a buffer layer included in the electron transport region 140.

The photoactive layer 135 may include a first layer 131 adjacent to the hole transport region 120, and a second layer 132 adjacent to the electron transport region 140. For example, the first layer 131 may contact (e.g., directly contact) the second layer 132.

For example, the first layer 131 may contact (e.g., directly contact) the hole transport layer included in the hole transport region 120. In an embodiment, the first layer 131 may contact (e.g., directly contact) an emission auxiliary layer disposed on the hole transport layer.

In an embodiment, the second layer 132 may contact (e.g., directly contact) a buffer layer included in the electron transport region 140.

The first layer 131 may include the first compound. The first layer 131 may consist of the first compound. For example, the first layer 131 may not include the second compound. The first layer 131 may also be referred to as a p-type photoactive layer or a donor layer.

In an embodiment, the second layer 132 may include the second compound.

The second layer 132 may consist of the second compound. For example, the second layer 132 may not include the first compound. The second layer 132 may also be referred to as an n-type photoactive layer or an acceptor layer.

For example, the photoactive layer 135 may have a two-layer structure of a first layer 131 including the first compound and a second layer 132 including the second compound.

The photoactive layer 135 may absorb incident light and form excitons. The excitons may generate holes and electrons. The holes generated by the photoactive layer 135 may move to the first electrode 110 through the hole transport region 120.

The electrons generated by the photoactive layer 135 may move to the second electrode 150 through the electron transport region 140.

In an embodiment, the photoactive layer 135 may absorb light to generate an electric signal. The first compound included in the first layer 131 may serve as a donor to supply electrons, and the second compound included in the second layer 132 may serve as an acceptor to receive electrons. Thus, the opto-electronic device 30 including the photoactive layer 135 may serve as an optical sensor. For example, the opto-electronic device 31 may serve as a fingerprint recognition sensor, which will be described later with reference to FIG. 5.

### [Description of FIG. 4]

FIG. 4 is a schematic cross-sectional view of an opto-electronic device according to another embodiment;

The opto-electronic device 32 shown in FIG. 4 may differ from the opto-electronic device 31 shown in FIG. 1 at least with respect to the photoactive layer 135.

In an embodiment, the opto-electronic device 32 may include the photoactive layer 135 disposed between the hole transport region 120 and the electron transport region 140. For example, the photoactive layer 135 may be disposed between a hole transport layer included in the hole transport region 120 and a buffer layer included in the electron transport region 140. In an embodiment, the photoactive layer 135 may be disposed between an emission auxiliary layer included in the hole transport region 120 and a buffer layer included in the electron transport region 140.

The photoactive layer 135 may include a first layer 131 adjacent to the hole transport region 120, a second layer 132 adjacent to the electron transport region 140, and a third layer 133 disposed between the first layer 131 and the second layer 132. For example, the third layer 133 may contact (e.g., directly contact) the first layer 131 and/or the second layer 132.

For example, the first layer 131 may contact (e.g., directly contact) a hole transport layer included in the hole transport region 120. In an embodiment, the first layer 131 may contact (e.g., directly contact) an emission auxiliary layer disposed on the hole transport layer.

In an embodiment, the second layer 132 may contact (e.g., directly contact) a buffer layer included in the electron transport region 140.

The first layer 131 may include the first compound. The first layer 131 may consist of the first compound. In an embodiment, the first layer 131 may not include the second compound. The first layer 131 may also be referred to as a p-type photoactive layer or a donor layer.

The second layer 132 may include the second compound. The second layer 132 may consist of the second compound. In an embodiment, the second layer 132 may not include the first compound. The second layer 132 may also be referred to as an n-type photoactive layer or an acceptor layer.

The third layer 133 may include the first compound and the second compound.

For example, the first compound and the second compound may be mixed and included in the third layer 133. The third layer 133 may also be referred to as a mixing layer.

In an embodiment, the photoactive layer 135 may have a three-layer structure including the first layer 131 including the first compound, the third layer 133 including the first compound and the second compound, and the second layer 132 including the second compound.

In an embodiment, the photoactive layer 135 may generate excitons by absorbing incident light. The excitons may generate holes and electrons. The holes generated by the photoactive layer 135 may move to the first electrode 110 through the hole transport region 120. The electrons generated by the photoactive layer 135 may move to the second electrode 150 through the electron transport region 140.

In an embodiment, the photoactive layer 135 may absorb light to generate electric signal. The first compound included in each of the first layer 131 and the third layer 133 may serve as a donor to supply electrons, and the second compound included in each of the second layer 132 and the third layer 133 may serve as an acceptor to receive electrons. Thus, the opto-electronic device 30 including the photoactive layer 135 may serve as an optical sensor. For example, the opto-electronic device 30 may serve as a fingerprint recognition sensor, which will be described later with reference to FIG. 5.

### [Electronic Device]

The light-emitting device 10, and the opto-electronic devices 30, 31, and 32 may be included in various electronic devices. For example, the electronic device may be a display device, a light-emitting device, an authentication device, etc.

The electronic device (for example, a light-emitting device) may further include, in addition to the light-emitting device 10, and the opto-electronic devices 30, 31, and 32, a color filter, a color conversion layer, or a color filter and a color conversion layer.

The color filter and/or the color conversion layer may be arranged in at least one traveling direction of light emitted from the light-emitting device. For example, the light emitted from the light-emitting device may be blue light or white light. The light-emitting device may be the same as described herein. In an embodiment, the color conversion layer may include quantum dots. The quantum dot may be, for example, a quantum dot as described herein.

The electronic apparatus may include a first substrate. The first substrate may include subpixels, the color filter may include color filter areas respectively corresponding to the subpixels, and the color conversion layer may include color conversion areas respectively corresponding to the subpixels.

A pixel-defining film may be arranged between the subpixels to define each subpixel.

The color filter may further include color filter areas and light-shielding patterns arranged between the color filter areas, and the color conversion layer may further include color conversion areas and light-shielding patterns arranged between the color conversion areas.

The color filter areas (or the color conversion areas) may include a first area emitting first color light, a second area emitting second color light, and/or a third area emitting third color light, wherein the first color light, the second color light, and/or the third color light may have different maximum emission wavelengths from one another. In an embodiment, the first color light may be red light, the second color light may be green light, and the third color light may be blue light. In an embodiment, the color filter areas (or the color conversion areas) may include quantum dots. In an embodiment, the first area may include red quantum dots, the second area may include green quantum dots, and the third area may not include quantum dots. The quantum dots may be the same as described herein. The first area, the second area, and/or the third area may each further include a scatterer.

In an embodiment, the light-emitting device may emit first light, the first area may absorb the first light to emit first-first color light, the second area may absorb the first light to emit second-first color light, and the third area may absorb the first light to emit third-first color light. In an embodiment, the first-first color light, the second-first color light, and the third-first color light may have different maximum emission wavelengths. For example, the first light may be blue light, the first-first color light may be red light, the second-first color light may be green light, and the third-first color light may be blue light.

The electronic device may further include a thin film transistor in addition to the opto-electronic device and the light-emitting device described above. The thin-film transistor may include a source electrode, a drain electrode, and an active layer, wherein any one of the source electrode and the drain electrode may be electrically connected to any one of the first electrode and the second electrode of the light-emitting device.

The thin-film transistor may further include a gate electrode, a gate insulating film, and the like.

The active layer may include crystalline silicon, amorphous silicon, an organic semiconductor, an oxide semiconductor, and the like.

The electronic device may further include a sealing portion that seals the opto-electronic device, and the light-emitting device. The sealing portion may be arranged between the color filter and/or the color conversion layer and the light-emitting device. The sealing portion may allow light from the light-emitting device to be extracted to the outside, and may prevent ambient air and/or moisture from penetrating into the opto-electronic device and the light-emitting device. The sealing portion may be a sealing substrate including a transparent glass substrate or a plastic substrate. The sealing portion may be a thin-film encapsulation layer that includes at least one layer of an organic layer and/or an inorganic layer. When the sealing portion is a thin film encapsulation layer, the electronic apparatus may be flexible.

Various functional layers may be further included on the sealing portion, in addition to the color filter and/or the color conversion layer, according to the use of the electronic apparatus. Examples of the functional layers may include a touch screen layer and a polarizing layer. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication device may further include a biometric information collection member, in addition to the opto-electronic device and the light-emitting device described above.

The electronic device may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, sensors (for example, sensors for vehicles and sensors for home), solar cells, and the like.

### [Electronic Apparatus]

The opto-electronic device may be included in various types of electronic apparatuses.

In an embodiment, an electronic apparatus including the opto-electronic device may be a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a mobile phone, a tablet computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall including multiple displays tiled together, a theater screen, a stadium screen, a phototherapy device, a signboard, an automotive sensor, a home sensor, or a solar cell.

Since the opto-electronic device has excellent photoelectric properties and the like, an electronic apparatus including the opto-electronic device may have an optical sensor function such as a fingerprint recognition sensor or the like.

### [Description of FIGS. 5 and 6]

FIG. 5 is a schematic cross-sectional view of an electronic device according to an embodiment.

The electronic device of FIG. 5 includes a substrate 100, a thin film transistor (TFT), a light-emitting device 10, an opto-electronic device 30, and an encapsulation portion 300. The opto-electronic device 30 of FIG. 5 may be the opto-electronic device 30 described with reference to FIG. 1, but is not limited thereto. For example, the opto-electronic device 30 of FIG. 5 may be the opto-electronic device 31 of FIG. 3 or the opto-electronic device 32 of FIG. 4.

The substrate 100 may be a flexible substrate, a glass substrate, or a metal substrate. A buffer layer 210 may be arranged on the substrate 100. The buffer layer 210 may prevent penetration of impurities through the substrate 100 and may provide a flat surface on the substrate 100.

A TFT may be arranged on the buffer layer 210. The TFT may include an active layer 220, a gate electrode 240, a source electrode 260, and a drain electrode 270.

The active layer 220 may include an inorganic semiconductor, such as silicon or polysilicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 to insulate the gate electrode 240 from the source electrode 260 and between the gate electrode 240 and the drain electrode 270 to insulate the gate electrode 140 from the drain electrode 270.

The source electrode 260 and the drain electrode 270 may be arranged on the interlayer insulating film 250. The interlayer insulating film 250 and the gate insulating film 230 may be formed to expose a source region and a drain region of the active layer 220, and the source electrode 260 and the drain electrode 270 may respectively contact the exposed portions of the source region and the drain region of the active layer 220.

The light-emitting device 10 and the opto-electronic device 30 may be disposed on a thin film transistor TFT.

The thin film transistor TFT electrically connected to the light-emitting device 10 may transmit electrical signals for driving the light-emitting device 10. The thin film transistor TFT electrically connected to the opto-electronic device 30 may transmit an electrical signal generated by the opto-electronic device 30. The thin film transistor TFT is covered and protected by a passivation layer 280. The passivation layer 280 may include an inorganic insulating film, an organic insulating film, or any combination thereof. The light-emitting device 10 and the opto-electronic device 30 may be arranged on the passivation layer 280.

The light-emitting device 10 may include a first electrode 110, a hole transport region 120, an emission layer 130, an electron transport region 140, and a second electrode 150. The opto-electronic device 30 may include the first electrode 110, the hole transport region 120, a photoactive layer 135, the electron transport region 140, and the second electrode 150. The first electrode 110 may be arranged on the passivation layer 280. The passivation layer 280 may not completely cover the source electrode 260 and the drain electrode 270 and may expose a portion of the source electrode 260 and the drain electrode 270. The first electrode 110 may be electrically connected to the exposed portion of the source electrode 260 and the drain electrode 270.

A pixel-defining film 290 including an insulating material may be located on the first electrode 110. The pixel-defining film 290 may expose an area of the first electrode 110. The pixel-defining film 290 may be a polyimide-based organic film or a polyacrylic organic film.

The hole transport region 120 may be disposed on the pixel-defining film 290. The hole transport region 120 included in the light-emitting device 10 and the hole transport region 120 included in the opto-electronic device 30 may be integrally formed as one body. The hole transport region 120 included in the light-emitting device 10, and the hole transport region 120 included in the opto-electronic device 30 may be disposed on the pixel-defining film 290, may be connected to each other, and may include substantially a same material, and may be formed substantially at about a same time.

The emission layer 130 and photoactive layer 135 may each be disposed on the hole transport region 120. The emission layer 130, and photoactive layer 135 may overlap each other in a selected area of the first electrode 110 that is exposed by the pixel-defining film 290.

The electron transport region 140 may be disposed on the emission layer 130 and the photoactive layer 135. The electron transport region 140 included in the light-emitting device 10 and the electron transport region 140 included in the opto-electronic device 30 may be integrally formed as one body. The electron transport region 140 included in the light-emitting device 10, and the electron transport region 140 included in the opto-electronic device 30 may be disposed on the pixel-defining film 290, may be connected to each other, and may include substantially a same material, and may be formed substantially at about a same time.

The second electrode 150 may be disposed on the electron transport region 140. The second electrode 150 included in the light-emitting device 10 and the second electrode 150 included in the opto-electronic device 30 may be integrally formed as one body. The second electrode 150 included in the light-emitting device 10, and the second electrode 150 included in the opto-electronic device 30 may be disposed on the pixel-defining film 290, may be connected to each other, and may include substantially a same material, and may be formed substantially at about a same time.

A capping layer 170 may be further included on the second electrode 150. The capping layer 170 may cover the second electrode 150.

The encapsulation portion 300 may be disposed on the capping layer 170. The encapsulation portion 300 may be arranged on the light-emitting device 10 and the opto-electronic device 30 to protect the light-emitting device 10 and the opto-electronic device 30 from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or any combination thereof; or any combination of the inorganic film and the organic film.

In an embodiment, the light-emitting device 10 may emit light L1, light L2, or light L3. For example, light L1, light L2, or light L3 may each independently be red light, green light, blue light, or near-infrared light.

For example, light L3, which may be emitted from the light-emitting device 10, may be incident on an object 600 outside the electronic device. For example, the object 600 may be a finger of a user who uses the electronic device. Light L3′ reflected by the object 600 may be incident onto the opto-electronic device 30.

The photoactive layer 135 may absorb light L3′ and excitons are formed therein. The excitons may generate holes and electrons. For example, the photoactive layer 135 may absorb light to generate electric signal. In an embodiment, the first compound included in the photoactive layer 135 may serve as a donor to supply electrons, and the second compound included in the photoactive layer 135 may serve as an acceptor to receive electrons. For example, the opto-electronic device 30 may detect energy of light L3′ and convert the same into an electrical signal. Accordingly, the opto-electronic device 30 may recognize the object 600 that touches (or approaches) the electronic device. Therefore, the opto-electronic device 30 including the photoactive layer 135 may serve as an optical sensor (for example, a fingerprint recognition sensor).

FIG. 6 is a schematic cross-sectional view of an electronic device according to another embodiment.

The electronic device of FIG. 6 may differ from the electronic device of FIG. 5, at least in that a light-blocking pattern 500 and a functional area 400 are further included on the encapsulation portion 300. The functional area 400 may be a color filter area, a color conversion area, or a combination of the color filter area and the color conversion area. According to an embodiment, the light-emitting device included in the electronic device of FIG. 6 may be a tandem light-emitting device.

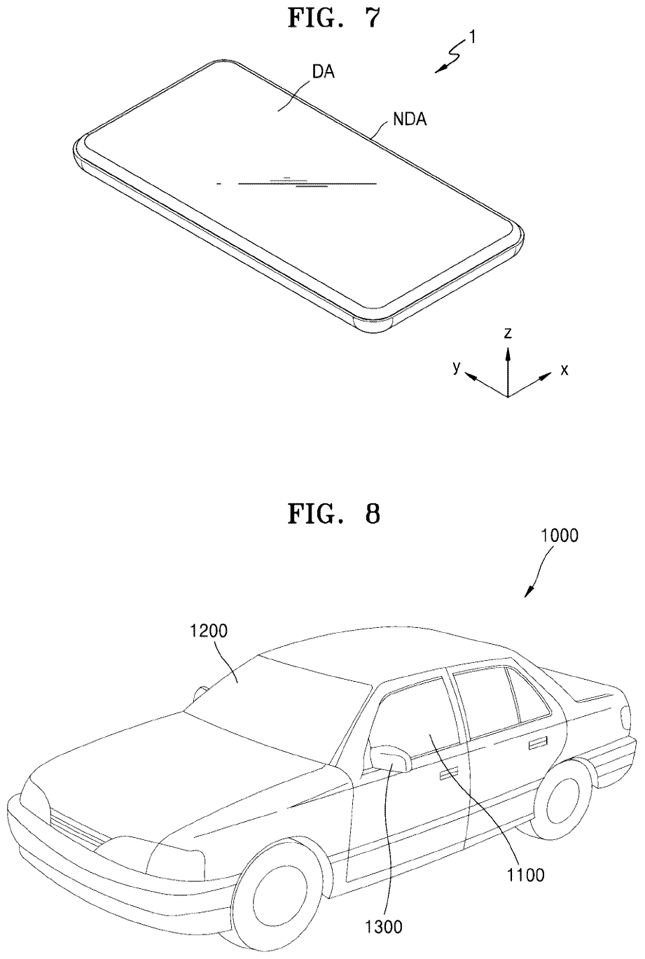

### [Description of FIG. 7]

FIG. 7 is a schematic perspective view of an electronic apparatus 1 including an opto-electronic device according to an embodiment.

The electronic apparatus 1 may be a device that displays a moving image or still image, and examples thereof may not only be a portable electronic apparatus, such as a mobile phone, a smart phone, a tablet personal computer (PC), a mobile communication terminal, an electronic notebook, an electronic book, a portable multimedia player (PMP), a navigation device, or a ultra-mobile PC (UMPC), but may also be various products or a part thereof, such as a television, a laptop, a monitor, a billboard, or an Internet of things (IoT). In an embodiment, the electronic apparatus 1 may be a wearable device or a part thereof, such as a smart watch, a watch phone, a glasses-type display, or a head mounted display (HMD). However, embodiments are not limited thereto.

Examples of, the electronic apparatus 1 may include a dashboard of a vehicle, a center information display (CID) arranged on a center fascia or dashboard of a vehicle, a room mirror display instead of a side-view mirror of a vehicle, an entertainment display for a back seat of a vehicle, or a display arranged on the back of a front seat of a vehicle, a head up display (HUD) installed on the front of a vehicle or projected on a front window glass, or a computer generated hologram augmented reality head up display (CGH AR HUD). FIG. 7 shows a case where the electronic apparatus 1 is a smart phone for convenience of explanation.

The electronic apparatus 1 may include a display area DA and a non-display area NDA outside the display area DA. A display apparatus may implement an image through a two-dimensional array of pixels that are arranged in the display area DA.

The non-display area NDA may be an area that does not display an image, and may surround the display area DA. A driver for providing electrical signals or power to display devices arranged on the display area DA may be arranged in the non-display area (NDA). A pad, which is an area to which an electronic element or a printed circuit board may be electrically connected, may be arranged in the non-display area (NDA).

In the electronic apparatus 1, a length in an x-axis direction and a length in a y-axis direction may be different from each other. In an embodiment, as shown in FIG. 7, a length in the x-axis direction may be shorter than a length in the y-axis direction.

In an embodiment, a length in the x-axis direction may be the same as a length in the y-axis direction. In an embodiment, a length in the x-axis direction may be greater than a length in the y-axis direction.

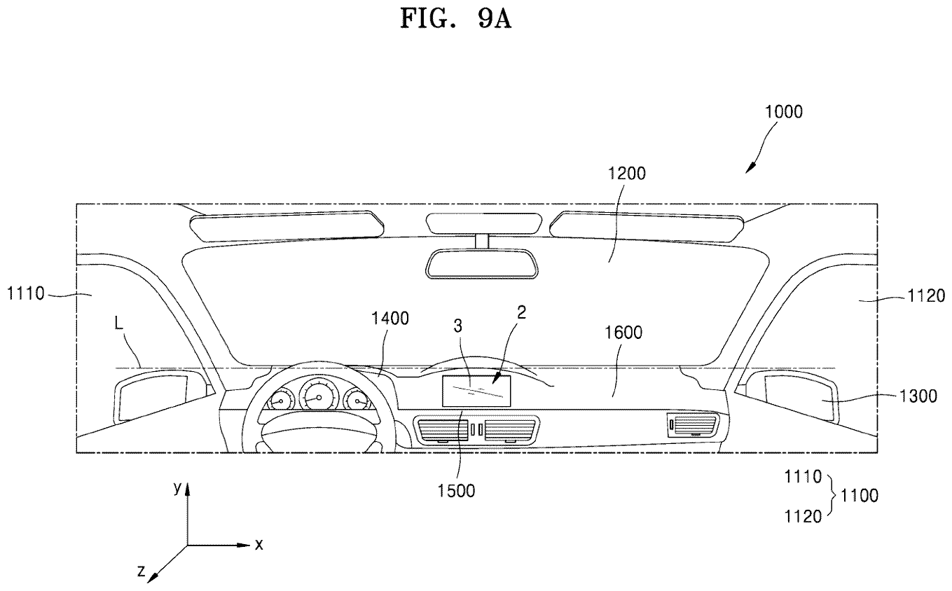

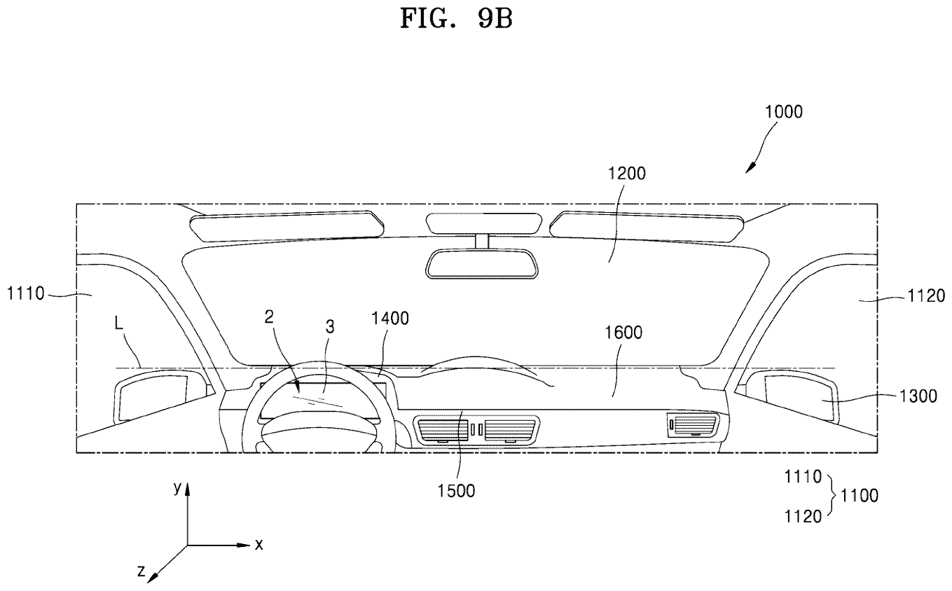

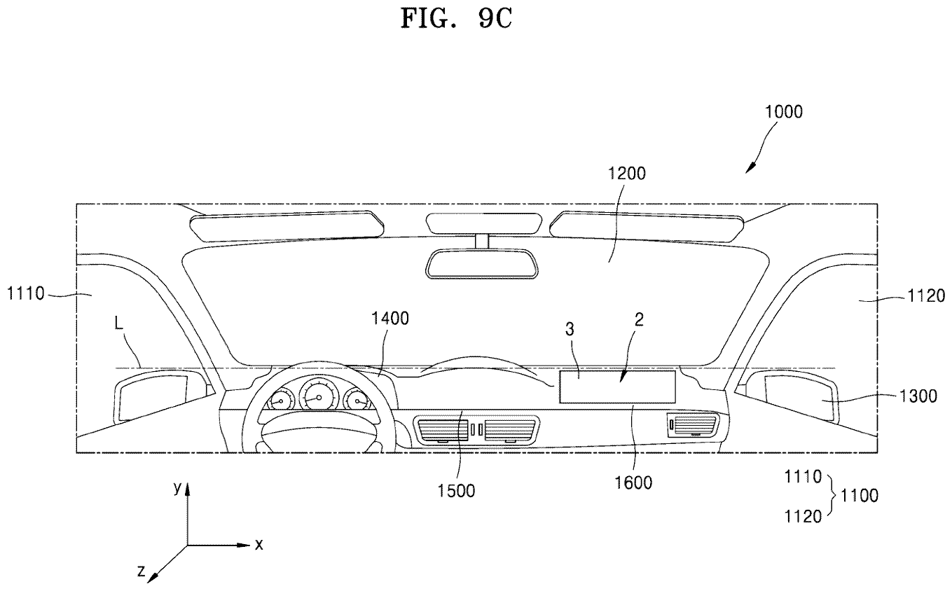

### [Descriptions of FIGS. 8 and 9A to 9C]

FIG. 8 is a schematic view of an exterior of a vehicle 1000 as an electronic apparatus including an opto-electronic device according to an embodiment. FIGS. 9A to 9C are each a schematic diagram of an interior of the vehicle 1000 according to embodiments.

Referring to FIGS. 8, 9A, 9B, and 9C, the vehicle 1000 may include various apparatuses for moving a subject to be transported, such as a person, an object, or an animal, from a departure point to a destination point. The vehicle 1000 may include a vehicle traveling on a road or track, a vessel moving over the sea or river, an airplane flying in the sky using the action of air, and the like.

The vehicle 1000 may travel on a road or a track. The vehicle 1000 may move in a selectable direction according to the rotation of at least one wheel. In an embodiment, the vehicle 1000 may include a three-wheeled or four-wheeled vehicle, a construction machine, a two-wheeled vehicle, a prime mover device, a bicycle, and a train running on a track.

The vehicle 1000 may include a vehicle body having an interior and an exterior, and a chassis that is a portion of the body in which mechanical apparatuses necessary for driving are installed as other parts except for the vehicle body. The exterior of the vehicle body may include a front panel, a bonnet, a roof panel, a rear panel, a trunk, a pillar provided at a boundary between doors, and the like. The chassis of the vehicle 1000 may include a power generating device, a power transmitting device, a driving device, a steering device, a braking device, a suspension device, a transmission device, a fuel device, front and rear wheels, left and right wheels, and the like.

The vehicle 1000 may include a side window glass 1100, a front window glass 1200, a side-view mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on a side of the vehicle 1000. In an embodiment, the side window glass 1100 may be installed on a door of the vehicle 1000. Multiple side window glasses 1100 may be provided and may face each other.

In an embodiment, the side window glass 1100 may include a first side window glass 1110 and a second side window glass 1120. In an embodiment, the first side window glass 1110 may be arranged adjacent to the cluster 1400. The second side window glass 1120 may be arranged adjacent to the passenger seat dashboard 1600.

In an embodiment, the side window glasses 1100 may be spaced apart from each other in an x direction or a −x direction. In an embodiment, the first side window glass 1110 and the second side window glass 1120 may be spaced apart from each other in the x direction or the −x direction. For example, a virtual straight line L connecting the side window glasses 1100 may extend in the x direction or the −x direction. In an embodiment, a virtual straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side-view mirror 1300 may provide a rear view of the vehicle 1000. The side-view mirror 1300 may be installed on the exterior of the vehicle body. In an embodiment, multiple side-view mirrors 1300 may be provided. Any one of the side-view mirrors 1300 may be arranged outside the first side window glass 1110 and another of the side-view mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning light, a seat belt warning light, an odometer, a tachograph, an automatic shift selector indicator lamp, a door open warning light, an engine oil warning light, and/or a low fuel warning light.

The center fascia 1500 may include a control panel on which buttons for adjusting an audio device, an air conditioning device, and a seat heater are disposed. The center fascia 1500 may be arranged on one side of the cluster 1400.

The passenger seat dashboard 1600 may be spaced apart from the cluster 1400, and the center fascia 1500 arranged between the cluster 1400 and the passenger seat dashboard 1600. In an embodiment, the cluster 1400 may be arranged to correspond to a driver seat (not shown), and the passenger seat dashboard 1600 may be arranged to correspond to a passenger seat (not shown). In an embodiment, the cluster 1400 may be adjacent to the first side window glass 1110, and the passenger seat dashboard 1600 may be adjacent to the second side window glass 1120.

In an embodiment, the display apparatus 2 may include a display panel 3, and the display panel 3 may display an image. The display apparatus 2 may be arranged inside the vehicle 1000. In an embodiment, the display apparatus 2 may be arranged between the side window glasses 1100 facing each other. The display apparatus 2 may be arranged on at least one of the cluster 1400, the center fascia 1500, and the passenger seat dashboard 1600.

The display apparatus 2 may include an organic light-emitting display, an inorganic electroluminescent display, a quantum dot display, and the like. Hereinafter, an organic light-emitting display apparatus including the light-emitting device according to an embodiment will be described as an example of the display apparatus 2. However, various types of display apparatuses as described above may be used in embodiments.

Referring to FIG. 9A, the display device 2 may be arranged on the center fascia 1500. In an embodiment, the display apparatus 2 may display navigation information. In an embodiment, the display apparatus 2 may display information regarding audio settings, video settings, or vehicle settings.

Referring to FIG. 9B, the display device 2 may be arranged on the cluster 1400. In an embodiment, the cluster 1400 may display driving information and the like through the display apparatus 2. For example, the cluster 1400 may digitally implement driving information. The digital cluster 1400 may display vehicle information and driving information as images. In an embodiment, a needle and a gauge of a tachometer and various warning light icons may be displayed by a digital signal.

Referring to FIG. 9C, the display device 2 may be arranged on the passenger seat dashboard 1600. The display apparatus 2 may be embedded in the passenger seat dashboard 1600 or arranged on the passenger seat dashboard 1600. In an embodiment, the display apparatus 2 arranged on the passenger seat dashboard 1600 may display an image related to information displayed on the cluster 1400 and/or information displayed on the center fascia 1500. In an embodiment, the display apparatus 2 arranged on the passenger seat dashboard 1600 may display information different from information displayed on the cluster 1400 and/or information displayed on the center fascia 1500.

### [Manufacturing Method]

The layers constituting the hole transport region, the emission layer, the layers constituting the photoactive layer, and/or the layers constituting the electron transport region may be formed in a selected region by using various methods such as vacuum deposition, spin coating, casting, Langmuir-Blodgett (LB) deposition, ink-jet printing, laser-printing, laser-induced thermal imaging, and the like.

When the layers constituting the hole transport region, the emission layer, the layers constituting the photoactive layer, and/or the layers constituting the electron transport region are formed by vacuum deposition, the deposition may be performed at a deposition temperature of about 100° C. to about 500° C., a vacuum degree of about 10⁻⁸ to π to about 10⁻³ torr, and a deposition speed of about 0.01 Å/sec to about 100 Å/sec, depending on a material to be included in a layer to be formed and the structure of a layer to be formed.

### [Definitions of Terms]

The term “C₃-C₆₀ carbocyclic group” as used herein may be a cyclic group consisting of only carbon atoms as ring-forming atoms and having 3 to 60 carbon atoms. The term “C₁-C₆₀ heterocyclic group” as used herein may be a cyclic group that has 1 to 60 carbon atoms and further has, in addition to carbon, at least one heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. In an embodiment, the number of ring-forming atoms of the C₁-C₆₀ heterocyclic group may be 3 to 61.

The “cyclic group” as used herein may be a C₃-C₆₀ carbocyclic group or a C₁-C₆₀ heterocyclic group.

The term “π electron-rich C₃-C₆₀ cyclic group” as used herein may be a cyclic group that has 3 to 60 carbon atoms and may not include *—N═*′ as a ring-forming moiety.

The term “π electron-deficient nitrogen-containing C₁-C₆₀ cyclic group” as used herein may be a heterocyclic group that has 1 to 60 carbon atoms and may include *—N═*′ as a ring-forming moiety.

In an embodiment,
a C₃-C₆₀ carbocyclic group may be a T1 group, or a group in which two or more T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
a C₁-C₆₀ heterocyclic group may be a T2 group, a group in which two or more T2 groups are condensed with each other, or a group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
a π electron-rich C₃-C₆₀ cyclic group may be a T1 group, a group in which two or more T1 groups are condensed with each other, a T3 group, a group in which two or more T3 groups are condensed with each other, or a group in which at least one T3 group and at least one T1 group are condensed with each other (for example, a C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, or the like),
a π electron-deficient nitrogen-containing C₁-C₆₀ cyclic group may be a T4 group, a group in which two or more T4 groups are condensed with each other, a group in which at least one T4 group and at least one T1 group are condensed with each other, a group in which at least one T4 group and at least one T3 group are condensed with each other, or a group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like).

The T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group.

The T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

The T3 group may be a furan group, a thiophene group, a 1H-pyrrole group, a silole group, or a borole group.

The T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group” as used herein may each be a group condensed to any cyclic group, a monovalent group, or a polyvalent group (for example, a divalent group, a trivalent group, a tetravalent group, etc.) according to the structure of a formula for which the corresponding term is used.

For example, “benzene group” may be a benzo group, a phenyl group, a phenylene group, or the like, which may be readily understood by one of ordinary skill in the art according to the structure of a formula including the “benzene group.”

Examples of a monovalent C₃-C₆₀ carbocyclic group or a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of a divalent C₃-C₆₀ carbocyclic group or a divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein may be a linear or branched monovalent aliphatic hydrocarbon group that has one to sixty carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group.

The term “C₁-C₆₀ alkylene group” as used herein may be a divalent group having a same structure as the C₁-C₆₀ alkyl group.

The term “C₂-C₆₀ alkenyl group” as used herein may be a monovalent hydrocarbon group having at least one carbon-carbon double bond in the middle or at a terminus of a C₂-C₆₀ alkyl group, and examples thereof may include an ethenyl group, a propenyl group, and a butenyl group.

The term “C₂-C₆₀ alkenylene group” as used herein may be a divalent group having a same structure as the C₂-C₆₀ alkenyl group.

The term “C₂-C₆₀ alkynyl group” as used herein may be a monovalent hydrocarbon group having at least one carbon-carbon triple bond in the middle or at a terminus of a C₂-C₆₀ alkyl group, and examples thereof may include an ethynyl group and a propynyl group.

The term “C₂-C₆₀ alkynylene group” as used herein may be a divalent group having a same structure as the C₂-C₆₀ alkynyl group.

The term “C₁-C₆₀ alkoxy group” as used herein may be a monovalent group represented by —O(A₁₀₁) (wherein A₁₀₁ may be a C₁-C₆₀ alkyl group), and examples thereof may include a methoxy group, an ethoxy group, and an isopropyloxy group.

The term “C₃-C₁₀ cycloalkyl group” as used herein may be a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and the like.

The term “C₃-C₁₀ cycloalkylene group” as used herein may be a divalent group having a same structure as the C₃-C₁₀ cycloalkyl group.

The term “C₁-C₁₀ heterocycloalkyl group” as used herein may be a monovalent cyclic group that has one to ten carbon atoms and further includes, in addition to the carbon atoms, at least one heteroatom as a ring-forming atom, and examples thereof may include a 1,2,3,4-oxatriazolidinyl group, a tetrahydrofuranyl group, and a tetrahydrothiophenyl group.

The term “C₁-C₁₀ heterocycloalkylene group” as used herein may be a divalent group having a same structure as the C₁-C₁₀ heterocycloalkyl group.

The term “C₃-C₁₀ cycloalkenyl group” as used herein refers to a monovalent cyclic group that has three to ten carbon atoms and at least one carbon-carbon double bond in the cyclic structure thereof and no aromaticity, and examples thereof may include a cyclopentenyl group, a cyclohexenyl group, and a cycloheptenyl group.

The term “C₃-C₁₀ cycloalkenylene group” as used herein may be a divalent group having a same structure as the C₃-C₁₀ cycloalkenyl group.

The term “C₁-C₁₀ heterocycloalkenyl group” as used herein may be a monovalent cyclic group that has one to ten carbon atoms, further includes, in addition to the carbon atoms, at least one heteroatom as a ring-forming atom, and has at least one double bond in the cyclic structure thereof. Examples of a C₁-C₁₀ heterocycloalkenyl group may include a 4,5-dihydro-1,2,3,4-oxatriazolyl group, a 2,3-dihydrofuranyl group, and a 2,3-dihydrothiophenyl group.

The term “C₁-C₁₀ heterocycloalkenylene group” as used herein may be a divalent group having a same structure as the C₁-C₁₀ heterocycloalkenyl group.

The term “C₆-C₆₀ aryl group” as used herein may be a monovalent group having a carbocyclic aromatic system of 6 to 60 carbon atoms.

The term “C₆-C₆₀ arylene group” as used herein may be a divalent group having a carbocyclic aromatic system of 6 to 60 carbon atoms.

Examples of a C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group.

When the C₆-C₆₀ aryl group and the C₆-C₆₀ arylene group each include two or more rings, the respective two or more rings may be condensed with each other.

The term “C₁-C₆₀ heteroaryl group” as used herein may be a monovalent group having a heterocyclic aromatic system of 1 to 60 carbon atoms, further including, in addition to carbon atoms, at least one heteroatom, as ring-forming atoms.

The term “C₁-C₆₀ heteroarylene group” as used herein may be a divalent group having a heterocyclic aromatic system of 1 to 60 carbon atoms, further including, in addition to carbon atoms, at least one heteroatom, as ring-forming atoms.

Examples of a C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group.

When the C₁-C₆₀ heteroaryl group and the C₁-C₆₀ heteroarylene group each include two or more rings, the respective two or more rings may be condensed with each other.

The term “monovalent non-aromatic condensed polycyclic group” as used herein may be a monovalent group having two or more rings condensed with each other, only carbon atoms (for example, eight to sixty carbon atoms) as ring-forming atoms, and no aromaticity in its molecular structure when considered as a whole.

Examples of a monovalent non-aromatic condensed polycyclic group include an indenyl group, a fluorenyl group, a spiro-bifluorenyl group, a benzofluorenyl group, an indenophenanthrenyl group, and an indeno anthracenyl group.

The term “divalent non-aromatic condensed polycyclic group” as used herein may be a divalent group having a same structure as the monovalent non-aromatic condensed polycyclic group.

The term “monovalent non-aromatic condensed heteropolycyclic group” as used herein may be a monovalent group that has two or more rings condensed with each other, further includes, in addition to carbon atoms (for example, one to sixty carbon atoms), at least one heteroatom as a ring-forming atom, and has no aromaticity in its molecular structure when considered as a whole. Examples of a monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group.

The term “divalent non-aromatic condensed heteropolycyclic group” as used herein may be a divalent group having a same structure as the monovalent non-aromatic condensed heteropolycyclic group.

The term “C₆-C₆₀ aryloxy group” as used herein may be a group represented by —O(A₁₀₂) (wherein A₁₀₂ may be a C₆-C₆₀ aryl group).

The term “C₆-C₆₀ arylthio group” as used herein may be a group represented by —S(A₁₀₃) (wherein A₁₀₃ may be a C₆-C₆₀ aryl group).

The term “C₇-C₆₀ arylalkyl group” as used herein may be a group represented by -(A₁₀₄)(A₁₀₅) (wherein A₁₀₄ may be a C₁-C₅₄ alkylene group, and A₁₀₅ may be a C₆-C₅₉ aryl group).

The term “C₂-C₆₀ heteroarylalkyl group” as used herein may be a group represented by -(A₁₀₆)(A₁₀₇) (wherein A₁₀₆ may be a C₁-C₅₉ alkylene group, and A₁₀₇ may be a C₁-C₅₉ heteroaryl group).

In the specification, the term “R₁₀ₐ” may be:
deuterium, —F, —CI, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —CI, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In the specification, the groups Q₁ to Q₃, Q₁₁ to Q₁₃, Q₂₁ to Q₂₃, and Q₃₁ to Q₃₃ used herein may each independently be: hydrogen; deuterium; —F; —CI; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as used herein may be any atom other than a carbon atom or a hydrogen atom. Examples of a heteroatom may include O, S, N, P, Si, B, Ge, Se, or any combination thereof.

The term “third-row transition metal” used herein may include hafnium (Hf), tantalum (Ta), tungsten (W), rhenium (Re), osmium (Os), iridium (Ir), platinum (Pt), gold (Au), and the like.

In the specification, the term “Ph” refers to a phenyl group, the term “Me” refers to a methyl group, the term “Et” refers to an ethyl group, the terms “tert-Bu” and “Bu^{t}” each refer to a tert-butyl group, and the term “OMe” refers to a methoxy group.

The term “biphenyl group” as used herein may be a “phenyl group substituted with a phenyl group.” For example, a “biphenyl group” may be a substituted phenyl group having a C₆-C₆₀ aryl group as a substituent.

The term “terphenyl group” as used herein may be a “phenyl group substituted with a biphenyl group”. For example, a “terphenyl group” may be a substituted phenyl group having, as a substituent, a C₆-C₆₀ aryl group substituted with a C₆-C₆₀ aryl group.

The symbols * and *′ as used herein, unless defined otherwise, each refer to a binding site to a neighboring atom in a corresponding formula or moiety.

The x-axis, y-axis, and z-axis as used herein are not limited to three axes in an orthogonal coordinate system (e.g., a Cartesian coordinate system), and may be interpreted in a broader sense than the aforementioned three axes in an orthogonal coordinate system. For example, the x-axis, y-axis, and z-axis may describe axes that are orthogonal to each other, or may describe axes that are in different directions that are not orthogonal to each other.

Hereinafter, compounds according to embodiments and light-emitting devices according to embodiments will be described in detail with reference to the following Synthesis Examples and Examples. The wording “B was used instead of A” used in describing Synthesis Examples refers to that an identical molar equivalent of B was used in place of A.

### Synthesis Examples and Examples

### Synthesis Example 1: Synthesis of Compound P2

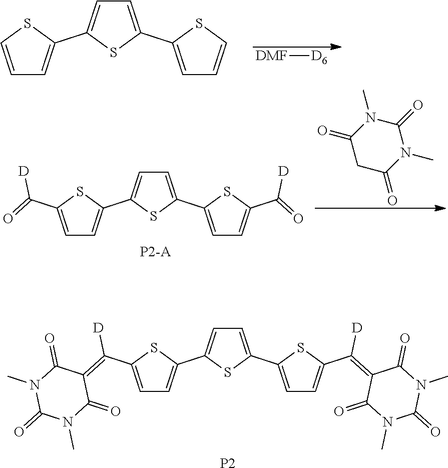

### Synthesis of Intermediate P2-A

1.24 g (5 mmol) of 2,2′:5′,2″-terthiophene was dissolved in 50 ml of dehydrated tetrahydrofuran. 4 ml (16.0 mmol) of 2.76 M n-butyl lithium (n-BuLi) hexane solution was added dropwise thereto at −78° C. for 5 minutes, and stirred at room temperature for 30 minutes. The temperature was lowered again to −78° C., and 0.7 g (10 mmol) of dehydrated N,N′-dimethyl-D6-formamide was added thereto and stirred for 30 minutes.

The temperature was raised to room temperature. After adding water to terminate the reaction, an extraction process was performed thereon with ethyl acetate three times, and the organic layer thus extracted was dried by adding anhydrous magnesium sulfate thereto. The product obtained at this time was separated and purified through silica gel column chromatography (hexane:dichloromethane=1:1 volume ratio) to obtain 0.98 g (yield: 64%) of Intermediate P2-A. The resultant compound was identified through MS/FAB.

C₁₄H₆D₂O₂S₃: calc. 306.41, found 306.56

### Synthesis of Compound P2

1.07 g (3.5 mmol) of Intermediate P2-A was dissolved in 20 ml of ethanol, and 1.11 g (7.14 mmol) of 1,3-dimethyl-2-barbituric acid was added thereto and stirred at 50° C. for 2 hours, followed by concentrating under reduced pressure. After recrystallization using chloroform and ethanol, the organic layer of Compound 1 was dried with magnesium sulfate, the solvent was evaporated, and the resulting residue was purified by silica gel column chromatography to obtain 1.29 g (yield: 68%) of Compound P2. The resultant compound was identified by ¹H NMR (CDCl₃, 400 MHz) and MS/FAB.

δ=7.89-7.86 (s, 4H), 7.55 (s, 2H), 3.22 (s, 12H)

C₂₆H₁₈D₂N₄O₆S₃: calc. 582.66, found 582.62 Synthesis Example 2: Synthesis of Compound P3

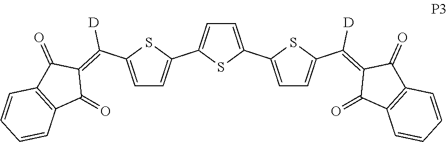 [SMILES: [2H]C(=C1C(=O)c2ccccc2C1=O)c1ccc(-c2ccc(-c3ccc(C([2H])=C4C(=O)c5ccccc5C4=O)s3)s2)s1]

Compound P3 was synthesized in the same method as used to synthesize Compound P2, except that 1,3-indandione was used instead of 1,3-dimethyl-2-barbituric acid in the synthesis of Compound P2 in Synthesis Example 1. The resultant compound was identified by ¹H NMR (CDCl₃, 400 MHz) and MS/FAB.

δ=7.89-7.86 (m, 4H), 7.73-7.71 (m, 8H), 7.55 (s, 2H)

C₃₂H₁₄D₂O₄S₃: calc. 562.67, found 562.83

### Synthesis Example 3: Synthesis of Compound P8

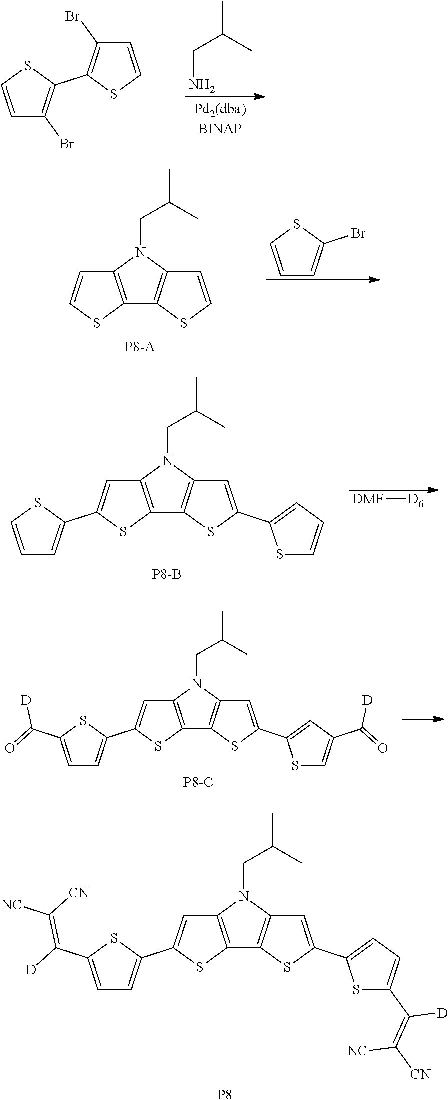

### Synthesis of Intermediate P8-A

6.48 g (20.0 mmol) of 3,4′-dibromo-2,2′-bithiophene, 1.46 g (20.0 mmol) of 2-methylbutan-1-amine, 0.37 g (0.4 mmol) of Pd₂(dba)₃, 0.75 g (1.2 mmol) of BINAP, and 5.76 g (60.0 mmol) of t-BuOK were dissolved in 90 ml of toluene and stirred at 120° C. for 24 hours. After the reaction solution was cooled to room temperature, an extraction process was performed thereon with 50 ml of water and 50 ml of diethyl ether three times. The organic layer thus collected was dried with magnesium sulfate, and a residue obtained by evaporating the solvent was subjected to separation and purification through silica gel chromatography, so as to obtain 2.58 g (yield: 55%) of Intermediate P8-A. The resultant compound was identified through MS/FAB.

C₁₂H₁₃NS₂: calc. 235.36, found 235.41

### Synthesis of Intermediate P8-B

Intermediate P8-B was synthesized in the same manner as the synthesis of Intermediate P2-A in Synthesis Example 1, except that Intermediate P8-A was used instead of 2,2′:5′,2″-terthiophene and 2-bromothiophene was used instead of dehydrated N,N′-dimethyl-D6-formamide. The resultant compound was identified through MS/FAB.

C₂₀H₁₇NS₄: calc. 399.60, found 399.69

### Synthesis of Intermediate P8-C

Intermediate P8-C was synthesized in the same manner as the synthesis of Intermediate P2-A in Synthesis Example 1, except that Intermediate P8-B was used instead of 2,2′:5′,2″-terthiophene. The resultant compound was identified through MS/FAB.

C₂₂H₁₅D₂NO₂S₄: calc. 457.64, found 457.71

### Synthesis of Compound P8

Compound P8 was synthesized in the same manner as the synthesis of Compound P2 in Synthesis Example 1, except that P8-C was used instead of intermediate P2-A and malonitrile was used instead of 1,3-dimethyl-2-barbituric acid. The resultant compound was identified by ¹H NMR (CDCl₃, 400 MHz) and MS/FAB.

δ=7.51-7.49 (m, 2H), 7.21-7.17 (m, 4H), 4.05-4.03 (m, 2H), 2.05-2.02 (m, 1H), 0.91 (s, 6H)

C₂₈H₁₅D₂N₅S₄: calc. 553.73, found 553.83

### Synthesis Example 4: Synthesis of Compound P9

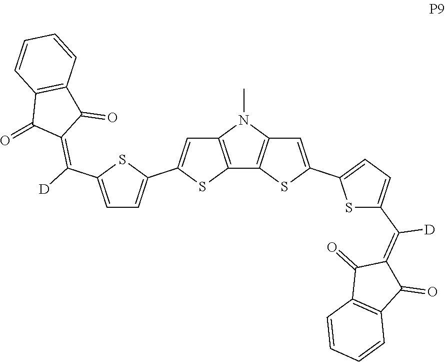 [SMILES: [2H]C(=C1C(=O)c2ccccc2C1=O)c1ccc(-c2cc3c(s2)c2sc(-c4ccc(C([2H])=C5C(=O)c6ccccc6C5=O)s4)cc2n3C)s1]

Compound P9 was synthesized in the same manner as the synthesis of Compound P8 above, except that in the synthesis of Intermediate P8-A in Synthetic Example 3, methylamine was used instead of 2-methylbutan-1-amine and the synthesis of Compound P8, indandione was used instead of malonitrile in. The resultant compound was identified by ¹H NMR (CDCl₃, 400 MHz) and MS/FAB.

δ=7.89-7.86 (m, 4H), 7.73-7.71 (m, 8H), 7.17 (s, 2H), 3.72 (s, 3H)

C₃₇H₁₇D₂NO₄S₄: calc. 671.81, found 671.89

### Synthesis Example 5: Synthesis of Compound P12

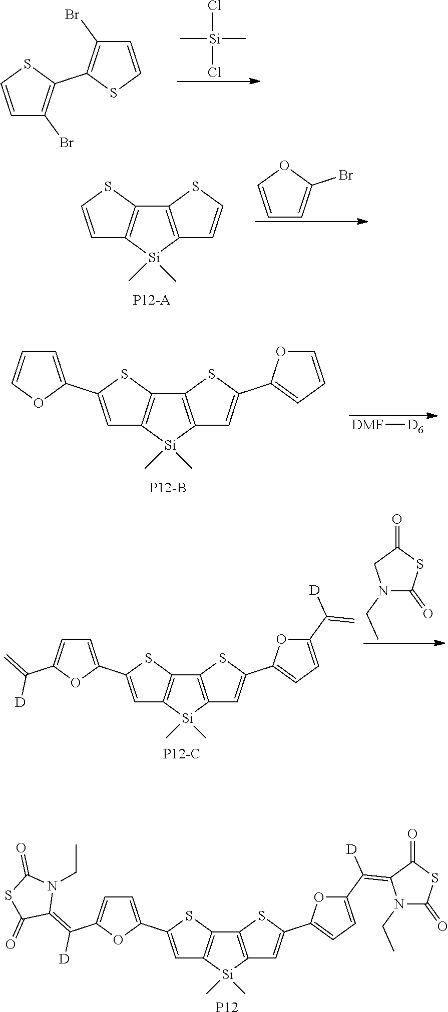

### Synthesis of Intermediate P12-A

6.48 g (20.0 mmol) of 3,4′-dibromo-2,2′-bithiophene was dissolved in 150 ml of dehydrated tetrahydrofuran. 8 ml (32.0 mmol) of 2.76 M n-butyl lithium (n-BuLi) hexane solution was added dropwise thereto at −78° C. for 5 minutes, and stirred for 30 minutes. At this temperature, 2.71 g (21.0 mmol) of dichlorodimethylsilance was added and stirred for 30 minutes, and the temperature was raised to room temperature. After twenty four hours of stirring, water was added thereto to terminate the reaction. An extraction process was performed thereon using ethyl acetate three times, and the organic layer thus extracted was dried by adding anhydrous magnesium sulfate thereto. The product obtained at this time was separated and purified through silica gel column chromatography (hexane:dichloromethane=1:1 volume ratio) to obtain 3.34 g (yield: 75%) of Intermediate P12-A. The resultant compound was identified through MS/FAB.

C₁₀H₁₀S₂Si: calc. 222.40, found 222.48

### Synthesis of Intermediate P12-B

Intermediate P12-B was synthesized in the same manner as used to synthesize Intermediate P2-A, except that the synthesis of Intermediate P2-A in Synthesis Example 1, Intermediate P12-A was used instead of 2,2′:5′,2″-terthiophene and 2-bromofuran was used instead of dehydrated N,N′-dimethyl-D6-formamide. The resultant compound was identified through MS/FAB.

C₁₈H₁₄O₂S₂Si: calc. 399.60, found 399.69

### Synthesis of Intermediate P12-C

Intermediate P12-C was synthesized in the same manner as used to synthesize Intermediate P2-A, except that in the synthesis of Intermediate P2-A in Synthesis Example 1, Intermediate P12-B was used instead of 2,2′:5′,2″-terthiophene.

The resultant compound was identified through MS/FAB.

C₂₂H₁₆D₂O₂S₂Si: calc. 408.60, found 408.67

### Synthesis of Compound P12

Compound P12 was synthesized in the same manner as used to synthesize Compound P2, except that the synthesis of Compound P2 in Synthesis Example 1, P12-C was used instead of Intermediate P2-A and 3-ethyl-2,5-thiazolidinedione was used instead of 1,3-dimethyl-2-barbituric acid. The resultant compound was identified by ¹H NMR (CDCl₃, 400 MHz) and MS/FAB.

δ=7.44-7.42 (m, 2H), 7.24-7.22 (m, 2H), 7.14 (s, 2H), 3.72-3.70 (m, 4H), 1.28-1.25 (m, 6H), 0.14 (s, 6H)

C₃₀H₂₂D₂N₂O₆S₄Si: calc. 666.87, found 666.91

### Synthesis Example 6: Synthesis of Compound P15

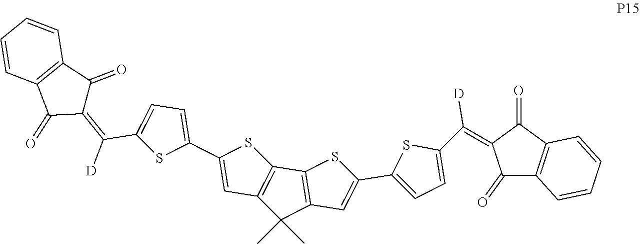 [SMILES: [2H]C(=C1C(=O)c2ccccc2C1=O)c1ccc(-c2cc3c(s2)-c2sc(-c4ccc(C([2H])=C5C(=O)c6ccccc6C5=O)s4)cc2C3(C)C)s1]

The same method as used to synthesize Intermediate P12-B was used, except that in the synthesis of Intermediate P12-B in Synthesis Example 5, 4,4-dimethyl-4H-cyclopenta[2,1-b:3,4-b′]dithiophene was used instead of Intermediate P12-A and 2-bromothiophene was used instead of 2-bromofuran, and the same method as used to synthesize Compound P12 was used, except that in the synthesis of Compound P12, indandione was used instead of 3-ethyl-2,5-thiazolidinedione, so as to synthesize Compound P15. The resultant compound was identified by ¹H NMR (CDCl₃, 400 MHz) and MS/FAB.

δ=7.89-7.86 (m, 4H), 7.72-7.70 (m, 8H), 7.16 (s, 2H), 1.69 (s, 6H)

C₃₉H₂₀D₂O₄S₄: calc. 684.85, found 684.90

### Synthesis Example 7: Synthesis of Compound P17

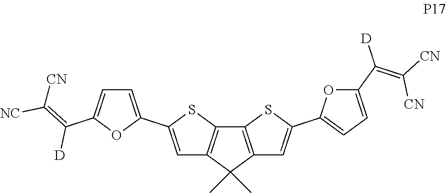 [SMILES: [2H]C(=C(C#N)C#N)c1ccc(-c2cc3c(s2)-c2sc(-c4ccc(C([2H])=C(C#N)C#N)o4)cc2C3(C)C)o1]

Compound P17 was synthesized in the same manner as used to synthesize Compound P15, except that, in the synthesis of Compound P15 in Synthetic Example 6, 2-bromofuran was used instead of 2-bromothiophene and malonitrile was used instead of 3-ethyl-2,5-thiazolidinedione. The resultant compound was identified by ¹H NMR (CDCl₃, 400 MHz) and MS/FAB.

δ=7.09-7.07 (m, 4H), 6.54-6.52 (m, 2H), 1.69 (s, 6H)

C₂₇H₁₂D₂N₄O₄S₂: calc. 492.57, found 492.63

### Synthesis Example 8: Synthesis of Compound P20

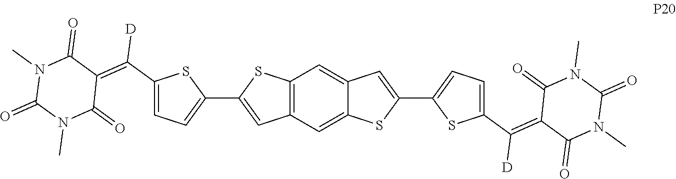 [SMILES: [2H]C(=C1C(=O)N(C)C(=O)N(C)C1=O)c1ccc(-c2cc3cc4sc(-c5ccc(C([2H])=C6C(=O)N(C)C(=O)N(C)C6=O)s5)cc4cc3s2)s1]

The same method as used to synthesize Intermediate P12-B was used, except that in the synthesis of Intermediate P12-B in Synthesis Example 5, benzo[1,2-b:4,5-b′]dithiophene was used instead of Intermediate P12-A and 2-bromothiophene was used instead of 2-bromofuran, and the same method as used to synthesize Compound P12 was used, except that in the synthesis of Compound P12, 1,3-dimethyl-2-barbituric acid was used instead of 3-ethyl-2,5-thiazolidinedione, so as to synthesize Compound P20. The resultant compound was identified by ¹H NMR (CDCl₃, 400 MHz) and MS/FAB.

δ=7.89-7.82 (m, 6H), 7.50-7.48 (m, 2H), 3.23 (s, 12H)

C₃₂H₂₀D₂N₄O₆S₄: calc. 688.80, found 688.94

### Synthesis Example 9: Synthesis of Compound P21

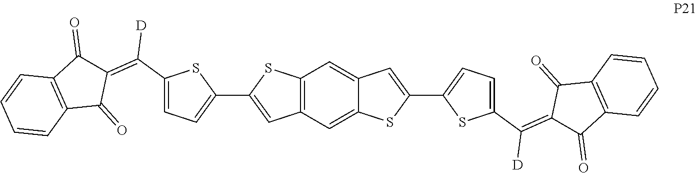 [SMILES: [2H]C(=C1C(=O)c2ccccc2C1=O)c1ccc(-c2cc3cc4sc(-c5ccc(C([2H])=C6C(=O)c7ccccc7C6=O)s5)cc4cc3s2)s1]

Compound P21 was synthesized in the same manner as used to synthesize Compound P20, except that in the synthesis of Compound 20 of Synthesis Example 8, indandione was used instead of 1,3-dimethyl-2-barbituric acid. The resultant compound was identified by ¹H NMR (CDCl₃, 400 MHz) and MS/FAB.

δ=7.90-7.82 (m, 6H), 7.72-7.68 (m, 8H), 7.50-7.49 (m, 2H)

C₃₈H₁₆D₂O₄S₄: calc. 764.91, found 946.96

### Synthesis Example 10: Synthesis of Compound P25

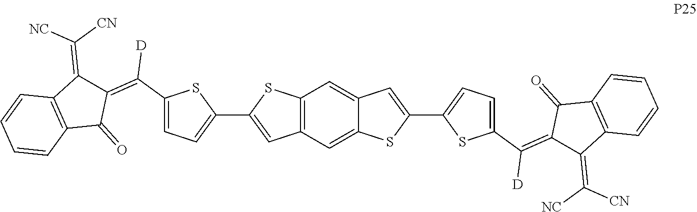 [SMILES: [2H]/C(=C1/C(=O)c2ccccc2C1=C(C#N)C#N)c1ccc(-c2cc3cc4sc(-c5ccc(/C([2H])=C6\C(=O)c7ccccc7C6=C(C#N)C#N)s5)cc4cc3s2)s1]

Compound P25 was synthesized in the same manner as used to synthesize Compound P20, except that in the synthesis of Compound 20 of Synthesis Example 8, 3-(dicyanomethylidene)indan-1-one was used instead of 1,3-dimethyl-2-barbituric acid. The resultant compound was identified by ¹H NMR (CDCl₃, 400 MHz) and MS/FAB.

δ=7.89-7.82 (m, 6H), 7.72-7.70 (m, 2H), 7.50-7.48 (m, 2H), 7.36-7.32 (m, 6H)

C₄₄H₁₆D₂N₄O₂S₄: calc. 688.81, found 688.86

### Synthesis Example 11: Synthesis of Compound P33

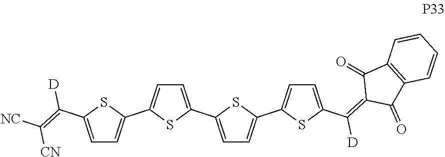 [SMILES: [2H]C(=C(C#N)C#N)c1ccc(-c2ccc(-c3ccc(-c4ccc(C([2H])=C5C(=O)c6ccccc6C5=O)s4)s3)s2)s1]

The same method as used to synthesize Intermediate P2-A was used, except that in the synthesis of Intermediate P2-A of Synthesis Example 1, 2,2′:5′,2″:5″,2′″-quaterthiophene was used instead of 2,2′:5′,2″-terthiophene. The same method as used to synthesize Compound P2 was used except that, in the synthesis of Compound 2, indandione and malonitrile were used instead of 1,3-dimethyl-2-barbituric acid, so as to synthesize Compound P33. The resultant compound was identified by ¹H NMR (CDCl₃, 400 MHz) and MS/FAB.

δ=7.89-7.86 (m, 2H), 7.72-7.70 (m, 4H), 7.55-7.51 (m, 5H), 7.19 (d 1H)

C₃₀H₁₂D₂N₂O₂S₄: calc. 564.71, found 564.72

### Synthesis Example 12: Synthesis of Compound P36

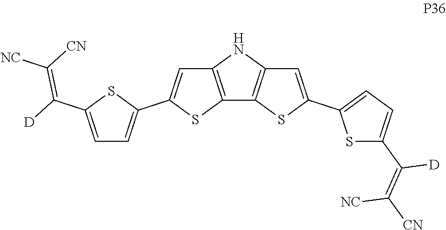 [SMILES: [2H]C(=C(C#N)C#N)c1ccc(-c2cc3[nH]c4cc(-c5ccc(C([2H])=C(C#N)C#N)s5)sc4c3s2)s1]

The same method as used to synthesize Intermediate P12-B was used, except that in the synthesis of Intermediate P12-B in Synthesis Example 5, 4H-dithieno[3,2-b:2′,3′-d]pyrrole was used instead of Intermediate P12-A, and the same method as used to synthesize Compound P12 was used, except that, in the synthesis of Compound P12, malonitrile was used instead of 3-ethyl-2,5-thiazolidinedione, so as to synthesize Compound P36. The resultant compound was identified by ¹H NMR (CDCl₃, 400 MHz) and MS/FAB.

δ=11.12 (s, 1H), 7.51-7.49 (m, 2H), 7.19-7.17 (m, 4H)

C₂₄H₇D₂N₅S₄: calc. 497.62, found 497.73

### Synthesis Example 13: Synthesis of Compound P41

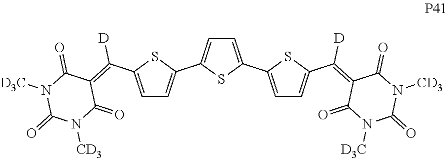 [SMILES: [2H]C(=C1C(=O)N(C([2H])([2H])[2H])C(=O)N(C([2H])([2H])[2H])C1=O)c1ccc(-c2ccc(-c3ccc(C([2H])=C4C(=O)N(C([2H])([2H])[2H])C(=O)N(C([2H])([2H])[2H])C4=O)s3)s2)s1]

Compound P41 was synthesized in the same manner as used to synthesize Compound P2, except that in the synthesis of Compound P2 in Synthesis Example 1, 1,3-bis(methyl-d₃)pyrimidine-2,4,6(1H,3H,5H)-trione was used instead of 1,3-dimethyl-2-barbituric acid. The resultant compound was identified by ¹H NMR (CDCl₃, 400 MHz) and MS/FAB.

δ=7.89-7.86 (m, 4H), 7.55-7.53 (m, 2H)

C₂₆H₆D₁₄N₄O₆S₃: calc. 594.73, found 594.79

### Synthesis Example 14: Synthesis of Compound P44

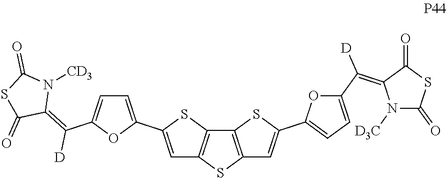 [SMILES: [2H]/C(=C1\C(=O)SC(=O)N1C([2H])([2H])[2H])c1ccc(-c2cc3sc4cc(-c5ccc(/C([2H])=C6/C(=O)SC(=O)N6C([2H])([2H])[2H])o5)sc4c3s2)o1]

The same method as used to synthesize Intermediate P12-B was used, except that in the synthesis of Intermediate P12-B in Synthesis Example 5, dithieno[3,2-b:2′,3′-d]thiophene was used instead of Intermediate P12-A, and the same method as used to synthesize Compound P12 was used, except that, in the synthesis of Compound P12, 3-(methyl-d₃)thiazolidine-2,5-dione) was used instead of 3-ethyl-2,5-thiazolidinedione, so as to synthesize Compound P44. The resultant compound was identified by ¹H NMR (CDCl₃, 400 MHz) and MS/FAB.

δ=7.42-7.40 (m, 2H), 7.24-7.20 (m, 2H), 7.10 (s, 2H)

C₂₆H₆D₈N₂O₆S₅: calc. 618.75, found 618.90

### Synthesis Example 15: Synthesis of Compound P46

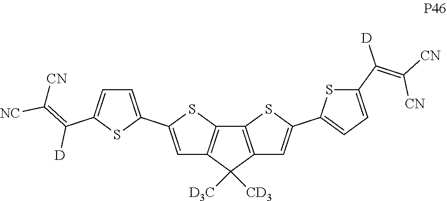 [SMILES: [2H]C(=C(C#N)C#N)c1ccc(-c2cc3c(s2)-c2sc(-c4ccc(C([2H])=C(C#N)C#N)s4)cc2C3(C([2H])([2H])[2H])C([2H])([2H])[2H])s1]

The same method as used to synthesize Intermediate P12-B was used, except that in the synthesis of Intermediate P12-B in Synthesis Example 5, 4,4-bis(methyl-d3)-4H-cyclopenta[2,1-b:3,4-b]dithiophene was used instead of Intermediate P12-A. The same method as used to synthesize Compound P12 was used, except that in the synthesis of Compound P12, malonitrile was used instead of 3-ethyl-2,5-thiazolidinedione, so as to synthesize Compound P46. The resultant compound was identified by ¹H NMR (CDCl₃, 400 MHz) and MS/FAB.

δ=7.51-7.49 (m, 2H), 7.19-7.16 (m, 4H)

C₂₇H₆D₈N₄S₄ calc. 530.73, found 530.76

### Synthesis Example 16: Synthesis of Compound P53

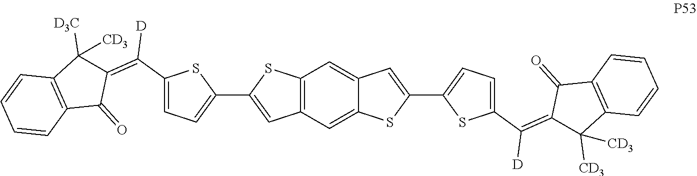 [SMILES: [2H]/C(=C1/C(=O)c2ccccc2C1(C([2H])([2H])[2H])C([2H])([2H])[2H])c1ccc(-c2cc3cc4sc(-c5ccc(/C([2H])=C6\C(=O)c7ccccc7C6(C([2H])([2H])[2H])C([2H])([2H])[2H])s5)cc4cc3s2)s1]

Compound P53 was synthesized in the same manner as used to synthesize Compound P20, except that in the synthesis of Compound P20 of Synthesis Example 8, 3,3-bis(methyl-d₃)-2,3-dihydro-1H-inden-1-one was used instead of 1,3-dimethyl-2-barbituric acid. The resultant compound was identified by ¹H NMR (CDCl₃, 400 MHz) and MS/FAB.

δ=7.89-7.82 (m, 6H), 7.66-7.50 (m, 8H), 7.41-7.39 (m, 2H)

C₄₂H₁₆D₁₄O₂S₄: calc. 709.03, found 709.11

### Example 1

As an anode, a Corning 15 Ω/cm² (1,200 Å) ITO glass substrate was cut to a size of 50 mm×50 mm×0.7 mm, sonicated with isopropyl alcohol and pure water each for 5 minutes, and cleaned by exposure to ultraviolet rays and ozone for 30 minutes. The ITO glass substrate was provided to a vacuum deposition apparatus.

2-TNATA was vacuum-deposited on the substrate to form a hole injection layer having a thickness of 600 Å, and 4,4′-bis[N-(1-naphthyl)-N-phenylamino]biphenyl (hereinafter, referred to as NPB), which is a hole-transporting material, was vacuum-deposited thereon to form a hole transport layer having a thickness of 300 Å.

A red auxiliary layer having a thickness of 300 Å was deposited on the hole transport layer, and a photoactive layer having a thickness of 500 Å was deposited thereon. The photoactive layer consists of a light absorption layer and a photoelectric conversion layer. Compound P2, which is a p-type semiconductor compound (p-type), was deposited as the light absorption material, and Compound N5, which is an n-type semiconductor compound (n-type), was deposited as the photoelectric conversion organic material. Alq₃ was deposited on the photoactive layer to form an electron transport layer having a thickness of 300 Å, and LiF, which is a halogenated alkali metal, was deposited on the electron transport layer to form an electron injection layer having a thickness of 10 Å, and Al was vacuum-deposited thereon to form a cathode having a thickness of 3,000 Å, to form a LiF/Al electrode, thereby completing the manufacture of an opto-electronic device.

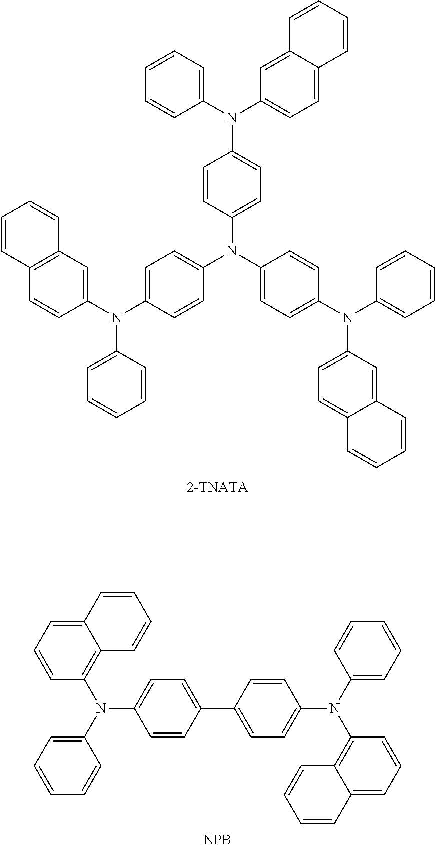 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

### Examples 2 to 16, and Comparative Examples 1 to 4

Opto-electronic devices were manufactured in the same manner as Example 1, except that the p-type Compound and n-type Compound shown in Table 2 were used when forming a photoactive layer.

### Evaluation Example 1: Evaluation of Compound Characteristics

The deposition temperature (Tₚ) at the standard internal pressure of the compounds synthesized in the Synthesis Example was measured using vacuum-thermogravimetric analysis (v-TGA), and results thereof are shown in Table 1.

In order to pre-verify the stability of a material in mass production, the thermal stability of the material was evaluated and verified over a long period of time under high vacuum and high temperature conditions using thermal stability evaluation equipment.

The deposition temperature Tₚ was measured by using a thermogravimetric analyzer, and the material in an ampoule was placed in a thermal stability evaluation facility, and thermal stability evaluation was performed thereon at the measured temperature for 100 hours. In order to confirm the degree of denaturation of the material, the material was measured by high performance liquid chromatography (HPLC) before and after being placed in the thermal stability evaluation equipment, and the change in HPLC purity is shown in Table 1. HPLC is an instrument that separates organic compounds in a solution by component and measures their content.

TABLE 1

| | Tₚ (° C., standard | HPLC purity change (%) |
|---|---|---|
| | internal | 100 hours after thermal |
| Compound | pressure) | stability evaluation |
| P2 | 264 | 0.03% |
| P3 | 275 | 0.05% |
| P8 | 287 | 0.08% |
| P9 | 288 | 0.06% |
| P12 | 294 | 0.1% |
| P15 | 262 | 0.05% |
| P17 | 264 | 0.1% |
| P20 | 295 | 0.1% |
| P21 | 288 | 0.07% |
| P25 | 275 | 0.09% |
| P33 | 286 | 0.06% |
| P36 | 290 | 0.1% |
| P41 | 285 | 0.07% |
| P44 | 280 | 0.1% |
| P46 | 269 | 0.05% |
| P53 | 289 | 0.06% |
| A | 301 | 28% |
| B | 305 | 34% |
| SubNC | 385 | 39% |

From Table 1, it can be seen that the compounds according to embodiments showed a small decrease in HPLC purity 100 hours after thermal stability evaluation, compared to Comparative Example Compounds A and B, and SubNC. Therefore, it can be confirmed that the compounds according to embodiments have relatively high heat resistance.

### Evaluation Example 2: Evaluation of Characteristics of Opto-Electronic Device

In order to evaluate the characteristics of the opto-electronic devices manufactured in Examples 1 to 16 and Comparative Examples 1 to 4, external quantum efficiency (EQE) and dark current density (J_{dark}) were measured. The measurements are shown in Table 2 below.

Light (550 nm to 650 nm) was radiated to the opto-electronic device using a xenon lamp device. The maximum absorption wavelength (λₘₐₓ) upon light radiation was measured using an ammeter (Keithley, Tektronix, USA), and the converted current was measured. The EQE was calculated using the radiated light and the measured current.

Voltage (−3 V) was applied to the anode using an IVL-25CH device. The current flowing when voltage was applied was measured using an ammeter (Keithley, Tektronix, USA). The dark current density (J_{dark}) was calculated using the measured current.

TABLE 2

| | p-type | n-type | EQE | J_{dark} | λₘₐₓ |
|---|---|---|---|---|---|
| | Compound | Compound | (%) | (mA@ −3 V) | (nm) |
| Example 1 | P2 | N5 | 31 | 3.6E−06 | 620 |
| Example 2 | P3 | N5 | 38 | 3.4E−06 | 625 |
| Example 3 | P8 | N5 | 30 | 2.4E−06 | 610 |
| Example 4 | P9 | N6 | 35 | 2.9E−06 | 630 |
| Example 5 | P12 | N6 | 36 | 2.5E−06 | 620 |
| Example 6 | P15 | N6 | 39 | 2.9E−06 | 630 |
| Example 7 | P17 | N6 | 31 | 4.1E−06 | 612 |
| Example 8 | P20 | N6 | 35 | 3.8E−06 | 628 |
| Example 9 | P21 | N8 | 34 | 4.06E−6 | 625 |
| Example 10 | P25 | N15 | 36 | 4.3E−06 | 632 |
| Example 11 | P33 | N16 | 30 | 3.2E−06 | 633 |
| Example 12 | P36 | N5 | 31 | 2.5E−06 | 618 |
| Example 13 | P41 | N5 | 33 | 2.0E−06 | 631 |
| Example 14 | P44 | N8 | 35 | 2.4E−06 | 625 |
| Example 15 | P46 | N8 | 34 | 3.1E−06 | 621 |
| Example 16 | P53 | N6 | 31 | 2.1E−06 | 630 |
| Comparative | A | N5 | 15 | 4.1E−06 | 620 |
| Example 1 | | | | | |
| Comparative | B | N5 | 11 | 5.3E−06 | 615 |
| Example 2 | | | | | |
| Comparative | SubNC | N5 | 5 | 7.7E−06 | 645 |
| Example 3 | | | | | |
| Comparative | SubNC | C60 | 10 | 8.0E−06 | 650 |
| Example 4 | | | | | |

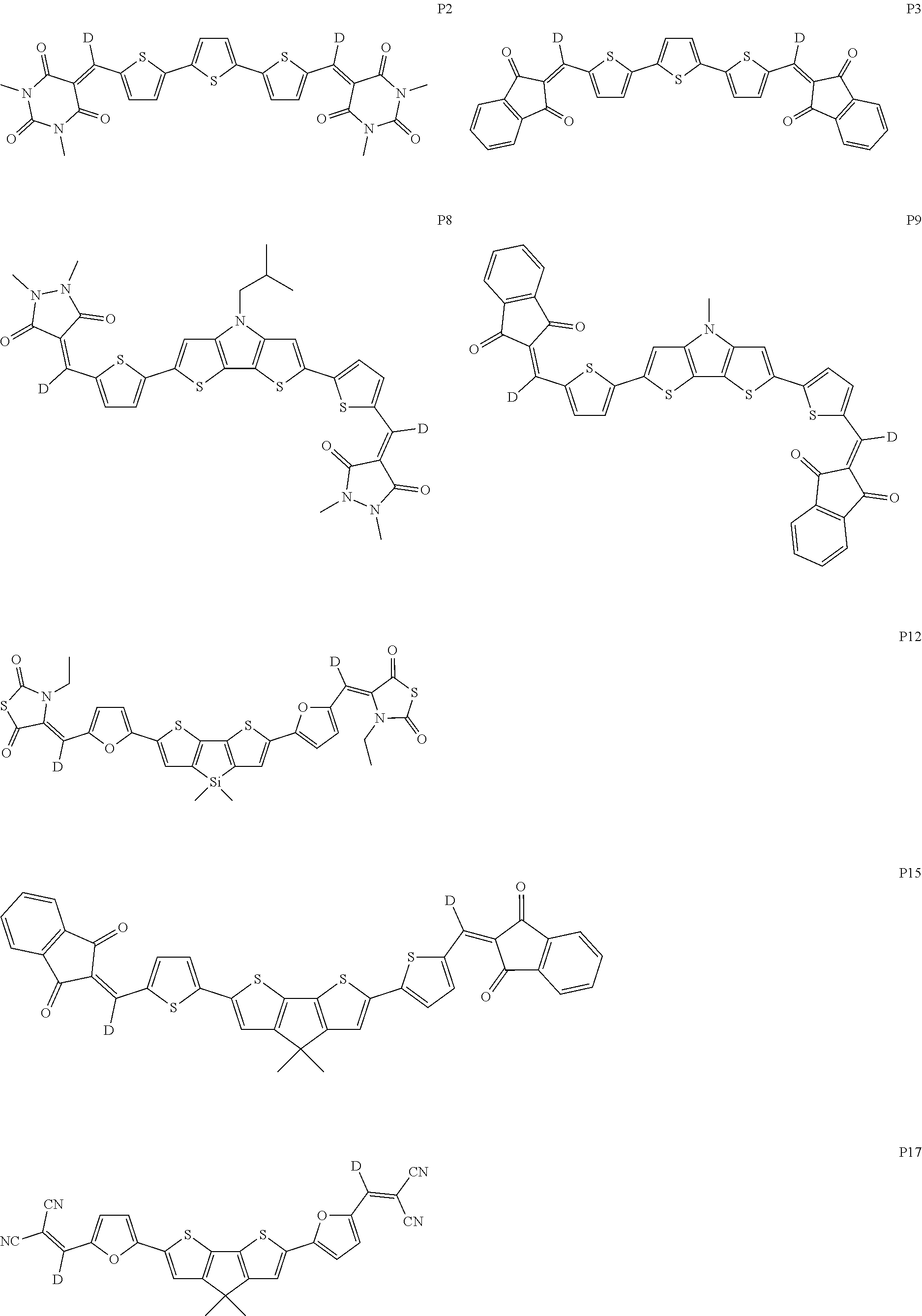

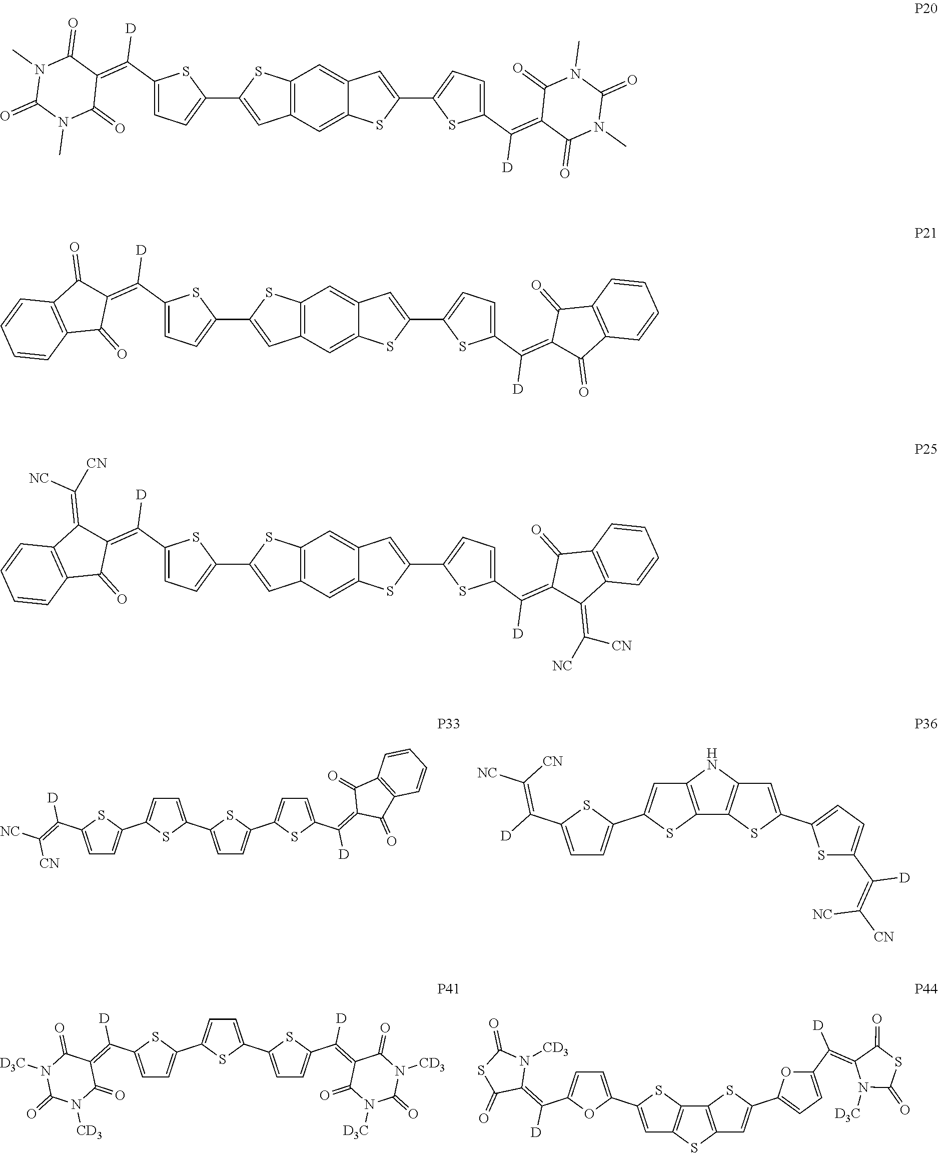

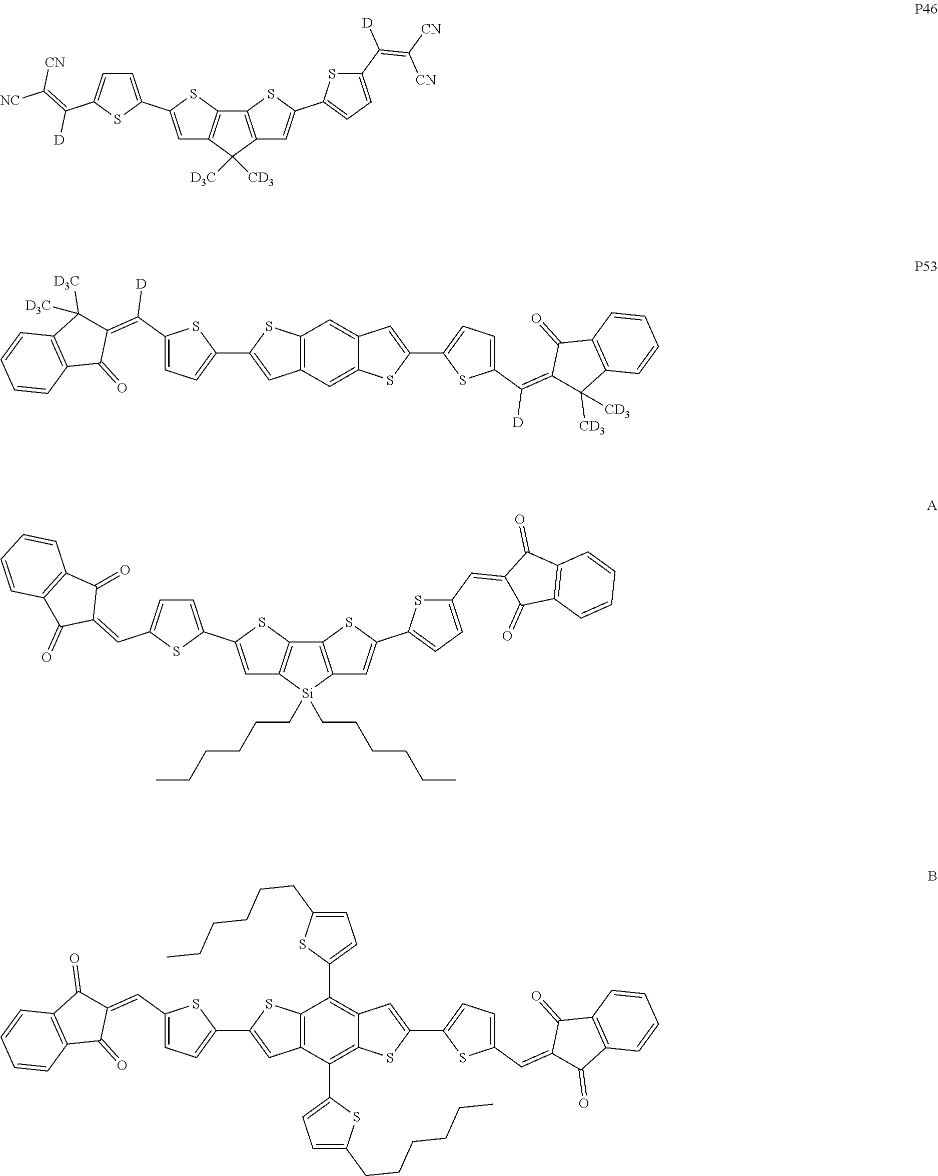

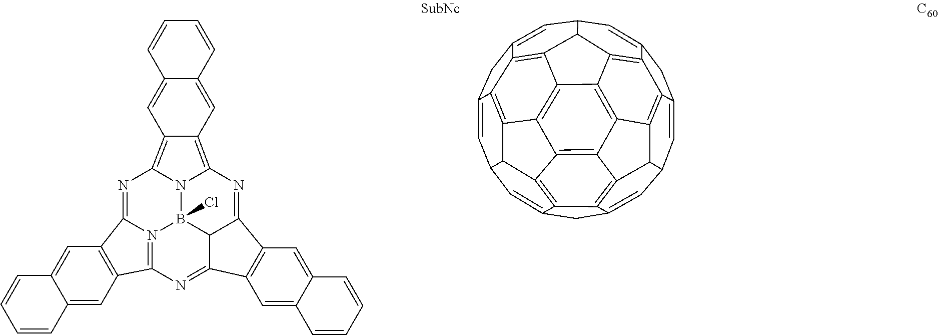

From Table 2, it can be seen that the opto-electronic devices according to Examples 1 to 16 have a maximum absorption wavelength at a wavelength corresponding to green light or red light, and compared to the opto-electronic devices according to Comparative Examples 1 to 4, high external quantum efficiency (EQE) and low dark current density (J_{dark}). As a result, it was confirmed that the opto-electronic devices according to Examples 1 to 16 may have higher photoelectric characteristics and lower noise than the opto-electronic devices according to Comparative Examples 1 to 4.

The organic compound represented by Formula 1 effectively absorbs green light or red light due to the inclusion of an electron-donor group, an electron-acceptor group, and a conjugation group of an appropriate length between the electron-donor group and the electron-acceptor group. The organic compound represented by Formula 1 can have excellent deposition stability and heat resistance due to the inclusion of a vinyl deuterium. Accordingly, an opto-electronic device using the organic compound can have excellent external quantum efficiency and dark current density.

Embodiments have been disclosed herein, and although terms are employed, they are used and are to be interpreted in a generic and descriptive sense only and not for purposes of limitation. In some instances, as would be apparent to one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. An organic compound represented by Formula 1:
[Formula 1] 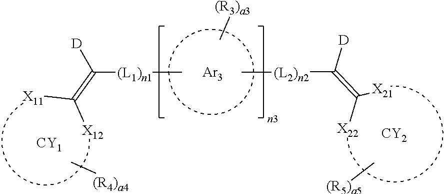
wherein in Formula 1,
Ar₃ is a C₁-C₆₀ heteroarylene group or a divalent non-aromatic condensed heteropolycyclic group,
n3 is an integer from 1 to 3,
L₁ is a C₃-C₆₀ carbocyclic group unsubstituted or substituted with at least one R₁ or a C₁-C₆₀ heterocyclic group unsubstituted or substituted with at least one R₁,
L₂ is a C₃-C₆₀ carbocyclic group unsubstituted or substituted with at least one R₂ or a C₁-C₆₀ heterocyclic group unsubstituted or substituted with at least one R₂,
n1 and n2 are each independently an integer from 1 to 3,
CY₁ and CY₂ are each independently a C₃-C₆₀ carbocyclic group or a C₁-C₆a heterocyclic group,
X₁₁ and X₁₂ are each independently C(R₄₁)(R₄₂), Si(R₄₁)(R₄₂), N(R₄₁), P(R₄₁), O, S, C(R₄₁), Si(R₄₁), N, P, C(═O), C(═S), or C═C(R₄₃)(R₄₄), at least one of X₁₁ and X₁₂ is each independently C(═O), C(═S), or C═C(R₄₃) (R₄₄),
X₂₁ and X₂₂ are each independently C(R₅₁)(R₅₂), Si(R₅₁)(R₅₂), N(R₅₁), P(R₅₁), O, S, C(R₅₁), Si(R₅₁), N, P, C(═O), C(═S), or C═C(R₅₃)(R₅₄),
at least one of X₂₁ and X₂₂ is each independently C(═O), C(═S), or C═C(R₅₃)(R₅₄),
a3, a4, and a5 are each independently an integer from 0 to 10,
R₁ to R₅, R₄₁, R₄₂, R₅₁, and R₅₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₄₃, R₄₄, R₅₃, and R₅₄ are each independently an electron-accepting group,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof.

2. The organic compound of claim 1, wherein the organic compound is represented by Formula 1-1: 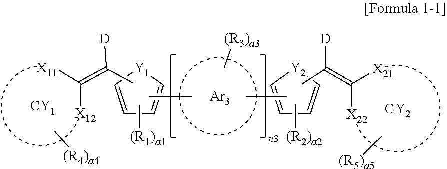
wherein in Formula 1-1,
Y₁ and Y₂ are each independently C(R₆)(R₇), Si(R₆)(R₇), N(R₆), P(R₆), 0, or S,
a1 and a2 are each independently an integer from 0 to 2,
R₆ and R₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
Ar₃, n3, CY₁, CY₂, X₁₁, X₁₂, X₂₁, X₂₂, a3, a4, a5, R₁ to R₅, Q₁ to Q₃, and R₁₀ₐ are each the same as defined in Formula 1.

3. The organic compound of claim 1, wherein the organic compound is represented by Formula 1-2: 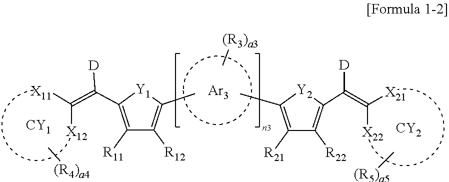
wherein in Formula 1-2,
Y₁ and Y₂ are each independently C(R₆)(R₇), Si(R₆)(R₇), N(R₆), P(R₆), O, or S,
R₆ and R₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₁ and R₁₂ are each independently the same as described in connection with R₁ in Formula 1,
R₂₁ and R₂₂ are each independently the same as described in connection with R₂ in Formula 1, and
Ar₃, n3, CY₁, CY₂, X₁₁, X₁₂, X₂₁, X₂₂, a3, a4, a5, R₃ to R₅, Q₁ to Q₃, and R₁₀ₐ are each the same as defined in Formula 1.

4. The organic compound of claim 1, wherein R₄₃, R₄₄, R₅₃, and R₅₄ are each independently —F, —Cl, —Br, —I, a cyano group, a nitro group, —C(═O)(Q₁), —S(═O)₂(Q₁), or —P(═O)(Q₁)(Q₂).

5. The organic compound of claim 1, wherein in Formula 1, a moiety represented by 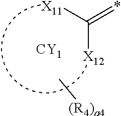
is a moiety represented by one of Formulae 1A to 1K: 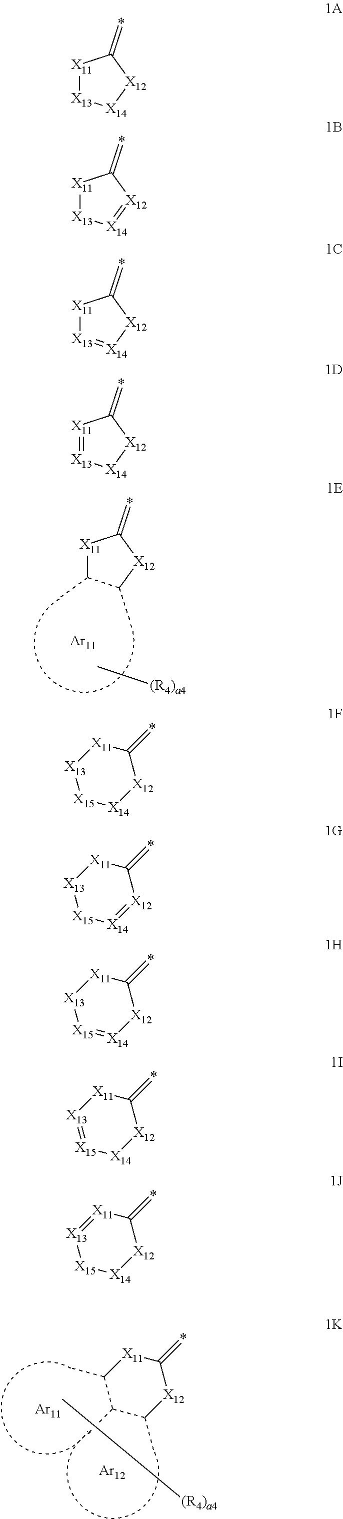
wherein in Formulae 1A to 1K,
Ar₁₁ and Ar₁₂ are each independently a C₃-C₃₀ carbocyclic group or a C₁-C₃₀ heterocyclic group,
X₁₃, X₁₄, and X₁₅ are each independently C(R₄₅)(R₄₆), Si(R₄₅)(R₄₆), N(R₄₅), P(R₄₅), O, S, C(R₄₅), Si(R₄₅), N, P, C(═O), C(═S), or C═C(R₄₃)(R₄₄),
X₁₁, X₁₂, R₄, a4, R₄₃, and R₄₄ are each the same as defined in Formula 1,
R₄₅ and R₄₆ are each independently the same as defined in connection with R₄ in Formula 1, and
indicates a binding site to a neighboring atom.

6. The organic compound of claim 1, wherein in Formula 1, a moiety represented by 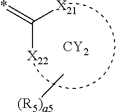
is a moiety represented by one of Formulae 2A to 2K: 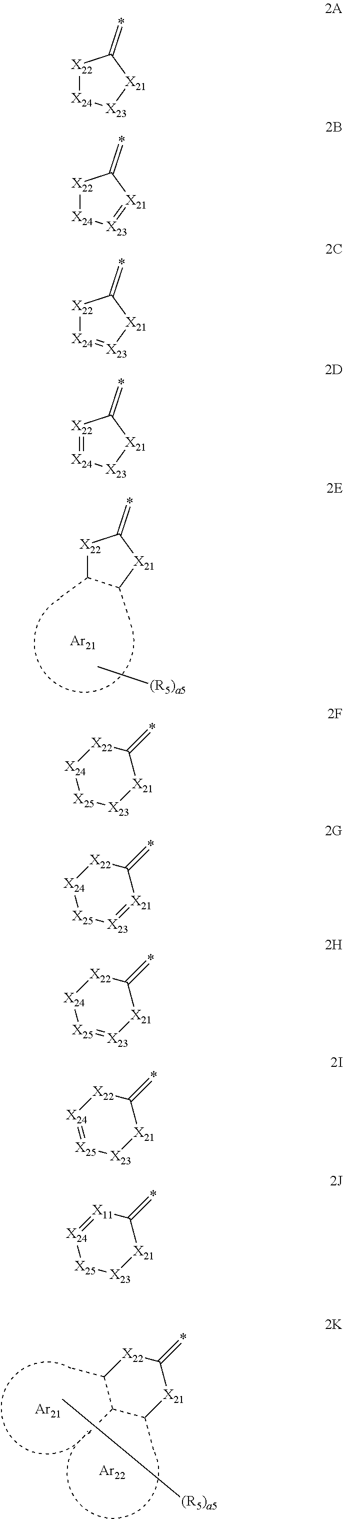
wherein in Formulae 2A to 2K,
Ar₂₁ and Ar₂₂ are each independently a C₃-C₃₀ carbocyclic group or a C₁-C₃₀ heterocyclic group,
X₂₃, X₂₄, and X₂₅ are each independently C(R₅₅)(R₅₆), Si(R₅₅)(R₅₆), N(R₅₅), P(R₅₅), O, S, C(R₅₅), Si(R₅₅), N, P, C(═O), C(═S), or C═C(R₅₃)(R₅₄),
X₂₁, X₂₂, R₅, a5, R₅₃, and R₅₄ are each the same as described in Formula 1,
R₅₅ and R₅₆ are each independently the same as defined in connection with R₅ in Formula 1, and
indicates a binding site to a neighboring atom.

7. The organic compound of claim 2, wherein Y₁ and Y₂ are each independently N(R₆), P(R₆), O, or S.

8. The organic compound of claim 1, wherein in Formula 1, a moiety represented by 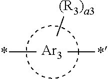
is a moiety represented by one of Formulae 3A to 3D: 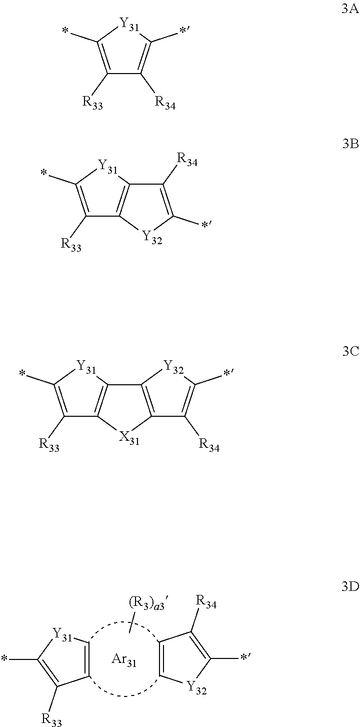
wherein in Formulae 3A to 3D,
Ar₃₁ is a C₃-C₃₀ carbocyclic group or C₁-C₃₀ heterocyclic group,
Y₃₁ and Y₃₂ are each independently N(R₃₁), P(R₃₁), O, or S,
X₃₁ is C(R₃₁)(R₃₂), Si(R₃₁)(R₃₂), N(R₃₁), P(R₃₁), O or S,
a3′ is an integer from 0 to 8,
R₃ is the same as defined in Formula 1,
R₃₁ to R₃₄ are each independently the same as defined in connection with R₃ in Formula 1, and
and *′ each indicate a binding site to a neighboring atom.

9. The organic compound of claim 1, wherein
R₁ to R₅, R₄₁, R₄₂, R₅₁, and R₅₂ are each independently hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkenyl group unsubstituted or substituted with at least one R₁₀ₐ, a C₁-C₂₀ alkynyl group unsubstituted or substituted with at least one R₁₀ₐ, a C₃-C₃₀ carbocyclic group unsubstituted or substituted with at least one R₁₀ₐ, a C₁-C₃₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
Q₁ to Q₃ and R₁₀ₐ are each the same as defined in Formula 1.

10. The organic compound of claim 1, wherein in Formula 1, a moiety represented by 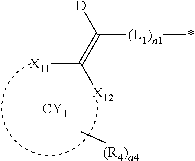
and a moiety represented by 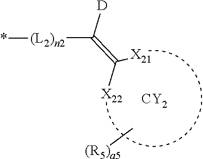
are identical to each other.

11. The organic compound of claim 1, wherein the organic compound is one of Compounds P1 to P55:

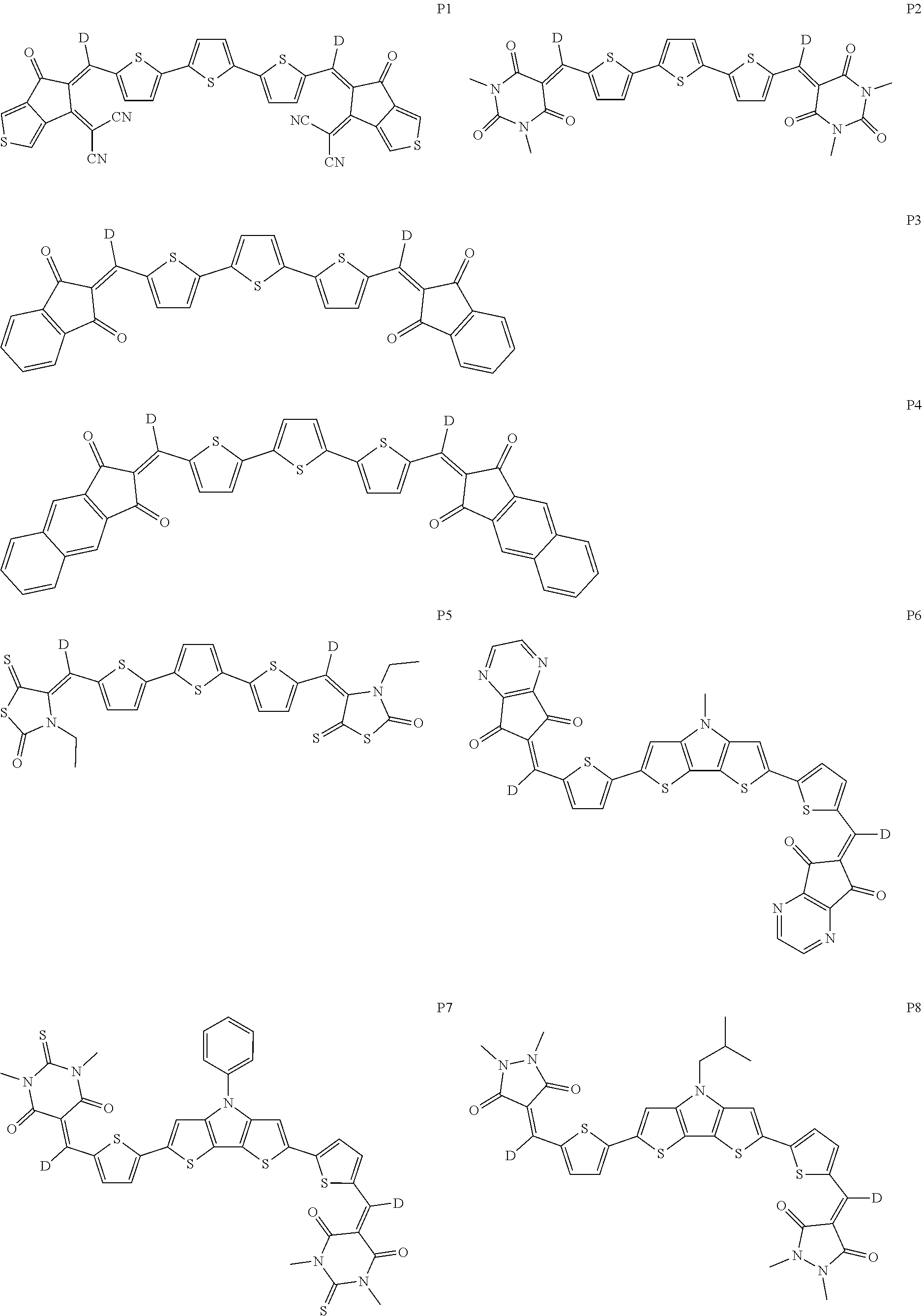

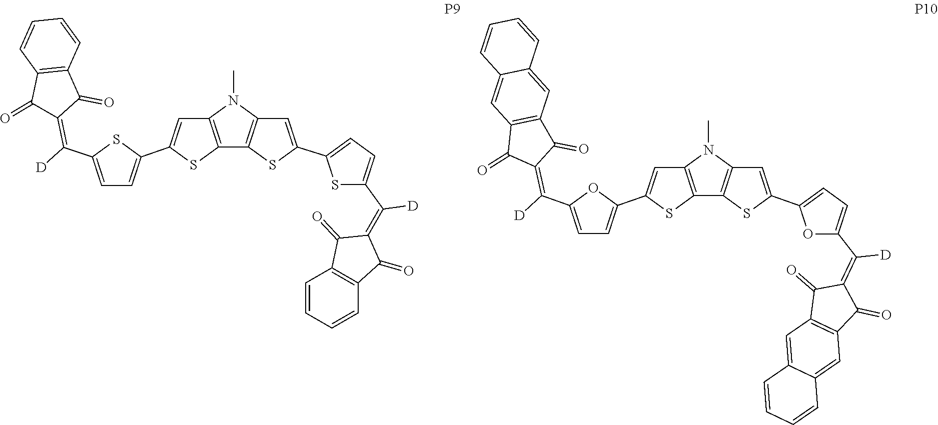 [SMILES: [2H]C(=C1C(=O)c2ccccc2C1=O)c1ccc(-c2cc3c(s2)c2sc(-c4ccc(C([2H])=C5C(=O)c6ccccc6C5=O)s4)cc2n3C)s1 ; [2H]C(=C1C(=O)c2cc3ccccc3cc2C1=O)c1ccc(-c2cc3c(s2)c2sc(-c4ccc(C([2H])=C5C(=O)c6cc7ccccc7cc6C5=O)o4)cc2n3C)o1]

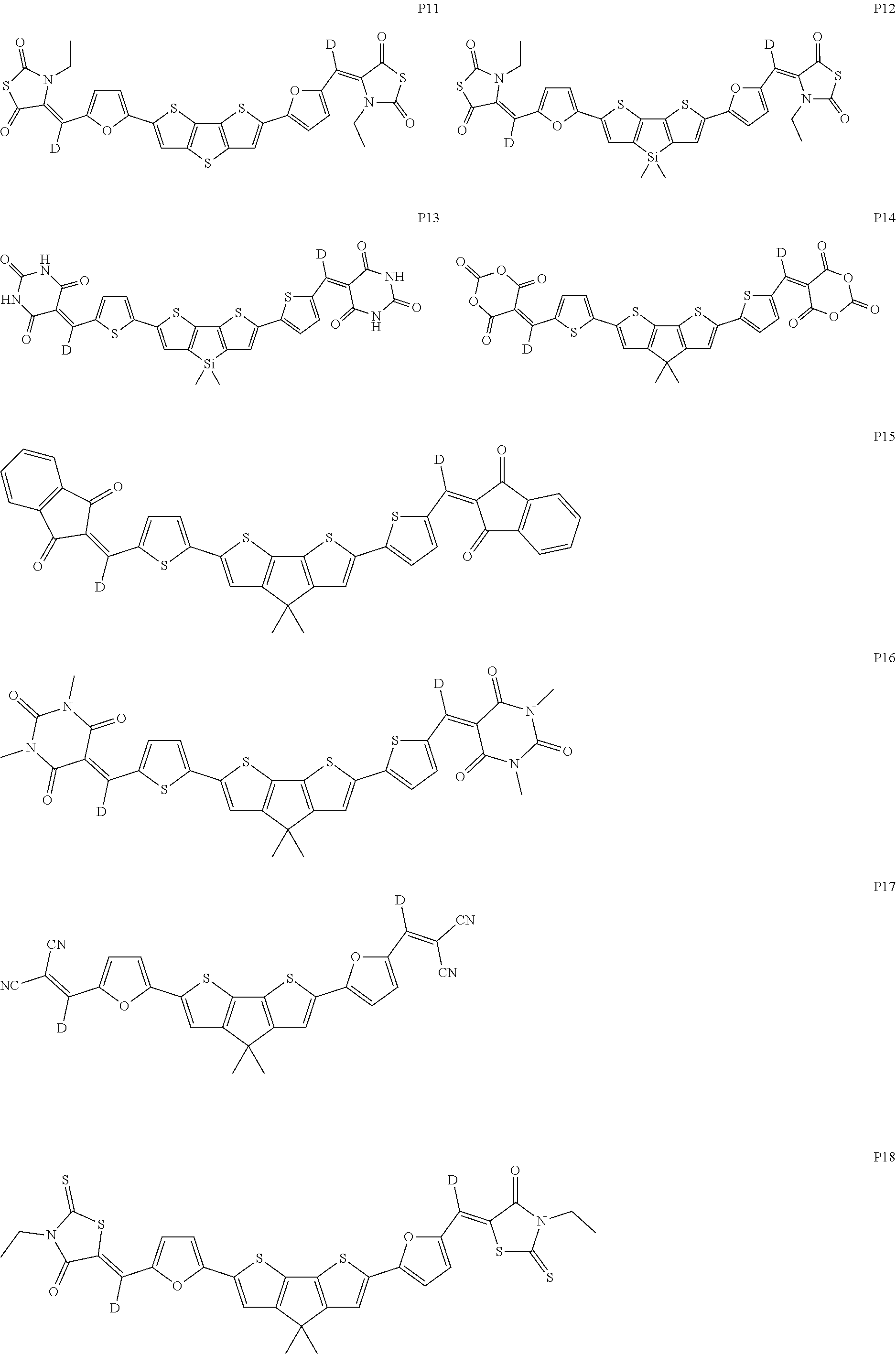

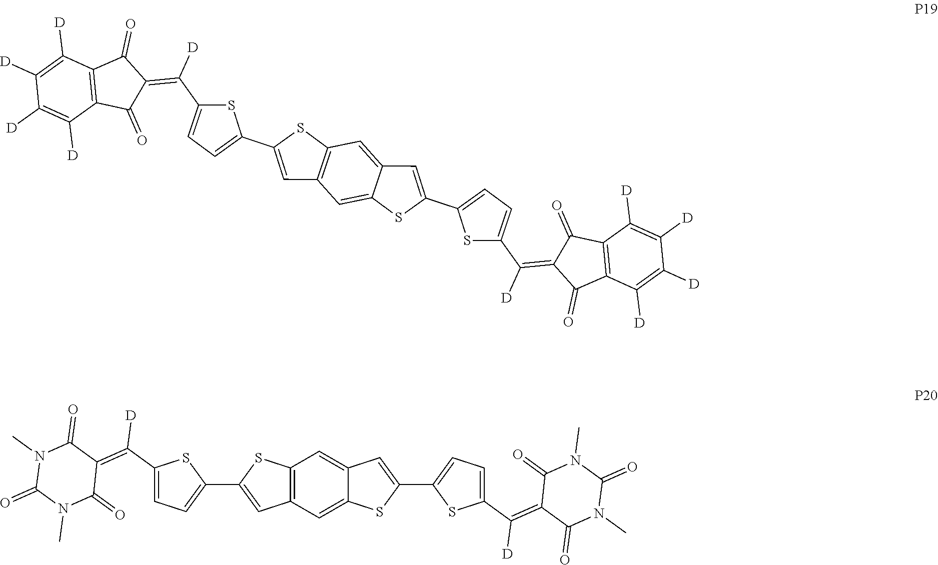 [SMILES: [2H]C(=C1C(=O)c2c([2H])c([2H])c([2H])c([2H])c2C1=O)c1ccc(-c2cc3cc4sc(-c5ccc(C([2H])=C6C(=O)c7c([2H])c([2H])c([2H])c([2H])c7C6=O)s5)cc4cc3s2)s1 ; [2H]C(=C1C(=O)N(C)C(=O)N(C)C1=O)c1ccc(-c2cc3cc4sc(-c5ccc(C([2H])=C6C(=O)N(C)C(=O)N(C)C6=O)s5)cc4cc3s2)s1]

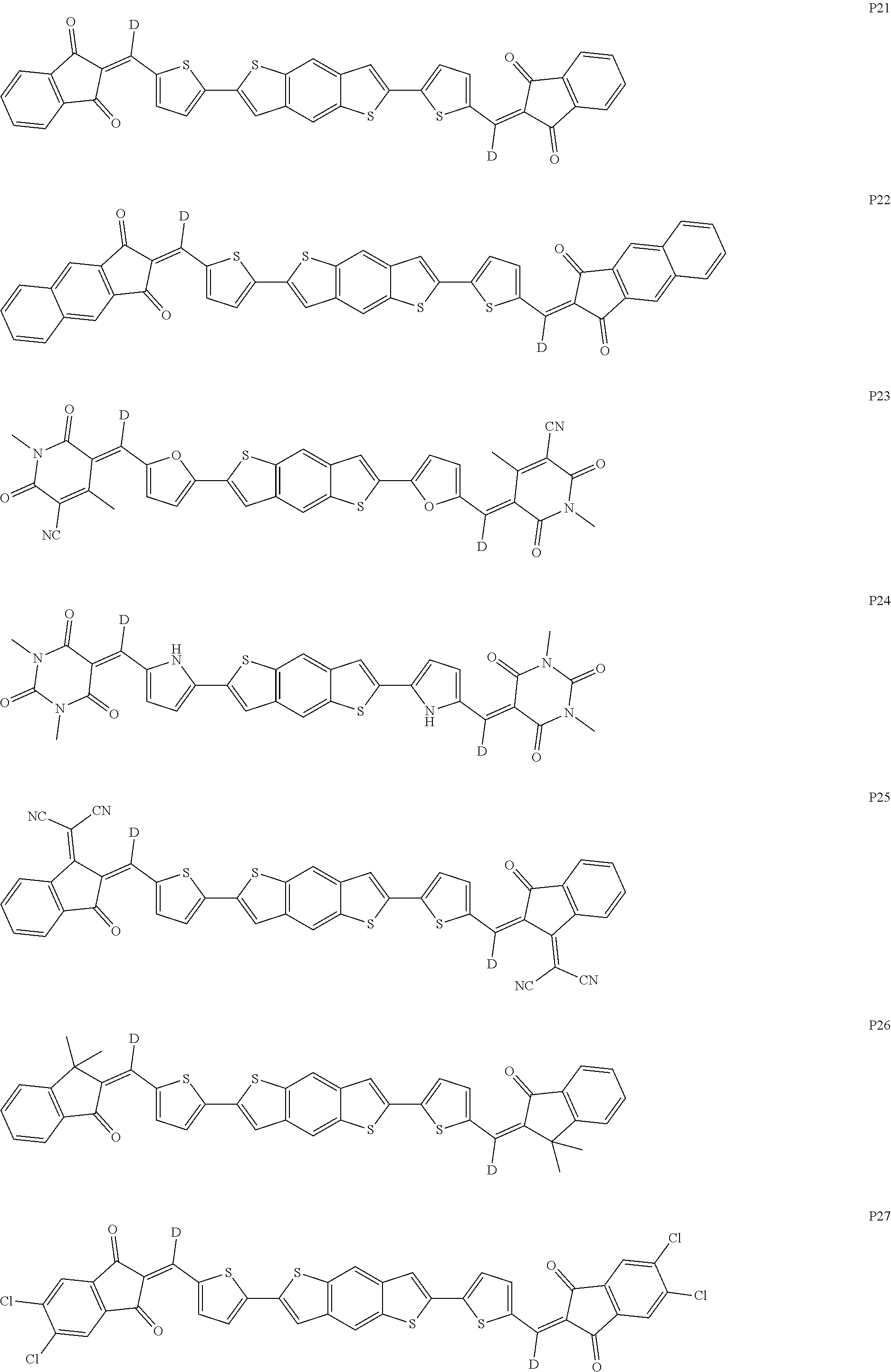

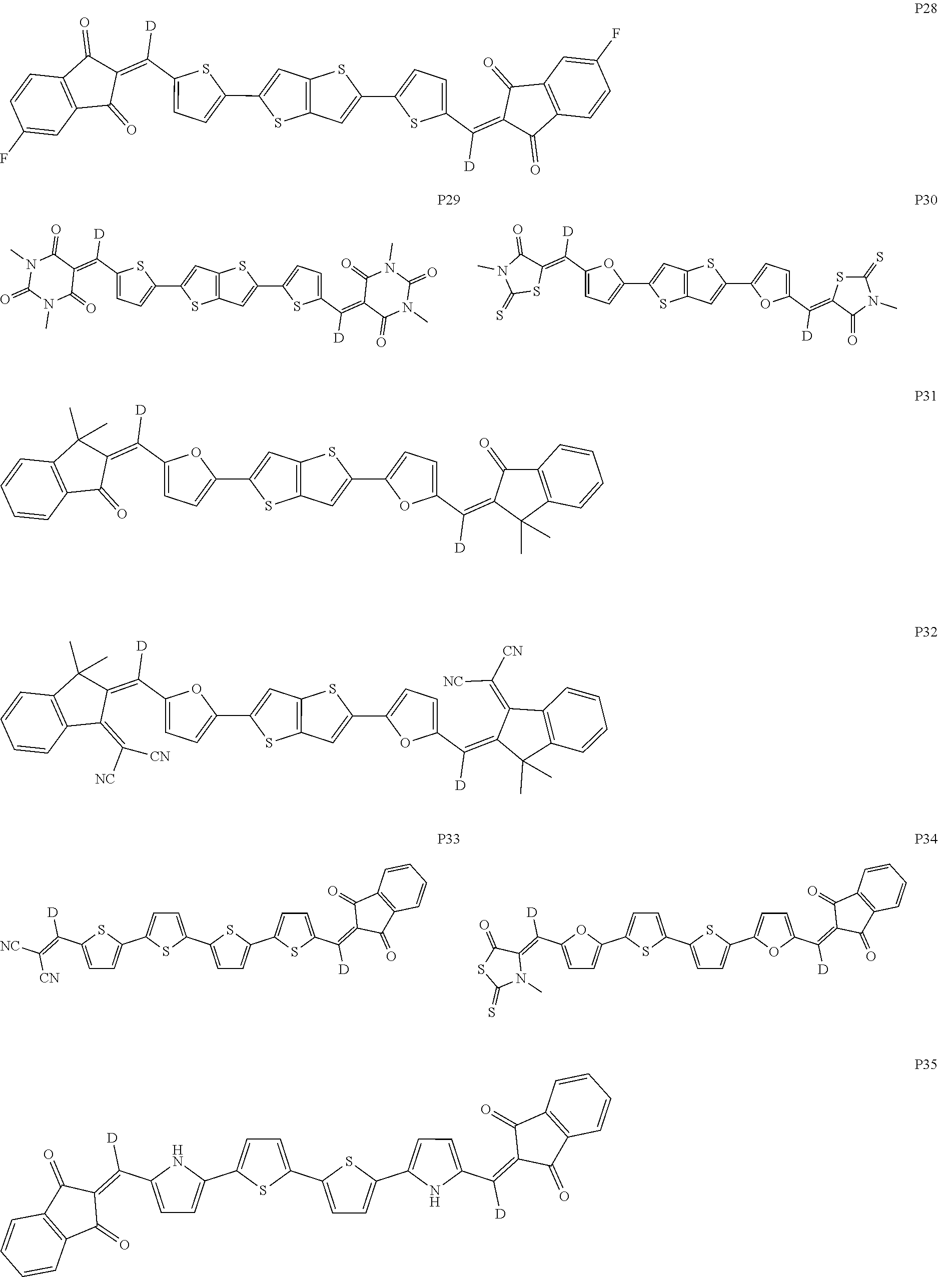

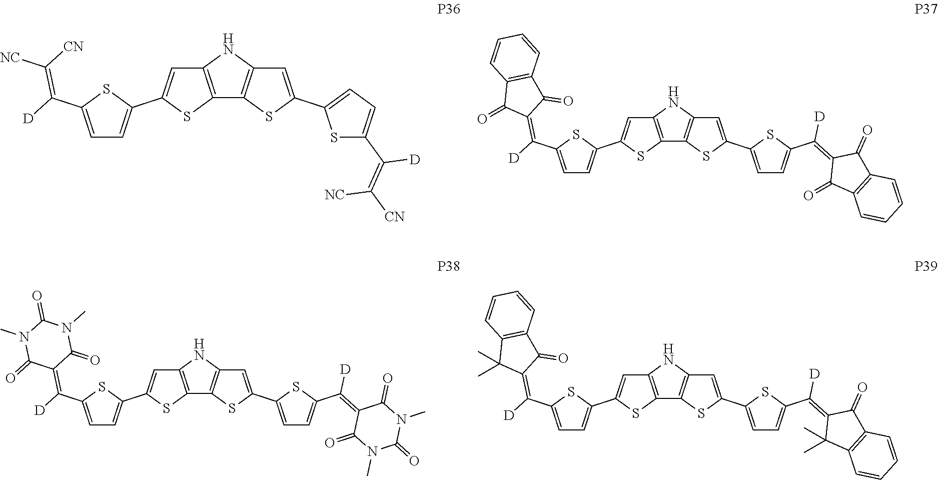

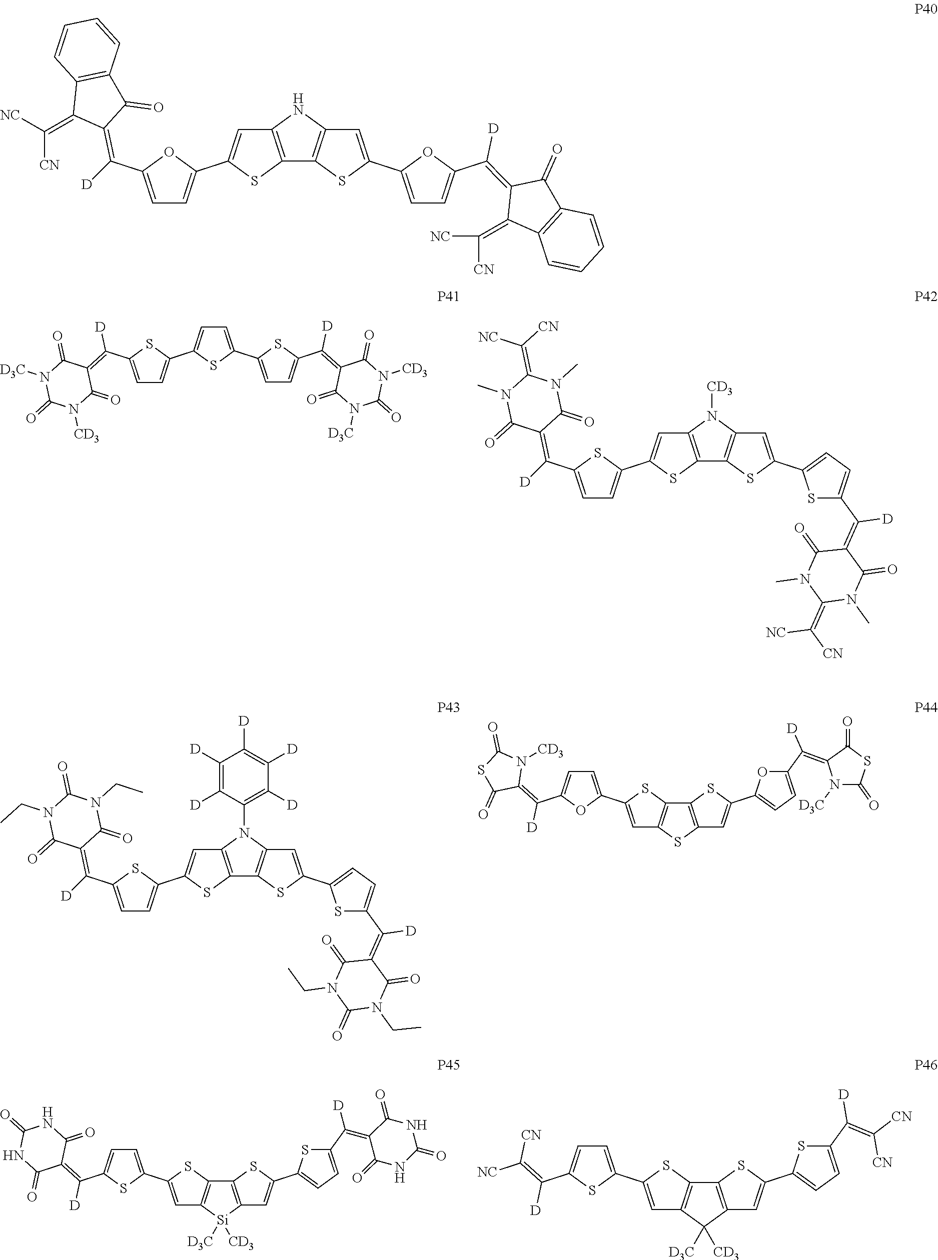

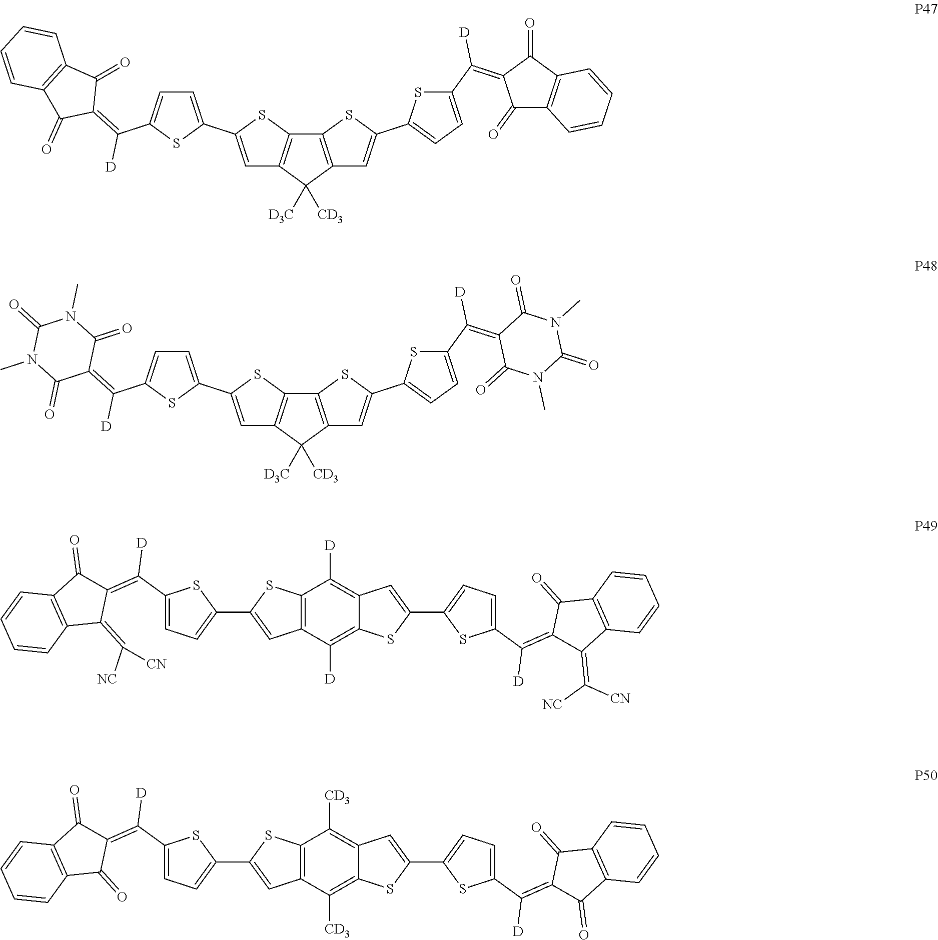

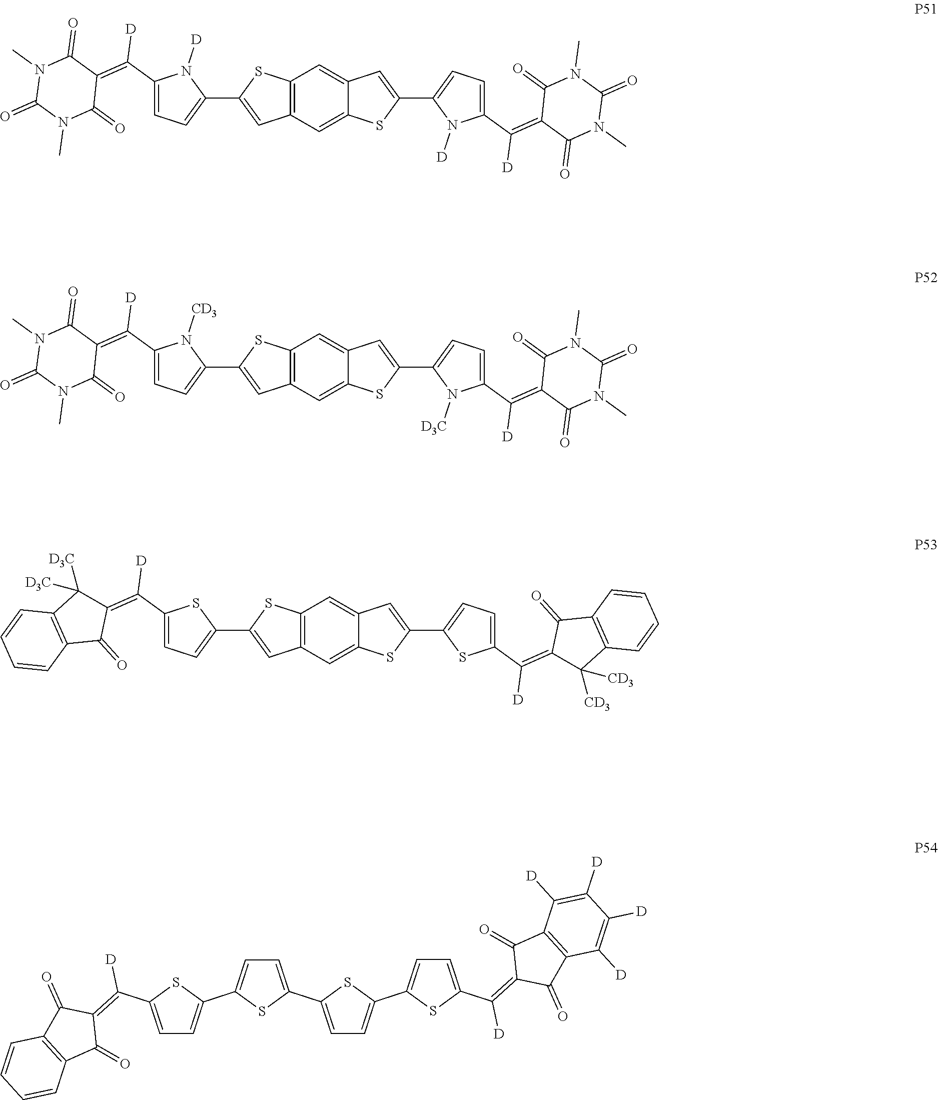

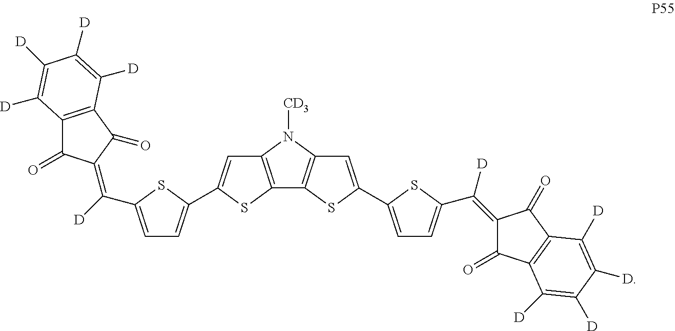

12. An opto-electronic device comprising:
a first electrode;
a second electrode facing the first electrode;
a photoactive layer between the first electrode and the second electrode; and
the organic compound of claim 1, wherein
the organic compound is a first compound.

13. The opto-electronic device of claim 12, further comprising:
a second compound represented by one of Formulae 2-1 to 2-6: 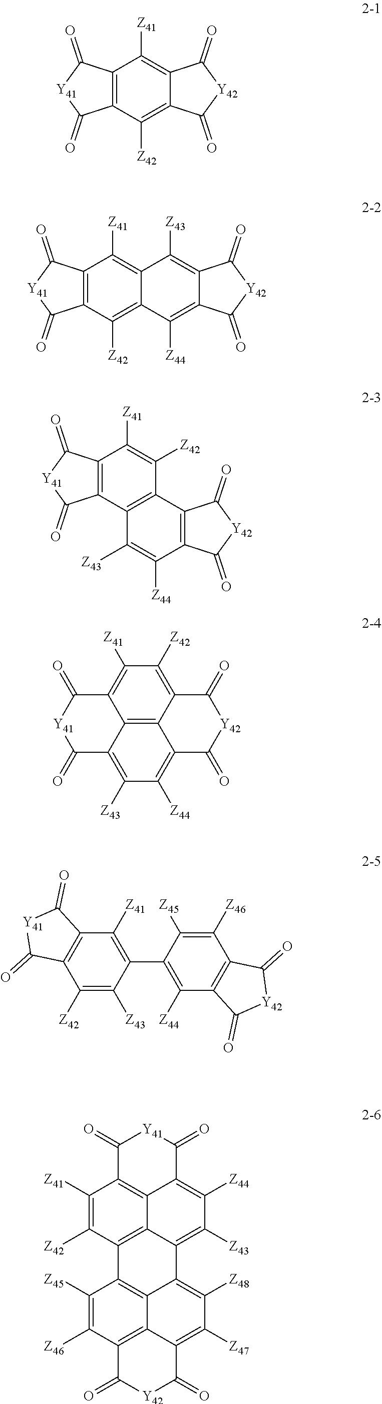
wherein in Formulae 2-1 to 2-6,
Y₄₁ and Y₄₂ are each independently C(Z₅₁)(Z₅₂), Si(Z₅₁)(Z₅₂), N(Z₅₁), P(Z₅₁), O, S, C(═O), C(═S), or C═C(Z₅₁)(Z₅₂),
Z₄₁ to Z₄₈, Z₅₁, and Z₅₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —CI, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof.

14. The opto-electronic device of claim 13, wherein Z₅₁ and Z₅₂ are each independently a C₁-C₆₀ alkyl group, a C₃-C₁₀ cycloalkyl group, a C₆-C₆₀ aryl group, or a C₁-C₆₀ heteroaryl group, each unsubstituted or substituted with deuterium, —F, —Cl, —Br, —I, a cyano group, a C₁-C₆₀ alkyl group, a C₁-C₆₀ alkoxy group, or a combination thereof.

15. The opto-electronic device of claim 13, wherein the second compound is one of Compounds N1 to N43:

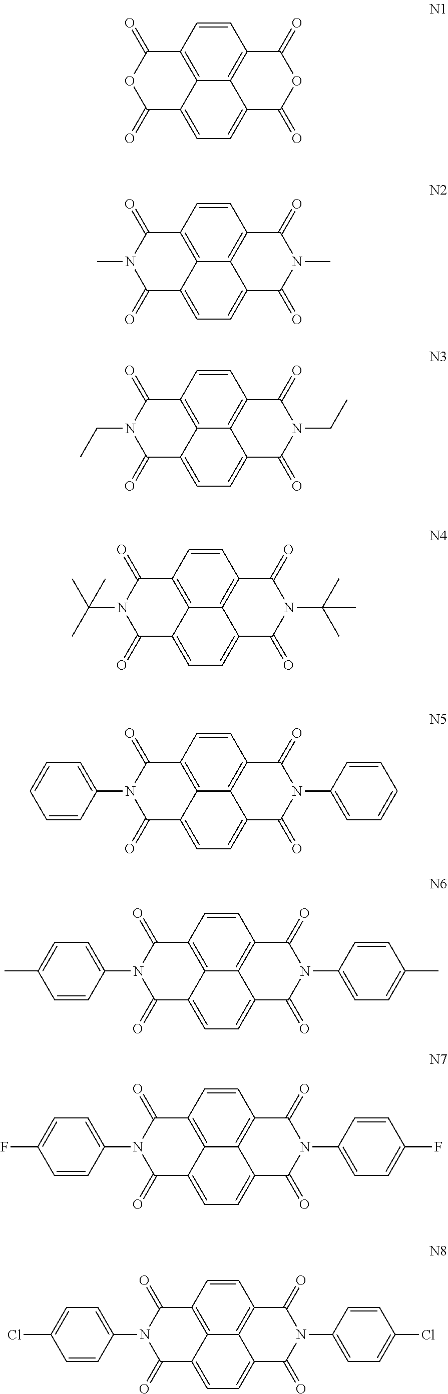

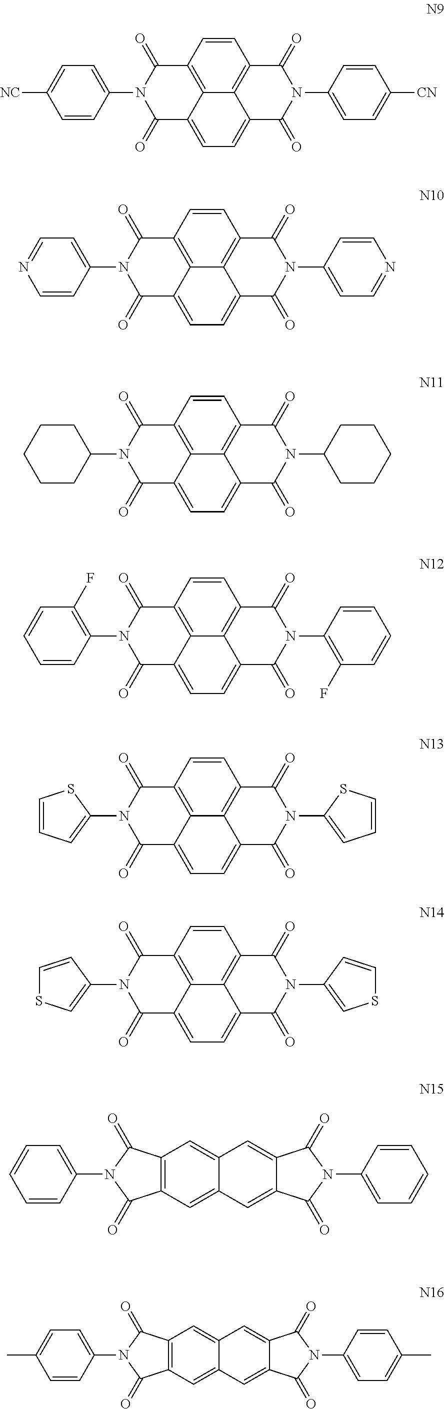

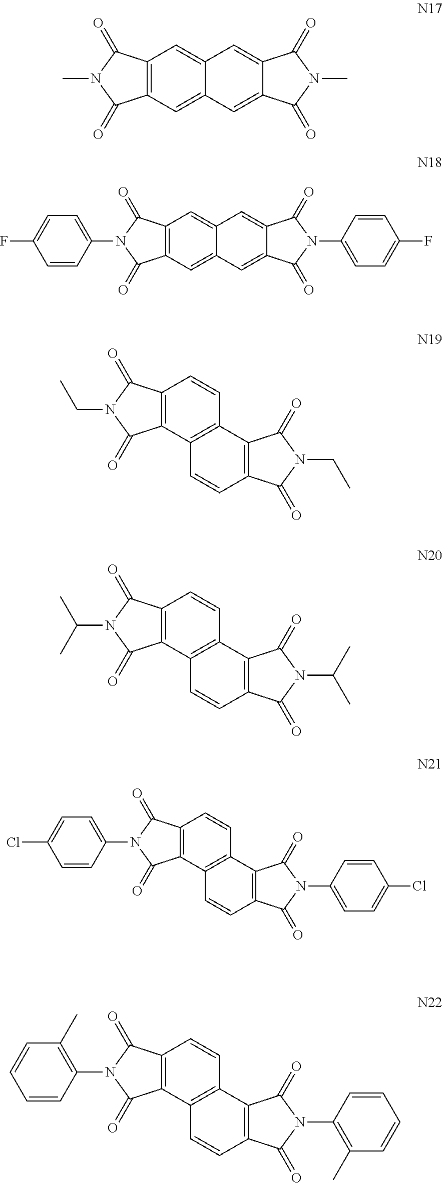

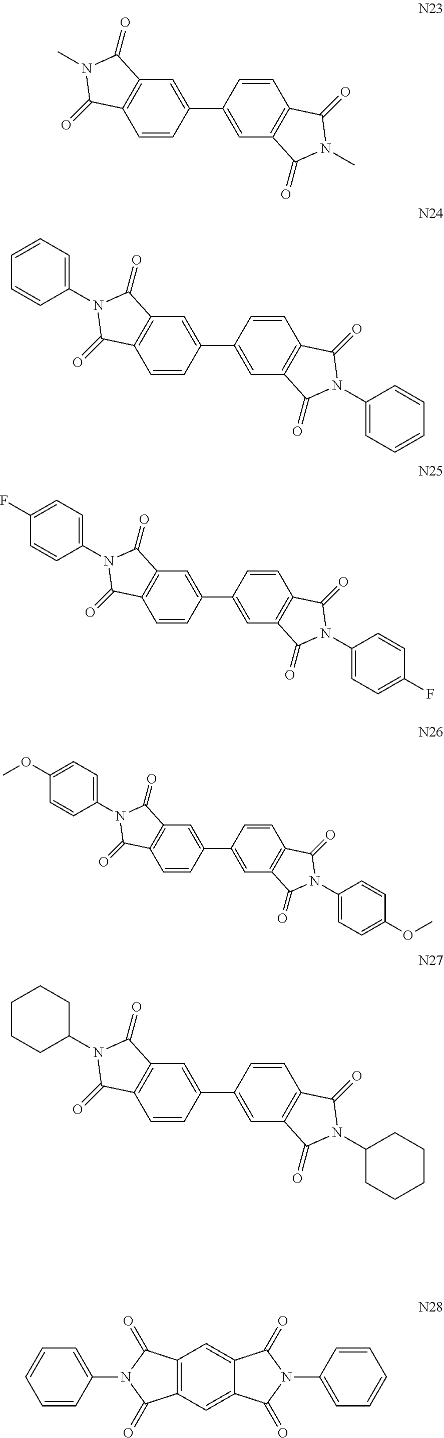

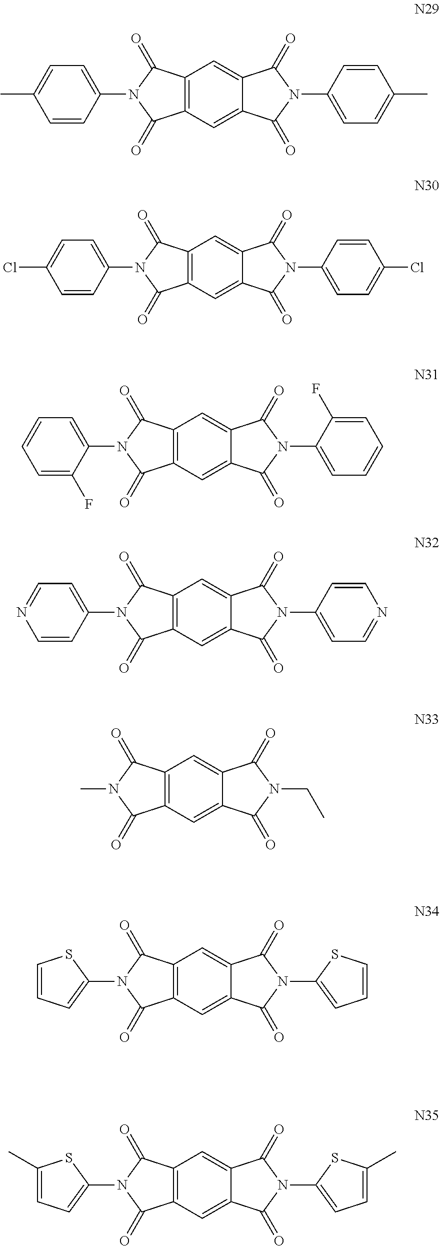

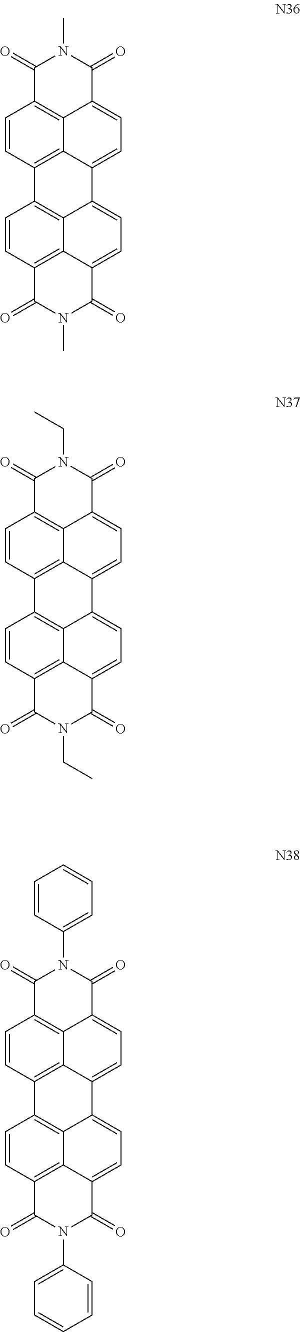 [SMILES: CN1C(=O)c2ccc3c4ccc5c6c(ccc(c7ccc(c2c37)C1=O)c64)C(=O)N(C)C5=O ; CCN1C(=O)c2ccc3c4ccc5c6c(ccc(c7ccc(c2c37)C1=O)c64)C(=O)N(CC)C5=O ; O=C1c2ccc3c4ccc5c6c(ccc(c7ccc(c2c37)C(=O)N1c1ccccc1)c64)C(=O)N(c1ccccc1)C5=O]

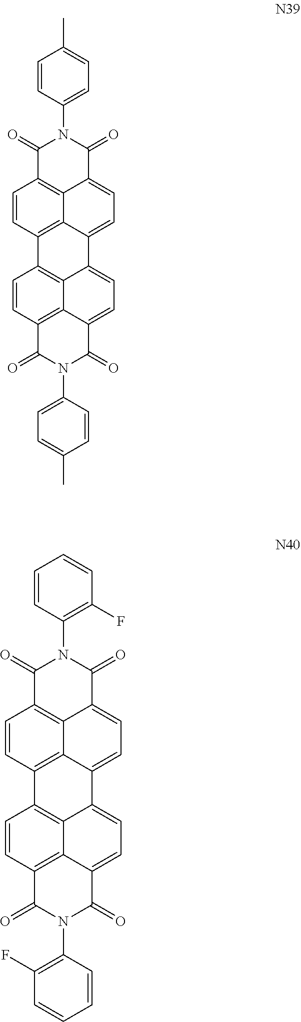 [SMILES: Cc1ccc(N2C(=O)c3ccc4c5ccc6c7c(ccc(c8ccc(c3c48)C2=O)c75)C(=O)N(c2ccc(C)cc2)C6=O)cc1 ; O=C1c2ccc3c4ccc5c6c(ccc(c7ccc(c2c37)C(=O)N1c1ccccc1F)c64)C(=O)N(c1ccccc1F)C5=O]

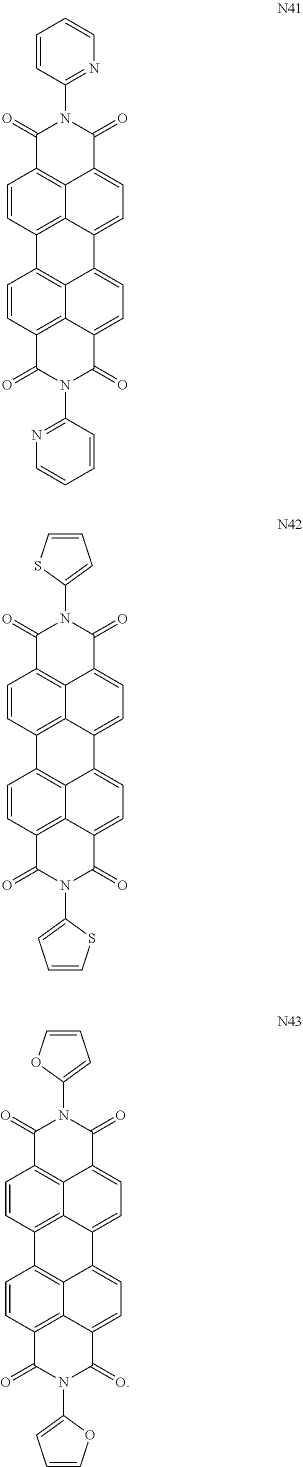 [SMILES: O=C1c2ccc3c4ccc5c6c(ccc(c7ccc(c2c37)C(=O)N1c1ccccn1)c64)C(=O)N(c1ccccn1)C5=O ; O=C1c2ccc3c4ccc5c6c(ccc(c7ccc(c2c37)C(=O)N1c1cccs1)c64)C(=O)N(c1cccs1)C5=O ; O=C1c2ccc3c4ccc5c6c(ccc(c7ccc(c2c37)C(=O)N1c1ccco1)c64)C(=O)N(c1ccco1)C5=O]

16. The opto-electronic device of claim 12, wherein the photoactive layer includes the first compound.

17. The opto-electronic device of claim 12, wherein the photoactive layer includes a first layer adjacent to the first electrode and a second layer adjacent to the second electrode.

18. The opto-electronic device of claim 17, wherein the first layer includes the first compound.

19. An electronic device comprising:
the opto-electronic device of claim 12;
a light-emitting device; and
a color filter, a color conversion layer, a touch screen layer, a polarizing layer, or a combination thereof, wherein
the light-emitting device and the opto-electronic device do not overlap each other.

20. An electronic apparatus comprising the opto-electronic device of claim 12, wherein
the electronic apparatus is a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a mobile phone, a tablet computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall including multiple displays tiled together, a theater screen, a stadium screen, a phototherapy device, a signboard, an automotive sensor, a home sensor, or a solar cell.
